(19) United States
(12) Patent Application Publication
(10) Pub. No.: US 2021/0151683 A1
(43) Pub. Date: May 20, 2021

(54) LIGHT-EMITTING ELEMENT, AND DISPLAY, ILLUMINATOR, AND SENSOR EACH INCLUDING SAME

(71) Applicant: Toray Industries, Inc., Tokyo (JP)

(72) Inventors: Hirotoshi Sakaino, Otsu-shi, Shiga (JP); Daisaku Tanaka, Otsu-shi, Shiga (JP); Takashi Tokuda, Otsu-shi, Shiga (JP)

(73) Assignee: Toray Industries, Inc., Tokyo (JP)

(21) Appl. No.: 16/622,464

(22) PCT Filed: Jul. 4, 2018
(86) PCT No.: PCT/JP2018/025323

§ 371 Date: Dec. 13, 2019

(30) Foreign Application Priority Data
Jul. 10, 2017 (JP) 2017-134356
Apr. 17, 2018 (JP) 2018-078908

(51) Int. Cl. H01L 51/00; C07D 413/14; C09K 11/06; C07D 487/04; C07D 403/14; C07D 403/04; C07F 5/02

(52) U.S. Cl. CPC H01L 51/008; C07D 413/14; C09K 11/06; H01L 51/0067; H01L 51/0071; H01L 51/5012; H01L 51/0072; C07D 403/14; C07D 403/04; C07F 5/022; C07D 487/04

(57) ABSTRACT

An object of the present invention is to provide an organic thin-film light-emitting element which achieves both a high luminous efficiency and light emission having high color purity. The present invention is a light-emitting element including: an anode; a cathode; and a plurality of organic layers including an emissive layer between the anode and the cathode, and emitting light by means of electrical energy. The emissive layer contains a compound represented by general formula (1) and a delayed fluorescent compound:

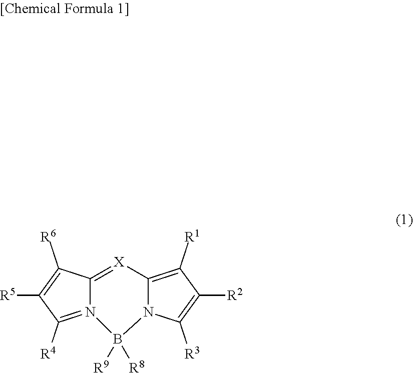

wherein X represents C—R⁷ or N; R¹ to R⁹ are the same or different from each other, and each are selected from a hydrogen atom, an alkyl group, a cycloalkyl group, a heterocyclic group, an alkenyl group, a cycloalkenyl group, an alkynyl group, a hydroxyl group, a thiol group, an alkoxy group, an alkylthio group, an aryl ether group, an aryl thioether group, an aryl group, a heteroaryl group, a halogen, a cyano group, an aldehyde group, a carbonyl group, a carboxyl group, an ester group, a carbamoyl group, an amino group, a nitro group, a silyl group, a siloxanyl group, a boryl group, —P(═0)R¹⁰R¹¹, and a fused ring and an aliphatic ring formed with an adjacent substituent; and R¹⁰ and R¹¹ each are an aryl group or a heteroaryl group.

### CROSS REFERENCE TO RELATED APPLICATIONS

This is the U.S. National Phase application of PCT/JP2018/025323, filed Jul. 4, 2018, which claims priority to Japanese Patent Application No. 2017-134356, filed Jul. 10, 2017 and Japanese Patent Application No. 2018-078908, filed Apr. 17, 2018, the disclosures of these applications being incorporated herein by reference in their entireties for all purposes.

### FIELD OF THE INVENTION

The present invention relates to a light-emitting element, and a display, an illuminator, and a sensor each including the same.

### BACKGROUND OF THE INVENTION

In an organic thin-film light-emitting element, electrons injected from a cathode and holes injected from an anode emit light when they are recombined in an emissive material in an organic layer sandwiched between both the electrodes. This light-emitting element is characteristic for high luminance light emission in the form of a thin type and under a low driving voltage, and multicolor light emission due to selection of an emissive material, and has been paid attention.

Electrons and holes are recombined to form excitons. At this time, it is known that singlet excitons and triplet excitons are generated at a ratio of 25%:75%. Therefore, in a fluorescent organic thin-film light-emitting element which uses light emission provided by singlet excitons, the theoretical limit of an internal quantum efficiency thereof is considered to be 25%. Meanwhile, in a phosphorescent organic thin-film light-emitting element which uses light emission provided by triplet excitons, the theoretical limit of an internal quantum efficiency thereof is considered to be 75%. The fluorescent organic thin-film light-emitting element has disadvantageously had a low luminous efficiency based on this light emission principle.

In order to solve this problem, in recent years, a fluorescent organic thin-film light-emitting element utilizing delayed fluorescence has been proposed. Among these, fluorescent organic thin-film light-emitting elements utilizing a TADF (Thermally Activated Delayed Fluorescence) phenomenon have been proposed and developed (see Non-Patent Documents 1 and 2, and Patent Documents 1 and 2, for example). This TADF phenomenon is a phenomenon in which reverse intersystem crossing from triplet excitons to singlet excitons occurs when a material having a small energy difference (AST) between the singlet level and the triplet level is used. When this TADF phenomenon is utilized, 75% of triplet excitons among excitons generated by the recombination of electrons and holes can be converted into singlet excitons, and the singlet excitons can be utilized. Therefore, also in the fluorescent organic thin-film light-emitting element, the internal quantum efficiency can theoretically be improved to 100%.

### Patent Documents

Patent Document 1: Japanese Patent Laid-open Publication No. 2014-045179

Patent Document 2: Japanese Patent Laid-open Publication No. 2014-022666

### Non-Patent Documents

Non-Patent Document 1: Nature Communications, 492, 234, 2012.

Non-Patent Document 2: Nature Communications, 5, 4016, 2014.

### SUMMARY OF THE INVENTION

Non-Patent Document 1 discloses a fluorescent organic thin-film light-emitting element using a TADF material as a dopant material of an emissive layer. By using the TADF dopant, a higher luminous efficiency than that of a conventional fluorescent organic thin-film light-emitting element is achieved. However, since the TADF dopant exhibits light emission having a large half-value width, problems remain in terms of color purity.

Non-Patent Document 2 discloses a fluorescent organic thin-film light-emitting element in which a TADF material is mixed in an emissive layer. In this case, triplet excitons are converted into singlet excitons by the TADF material, and a fluorescent dopant then receives the singlet excitons, thereby achieving a high luminous efficiency. However, problems still remain, such as the efficiency of delivery and receipt of the singlet excitons from the TADF material to the fluorescent dopant, and the color purity of light emission.

Similarly, Patent Document 1 discloses a fluorescent organic thin-film light-emitting element containing a TADF material and a fluorescent dopant in an emissive layer. In Patent Document 2, regarding an emissive layer containing a first host material having TADF properties, a second host material, and a fluorescent dopant material, a magnitude relationship among the singlet energies of these materials and a preferable relationship of the magnitude of an energy difference are disclosed. However, even in these examples, problems still remain in the efficiency of delivery and receipt of the singlet excitons from the TADF material to the fluorescent dopant, and the color purity of light emission.

Thus, the development of the highly efficient fluorescent organic thin-film light-emitting element has been advanced, but it has not been sufficient. Further, even if the luminous efficiency can be improved, the color purity as an advantage of the fluorescent organic thin-film light-emitting element has been deteriorated. Thus, there has not been yet found a technique which achieves both a high luminous efficiency and light emission having high color purity.

An object of the present invention is to provide an organic thin-film light-emitting element which solves the problems of the conventional technique and achieves both a high luminous efficiency and light emission having high color purity.

That is, the present invention is a light-emitting element including: an anode; a cathode; and a plurality of organic layers including an emissive layer between the anode and the cathode, and emitting light by means of electrical energy, wherein the emissive layer contains a compound represented by general formula (1) and a delayed fluorescent compound:

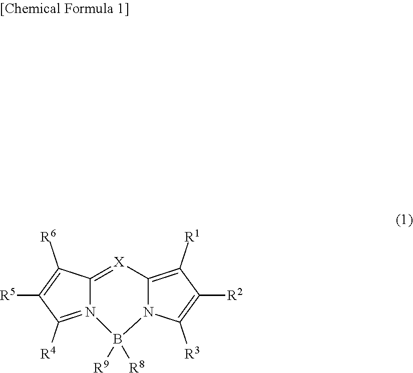

wherein X represents C—R⁷ or N; R¹ to R⁹ are the same or different from each other, and each are selected from a hydrogen atom, an alkyl group, a cycloalkyl group, a heterocyclic group, an alkenyl group, a cycloalkenyl group, an alkynyl group, a hydroxyl group, a thiol group, an alkoxy group, an alkylthio group, an aryl ether group, an aryl thioether group, an aryl group, a heteroaryl group, a halogen, a cyano group, an aldehyde group, a carbonyl group, a carboxyl group, an ester group, a carbamoyl group, an amino group, a nitro group, a silyl group, a siloxanyl group, a boryl group, —P(═O)R¹⁰R¹¹, and a fused ring and an aliphatic ring formed with an adjacent substituent; and R¹⁰ and R¹¹ each are an aryl group or a heteroaryl group.

The present invention can provide an organic thin-film light-emitting element which achieves both a high luminous efficiency and light emission having high color purity.

### DETAILED DESCRIPTION OF EMBODIMENTS OF THE INVENTION

Hereinafter, preferred embodiments of a light-emitting element according to the present invention, and a display, an illuminator, and a sensor including the light-emitting element will be described in detail. The present invention is not limited to the following embodiments, and can be variously modified and implemented according to purposes and applications.

A light-emitting element according to an embodiment of the present invention is a light-emitting element including: an anode; a cathode; and a plurality of organic layers including an emissive layer between the anode and the cathode, and emitting light by means of electrical energy, wherein the emissive layer contains a compound represented by general formula (1) described below and a delayed fluorescent compound:

<Compound Represented by General Formula (1)>

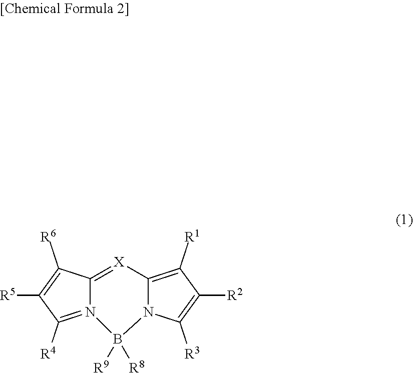

X represents C—R⁷ or N. R¹ to R⁹ are the same or different from each other, and each are selected from a hydrogen atom, an alkyl group, a cycloalkyl group, a heterocyclic group, an alkenyl group, a cycloalkenyl group, an alkynyl group, a hydroxyl group, a thiol group, an alkoxy group, an alkylthio group, an aryl ether group, an aryl thioether group, an aryl group, a heteroaryl group, a halogen, a cyano group, an aldehyde group, a carbonyl group, a carboxyl group, an ester group, a carbamoyl group, an amino group, a nitro group, a silyl group, a siloxanyl group, a boryl group, —P(═O)R¹⁰R¹¹, and a fused ring and an aliphatic ring formed with an adjacent substituent. R¹⁰ and R¹¹ each are an aryl group or a heteroaryl group.

In all the groups described above, hydrogen may be heavy hydrogen. The same applies to compounds or partial structures thereof described below.

In the following description, for example, a substituted or unsubstituted aryl group having 6 to 40 carbon atoms has 6 to 40 carbon atoms including carbon atoms contained in a substituent with which an aryl group is substituted. The same applies to other substituents which define the number of carbon atoms.

In all the above groups, as substituents when being substituted, an alkyl group, a cycloalkyl group, a heterocyclic group, an alkenyl group, a cycloalkenyl group, an alkynyl group, a hydroxyl group, a thiol group, an alkoxy group, an alkylthio group, an aryl ether group, an aryl thioether group, an aryl group, a heteroaryl group, a halogen, a cyano group, an aldehyde group, a carbonyl group, a carboxyl group, an ester group, a carbamoyl group, an amino group, a nitro group, a silyl group, a siloxanyl group, a boryl group, —P(═O)R¹⁰R¹¹ are preferable, and specific substituents mentioned as preferable substituents in the descriptions of the substituents are more preferable. R¹⁰ and R¹¹ each are an aryl group or a heteroaryl group. These substituents maybe further substituted with the substituents described above.

The term “unsubstituted” associated with the term “substituted or unsubstituted” means that a group is substituted with a hydrogen atom or a heavy hydrogen atom.

The same applies to the term “substituted or unsubstituted” for the compounds described below or substructures thereof.

The alkyl group represents a saturated aliphatic hydrocarbon group, such as a methyl group, an ethyl group, a n-propyl group, an isopropyl group, a n-butyl group, a sec-butyl group, or a tert-butyl group, and it may or may not have a substituent. When the alkyl group is substituted, the additional substituent is not particularly limited. Examples thereof include an alkyl group, a halogen, an aryl group, and a heteroaryl group, and the same holds true in the descriptions below. The number of carbon atoms in the alkyl group is not particularly limited, but from the viewpoints of easy availability and cost, it is preferably within the range of 1 or more and 20 or less, and more preferably 1 or more and 8 or less.

The cycloalkyl group represents a saturated alicyclic hydrocarbon group, such as a cyclopropyl group, a cyclohexyl group, a norbornyl group, and an adamantyl group, and this may or may not have a substituent. The number of carbon atoms in the alkyl group moiety is not particularly limited, but is preferably within the range of 3 or more and 20 or less.

The heterocyclic group represents an aliphatic ring having an atom other than carbon in the ring, such as a pyran ring, a piperidine ring, and a cyclic amide, and this may or may not have a substituent. The number of carbon atoms in the heterocyclic group is not particularly limited, but is preferably within the range of 2 or more and 20 or less.

The alkenyl group represents an unsaturated aliphatic hydrocarbon group containing a double bond, such as a vinyl group, an allyl group, and a butadienyl group, and this may or may not have a substituent. The number of carbon atoms in the alkenyl group is not particularly limited, but is preferably within the range of 2 or more and 20 or less.

The cycloalkenyl group represents an unsaturated alicyclic hydrocarbon group containing a double bond, such as a cyclopentenyl group, a cyclopentadienyl group, and a cyclohexenyl group, and this may or may not have a substituent.

The alkynyl group represents an unsaturated aliphatic hydrocarbon group containing a triple bond, such as an ethynyl group, and this may or may not have a substituent. The number of carbon atoms in the alkynyl group is not particularly limited, but is preferably within the range of 2 or more and 20 or less.

The alkoxy group represents a functional group with an aliphatic hydrocarbon group bonded via an ether bond, such as a methoxy group, an ethoxy group, and a propoxy group, and this aliphatic hydrocarbon group may or may not have a substituent. The number of carbon atoms in the alkoxy group is not particularly limited, but is preferably within the range of 1 or more and 20 or less.

The alkylthio group represents a group in which an oxygen atom of an ether bond in an alkoxy group is substituted with a sulfur atom. The hydrocarbon group of the alkylthio group may or may not have a substituent. The number of carbon atoms in the alkylthio group is not particularly limited, but is preferably within the range of 1 or more and 20 or less.

The aryl ether group represents a functional group with an aromatic hydrocarbon group bonded via an ether bond, such as a phenoxy group, and the aromatic hydrocarbon group may or may not have a substituent. The number of carbon atoms in the aryl ether group is not particularly limited, but is preferably within the range of 6 or more and 40 or less.

The aryl thioether group represents a group in which an oxygen atom of an ether bond in an aryl ether group is substituted with a sulfur atom. The aromatic hydrocarbon group in the aryl thioether group may or may not have a substituent. The number of carbon atoms in the aryl thioether group is not particularly limited, but is preferably within the range of 6 or more and 40 or less.

For example, the aryl group represents an aromatic hydrocarbon group such as a phenyl group, a biphenyl group, a terphenyl group, a naphthyl group, a fluorenyl group, a benzofluorenyl group, a dibenzofluorenyl group, a phenanthryl group, an anthracenyl group, a benzophenanthryl group, a benzoanthracenyl group, a chrysenyl group, a pyrenyl group, a fluoranthenyl group, a triphenylenyl group, a benzofluoranthenyl group, a dibenzoanthracenyl group, a perylenyl group, or a helicenyl group.

Among these, a phenyl group, a biphenyl group, a terphenyl group, a naphthyl group, a fluorenyl group, a phenanthryl group, an anthracenyl group, a pyrenyl group, a fluoranthenyl group, and a triphenylenyl group are preferable. The aryl group may or may not have a substituent. The number of carbon atoms in the aryl group is not particularly limited, but is preferably within the range of 6 or more and 40 or less, and more preferably within the range of 6 or more and 30 or less.

When R¹ to R⁹ each are a substituted or unsubstituted aryl group, the aryl group is preferably a phenyl group, a biphenyl group, a terphenyl group, a naphthyl group, a fluorenyl group, a phenanthryl group, or an anthracenyl group, more preferably a phenyl group, a biphenyl group, a terphenyl group, or a naphthyl group, still more preferably a phenyl group, a biphenyl group, or a terphenyl group, and particularly preferably a phenyl group.

When each substituent is further substituted with an aryl group, the aryl group is preferably a phenyl group, a biphenyl group, a terphenyl group, a naphthyl group, a fluorenyl group, a phenanthryl group, or an anthracenyl group, more preferably a phenyl group, a biphenyl group, a terphenyl group, or a naphthyl group, and particularly preferably a phenyl group.

The heteroaryl group represents a cyclic aromatic group having one or a plurality of atoms other than carbon in the ring, such as a pyridyl group, a furanyl group, a thienyl group, a quinolinyl group, an isoquinolinyl group, a pyrazinyl group, a pyrimidyl group, a pyridazinyl group, a triazinyl group, a naphthyridinyl group, a cinnolinyl group, a phthalazinyl group, a quinoxalinyl group, a quinazolinyl group, a benzofuranyl group, a benzothiophenyl group, an indolyl group, a dibenzofuranyl group, a dibenzothiophenyl group, a carbazolyl group, a benzocarbazolyl group, a carbonyl group, an indolocarbazolyl group, a benzofurocarbazolyl group, a benzothienocarbazolyl group, a dihydroindenocarbazolyl group, a benzoquinolinyl group, an acridinyl group, a dibenzoacridinyl group, a benzoimidazolyl group, an imidazopyridyl group, a benzoxazolyl group, a benzothiazolyl group or a phenanthrolinyl group. The naphthyridinyl group represents any one of a 1,5-naphthyridinyl group, a 1,6-naphthyridinyl group, a 1,7-naphthyridinyl group, a 1,8-naphthyridinyl group, a 2,6-naphthyridinyl group and a 2,7-naphthyridinyl group. The heteroaryl group may or may not have a substituent. The number of carbon atoms in the heteroaryl group is not particularly limited, but is preferably within the range of 2 or more and 40 or less, and more preferably within the range of 2 or more and 30 or less.

When R¹ to R⁹ each are a substituted or unsubstituted heteroaryl group, the heteroaryl group is preferably a pyridyl group, a furanyl group, a thienyl group, a quinolinyl group, a pyrimidyl group, a triazinyl group, a benzofuranyl group, a benzothienyl group, an indolyl group, a dibenzofuranyl group, a dibenzothienyl group, a carbazolyl group, a benzimidazolyl group, an imidazopyridyl group, a benzoxazolyl group, a benzothiazolyl group, or a phenanthrolinyl group, more preferably a pyridyl group, a furanyl group, a thienyl group, or a quinolinyl group, and particularly preferably a pyridyl group.

When each substituent is further substituted with a heteroaryl group, the heteroaryl group is preferably a pyridyl group, a furanyl group, a thienyl group, a quinolinyl group, a pyrimidyl group, a triazinyl group, a benzofuranyl group, a benzothienyl group, an indolyl group, a dibenzo furanyl group, a dibenzothienyl group, a carbazolyl group, a benzimidazolyl group, an imidazopyridyl group, a benzoxazolyl group, a benzothiazolyl group, or a phenanthrolinyl group, more preferably a pyridyl group, a furanyl group, a thienyl group, or a quinolinyl group, and particularly preferably a pyridyl group.

The electron-accepting nitrogen in the phrase “containing electron-accepting nitrogen” represents a nitrogen atom which forms a multiple bond with an adjacent atom. Examples of the aromatic heterocyclic ring containing electron-accepting nitrogen include a pyridine ring, a pyridazine ring, a pyrimidine ring, a pyrazine ring, a triazine ring, an oxadiazole ring, a thiazole ring, a quinoline ring, an isoquinoline ring, a naphthyridine ring, a cinnoline ring, a phthalazine ring, a quinazoline ring, a quinoxaline ring, a benzoquinoline ring, a phenanthroline ring, an acridine ring, a benzothiazole ring, and a benzoxazole ring. The naphthyridine represents any one of a 1,5-naphthyridine, 1,6-naphthyridine, 1,7-naphthyridine, 1,8-naphthyridine, 2,6-naphthyridine, and 2,7-naphthyridine.

The electron-donating nitrogen in the phrase “containing electron-donating nitrogen” represents a nitrogen atom which forms only a single bond with an adjacent atom. Examples of the aromatic heterocyclic ring containing electron-donating nitrogen include an aromatic heterocyclic ring having a pyrrole ring. Examples of the aromatic heterocyclic ring having a pyrrole ring include a pyrrole ring, an indole ring, and a carbazole ring.

The halogen represents an atom selected from fluorine, chlorine, bromine, and iodine.

The carbonyl group, the carboxyl group, the ester group, and the carbamoyl group may or may not have a substituent. Here, examples of the substituent include an alkyl group, a cycloalkyl group, an aryl group and a heteroaryl group, and these substituents may be further substituted.

The amino group is a substituted or unsubstituted amino group. Examples of the substituent include an aryl group, a heteroaryl group, a linear alkyl group, and a branched alkyl group. As the aryl group and the heteroaryl group, a phenyl group, a naphthyl group, a pyridyl group, and a quinolinyl group are preferable. These substituents maybe further substituted. The number of carbon atoms in the substituent moiety of the amino group is not particularly limited, but is preferably within the range of 2 or more and 50 or less, more preferably within the range of 6 or more and 40 or less, and particularly preferably within the range of 6 or more and 30 or less.

For example, the silyl group represents an alkylsilyl group such as a trimethylsilyl group, a triethylsilyl group, a tert-butyldimethylsilyl group, a propyldimethylsilyl group or a vinyldimethylsilyl group, or an arylsilyl group such as a phenyldimethylsilyl group, a tert-butyldiphenylsilyl group, a triphenylsilyl group or a trinaphthylsilyl group. The substituent on the silicon atom may be further substituted. The number of carbon atoms in the silyl group is not particularly limited, but is preferably within the range of 1 or more and 30 or less.

The siloxanyl group represents a silicon compound group via an ether bond such as a trimethylsiloxanyl group. The substituent on the silicon atom may be further substituted.

The boryl group is a substituted or unsubstituted boryl group. Examples of the substituent with which the boryl group is substituted include an aryl group, a heteroaryl group, a linear alkyl group, a branched alkyl group, an aryl ether group, an alkoxy group, and a hydroxy group. Among these, an aryl group and an aryl ether group are preferable.

In the phosphine oxide group —P(═O)R¹⁰R¹¹, R¹⁰ and R¹¹ each are an aryl group or a heteroaryl group. Specific examples thereof include, but are not particularly limited to, the following.

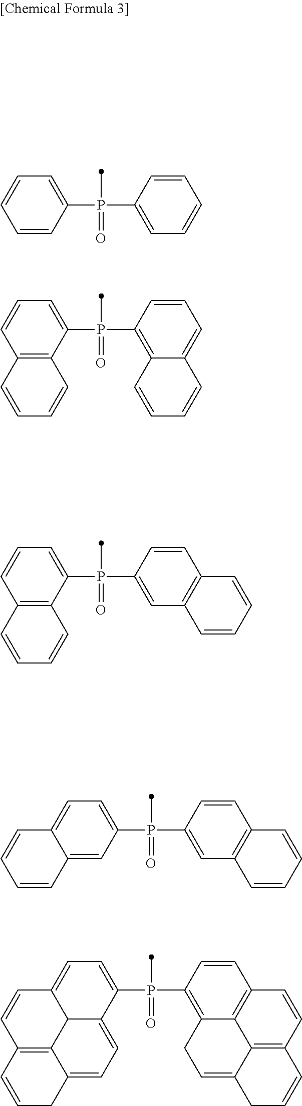

The fused ring and the aliphatic ring formed with an adjacent substituent refers to mutual bonding between any two adjacent substituents (R¹ and R² in general formula (1), for example) forming a conjugated or non-conjugated cyclic skeleton. As the constituent element of the fused ring and the aliphatic ring, an element selected from nitrogen, oxygen, sulfur, phosphorous, and silicon, besides carbon, may be contained. The fused ring and the aliphatic ring may be fused with another ring. The fused ring and the alicyclic ring may be further fused with another ring.

The compound represented by general formula (1) exhibits high fluorescence quantum yield, and has a small Stokes shift and a small peak half-value width of an emission spectrum, whereby the compound can be suitably used as a fluorescent dopant. The fluorescence spectrum exhibits a single peak in the range of 400 nm or more and 900 nm or less depending on material design, whereby most of excitation energy can be obtained as light having a desired wavelength. Therefore, the excitation energy can be efficiently utilized, whereby high color purity can also be achieved. Here, the single peak in a wavelength region represents a state in which in the wavelength region relative to a peak having the highest intensity there is no peak the intensity of which is 5% or more of the highest intensity. The same applies to the following description.

Further, the compound represented by general formula (1), by introducing an appropriate substituent to an appropriate position, enables various characteristics and physical properties such as a luminous efficiency, a light emission wavelength, color purity, heat resistance, and dispersibility to be adjusted.

For example, the compound represented by general formula (1) when at least one of R¹, R³, R⁴ and R⁶ is a substituted or unsubstituted alkyl group, a substituted or unsubstituted aryl group, or a substituted or unsubstituted heteroaryl group exhibits higher heat resistance and photostability than those when R¹, R³, R⁴ and R⁶ each are a hydrogen atom. When the heat resistance is improved, the decomposition of the compound during the production of the light-emitting element can be suppressed, which provides improved durability.

From the viewpoint of improving heat resistance and fluorescence quantum yield, it is also preferred that R¹ to R⁹ form a fused ring with an adjacent substituent.

When at least one of R¹, R³, R⁴, and R⁶ is a substituted or unsubstituted alkyl group, the alkyl group is preferably an alkyl group having 1 to 6 carbon atoms such as a methyl group, an ethyl group, a n-propyl group, an isopropyl group, a n-butyl group, a sec-butyl group, a tert-butyl group, a pentyl group, or a hexyl group. Further, from the viewpoint of excellent thermal stability, the alkyl group is preferably a methyl group, an ethyl group, a n-propyl group, an isopropyl group, a n-butyl group, a sec-butyl group, and a tert-butyl group. From the viewpoint of preventing concentration quenching to improve fluorescence quantum yield, the alkyl group is more preferably a tert-butyl group which is sterically bulky. From the viewpoints of the easiness of synthesis and raw material availability, a methyl group is also preferably used.

When at least one of R¹, R³, R⁴, and R⁶ is a substituted or unsubstituted aryl group, the aryl group is preferably a phenyl group, a biphenyl group, a terphenyl group, or a naphthyl group, more preferably a phenyl group or a biphenyl group, and particularly preferably a phenyl group.

When at least one of R¹, R³, R⁴, and R⁶ is a substituted or unsubstituted heteroaryl group, the heteroaryl group is preferably a pyridyl group, a quinolinyl group, or a thienyl group, more preferably a pyridyl group or a quinolinyl group, and particularly preferably a pyridyl group.

When all R¹, R³, R⁴, and R⁶ are the same or different from each other, and each are a substituted or unsubstituted alkyl group, color purity is particularly good, which is preferable. In this case, the alkyl group is preferably a methyl group from the viewpoints of the easiness of synthesis and raw material availability.

When all R¹, R³, R⁴, and R⁶ are the same or different from each other, and each are a substituted or unsubstituted aryl group or a substituted or unsubstituted heteroaryl group, higher thermal stability and photostability are exhibited, which is preferable. In this case, all R¹, R³, R⁴, and R⁶ are the same or different from each other, and are more preferably a substituted or unsubstituted aryl group.

Although some substituents improve a plurality of properties, substituents which exhibit sufficient performance in all are limited. In particular, it is difficult to achieve both a high luminous efficiency and high color purity. Therefore, a plurality of kinds of substituents are introduced to the compound represented by general formula (1), whereby a compound having a balance among light emission characteristics and color purity and the like can be obtained.

In particular, when all R¹, R³, R⁴, and R⁶ are the same or different from each other, and each are a substituted or unsubstituted aryl group, a plurality of kinds of substituents are preferably introduced, such as R¹ not equal to R⁴, R³ not equal to R⁶, R¹ not equal to R³, or R⁴ not equal to R⁶. Here, “not equal to” means that they are groups having different structures. R¹ not equal to R⁴ means that R¹ and R⁴ are groups having different structures, for example. A plurality of kinds of substituents are introduced as described above, whereby an aryl group which has an influence on color purity and an aryl group which has an influence on a luminous efficiency can be simultaneously introduced, whereby fine adjustment can be made.

Among these, R¹ not equal to R³ or R⁴ not equal to R⁶ is preferred from the viewpoint of improving a luminous efficiency and color purity with a good balance . In this case, to the compound represented by general formula (1), one or more aryl groups having an influence on color purity can be introduced to both pyrrole rings each, whereas an aryl group having an influence on a luminous efficiency can be introduced to any other position, whereby both of these properties can be improved to the maximum. In R¹ not equal to R³ or R⁴ not equal to R⁶, from the viewpoint of improving both heat resistance and color purity, R¹═R⁴ and R³═R⁶ are more preferable.

The aryl group which has an influence mainly on color purity is preferably an aryl group substituted with an electron-donating group. The electron-donating group is an atomic group which donates an electron to a substituted atomic group by the inductive effect and the resonance effect in the organic electron theory. Examples of the electron-donating group include those having a negative value as a substituent constant (σp (para)) of Hammett's Rule. The substituent constant (σp (para)) of Hammett's Rule can be cited from Kagaku Binran Kiso-Hen Revised 5th Edition (II, p. 380).

Specific examples of the electron-donating group include an alkyl group (σp of a methyl group: −0.17), an alkoxy group (σp of a methoxy group=−0.27), and an amino group (σp of —NH₂=−0.66). In particular, an alkyl group having 1 to 8 carbon atoms or an alkoxy group having 1 to 8 carbon atoms is preferred, and a methyl group, an ethyl group, a tert-butyl group, and a methoxy group are more preferable. From the viewpoint of dispersibility, a tert-butyl group and a methoxy group are particularly preferable. When these substituents are the electron-donating group, quenching caused by the flocculation of molecules can be prevented in the compound represented by general formula (1). Although the substitution position of the substituent is not particularly limited, the substituent is preferably bonded to the meta position or the para position relative to the position bonding to the pyrromethene skeleton, because the twist of bonding is required to be inhibited in order to improve the photostability of the compound represented by general formula (1).

Meanwhile, the aryl group which has an influence mainly on a luminous efficiency is preferably an aryl group having a bulky substituent such as a tert-butyl group, an adamantyl group, or a methoxy group.

When R¹, R³, R⁴, and R⁶ are the same or different from each other, and each are a substituted or unsubstituted aryl group, R¹, R³, R⁴, and R⁶ are the same or different from each other, and each are preferably a substituted or unsubstituted phenyl group. In this case, R¹, R³, R⁴, and R⁶ each are more preferably selected from the following Ar-1 to Ar-6. In this case, examples of a preferred combination of R¹, R³, R⁴, and R⁶ include, but are not limited to, combinations shown in Table 1-1 to Table 1-11.

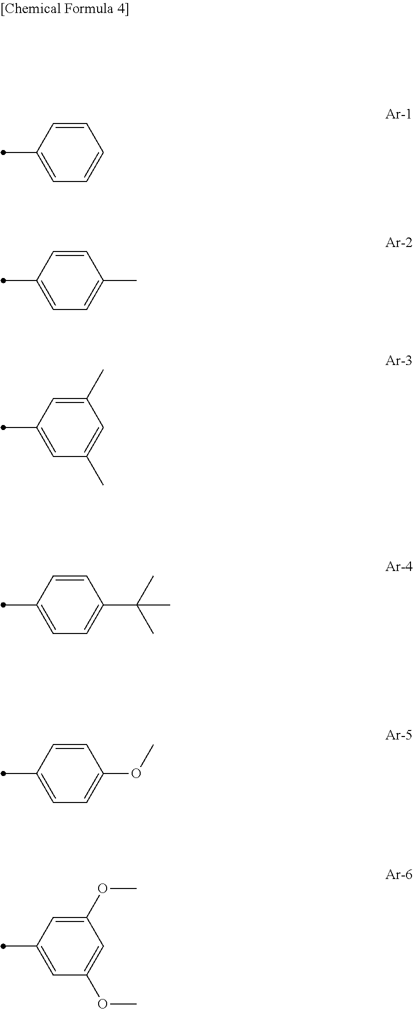

TABLE 1-1

| R1 | R3 | R4 | R6 | R1 | R3 | R4 | R6 |
|---|---|---|---|---|---|---|---|
| Ar-1 | Ar-1 | Ar-1 | Ar-1 | Ar-1 | Ar-1 | Ar-6 | Ar-1 |
| Ar-1 | Ar-1 | Ar-1 | Ar-2 | Ar-1 | Ar-1 | Ar-6 | Ar-2 |
| Ar-1 | Ar-1 | Ar-1 | Ar-3 | Ar-1 | Ar-1 | Ar-6 | Ar-3 |
| Ar-1 | Ar-1 | Ar-1 | Ar-4 | Ar-1 | Ar-1 | Ar-6 | Ar-4 |
| Ar-1 | Ar-1 | Ar-1 | Ar-5 | Ar-1 | Ar-1 | Ar-6 | Ar-5 |
| Ar-1 | Ar-1 | Ar-1 | Ar-6 | Ar-1 | Ar-1 | Ar-6 | Ar-6 |
| Ar-1 | Ar-1 | Ar-2 | Ar-1 | Ar-1 | Ar-2 | Ar-1 | Ar-2 |
| Ar-1 | Ar-1 | Ar-2 | Ar-2 | Ar-1 | Ar-2 | Ar-1 | Ar-3 |
| Ar-1 | Ar-1 | Ar-2 | Ar-3 | Ar-1 | Ar-2 | Ar-1 | Ar-4 |
| Ar-1 | Ar-1 | Ar-2 | Ar-4 | Ar-1 | Ar-2 | Ar-1 | Ar-5 |
| Ar-1 | Ar-1 | Ar-2 | Ar-5 | Ar-1 | Ar-2 | Ar-1 | Ar-6 |
| Ar-1 | Ar-1 | Ar-2 | Ar-6 | Ar-1 | Ar-2 | Ar-2 | Ar-1 |
| Ar-1 | Ar-1 | Ar-3 | Ar-1 | Ar-1 | Ar-2 | Ar-2 | Ar-2 |
| Ar-1 | Ar-1 | Ar-3 | Ar-2 | Ar-1 | Ar-2 | Ar-2 | Ar-3 |
| Ar-1 | Ar-1 | Ar-3 | Ar-3 | Ar-1 | Ar-2 | Ar-2 | Ar-4 |
| Ar-1 | Ar-1 | Ar-3 | Ar-4 | Ar-1 | Ar-2 | Ar-2 | Ar-5 |
| Ar-1 | Ar-1 | Ar-3 | Ar-5 | Ar-1 | Ar-2 | Ar-2 | Ar-6 |
| Ar-1 | Ar-1 | Ar-3 | Ar-6 | Ar-1 | Ar-2 | Ar-3 | Ar-1 |
| Ar-1 | Ar-1 | Ar-4 | Ar-1 | Ar-1 | Ar-2 | Ar-3 | Ar-2 |
| Ar-1 | Ar-1 | Ar-4 | Ar-2 | Ar-1 | Ar-2 | Ar-3 | Ar-3 |
| Ar-1 | Ar-1 | Ar-4 | Ar-3 | Ar-1 | Ar-2 | Ar-3 | Ar-4 |
| Ar-1 | Ar-1 | Ar-4 | Ar-4 | Ar-1 | Ar-2 | Ar-3 | Ar-5 |
| Ar-1 | Ar-1 | Ar-4 | Ar-5 | Ar-1 | Ar-2 | Ar-3 | Ar-6 |
| Ar-1 | Ar-1 | Ar-4 | Ar-6 | Ar-1 | Ar-2 | Ar-4 | Ar-1 |
| Ar-1 | Ar-1 | Ar-5 | Ar-1 | Ar-1 | Ar-2 | Ar-4 | Ar-2 |
| Ar-1 | Ar-1 | Ar-5 | Ar-2 | Ar-1 | Ar-2 | Ar-4 | Ar-3 |
| Ar-1 | Ar-1 | Ar-5 | Ar-3 | Ar-1 | Ar-2 | Ar-4 | Ar-4 |
| Ar-1 | Ar-1 | Ar-5 | Ar-4 | Ar-1 | Ar-2 | Ar-4 | Ar-5 |
| Ar-1 | Ar-1 | Ar-5 | Ar-5 | Ar-1 | Ar-2 | Ar-4 | Ar-6 |
| Ar-1 | Ar-1 | Ar-5 | Ar-6 | | | | |

TABLE 1-2

| R1 | R3 | R4 | R6 | R1 | R3 | R4 | R6 |
|---|---|---|---|---|---|---|---|
| Ar-1 | Ar-2 | Ar-5 | Ar-1 | Ar-1 | Ar-3 | Ar-4 | Ar-4 |
| Ar-1 | Ar-2 | Ar-5 | Ar-2 | Ar-1 | Ar-3 | Ar-4 | Ar-5 |
| Ar-1 | Ar-2 | Ar-5 | Ar-3 | Ar-1 | Ar-3 | Ar-4 | Ar-6 |
| Ar-1 | Ar-2 | Ar-5 | Ar-4 | Ar-1 | Ar-3 | Ar-5 | Ar-1 |
| Ar-1 | Ar-2 | Ar-5 | Ar-5 | Ar-1 | Ar-3 | Ar-5 | Ar-2 |
| Ar-1 | Ar-2 | Ar-5 | Ar-6 | Ar-1 | Ar-3 | Ar-5 | Ar-3 |
| Ar-1 | Ar-2 | Ar-6 | Ar-1 | Ar-1 | Ar-3 | Ar-5 | Ar-4 |
| Ar-1 | Ar-2 | Ar-6 | Ar-2 | Ar-1 | Ar-3 | Ar-5 | Ar-5 |
| Ar-1 | Ar-2 | Ar-6 | Ar-3 | Ar-1 | Ar-3 | Ar-5 | Ar-6 |
| Ar-1 | Ar-2 | Ar-6 | Ar-4 | Ar-1 | Ar-3 | Ar-6 | Ar-1 |
| Ar-1 | Ar-2 | Ar-6 | Ar-5 | Ar-1 | Ar-3 | Ar-6 | Ar-2 |
| Ar-1 | Ar-2 | Ar-6 | Ar-6 | Ar-1 | Ar-3 | Ar-6 | Ar-3 |
| Ar-1 | Ar-3 | Ar-1 | Ar-2 | Ar-1 | Ar-3 | Ar-6 | Ar-4 |
| Ar-1 | Ar-3 | Ar-1 | Ar-3 | Ar-1 | Ar-3 | Ar-6 | Ar-5 |
| Ar-1 | Ar-3 | Ar-1 | Ar-4 | Ar-1 | Ar-3 | Ar-6 | Ar-6 |
| Ar-1 | Ar-3 | Ar-1 | Ar-5 | Ar-1 | Ar-4 | Ar-1 | Ar-2 |
| Ar-1 | Ar-3 | Ar-1 | Ar-6 | Ar-1 | Ar-4 | Ar-1 | Ar-3 |
| Ar-1 | Ar-3 | Ar-2 | Ar-2 | Ar-1 | Ar-4 | Ar-1 | Ar-4 |
| Ar-1 | Ar-3 | Ar-2 | Ar-3 | Ar-1 | Ar-4 | Ar-1 | Ar-5 |
| Ar-1 | Ar-3 | Ar-2 | Ar-4 | Ar-1 | Ar-4 | Ar-1 | Ar-6 |
| Ar-1 | Ar-3 | Ar-2 | Ar-5 | Ar-1 | Ar-4 | Ar-2 | Ar-2 |
| Ar-1 | Ar-3 | Ar-2 | Ar-6 | Ar-1 | Ar-4 | Ar-2 | Ar-3 |
| Ar-1 | Ar-3 | Ar-3 | Ar-1 | Ar-1 | Ar-4 | Ar-2 | Ar-4 |
| Ar-1 | Ar-3 | Ar-3 | Ar-2 | Ar-1 | Ar-4 | Ar-2 | Ar-5 |
| Ar-1 | Ar-3 | Ar-3 | Ar-3 | Ar-1 | Ar-4 | Ar-2 | Ar-6 |
| Ar-1 | Ar-3 | Ar-3 | Ar-4 | Ar-1 | Ar-4 | Ar-3 | Ar-2 |
| Ar-1 | Ar-3 | Ar-3 | Ar-5 | Ar-1 | Ar-4 | Ar-3 | Ar-3 |
| Ar-1 | Ar-3 | Ar-3 | Ar-6 | Ar-1 | Ar-4 | Ar-3 | Ar-4 |
| Ar-1 | Ar-3 | Ar-4 | Ar-1 | Ar-1 | Ar-4 | Ar-3 | Ar-5 |
| Ar-1 | Ar-3 | Ar-4 | Ar-2 | Ar-1 | Ar-4 | Ar-3 | Ar-6 |
| Ar-1 | Ar-3 | Ar-4 | Ar-3 | | | | |

TABLE 1-3

| R1 | R3 | R4 | R6 | R1 | R3 | R4 | R6 |
|---|---|---|---|---|---|---|---|
| Ar-1 | Ar-4 | Ar-4 | Ar-1 | Ar-1 | Ar-5 | Ar-3 | Ar-4 |
| Ar-1 | Ar-4 | Ar-4 | Ar-2 | Ar-1 | Ar-5 | Ar-3 | Ar-5 |
| Ar-1 | Ar-4 | Ar-4 | Ar-3 | Ar-1 | Ar-5 | Ar-3 | Ar-6 |
| Ar-1 | Ar-4 | Ar-4 | Ar-4 | Ar-1 | Ar-5 | Ar-4 | Ar-2 |
| Ar-1 | Ar-4 | Ar-4 | Ar-5 | Ar-1 | Ar-5 | Ar-4 | Ar-3 |
| Ar-1 | Ar-4 | Ar-4 | Ar-6 | Ar-1 | Ar-5 | Ar-4 | Ar-4 |
| Ar-1 | Ar-4 | Ar-5 | Ar-1 | Ar-1 | Ar-5 | Ar-4 | Ar-5 |
| Ar-1 | Ar-4 | Ar-5 | Ar-2 | Ar-1 | Ar-5 | Ar-4 | Ar-6 |
| Ar-1 | Ar-4 | Ar-5 | Ar-3 | Ar-1 | Ar-5 | Ar-5 | Ar-1 |
| Ar-1 | Ar-4 | Ar-5 | Ar-4 | Ar-1 | Ar-5 | Ar-5 | Ar-2 |
| Ar-1 | Ar-4 | Ar-5 | Ar-5 | Ar-1 | Ar-5 | Ar-5 | Ar-3 |
| Ar-1 | Ar-4 | Ar-5 | Ar-6 | Ar-1 | Ar-5 | Ar-5 | Ar-4 |
| Ar-1 | Ar-4 | Ar-6 | Ar-1 | Ar-1 | Ar-5 | Ar-5 | Ar-5 |
| Ar-1 | Ar-4 | Ar-6 | Ar-2 | Ar-1 | Ar-5 | Ar-5 | Ar-6 |
| Ar-1 | Ar-4 | Ar-6 | Ar-3 | Ar-1 | Ar-5 | Ar-6 | Ar-1 |
| Ar-1 | Ar-4 | Ar-6 | Ar-4 | Ar-1 | Ar-5 | Ar-6 | Ar-2 |
| Ar-1 | Ar-4 | Ar-6 | Ar-5 | Ar-1 | Ar-5 | Ar-6 | Ar-3 |
| Ar-1 | Ar-4 | Ar-6 | Ar-6 | Ar-1 | Ar-5 | Ar-6 | Ar-4 |
| Ar-1 | Ar-5 | Ar-1 | Ar-2 | Ar-1 | Ar-5 | Ar-6 | Ar-5 |
| Ar-1 | Ar-5 | Ar-1 | Ar-3 | Ar-1 | Ar-5 | Ar-6 | Ar-6 |
| Ar-1 | Ar-5 | Ar-1 | Ar-4 | Ar-1 | Ar-6 | Ar-1 | Ar-2 |
| Ar-1 | Ar-5 | Ar-1 | Ar-5 | Ar-1 | Ar-6 | Ar-1 | Ar-3 |
| Ar-1 | Ar-5 | Ar-1 | Ar-6 | Ar-1 | Ar-6 | Ar-1 | Ar-4 |
| Ar-1 | Ar-5 | Ar-2 | Ar-2 | Ar-1 | Ar-6 | Ar-1 | Ar-5 |
| Ar-1 | Ar-5 | Ar-2 | Ar-3 | Ar-1 | Ar-6 | Ar-1 | Ar-6 |
| Ar-1 | Ar-5 | Ar-2 | Ar-4 | Ar-1 | Ar-6 | Ar-2 | Ar-2 |
| Ar-1 | Ar-5 | Ar-2 | Ar-5 | Ar-1 | Ar-6 | Ar-2 | Ar-3 |
| Ar-1 | Ar-5 | Ar-2 | Ar-6 | Ar-1 | Ar-6 | Ar-2 | Ar-4 |
| Ar-1 | Ar-5 | Ar-3 | Ar-2 | Ar-1 | Ar-6 | Ar-2 | Ar-5 |
| Ar-1 | Ar-5 | Ar-3 | Ar-3 | Ar-1 | Ar-6 | Ar-2 | Ar-6 |

TABLE 1-4

| R1 | R3 | R4 | R6 | R1 | R3 | R4 | R6 |
|---|---|---|---|---|---|---|---|
| Ar-1 | Ar-6 | Ar-3 | Ar-2 | Ar-2 | Ar-1 | Ar-2 | Ar-6 |
| Ar-1 | Ar-6 | Ar-3 | Ar-3 | Ar-2 | Ar-1 | Ar-3 | Ar-2 |
| Ar-1 | Ar-6 | Ar-3 | Ar-4 | Ar-2 | Ar-1 | Ar-3 | Ar-3 |
| Ar-1 | Ar-6 | Ar-3 | Ar-5 | Ar-2 | Ar-1 | Ar-3 | Ar-4 |
| Ar-1 | Ar-6 | Ar-3 | Ar-6 | Ar-2 | Ar-1 | Ar-3 | Ar-5 |
| Ar-1 | Ar-6 | Ar-4 | Ar-2 | Ar-2 | Ar-1 | Ar-3 | Ar-6 |
| Ar-1 | Ar-6 | Ar-4 | Ar-3 | Ar-2 | Ar-1 | Ar-4 | Ar-2 |
| Ar-1 | Ar-6 | Ar-4 | Ar-4 | Ar-2 | Ar-1 | Ar-4 | Ar-3 |
| Ar-1 | Ar-6 | Ar-4 | Ar-5 | Ar-2 | Ar-1 | Ar-4 | Ar-4 |
| Ar-1 | Ar-6 | Ar-4 | Ar-6 | Ar-2 | Ar-1 | Ar-4 | Ar-5 |
| Ar-1 | Ar-6 | Ar-5 | Ar-2 | Ar-2 | Ar-1 | Ar-4 | Ar-6 |
| Ar-1 | Ar-6 | Ar-5 | Ar-3 | Ar-2 | Ar-1 | Ar-5 | Ar-2 |
| Ar-1 | Ar-6 | Ar-5 | Ar-4 | Ar-2 | Ar-1 | Ar-5 | Ar-3 |
| Ar-1 | Ar-6 | Ar-5 | Ar-5 | Ar-2 | Ar-1 | Ar-5 | Ar-4 |
| Ar-1 | Ar-6 | Ar-5 | Ar-6 | Ar-2 | Ar-1 | Ar-5 | Ar-5 |
| Ar-1 | Ar-6 | Ar-6 | Ar-1 | Ar-2 | Ar-1 | Ar-5 | Ar-6 |
| Ar-1 | Ar-6 | Ar-6 | Ar-2 | Ar-2 | Ar-1 | Ar-6 | Ar-2 |
| Ar-1 | Ar-6 | Ar-6 | Ar-3 | Ar-2 | Ar-1 | Ar-6 | Ar-3 |
| Ar-1 | Ar-6 | Ar-6 | Ar-4 | Ar-2 | Ar-1 | Ar-6 | Ar-4 |
| Ar-1 | Ar-6 | Ar-6 | Ar-5 | Ar-2 | Ar-1 | Ar-6 | Ar-5 |
| Ar-1 | Ar-6 | Ar-6 | Ar-6 | Ar-2 | Ar-1 | Ar-6 | Ar-6 |
| Ar-2 | Ar-1 | Ar-1 | Ar-2 | Ar-2 | Ar-2 | Ar-1 | Ar-3 |
| Ar-2 | Ar-1 | Ar-1 | Ar-3 | Ar-2 | Ar-2 | Ar-1 | Ar-4 |
| Ar-2 | Ar-1 | Ar-1 | Ar-4 | Ar-2 | Ar-2 | Ar-1 | Ar-5 |
| Ar-2 | Ar-1 | Ar-1 | Ar-5 | Ar-2 | Ar-2 | Ar-1 | Ar-6 |
| Ar-2 | Ar-1 | Ar-1 | Ar-6 | Ar-2 | Ar-2 | Ar-2 | Ar-2 |
| Ar-2 | Ar-1 | Ar-2 | Ar-2 | Ar-2 | Ar-2 | Ar-2 | Ar-3 |
| Ar-2 | Ar-1 | Ar-2 | Ar-3 | Ar-2 | Ar-2 | Ar-2 | Ar-4 |
| Ar-2 | Ar-1 | Ar-2 | Ar-4 | Ar-2 | Ar-2 | Ar-2 | Ar-5 |
| Ar-2 | Ar-1 | Ar-2 | Ar-5 | Ar-2 | Ar-2 | Ar-2 | Ar-6 |

TABLE 1-5

| R1 | R3 | R4 | R6 | R1 | R3 | R4 | R6 |
|---|---|---|---|---|---|---|---|
| Ar-2 | Ar-2 | Ar-3 | Ar-2 | Ar-2 | Ar-3 | Ar-3 | Ar-4 |
| Ar-2 | Ar-2 | Ar-3 | Ar-3 | Ar-2 | Ar-3 | Ar-3 | Ar-5 |
| Ar-2 | Ar-2 | Ar-3 | Ar-4 | Ar-2 | Ar-3 | Ar-3 | Ar-6 |
| Ar-2 | Ar-2 | Ar-3 | Ar-5 | Ar-2 | Ar-3 | Ar-4 | Ar-2 |
| Ar-2 | Ar-2 | Ar-3 | Ar-6 | Ar-2 | Ar-3 | Ar-4 | Ar-3 |
| Ar-2 | Ar-2 | Ar-4 | Ar-2 | Ar-2 | Ar-3 | Ar-4 | Ar-4 |
| Ar-2 | Ar-2 | Ar-4 | Ar-3 | Ar-2 | Ar-3 | Ar-4 | Ar-5 |
| Ar-2 | Ar-2 | Ar-4 | Ar-4 | Ar-2 | Ar-3 | Ar-4 | Ar-6 |
| Ar-2 | Ar-2 | Ar-4 | Ar-5 | Ar-2 | Ar-3 | Ar-5 | Ar-2 |
| Ar-2 | Ar-2 | Ar-4 | Ar-6 | Ar-2 | Ar-3 | Ar-5 | Ar-3 |
| Ar-2 | Ar-2 | Ar-5 | Ar-2 | Ar-2 | Ar-3 | Ar-5 | Ar-4 |
| Ar-2 | Ar-2 | Ar-5 | Ar-3 | Ar-2 | Ar-3 | Ar-5 | Ar-5 |
| Ar-2 | Ar-2 | Ar-5 | Ar-4 | Ar-2 | Ar-3 | Ar-5 | Ar-6 |
| Ar-2 | Ar-2 | Ar-5 | Ar-5 | Ar-2 | Ar-3 | Ar-6 | Ar-2 |
| Ar-2 | Ar-2 | Ar-5 | Ar-6 | Ar-2 | Ar-3 | Ar-6 | Ar-3 |
| Ar-2 | Ar-2 | Ar-6 | Ar-2 | Ar-2 | Ar-3 | Ar-6 | Ar-4 |
| Ar-2 | Ar-2 | Ar-6 | Ar-3 | Ar-2 | Ar-3 | Ar-6 | Ar-5 |
| Ar-2 | Ar-2 | Ar-6 | Ar-4 | Ar-2 | Ar-3 | Ar-6 | Ar-6 |
| Ar-2 | Ar-2 | Ar-6 | Ar-5 | Ar-2 | Ar-4 | Ar-1 | Ar-3 |
| Ar-2 | Ar-2 | Ar-6 | Ar-6 | Ar-2 | Ar-4 | Ar-1 | Ar-4 |
| Ar-2 | Ar-3 | Ar-1 | Ar-3 | Ar-2 | Ar-4 | Ar-1 | Ar-5 |
| Ar-2 | Ar-3 | Ar-1 | Ar-4 | Ar-2 | Ar-4 | Ar-1 | Ar-6 |
| Ar-2 | Ar-3 | Ar-1 | Ar-5 | Ar-2 | Ar-4 | Ar-2 | Ar-3 |
| Ar-2 | Ar-3 | Ar-1 | Ar-6 | Ar-2 | Ar-4 | Ar-2 | Ar-4 |
| Ar-2 | Ar-3 | Ar-2 | Ar-3 | Ar-2 | Ar-4 | Ar-2 | Ar-5 |
| Ar-2 | Ar-3 | Ar-2 | Ar-4 | Ar-2 | Ar-4 | Ar-2 | Ar-6 |
| Ar-2 | Ar-3 | Ar-2 | Ar-5 | Ar-2 | Ar-4 | Ar-3 | Ar-3 |
| Ar-2 | Ar-3 | Ar-2 | Ar-6 | Ar-2 | Ar-4 | Ar-3 | Ar-4 |
| Ar-2 | Ar-3 | Ar-3 | Ar-2 | Ar-2 | Ar-4 | Ar-3 | Ar-5 |
| Ar-2 | Ar-3 | Ar-3 | Ar-3 | Ar-2 | Ar-4 | Ar-3 | Ar-6 |

TABLE 1-6

| R1 | R3 | R4 | R6 | R1 | R3 | R4 | R6 |
|---|---|---|---|---|---|---|---|
| Ar-2 | Ar-4 | Ar-4 | Ar-2 | Ar-2 | Ar-5 | Ar-5 | Ar-2 |
| Ar-2 | Ar-4 | Ar-4 | Ar-3 | Ar-2 | Ar-5 | Ar-5 | Ar-3 |
| Ar-2 | Ar-4 | Ar-4 | Ar-4 | Ar-2 | Ar-5 | Ar-5 | Ar-4 |
| Ar-2 | Ar-4 | Ar-4 | Ar-5 | Ar-2 | Ar-5 | Ar-5 | Ar-5 |
| Ar-2 | Ar-4 | Ar-4 | Ar-6 | Ar-2 | Ar-5 | Ar-5 | Ar-6 |
| Ar-2 | Ar-4 | Ar-5 | Ar-2 | Ar-2 | Ar-5 | Ar-6 | Ar-2 |
| Ar-2 | Ar-4 | Ar-5 | Ar-3 | Ar-2 | Ar-5 | Ar-6 | Ar-3 |
| Ar-2 | Ar-4 | Ar-5 | Ar-4 | Ar-2 | Ar-5 | Ar-6 | Ar-4 |
| Ar-2 | Ar-4 | Ar-5 | Ar-5 | Ar-2 | Ar-5 | Ar-6 | Ar-5 |
| Ar-2 | Ar-4 | Ar-5 | Ar-6 | Ar-2 | Ar-5 | Ar-6 | Ar-6 |
| Ar-2 | Ar-4 | Ar-6 | Ar-2 | Ar-2 | Ar-6 | Ar-1 | Ar-3 |
| Ar-2 | Ar-4 | Ar-6 | Ar-3 | Ar-2 | Ar-6 | Ar-1 | Ar-4 |
| Ar-2 | Ar-4 | Ar-6 | Ar-4 | Ar-2 | Ar-6 | Ar-1 | Ar-5 |
| Ar-2 | Ar-4 | Ar-6 | Ar-5 | Ar-2 | Ar-6 | Ar-1 | Ar-6 |
| Ar-2 | Ar-4 | Ar-6 | Ar-6 | Ar-2 | Ar-6 | Ar-2 | Ar-3 |
| Ar-2 | Ar-5 | Ar-1 | Ar-3 | Ar-2 | Ar-6 | Ar-2 | Ar-4 |
| Ar-2 | Ar-5 | Ar-1 | Ar-4 | Ar-2 | Ar-6 | Ar-2 | Ar-5 |
| Ar-2 | Ar-5 | Ar-1 | Ar-5 | Ar-2 | Ar-6 | Ar-2 | Ar-6 |
| Ar-2 | Ar-5 | Ar-1 | Ar-6 | Ar-2 | Ar-6 | Ar-3 | Ar-3 |
| Ar-2 | Ar-5 | Ar-2 | Ar-3 | Ar-2 | Ar-6 | Ar-3 | Ar-4 |
| Ar-2 | Ar-5 | Ar-2 | Ar-4 | Ar-2 | Ar-6 | Ar-3 | Ar-5 |
| Ar-2 | Ar-5 | Ar-2 | Ar-5 | Ar-2 | Ar-6 | Ar-3 | Ar-6 |
| Ar-2 | Ar-5 | Ar-2 | Ar-6 | Ar-2 | Ar-6 | Ar-4 | Ar-3 |
| Ar-2 | Ar-5 | Ar-3 | Ar-3 | Ar-2 | Ar-6 | Ar-4 | Ar-4 |
| Ar-2 | Ar-5 | Ar-3 | Ar-4 | Ar-2 | Ar-6 | Ar-4 | Ar-5 |
| Ar-2 | Ar-5 | Ar-3 | Ar-5 | Ar-2 | Ar-6 | Ar-4 | Ar-6 |
| Ar-2 | Ar-5 | Ar-3 | Ar-6 | Ar-2 | Ar-6 | Ar-5 | Ar-3 |
| Ar-2 | Ar-5 | Ar-4 | Ar-3 | Ar-2 | Ar-6 | Ar-5 | Ar-4 |
| Ar-2 | Ar-5 | Ar-4 | Ar-4 | Ar-2 | Ar-6 | Ar-5 | Ar-5 |
| Ar-2 | Ar-5 | Ar-4 | Ar-5 | Ar-2 | Ar-6 | Ar-5 | Ar-6 |
| Ar-2 | Ar-5 | Ar-4 | Ar-6 | | | | |

TABLE 1-7

| R1 | R3 | R4 | R6 | R1 | R3 | R4 | R6 |
|---|---|---|---|---|---|---|---|
| Ar-2 | Ar-6 | Ar-6 | Ar-2 | Ar-3 | Ar-2 | Ar-1 | Ar-6 |
| Ar-2 | Ar-6 | Ar-6 | Ar-3 | Ar-3 | Ar-2 | Ar-2 | Ar-3 |
| Ar-2 | Ar-6 | Ar-6 | Ar-4 | Ar-3 | Ar-2 | Ar-2 | Ar-4 |
| Ar-2 | Ar-6 | Ar-6 | Ar-5 | Ar-3 | Ar-2 | Ar-2 | Ar-5 |
| Ar-2 | Ar-6 | Ar-6 | Ar-6 | Ar-3 | Ar-2 | Ar-2 | Ar-6 |
| Ar-3 | Ar-1 | Ar-1 | Ar-3 | Ar-3 | Ar-2 | Ar-3 | Ar-3 |
| Ar-3 | Ar-1 | Ar-1 | Ar-4 | Ar-3 | Ar-2 | Ar-3 | Ar-4 |
| Ar-3 | Ar-1 | Ar-1 | Ar-5 | Ar-3 | Ar-2 | Ar-3 | Ar-5 |
| Ar-3 | Ar-1 | Ar-1 | Ar-6 | Ar-3 | Ar-2 | Ar-3 | Ar-6 |
| Ar-3 | Ar-1 | Ar-2 | Ar-3 | Ar-3 | Ar-2 | Ar-4 | Ar-3 |
| Ar-3 | Ar-1 | Ar-2 | Ar-4 | Ar-3 | Ar-2 | Ar-4 | Ar-4 |
| Ar-3 | Ar-1 | Ar-2 | Ar-5 | Ar-3 | Ar-2 | Ar-4 | Ar-5 |
| Ar-3 | Ar-1 | Ar-2 | Ar-6 | Ar-3 | Ar-2 | Ar-4 | Ar-6 |
| Ar-3 | Ar-1 | Ar-3 | Ar-3 | Ar-3 | Ar-2 | Ar-5 | Ar-3 |
| Ar-3 | Ar-1 | Ar-3 | Ar-4 | Ar-3 | Ar-2 | Ar-5 | Ar-4 |
| Ar-3 | Ar-1 | Ar-3 | Ar-5 | Ar-3 | Ar-2 | Ar-5 | Ar-5 |
| Ar-3 | Ar-1 | Ar-3 | Ar-6 | Ar-3 | Ar-2 | Ar-5 | Ar-6 |
| Ar-3 | Ar-1 | Ar-4 | Ar-3 | Ar-3 | Ar-2 | Ar-6 | Ar-3 |
| Ar-3 | Ar-1 | Ar-4 | Ar-4 | Ar-3 | Ar-2 | Ar-6 | Ar-4 |
| Ar-3 | Ar-1 | Ar-4 | Ar-5 | Ar-3 | Ar-2 | Ar-6 | Ar-5 |
| Ar-3 | Ar-1 | Ar-4 | Ar-6 | Ar-3 | Ar-2 | Ar-6 | Ar-6 |
| Ar-3 | Ar-1 | Ar-5 | Ar-3 | Ar-3 | Ar-3 | Ar-1 | Ar-4 |
| Ar-3 | Ar-1 | Ar-5 | Ar-4 | Ar-3 | Ar-3 | Ar-1 | Ar-5 |
| Ar-3 | Ar-1 | Ar-5 | Ar-5 | Ar-3 | Ar-3 | Ar-1 | Ar-6 |
| Ar-3 | Ar-1 | Ar-5 | Ar-6 | Ar-3 | Ar-3 | Ar-2 | Ar-4 |
| Ar-3 | Ar-1 | Ar-6 | Ar-3 | Ar-3 | Ar-3 | Ar-2 | Ar-5 |
| Ar-3 | Ar-1 | Ar-6 | Ar-4 | Ar-3 | Ar-3 | Ar-2 | Ar-6 |
| Ar-3 | Ar-1 | Ar-6 | Ar-5 | Ar-3 | Ar-3 | Ar-3 | Ar-3 |
| Ar-3 | Ar-1 | Ar-6 | Ar-6 | Ar-3 | Ar-3 | Ar-3 | Ar-4 |
| Ar-3 | Ar-2 | Ar-1 | Ar-4 | Ar-3 | Ar-3 | Ar-3 | Ar-5 |
| Ar-3 | Ar-2 | Ar-1 | Ar-5 | | | | |

TABLE 1-8

| R1 | R3 | R4 | R6 | R1 | R3 | R4 | R6 |
|---|---|---|---|---|---|---|---|
| Ar-3 | Ar-3 | Ar-3 | Ar-6 | Ar-3 | Ar-4 | Ar-6 | Ar-3 |
| Ar-3 | Ar-3 | Ar-4 | Ar-3 | Ar-3 | Ar-4 | Ar-6 | Ar-4 |
| Ar-3 | Ar-3 | Ar-4 | Ar-4 | Ar-3 | Ar-4 | Ar-6 | Ar-5 |
| Ar-3 | Ar-3 | Ar-4 | Ar-5 | Ar-3 | Ar-4 | Ar-6 | Ar-6 |
| Ar-3 | Ar-3 | Ar-4 | Ar-6 | Ar-3 | Ar-5 | Ar-1 | Ar-4 |
| Ar-3 | Ar-3 | Ar-5 | Ar-3 | Ar-3 | Ar-5 | Ar-1 | Ar-5 |
| Ar-3 | Ar-3 | Ar-5 | Ar-4 | Ar-3 | Ar-5 | Ar-1 | Ar-6 |
| Ar-3 | Ar-3 | Ar-5 | Ar-5 | Ar-3 | Ar-5 | Ar-2 | Ar-4 |
| Ar-3 | Ar-3 | Ar-5 | Ar-6 | Ar-3 | Ar-5 | Ar-2 | Ar-5 |
| Ar-3 | Ar-3 | Ar-6 | Ar-3 | Ar-3 | Ar-5 | Ar-2 | Ar-6 |
| Ar-3 | Ar-3 | Ar-6 | Ar-4 | Ar-3 | Ar-5 | Ar-3 | Ar-4 |
| Ar-3 | Ar-3 | Ar-6 | Ar-5 | Ar-3 | Ar-5 | Ar-3 | Ar-5 |
| Ar-3 | Ar-3 | Ar-6 | Ar-6 | Ar-3 | Ar-5 | Ar-3 | Ar-6 |
| Ar-3 | Ar-4 | Ar-1 | Ar-4 | Ar-3 | Ar-5 | Ar-4 | Ar-4 |
| Ar-3 | Ar-4 | Ar-1 | Ar-5 | Ar-3 | Ar-5 | Ar-4 | Ar-5 |
| Ar-3 | Ar-4 | Ar-1 | Ar-6 | Ar-3 | Ar-5 | Ar-4 | Ar-6 |
| Ar-3 | Ar-4 | Ar-2 | Ar-4 | Ar-3 | Ar-5 | Ar-5 | Ar-3 |
| Ar-3 | Ar-4 | Ar-2 | Ar-5 | Ar-3 | Ar-5 | Ar-5 | Ar-4 |
| Ar-3 | Ar-4 | Ar-2 | Ar-6 | Ar-3 | Ar-5 | Ar-5 | Ar-5 |
| Ar-3 | Ar-4 | Ar-3 | Ar-4 | Ar-3 | Ar-5 | Ar-5 | Ar-6 |
| Ar-3 | Ar-4 | Ar-3 | Ar-5 | Ar-3 | Ar-5 | Ar-6 | Ar-3 |
| Ar-3 | Ar-4 | Ar-3 | Ar-6 | Ar-3 | Ar-5 | Ar-6 | Ar-4 |
| Ar-3 | Ar-4 | Ar-4 | Ar-3 | Ar-3 | Ar-5 | Ar-6 | Ar-5 |
| Ar-3 | Ar-4 | Ar-4 | Ar-4 | Ar-3 | Ar-5 | Ar-6 | Ar-6 |
| Ar-3 | Ar-4 | Ar-4 | Ar-5 | Ar-3 | Ar-6 | Ar-1 | Ar-4 |
| Ar-3 | Ar-4 | Ar-4 | Ar-6 | Ar-3 | Ar-6 | Ar-1 | Ar-5 |
| Ar-3 | Ar-4 | Ar-5 | Ar-3 | Ar-3 | Ar-6 | Ar-1 | Ar-6 |
| Ar-3 | Ar-4 | Ar-5 | Ar-4 | Ar-3 | Ar-6 | Ar-2 | Ar-4 |
| Ar-3 | Ar-4 | Ar-5 | Ar-5 | Ar-3 | Ar-6 | Ar-2 | Ar-5 |
| Ar-3 | Ar-4 | Ar-5 | Ar-6 | Ar-3 | Ar-6 | Ar-2 | Ar-6 |

TABLE 1-9

| R1 | R3 | R4 | R6 | R1 | R3 | R4 | R6 |
|---|---|---|---|---|---|---|---|
| Ar-3 | Ar-6 | Ar-3 | Ar-4 | Ar-4 | Ar-2 | Ar-1 | Ar-5 |
| Ar-3 | Ar-6 | Ar-3 | Ar-5 | Ar-4 | Ar-2 | Ar-1 | Ar-6 |
| Ar-3 | Ar-6 | Ar-3 | Ar-6 | Ar-4 | Ar-2 | Ar-2 | Ar-4 |
| Ar-3 | Ar-6 | Ar-4 | Ar-4 | Ar-4 | Ar-2 | Ar-2 | Ar-5 |
| Ar-3 | Ar-6 | Ar-4 | Ar-5 | Ar-4 | Ar-2 | Ar-2 | Ar-6 |
| Ar-3 | Ar-6 | Ar-4 | Ar-6 | Ar-4 | Ar-2 | Ar-3 | Ar-4 |
| Ar-3 | Ar-6 | Ar-5 | Ar-4 | Ar-4 | Ar-2 | Ar-3 | Ar-5 |
| Ar-3 | Ar-6 | Ar-5 | Ar-5 | Ar-4 | Ar-2 | Ar-3 | Ar-6 |
| Ar-3 | Ar-6 | Ar-5 | Ar-6 | Ar-4 | Ar-2 | Ar-4 | Ar-4 |
| Ar-3 | Ar-6 | Ar-6 | Ar-3 | Ar-4 | Ar-2 | Ar-4 | Ar-5 |
| Ar-3 | Ar-6 | Ar-6 | Ar-4 | Ar-4 | Ar-2 | Ar-4 | Ar-6 |
| Ar-3 | Ar-6 | Ar-6 | Ar-5 | Ar-4 | Ar-2 | Ar-5 | Ar-4 |
| Ar-3 | Ar-6 | Ar-6 | Ar-6 | Ar-4 | Ar-2 | Ar-5 | Ar-5 |
| Ar-4 | Ar-1 | Ar-1 | Ar-4 | Ar-4 | Ar-2 | Ar-5 | Ar-6 |
| Ar-4 | Ar-1 | Ar-1 | Ar-5 | Ar-4 | Ar-2 | Ar-6 | Ar-4 |
| Ar-4 | Ar-1 | Ar-1 | Ar-6 | Ar-4 | Ar-2 | Ar-6 | Ar-5 |
| Ar-4 | Ar-1 | Ar-2 | Ar-4 | Ar-4 | Ar-2 | Ar-6 | Ar-6 |
| Ar-4 | Ar-1 | Ar-2 | Ar-5 | Ar-4 | Ar-3 | Ar-1 | Ar-5 |
| Ar-4 | Ar-1 | Ar-2 | Ar-6 | Ar-4 | Ar-3 | Ar-1 | Ar-6 |
| Ar-4 | Ar-1 | Ar-3 | Ar-4 | Ar-4 | Ar-3 | Ar-2 | Ar-5 |
| Ar-4 | Ar-1 | Ar-3 | Ar-5 | Ar-4 | Ar-3 | Ar-2 | Ar-6 |
| Ar-4 | Ar-1 | Ar-3 | Ar-6 | Ar-4 | Ar-3 | Ar-3 | Ar-4 |
| Ar-4 | Ar-1 | Ar-4 | Ar-4 | Ar-4 | Ar-3 | Ar-3 | Ar-5 |
| Ar-4 | Ar-1 | Ar-4 | Ar-5 | Ar-4 | Ar-3 | Ar-3 | Ar-6 |
| Ar-4 | Ar-1 | Ar-4 | Ar-6 | Ar-4 | Ar-3 | Ar-4 | Ar-4 |
| Ar-4 | Ar-1 | Ar-5 | Ar-4 | Ar-4 | Ar-3 | Ar-4 | Ar-5 |
| Ar-4 | Ar-1 | Ar-5 | Ar-5 | Ar-4 | Ar-3 | Ar-4 | Ar-6 |
| Ar-4 | Ar-1 | Ar-5 | Ar-6 | Ar-4 | Ar-3 | Ar-5 | Ar-4 |
| Ar-4 | Ar-1 | Ar-6 | Ar-4 | Ar-4 | Ar-3 | Ar-5 | Ar-5 |
| Ar-4 | Ar-1 | Ar-6 | Ar-5 | Ar-4 | Ar-3 | Ar-5 | Ar-6 |
| Ar-4 | Ar-1 | Ar-6 | Ar-6 | | | | |

TABLE 1-10

| R1 | R3 | R4 | R6 | R1 | R3 | R4 | R6 |
|---|---|---|---|---|---|---|---|
| Ar-4 | Ar-3 | Ar-6 | Ar-4 | Ar-4 | Ar-5 | Ar-6 | Ar-6 |
| Ar-4 | Ar-3 | Ar-6 | Ar-5 | Ar-4 | Ar-6 | Ar-1 | Ar-5 |
| Ar-4 | Ar-3 | Ar-6 | Ar-6 | Ar-4 | Ar-6 | Ar-1 | Ar-6 |
| Ar-4 | Ar-4 | Ar-1 | Ar-5 | Ar-4 | Ar-6 | Ar-2 | Ar-5 |
| Ar-4 | Ar-4 | Ar-1 | Ar-6 | Ar-4 | Ar-6 | Ar-2 | Ar-6 |
| Ar-4 | Ar-4 | Ar-2 | Ar-5 | Ar-4 | Ar-6 | Ar-3 | Ar-5 |
| Ar-4 | Ar-4 | Ar-2 | Ar-6 | Ar-4 | Ar-6 | Ar-3 | Ar-6 |
| Ar-4 | Ar-4 | Ar-3 | Ar-5 | Ar-4 | Ar-6 | Ar-4 | Ar-5 |
| Ar-4 | Ar-4 | Ar-3 | Ar-6 | Ar-4 | Ar-6 | Ar-4 | Ar-6 |
| Ar-4 | Ar-4 | Ar-4 | Ar-4 | Ar-4 | Ar-6 | Ar-5 | Ar-5 |
| Ar-4 | Ar-4 | Ar-4 | Ar-5 | Ar-4 | Ar-6 | Ar-5 | Ar-6 |
| Ar-4 | Ar-4 | Ar-4 | Ar-6 | Ar-4 | Ar-6 | Ar-6 | Ar-4 |
| Ar-4 | Ar-4 | Ar-5 | Ar-4 | Ar-4 | Ar-6 | Ar-6 | Ar-5 |
| Ar-4 | Ar-4 | Ar-5 | Ar-5 | Ar-4 | Ar-6 | Ar-6 | Ar-6 |
| Ar-4 | Ar-4 | Ar-5 | Ar-6 | Ar-5 | Ar-1 | Ar-1 | Ar-5 |
| Ar-4 | Ar-4 | Ar-6 | Ar-4 | Ar-5 | Ar-1 | Ar-1 | Ar-6 |
| Ar-4 | Ar-4 | Ar-6 | Ar-5 | Ar-5 | Ar-1 | Ar-2 | Ar-5 |
| Ar-4 | Ar-4 | Ar-6 | Ar-6 | Ar-5 | Ar-1 | Ar-2 | Ar-6 |
| Ar-4 | Ar-5 | Ar-1 | Ar-5 | Ar-5 | Ar-1 | Ar-3 | Ar-5 |
| Ar-4 | Ar-5 | Ar-1 | Ar-6 | Ar-5 | Ar-1 | Ar-3 | Ar-6 |
| Ar-4 | Ar-5 | Ar-2 | Ar-5 | Ar-5 | Ar-1 | Ar-4 | Ar-5 |
| Ar-4 | Ar-5 | Ar-2 | Ar-6 | Ar-5 | Ar-1 | Ar-4 | Ar-6 |
| Ar-4 | Ar-5 | Ar-3 | Ar-5 | Ar-5 | Ar-1 | Ar-5 | Ar-5 |
| Ar-4 | Ar-5 | Ar-3 | Ar-6 | Ar-5 | Ar-1 | Ar-5 | Ar-6 |
| Ar-4 | Ar-5 | Ar-4 | Ar-5 | Ar-5 | Ar-1 | Ar-6 | Ar-5 |
| Ar-4 | Ar-5 | Ar-4 | Ar-6 | Ar-5 | Ar-1 | Ar-6 | Ar-6 |
| Ar-4 | Ar-5 | Ar-5 | Ar-4 | Ar-5 | Ar-2 | Ar-1 | Ar-6 |
| Ar-4 | Ar-5 | Ar-5 | Ar-5 | Ar-5 | Ar-2 | Ar-2 | Ar-5 |
| Ar-4 | Ar-5 | Ar-5 | Ar-6 | Ar-5 | Ar-2 | Ar-2 | Ar-6 |
| Ar-4 | Ar-5 | Ar-6 | Ar-4 | Ar-5 | Ar-2 | Ar-3 | Ar-5 |
| Ar-4 | Ar-5 | Ar-6 | Ar-5 | Ar-5 | Ar-2 | Ar-3 | Ar-6 |

TABLE 1-11

| R1 | R3 | R4 | R6 | R1 | R3 | R4 | R6 |
|---|---|---|---|---|---|---|---|
| Ar-5 | Ar-2 | Ar-4 | Ar-5 | Ar-5 | Ar-5 | Ar-6 | Ar-5 |
| Ar-5 | Ar-2 | Ar-4 | Ar-6 | Ar-5 | Ar-5 | Ar-6 | Ar-6 |
| Ar-5 | Ar-2 | Ar-5 | Ar-5 | Ar-5 | Ar-6 | Ar-1 | Ar-6 |
| Ar-5 | Ar-2 | Ar-5 | Ar-6 | Ar-5 | Ar-6 | Ar-2 | Ar-6 |
| Ar-5 | Ar-2 | Ar-6 | Ar-5 | Ar-5 | Ar-6 | Ar-3 | Ar-6 |
| Ar-5 | Ar-2 | Ar-6 | Ar-6 | Ar-5 | Ar-6 | Ar-4 | Ar-6 |
| Ar-5 | Ar-3 | Ar-1 | Ar-6 | Ar-5 | Ar-6 | Ar-5 | Ar-6 |
| Ar-5 | Ar-3 | Ar-2 | Ar-6 | Ar-5 | Ar-6 | Ar-6 | Ar-5 |
| Ar-5 | Ar-3 | Ar-3 | Ar-5 | Ar-5 | Ar-6 | Ar-6 | Ar-6 |
| Ar-5 | Ar-3 | Ar-3 | Ar-6 | Ar-6 | Ar-1 | Ar-1 | Ar-6 |
| Ar-5 | Ar-3 | Ar-4 | Ar-5 | Ar-6 | Ar-1 | Ar-2 | Ar-6 |
| Ar-5 | Ar-3 | Ar-4 | Ar-6 | Ar-6 | Ar-1 | Ar-3 | Ar-6 |
| Ar-5 | Ar-3 | Ar-5 | Ar-5 | Ar-6 | Ar-1 | Ar-4 | Ar-6 |
| Ar-5 | Ar-3 | Ar-5 | Ar-6 | Ar-6 | Ar-1 | Ar-5 | Ar-6 |
| Ar-5 | Ar-3 | Ar-6 | Ar-5 | Ar-6 | Ar-1 | Ar-6 | Ar-6 |
| Ar-5 | Ar-3 | Ar-6 | Ar-6 | Ar-6 | Ar-2 | Ar-2 | Ar-6 |
| Ar-5 | Ar-4 | Ar-1 | Ar-6 | Ar-6 | Ar-2 | Ar-3 | Ar-6 |
| Ar-5 | Ar-4 | Ar-2 | Ar-6 | Ar-6 | Ar-2 | Ar-4 | Ar-6 |
| Ar-5 | Ar-4 | Ar-3 | Ar-6 | Ar-6 | Ar-2 | Ar-5 | Ar-6 |
| Ar-5 | Ar-4 | Ar-4 | Ar-5 | Ar-6 | Ar-2 | Ar-6 | Ar-6 |
| Ar-5 | Ar-4 | Ar-4 | Ar-6 | Ar-6 | Ar-3 | Ar-3 | Ar-6 |
| Ar-5 | Ar-4 | Ar-5 | Ar-5 | Ar-6 | Ar-3 | Ar-4 | Ar-6 |
| Ar-5 | Ar-4 | Ar-5 | Ar-6 | Ar-6 | Ar-3 | Ar-5 | Ar-6 |
| Ar-5 | Ar-4 | Ar-6 | Ar-5 | Ar-6 | Ar-3 | Ar-6 | Ar-6 |
| Ar-5 | Ar-4 | Ar-6 | Ar-6 | Ar-6 | Ar-4 | Ar-4 | Ar-6 |
| Ar-5 | Ar-5 | Ar-1 | Ar-6 | Ar-6 | Ar-4 | Ar-5 | Ar-6 |
| Ar-5 | Ar-5 | Ar-2 | Ar-6 | Ar-6 | Ar-4 | Ar-6 | Ar-6 |
| Ar-5 | Ar-5 | Ar-3 | Ar-6 | Ar-6 | Ar-5 | Ar-5 | Ar-6 |
| Ar-5 | Ar-5 | Ar-4 | Ar-6 | Ar-6 | Ar-5 | Ar-6 | Ar-6 |
| Ar-5 | Ar-5 | Ar-5 | Ar-5 | Ar-6 | Ar-6 | Ar-6 | Ar-6 |
| Ar-5 | Ar-5 | Ar-5 | Ar-6 | | | | |

R² and R⁵ each are preferably any of hydrogen, an alkyl group, a carbonyl group, an ester group, and an aryl group. Among these, hydrogen or an alkyl group is preferred from the viewpoint of thermal stability, and hydrogen is more preferable from the viewpoint of the easiness of obtaining a narrow half-value width in a light emission spectrum.

R⁸ and R⁹ each are preferably an alkyl group, an aryl group, a heteroaryl group, fluorine, a fluorine-containing alkyl group, a fluorine-containing heteroaryl group, or a fluorine-containing aryl group. In particular, because of being stable against excitation light and the capability of obtaining higher fluorescence quantum yield, R⁸ and R⁹ each are more preferably fluorine or a fluorine-containing aryl group. R⁸ and R⁹ each are still more preferably fluorine in view of the easiness of synthesis.

Here, the fluorine-containing aryl group is an aryl group containing fluorine, and examples thereof include a fluorophenyl group, a trifluoromethylphenyl group, and pentafluorophenyl group. The fluorine-containing heteroaryl group is a heteroaryl group containing fluorine, and examples thereof include a fluoropyridyl group, a trifluoromethylpyridyl group, and a trifluoropyridyl group. The fluorine-containing alkyl group is an alkyl group containing fluorine, and examples thereof include a trifluoromethyl group and a pentafluoroethyl group.

In general formula (1), X is preferably C—R⁷ from the viewpoint of photostability. When X is C—R⁷, from the viewpoint of preventing flocculation in the film and a decrease in light emission intensity due to the flocculation, R⁷ is preferably a group which is rigid, is small in the degree of freedom of motion, and is difficult to cause flocculation. Specifically, any of a substituted or unsubstituted aryl group and a substituted or unsubstituted heteroaryl group is preferred.

From the viewpoints of giving higher fluorescence quantum yield, being more resistant to thermal decomposition, and photostability, X is preferably C—R⁷ in which R⁷ is a substituted or unsubstituted aryl group. From the viewpoint that a light emission wavelength is not impaired, the aryl group is preferably a phenyl group, a biphenyl group, a terphenyl group, a naphthyl group, a fluorenyl group, a phenanthryl group, or an anthracenyl group.

Further, in order to improve the photostability of the compound represented by general formula (1), the twist of the carbon-carbon bond between R⁷ and the pyrromethene skeleton is required to be appropriately suppressed. This is because an excessively large twist causes a reduction in photostability, such as an increase in reactivity against the excitation light. From these viewpoints, R⁷ is preferably a substituted or unsubstituted phenyl group, a substituted or unsubstituted biphenyl group, a substituted or unsubstituted terphenyl group, or a substituted or unsubstituted naphthyl group, and more preferably a substituted or unsubstituted phenyl group, a substituted or unsubstituted biphenyl group, or a substituted or unsubstituted terphenyl group. Particularly preferred is a substituted or unsubstituted phenyl group.

R⁷ is preferably a moderately bulky substituent. R⁷ has bulkiness to some extent, whereby the flocculation of molecules can be prevented. Consequently, the luminous efficiency and durability are further improved.

More preferred examples of the bulky substituent include the structure of R⁷ represented by general formula (8).

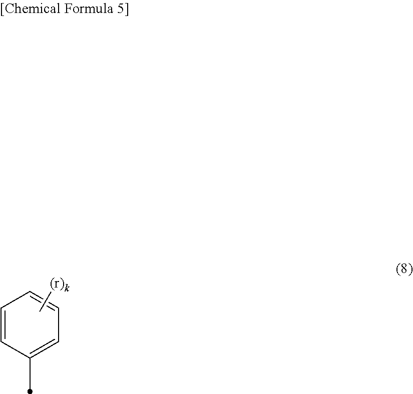

In general formula (8), r is selected from the group consisting of hydrogen, an alkyl group, a cycloalkyl group, a heterocyclic group, an alkenyl group, a cycloalkenyl group, an alkynyl group, a hydroxy group, a thiol group, an alkoxy group, an alkylthio group, an aryl ether group, an aryl thioether group, an aryl group, a heteroaryl group, a halogen, a cyano group, an aldehyde group, a carbonyl group, a carboxy group, an ester group, a carbamoyl group, an amino group, a nitro group, a silyl group, a siloxanyl group, a boryl group, and a phosphine oxide group. The symbol k is an integer of 1 to 3. When k is 2 or more, rs are the same or different from each other.

From the viewpoint that higher fluorescence quantum yield can be given, r is preferably a substituted or unsubstituted aryl group. In particular, preferred examples of the aryl group include a phenyl group and a naphthyl group. When r is an aryl group, k in general formula (8) is preferably 1 or 2, and more preferably 2 from the viewpoint of further preventing the flocculation of molecules. Further, when k is 2 or more, at least one of rs is preferably substituted with an alkyl group. Particularly preferred examples of the alkyl group in this case include a methyl group, an ethyl group, and a tert-butyl group from the viewpoint of thermal stability. More preferred examples thereof include a tert-butyl group.

From the viewpoint of controlling a fluorescence wavelength and an absorption wavelength and improving compatibility with the solvent, r is preferably a substituted or unsubstituted alkyl group, a substituted or unsubstituted alkoxy group, or a halogen, and more preferably a methyl group, an ethyl group, a tert-butyl group, or a methoxy group. From the viewpoint of dispersibility, r is particularly preferably a tert-butyl group or a methoxy group. The fact that r is a tert-butyl group or a methoxy group is more effective for the prevention of quenching caused by the flocculation of molecules.

As another mode of the compound represented by general formula (1), at least one of R¹ to R⁷ is preferably an electron withdrawing group. In particular, preferred is (1) at least one of R¹ to R⁶ being an electron withdrawing group, (2) R⁷ being an electron withdrawing group, or (3) at least one of R¹ to R⁶ being an electron withdrawing group and R⁷ being an electron withdrawing group. The electron withdrawing group is introduced to the pyrromethene skeleton of the compound, whereby the electron density of the pyrromethene skeleton can be significantly reduced. This provides further improved stability of the compound against oxygen. As a result, the durability of the compound can be further improved.

The electron withdrawing group is called also an electron-accepting group, and is an atomic group which attracts an electron from a substituted atomic group by the inductive effect and the resonance effect in the organic electron theory. Examples of the electron withdrawing group include those having a positive value as a substituent constant (σp (para)) of Hammett's Rule. The substituent constant (σp (para)) of Hammett's Rule can be cited from Kagaku Binran Kiso-Hen Revised 5th Edition (II, p. 380).

Although the phenyl group has an example taking a positive value as in the above, the electron withdrawing group does not include the phenyl group in the present invention.

Examples of the electron withdrawing group include —F (σp: +0.06), —Cl (σp: +0.23), —Br (σp: +0.23), —I (σp: +0.18), —CO₂R¹² (σp: +0.45 when R¹² is an ethyl group), —CONH₂ (σp: +0.38), —COR¹² (σp: +0.49 when R¹² is a methyl group), —CF₃ (σp: +0.50), —SO₂R¹² (σp: +0.69 when R¹² is a methyl group, and —NO₂ (σp: +0.81). R¹²s each independently represent a hydrogen atom, a substituted or unsubstituted aromatic hydrocarbon group having ring-forming carbon atoms of 6 to 30, a substituted or unsubstituted heterocyclic group having ring-forming carbon atoms of 5 to 30, a substituted or unsubstituted alkyl group having carbon atoms of 1 to 30, ora substituted or unsubstituted cycloalkyl group having carbon atoms of 1 to 30. Specific examples of these groups include examples similar to those described above.

Preferred examples of the electron withdrawing group include fluorine, a fluorine-containing aryl group, a fluorine-containing heteroaryl group, a fluorine-containing alkyl group, a substituted or unsubstituted acyl group, a substituted or unsubstituted ester group, a substituted or unsubstituted amide group, a substituted or unsubstituted sulfonyl group, or a cyano group. More preferred examples of the electron withdrawing group include fluorine, a fluorine-containing aryl group, a fluorine-containing heteroaryl group, a fluorine-containing alkyl group, and a substituted or unsubstituted ester group. This is because they are resistant to chemical decomposition.

One preferred example of the compound represented by general formula (1) include a case in which all R¹, R³, R⁴, and R⁶ are the same or different from each other and are substituted or unsubstituted alkyl groups; X is C—R⁷; and R⁷ is the group represented by general formula (8). In this case, R⁷ is particularly preferably the group represented by general formula (8) in which r is contained as a substituted or unsubstituted phenyl group.

Another preferred example of the compound represented by general formula (1) include a case in which all R¹, R³, R⁴, and R⁶ are the same or different from each other and are selected from Ar-1 to Ar-6 described above; X is C—R⁷; and R⁷ is the group represented by general formula (8). In this case, R⁷ is more preferably the group represented by general formula (8) in which r is contained as a tert-butyl group or a methoxy group, and particularly preferably the group represented by general formula (8) in which r is contained as a methoxy group.

The molecular weight of the compound is not particularly limited, but is preferably 1000 or less, and more preferably 800 or less from the viewpoints of heat resistance and film formability. Further, the molecular weight is more preferably 450 or more from the viewpoint that a sufficiently high sublimation temperature can be given to more stably control a deposition rate. Since the sublimation temperature becomes sufficiently high, contamination in the chamber to be prevented, whereby stable high luminance light emission is exhibited, which easily provides highly efficient light emission.

The compound represented by general formula (1) is not particularly limited, but specific examples thereof include the following.

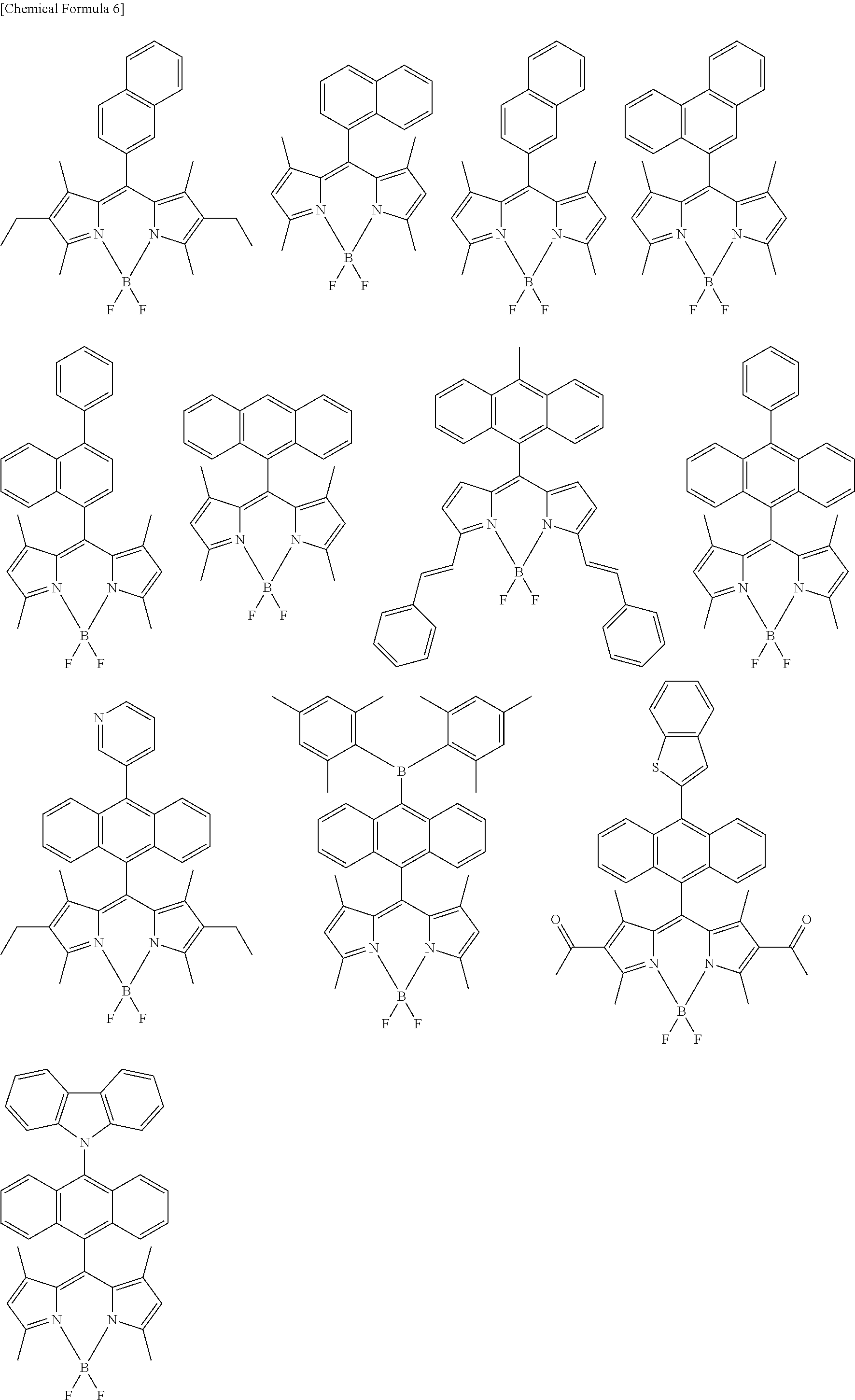

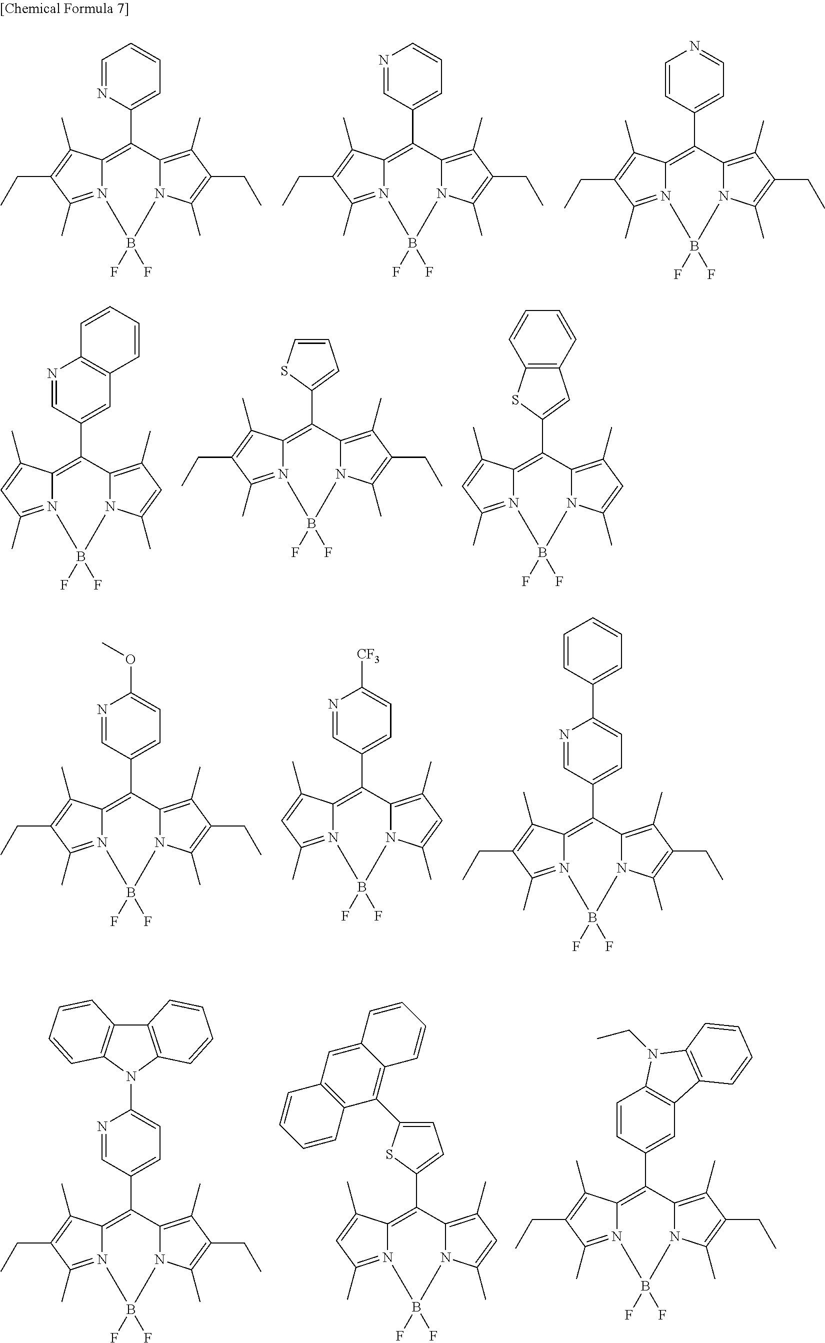

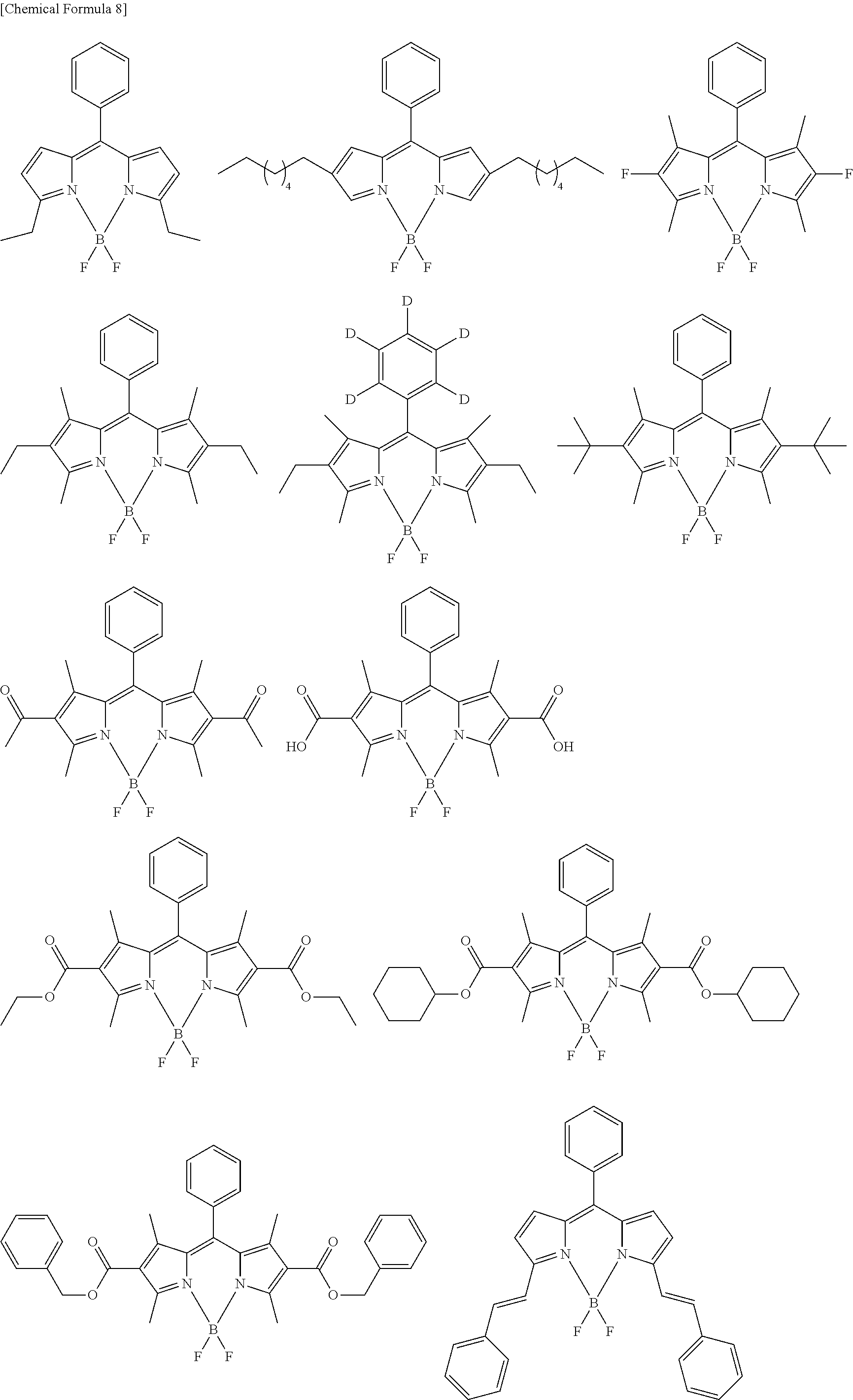

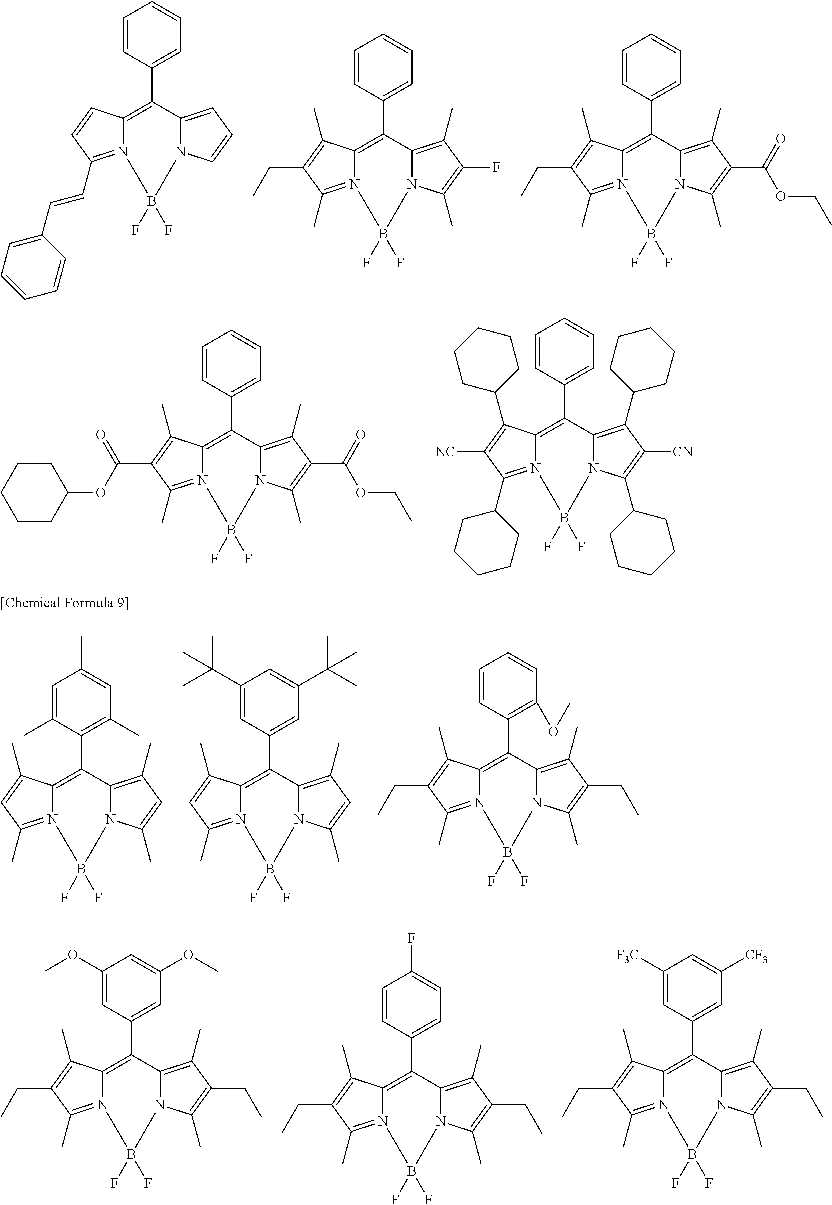

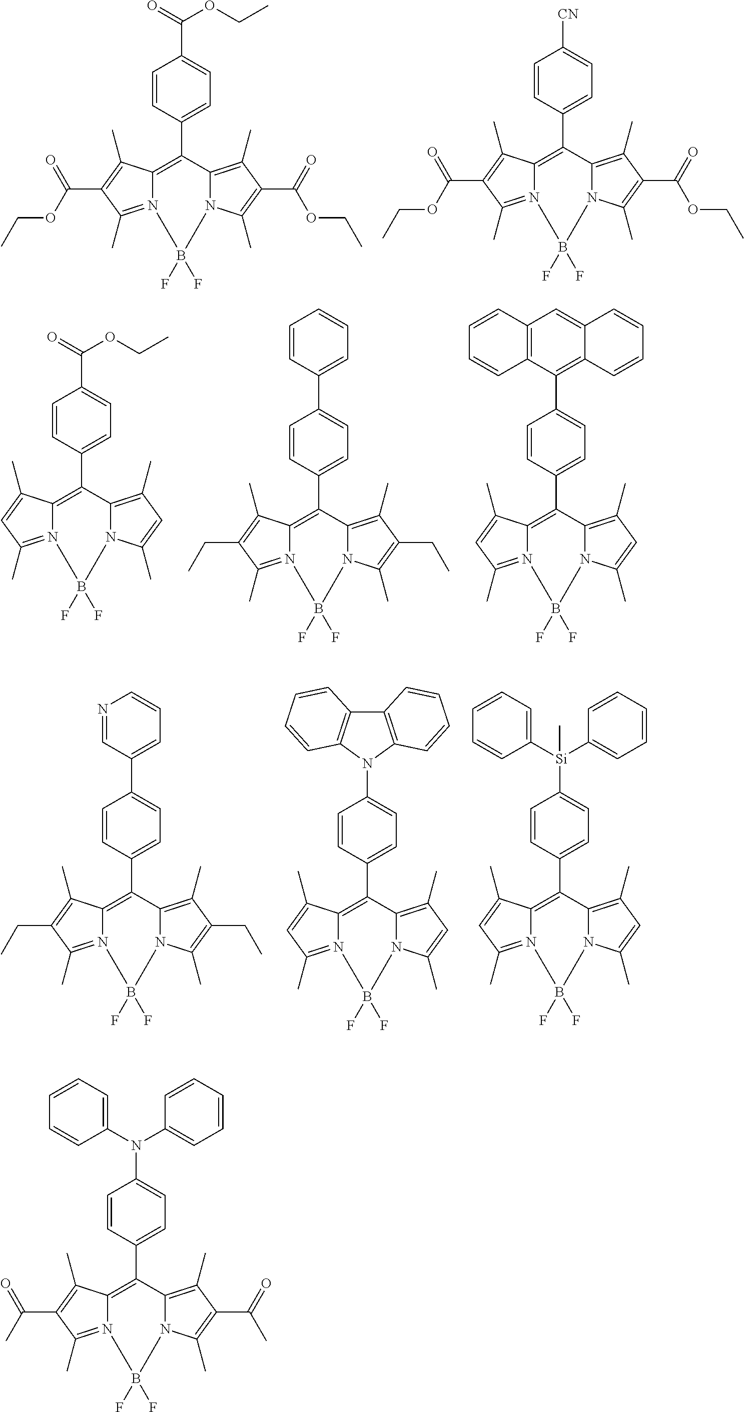

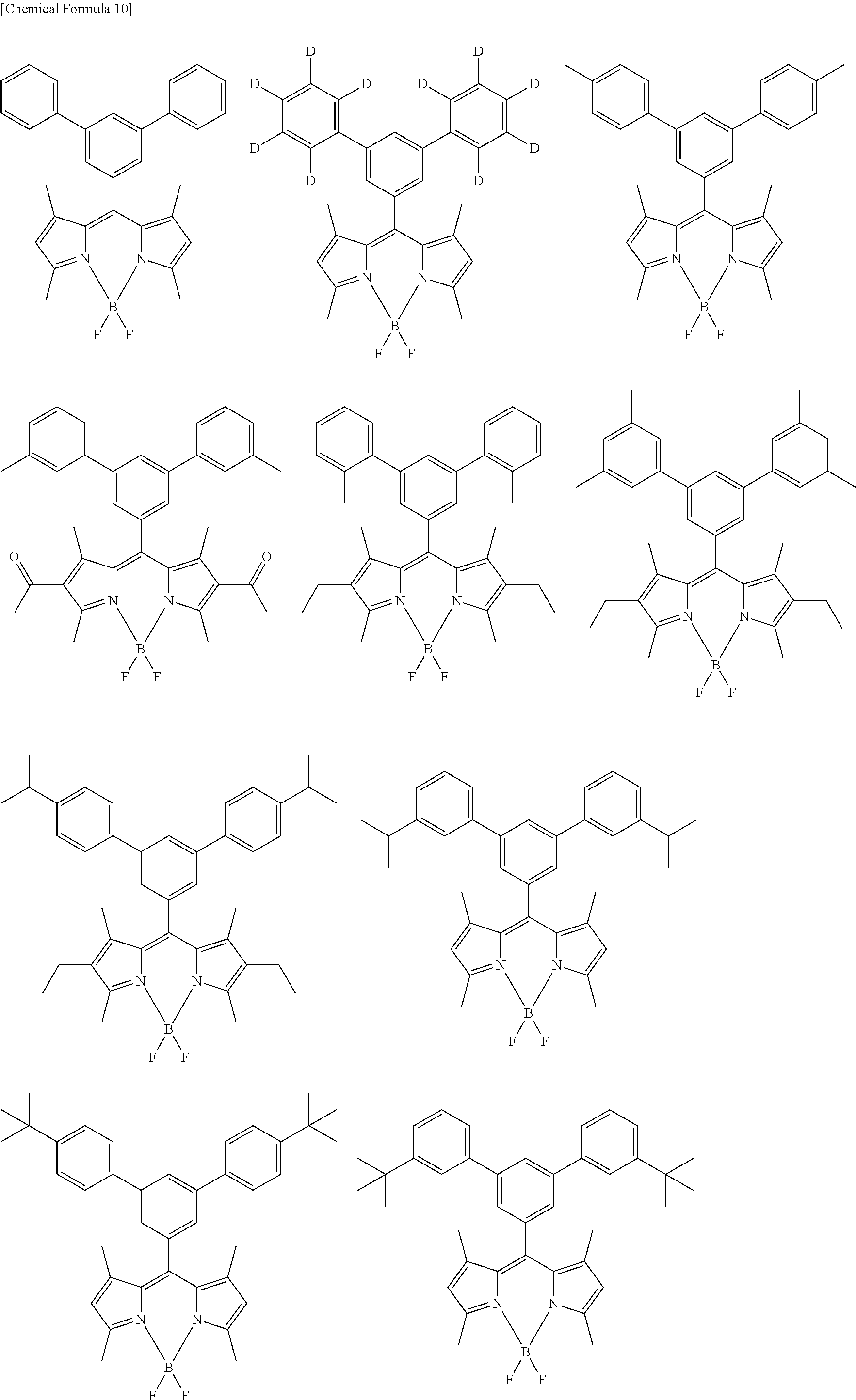

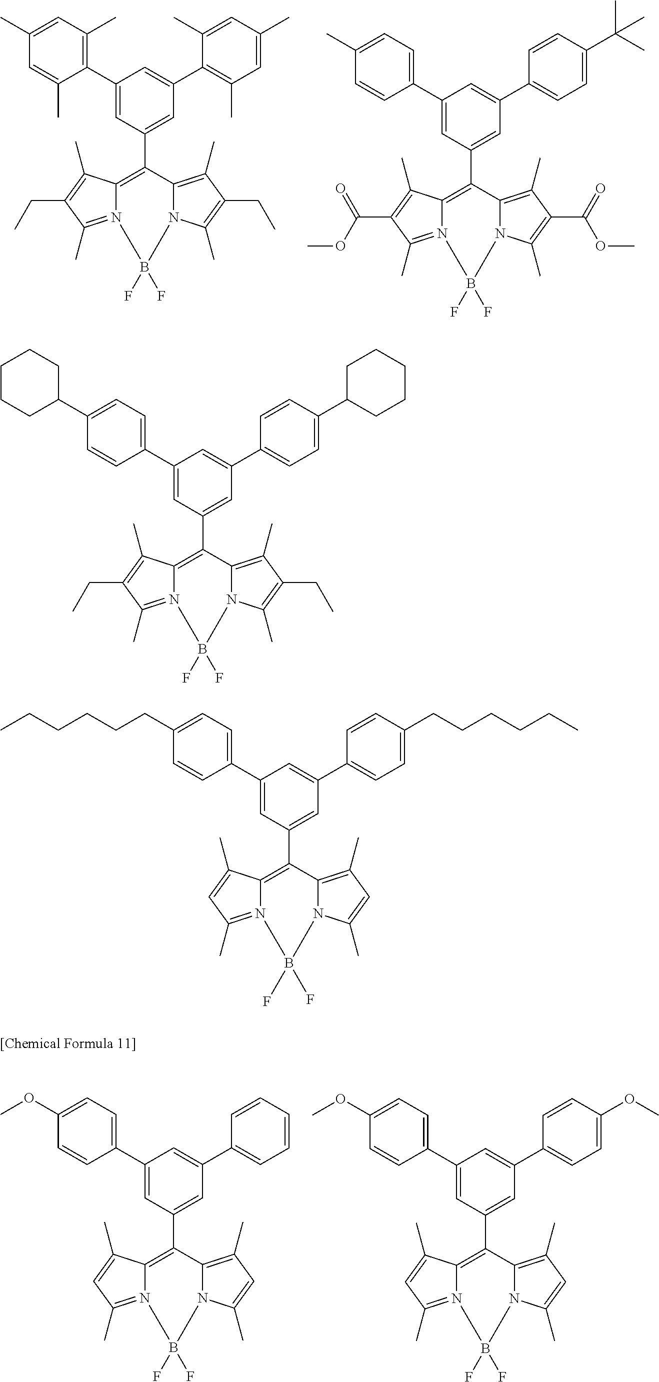

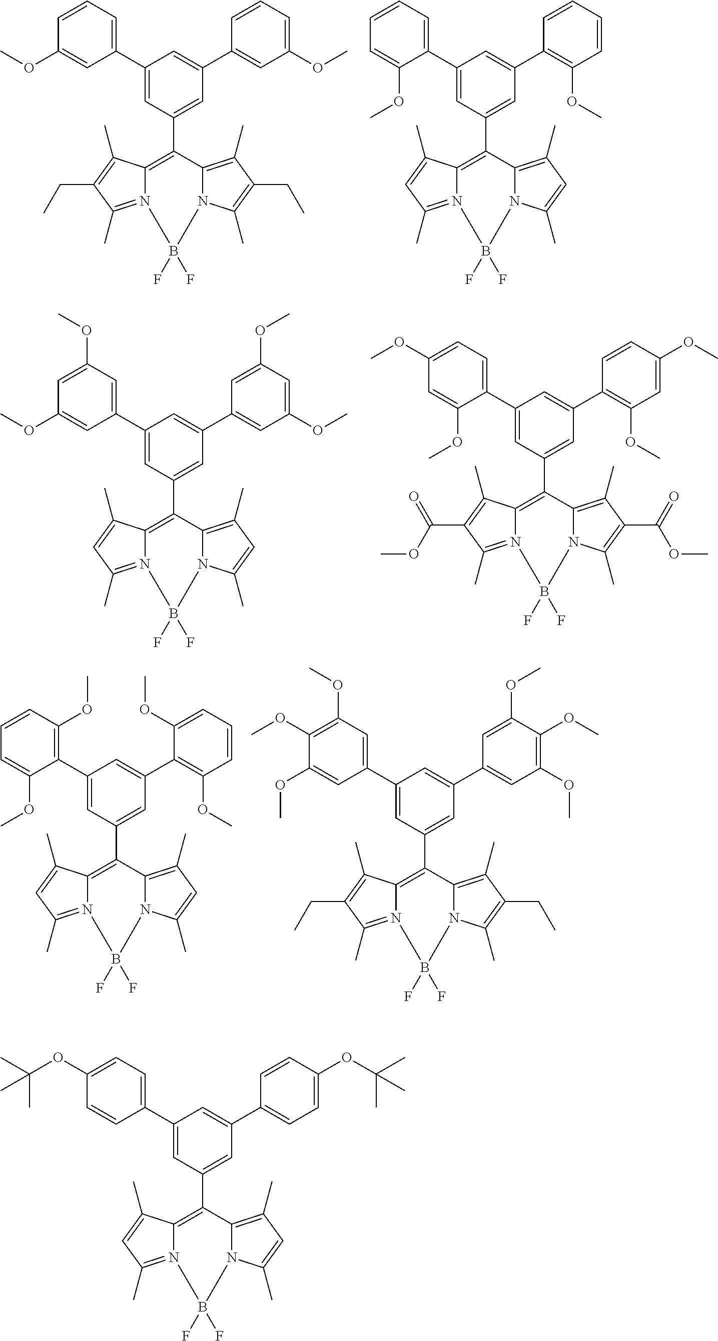

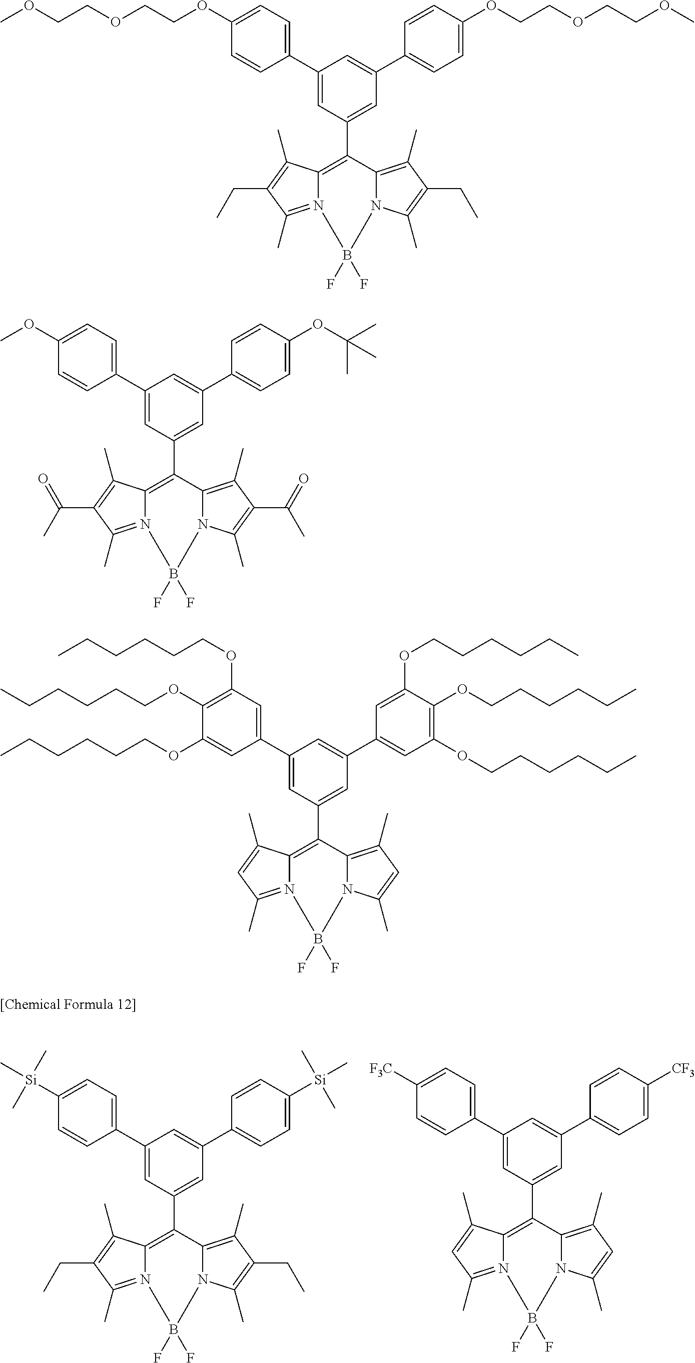

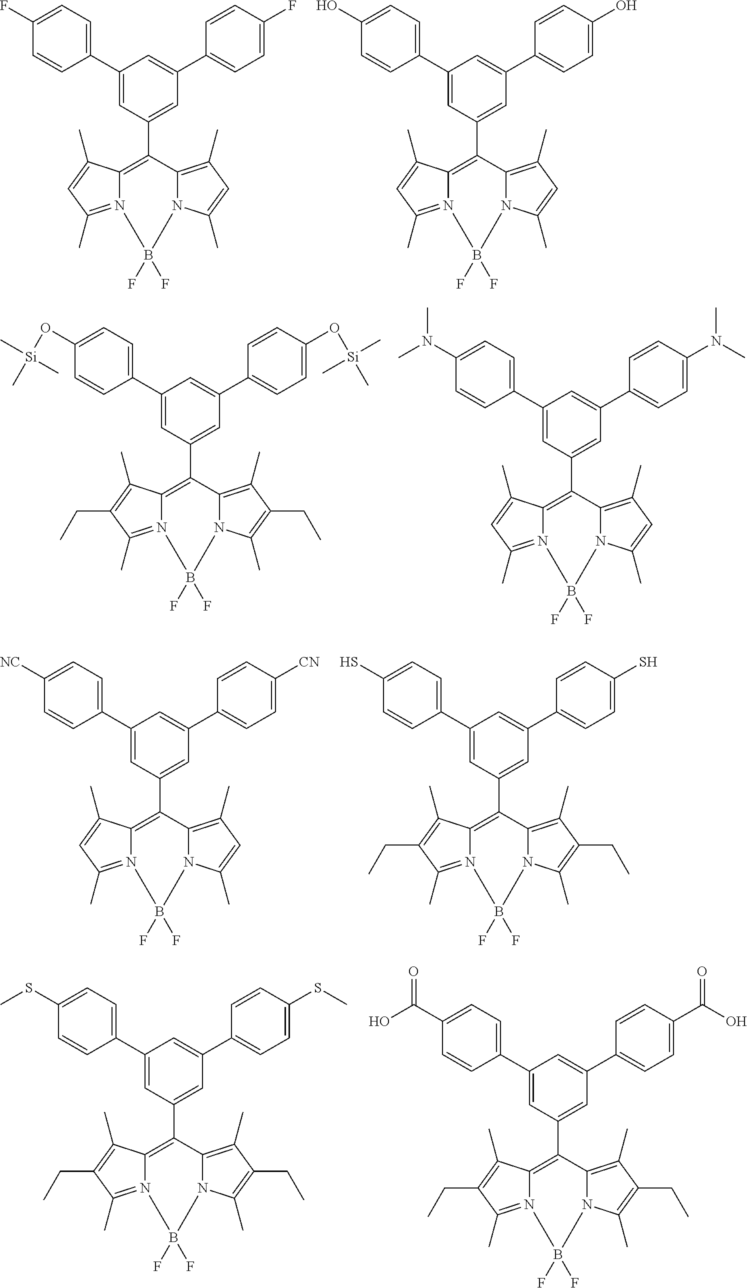

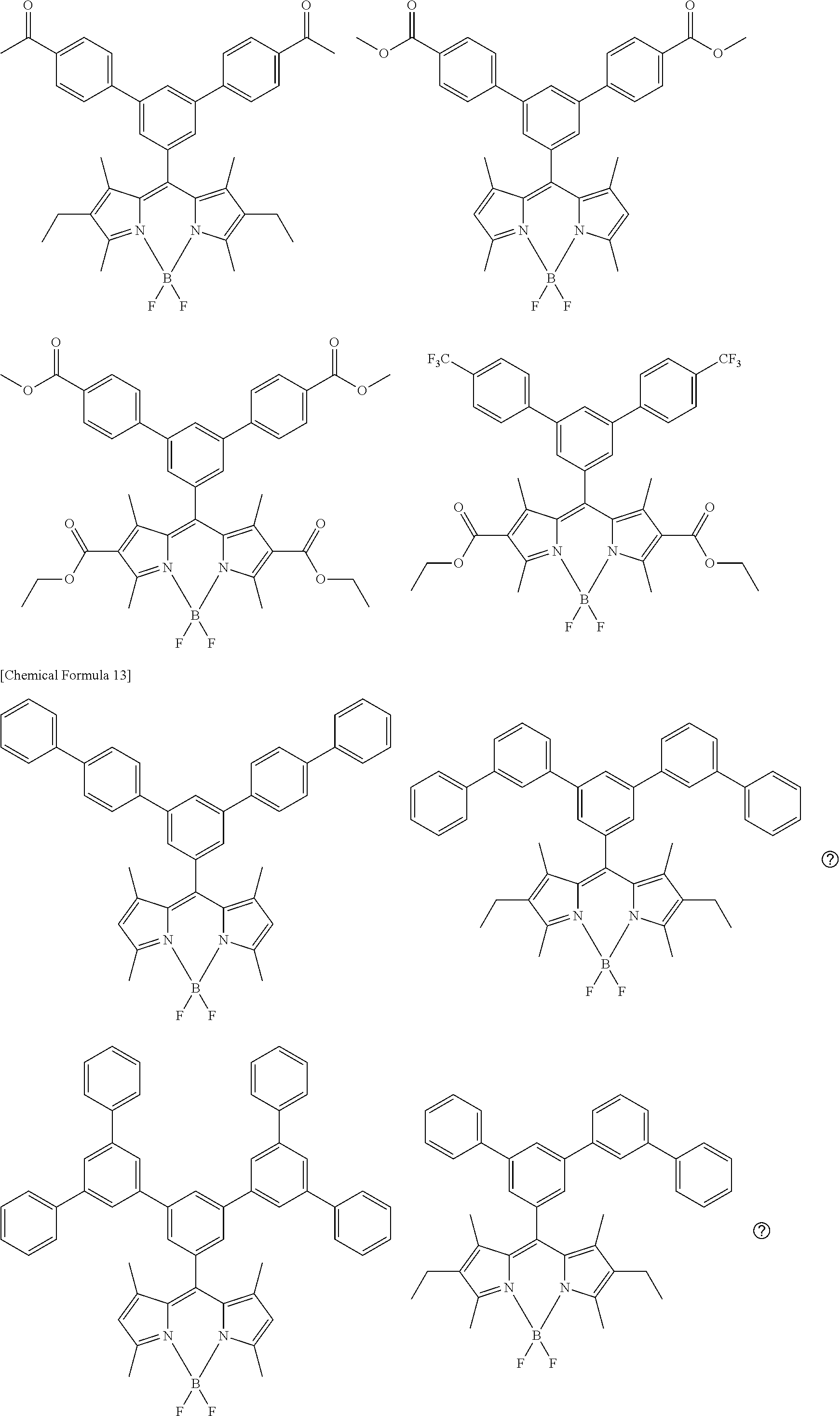

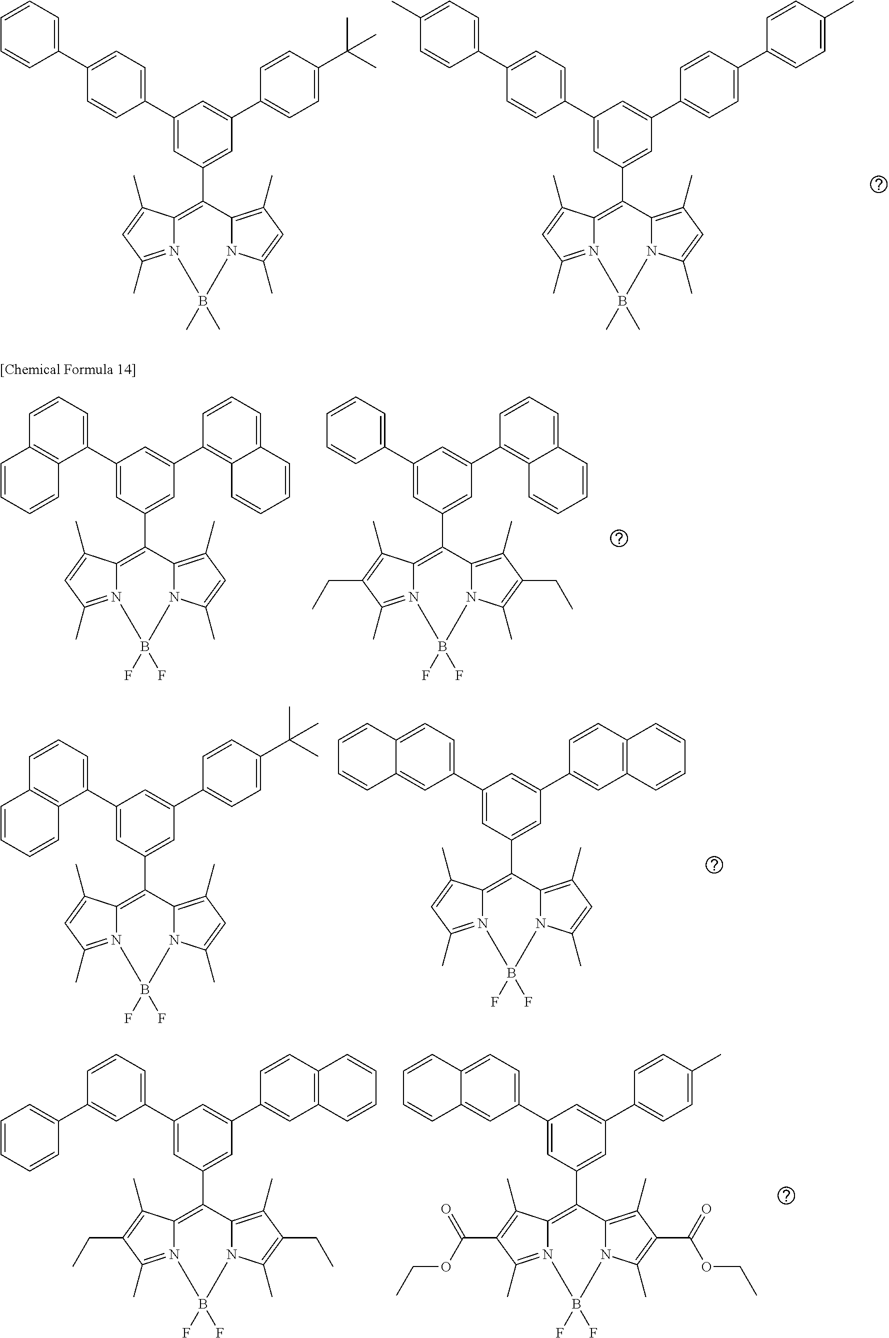

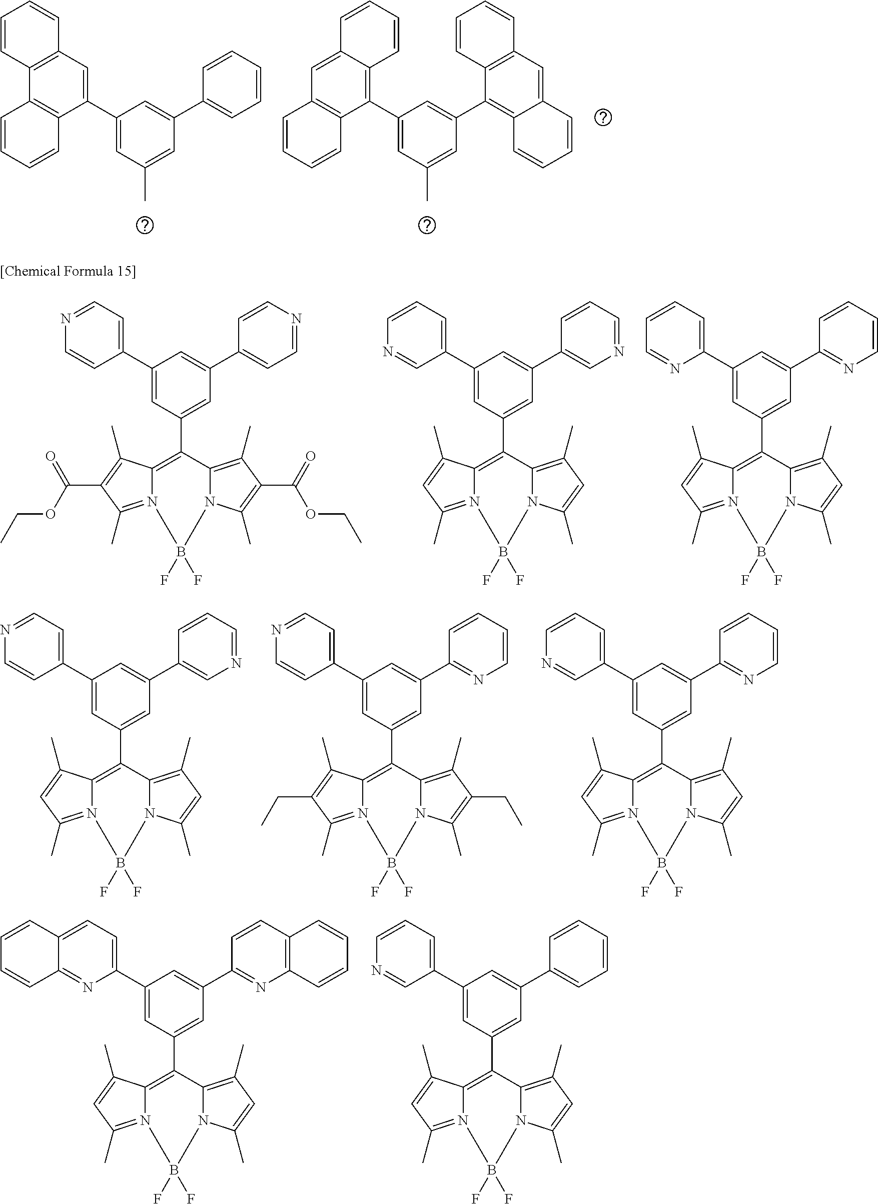

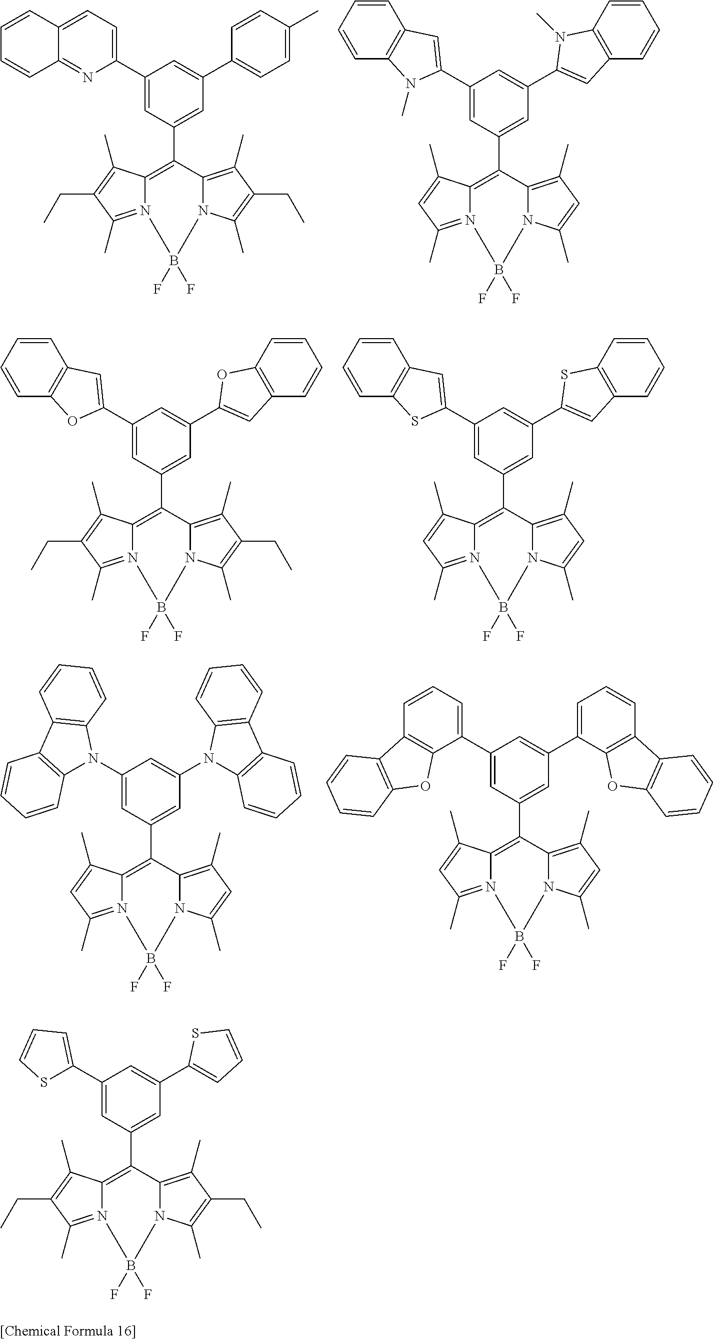

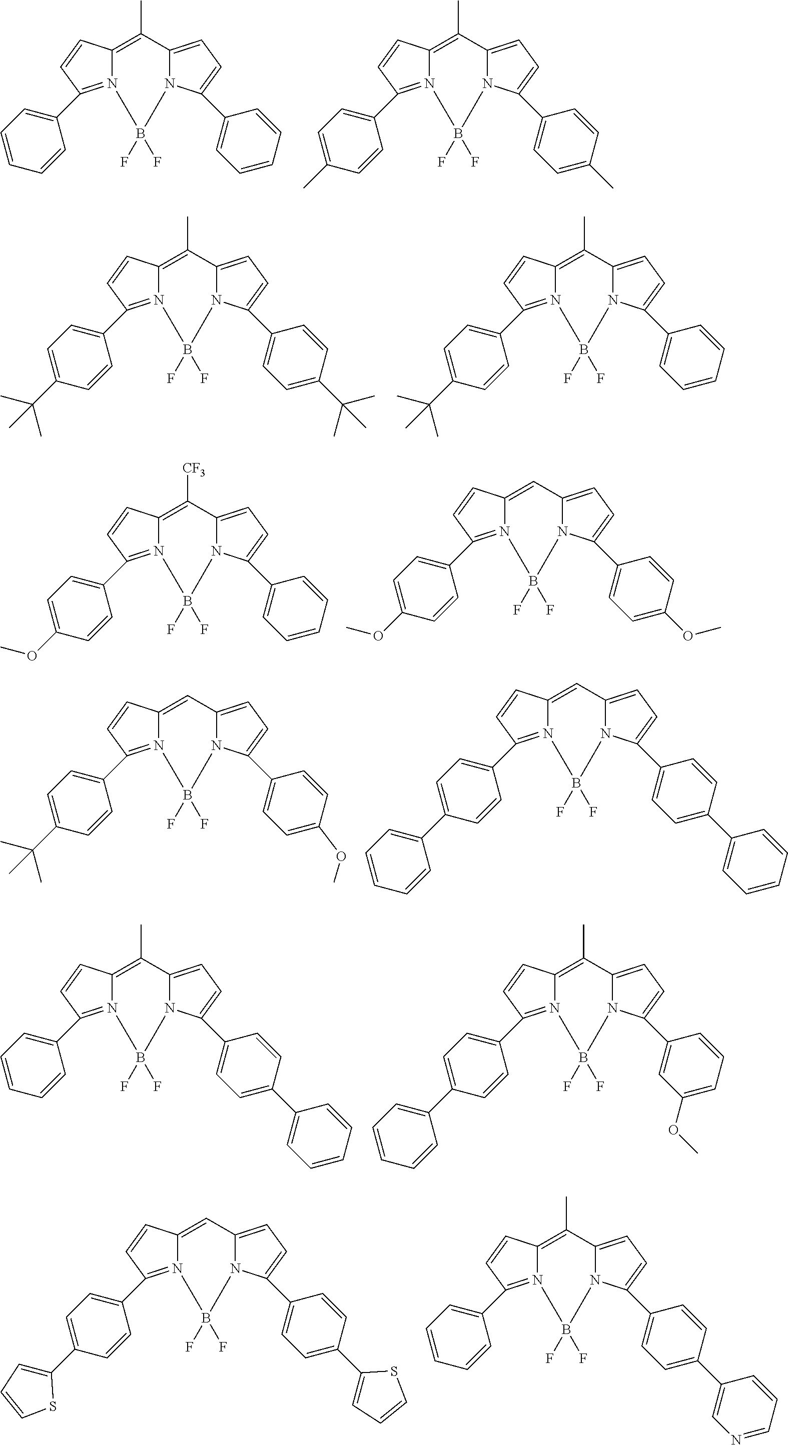

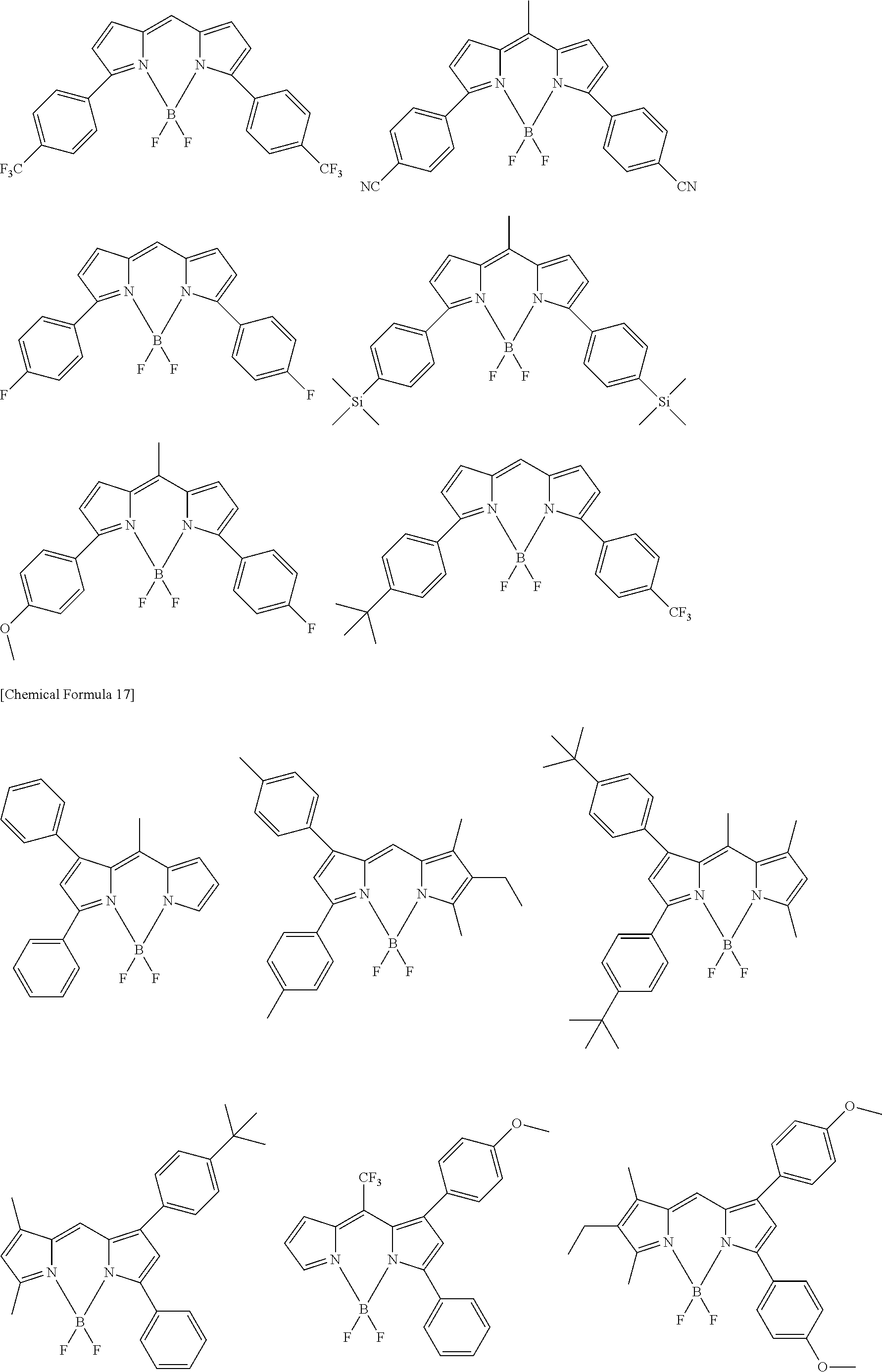

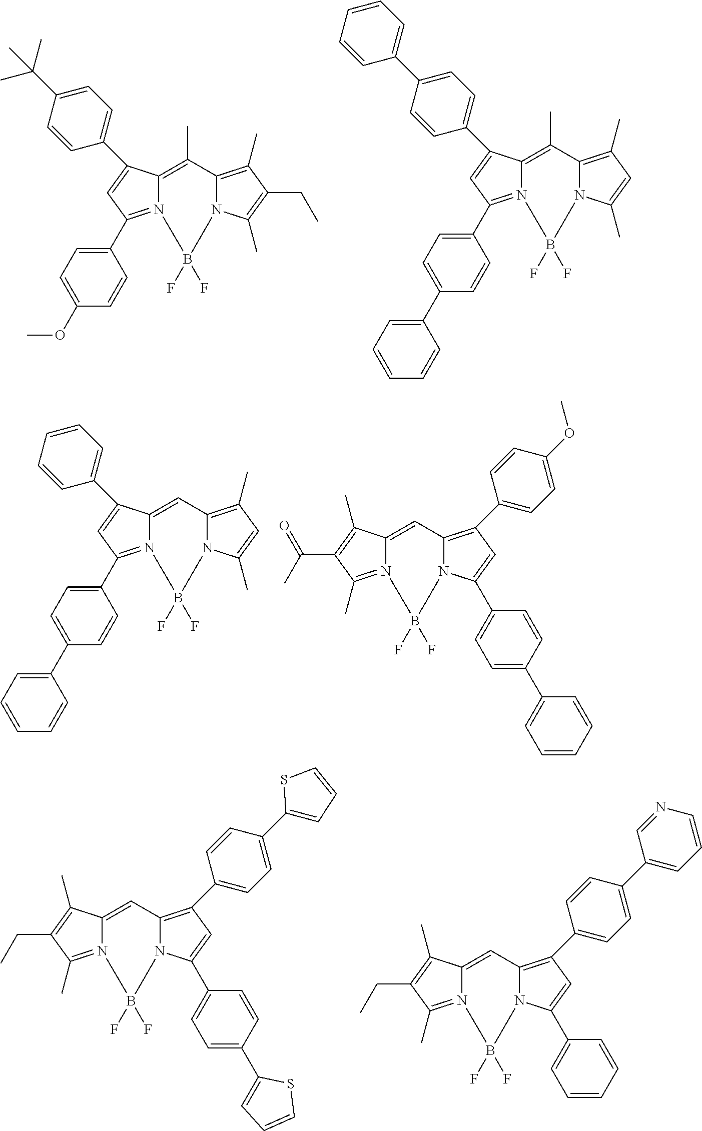

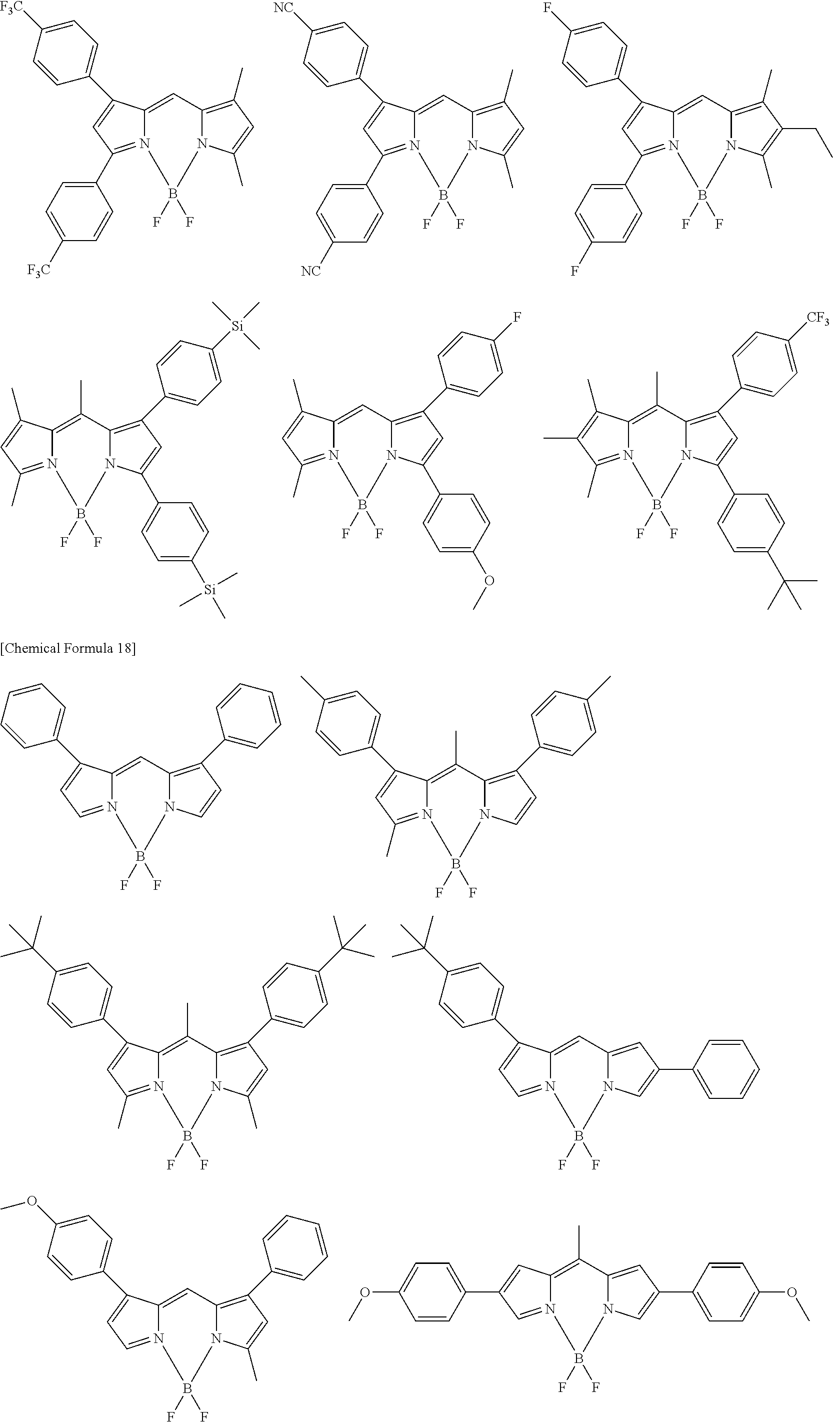

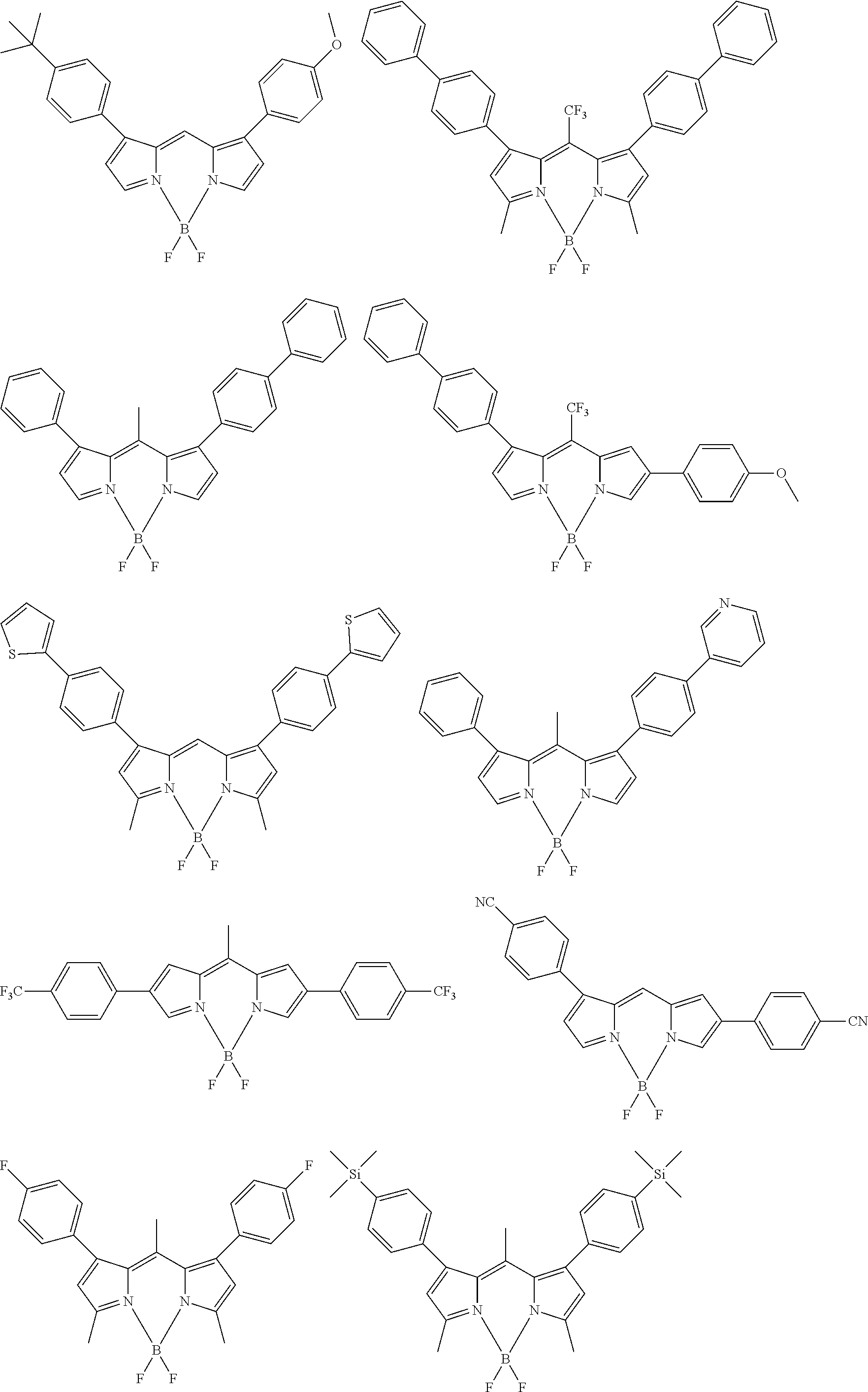

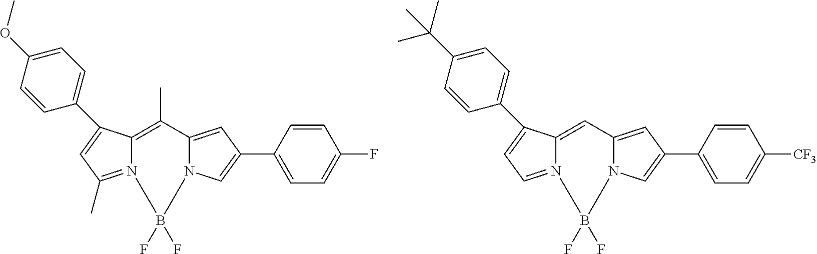

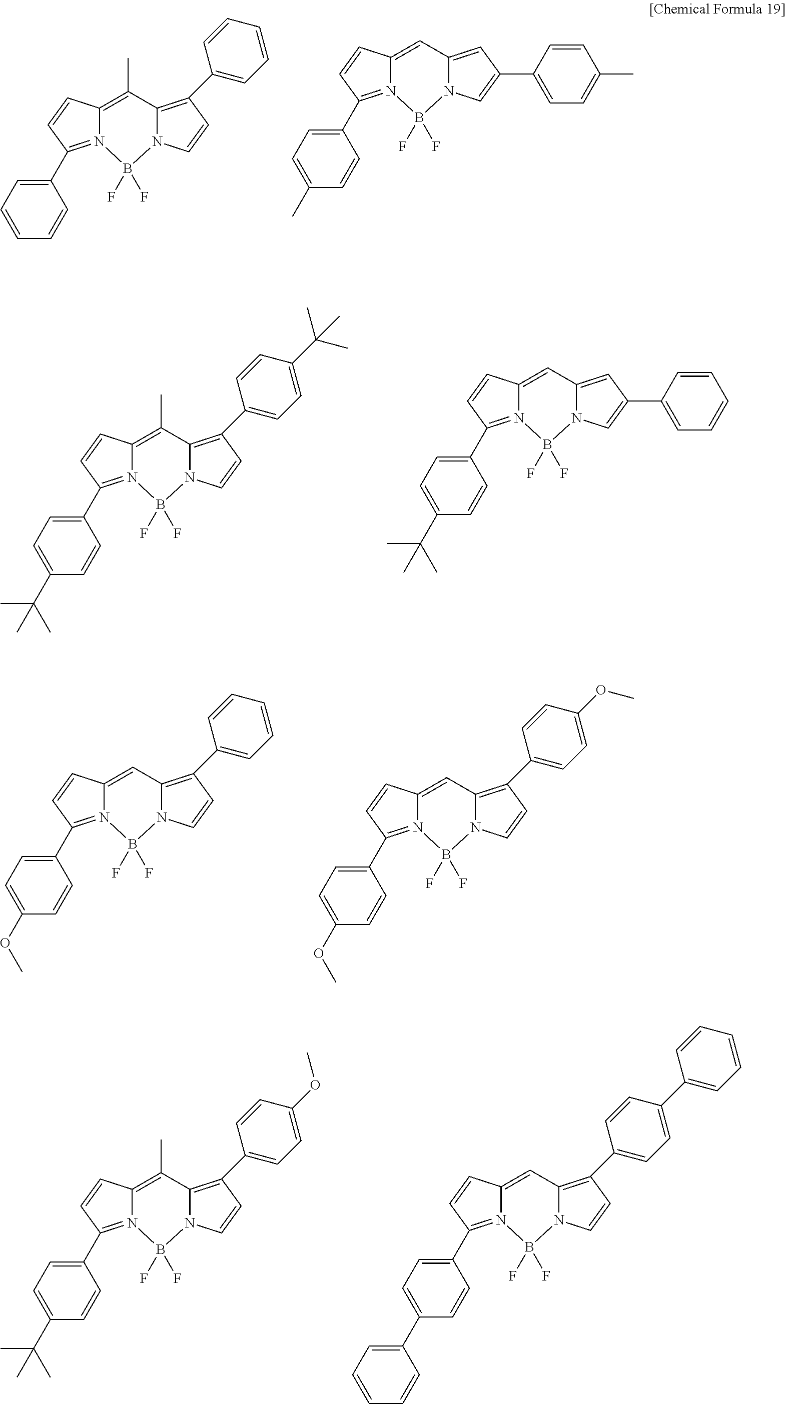

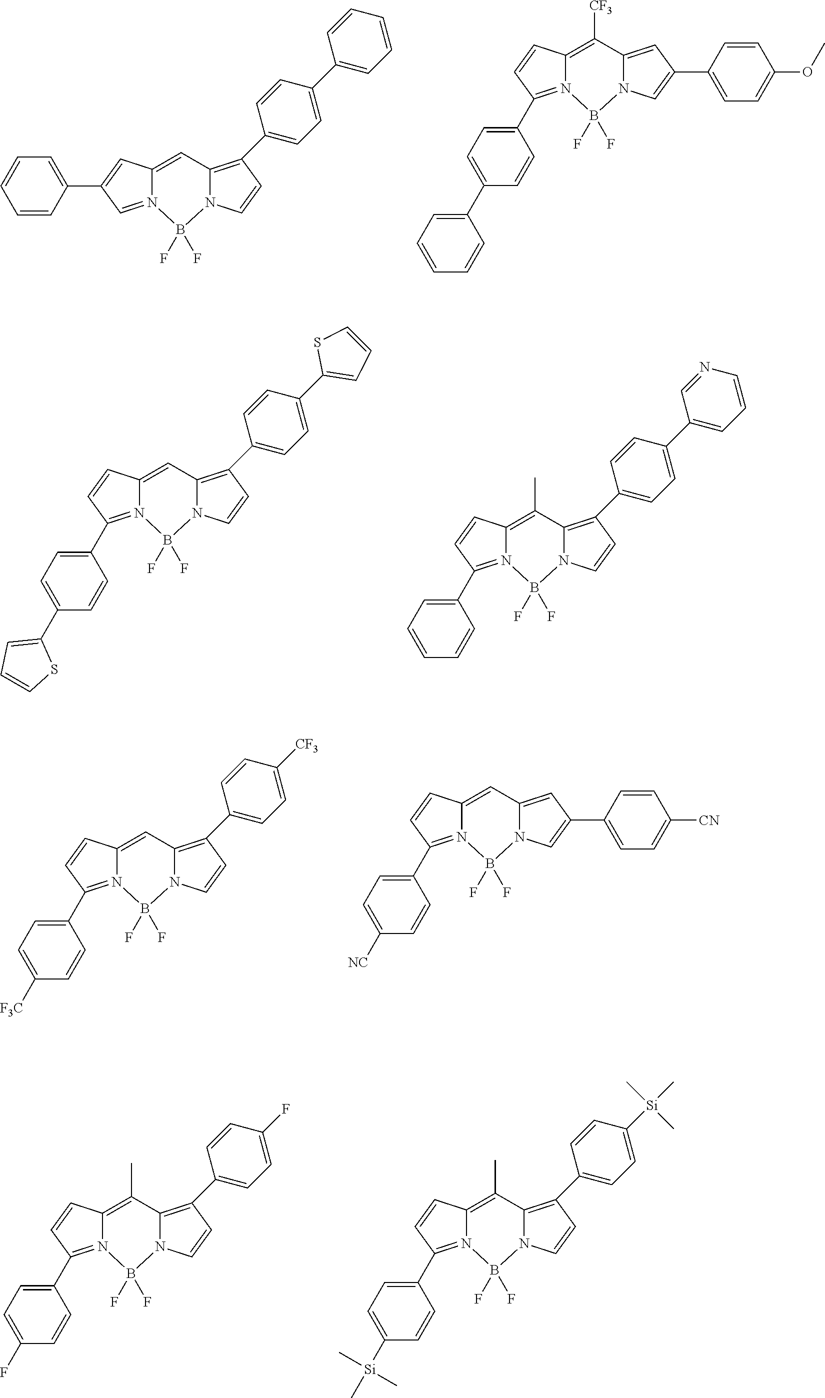

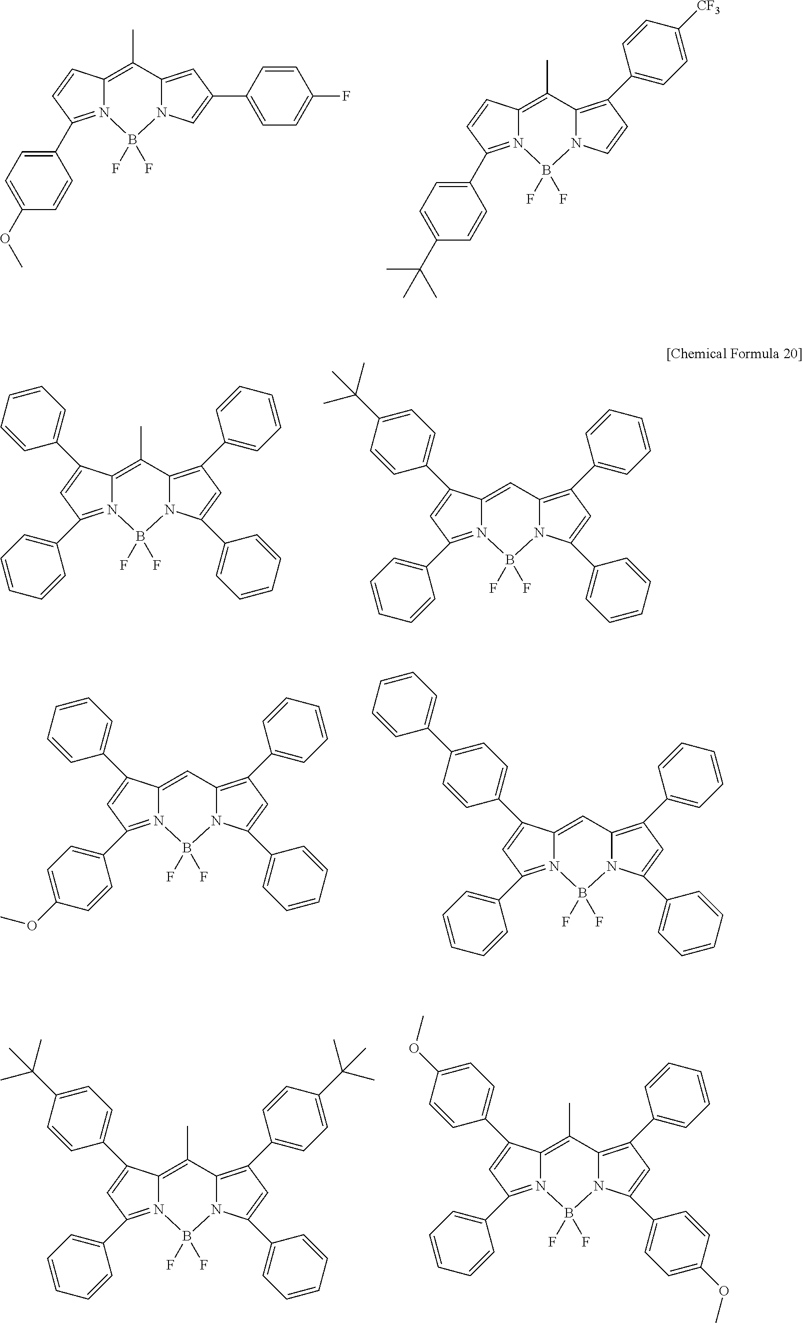

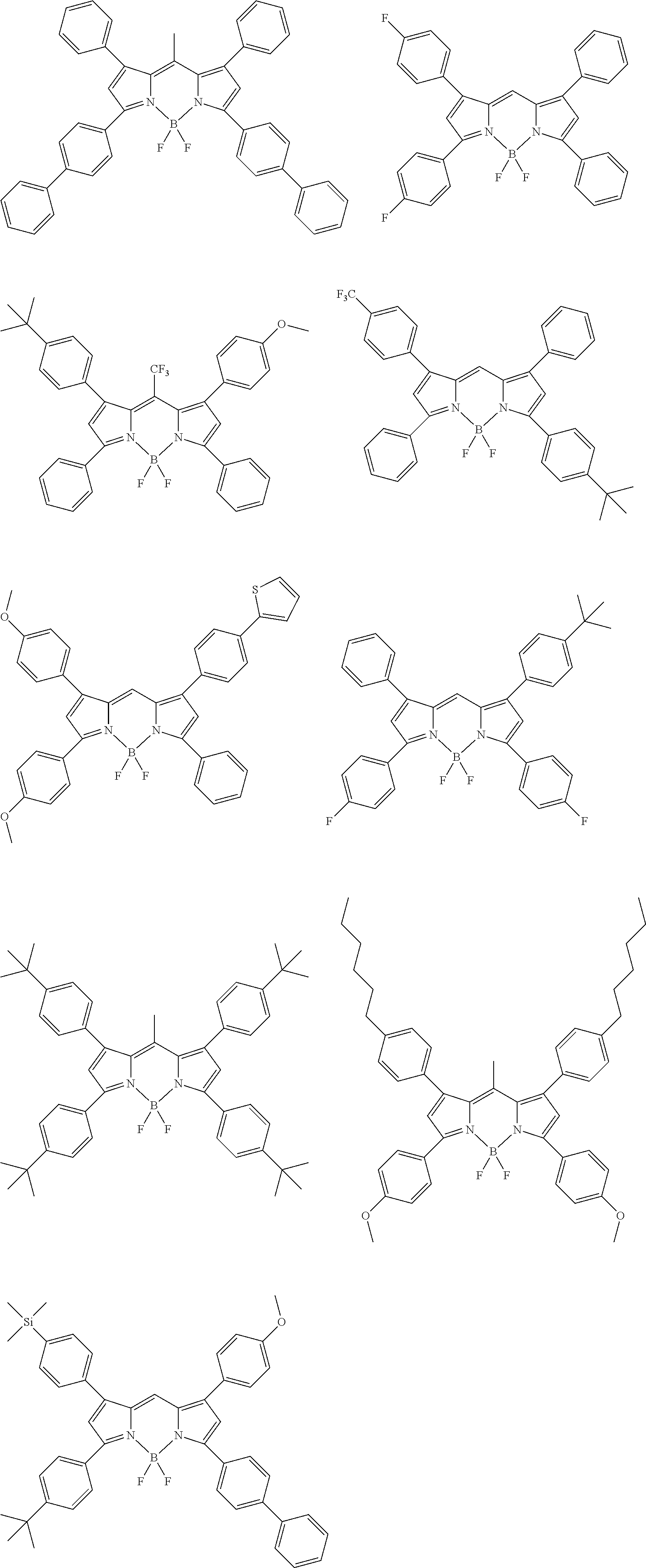

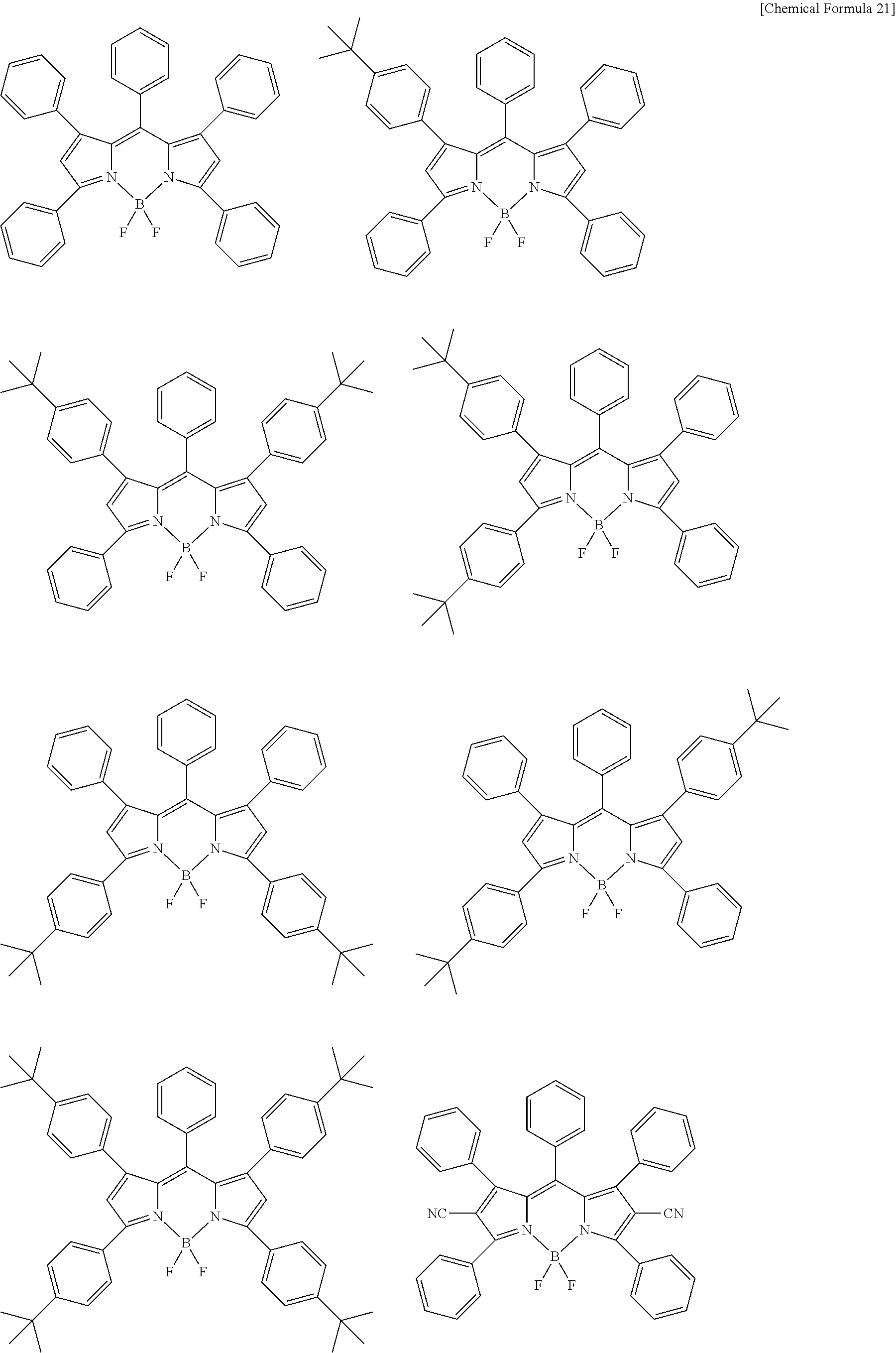

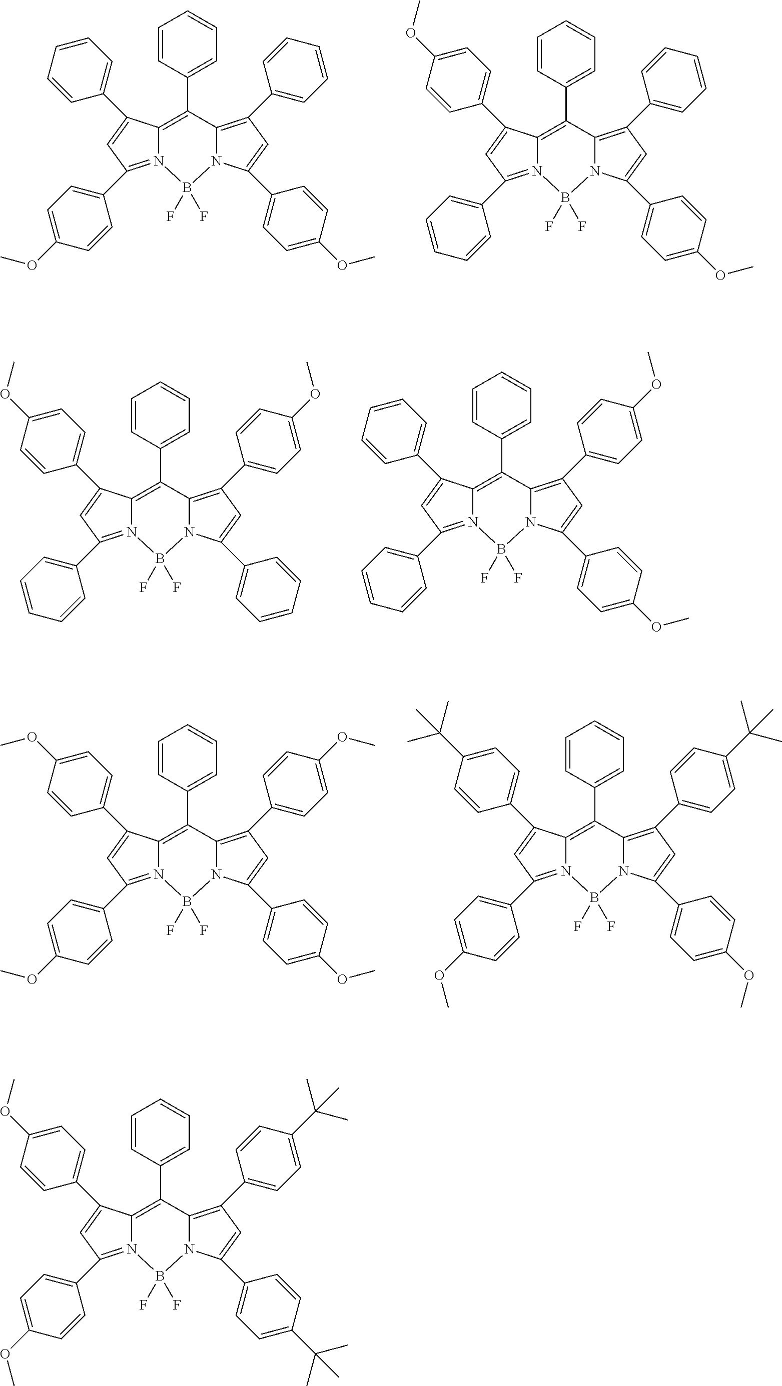

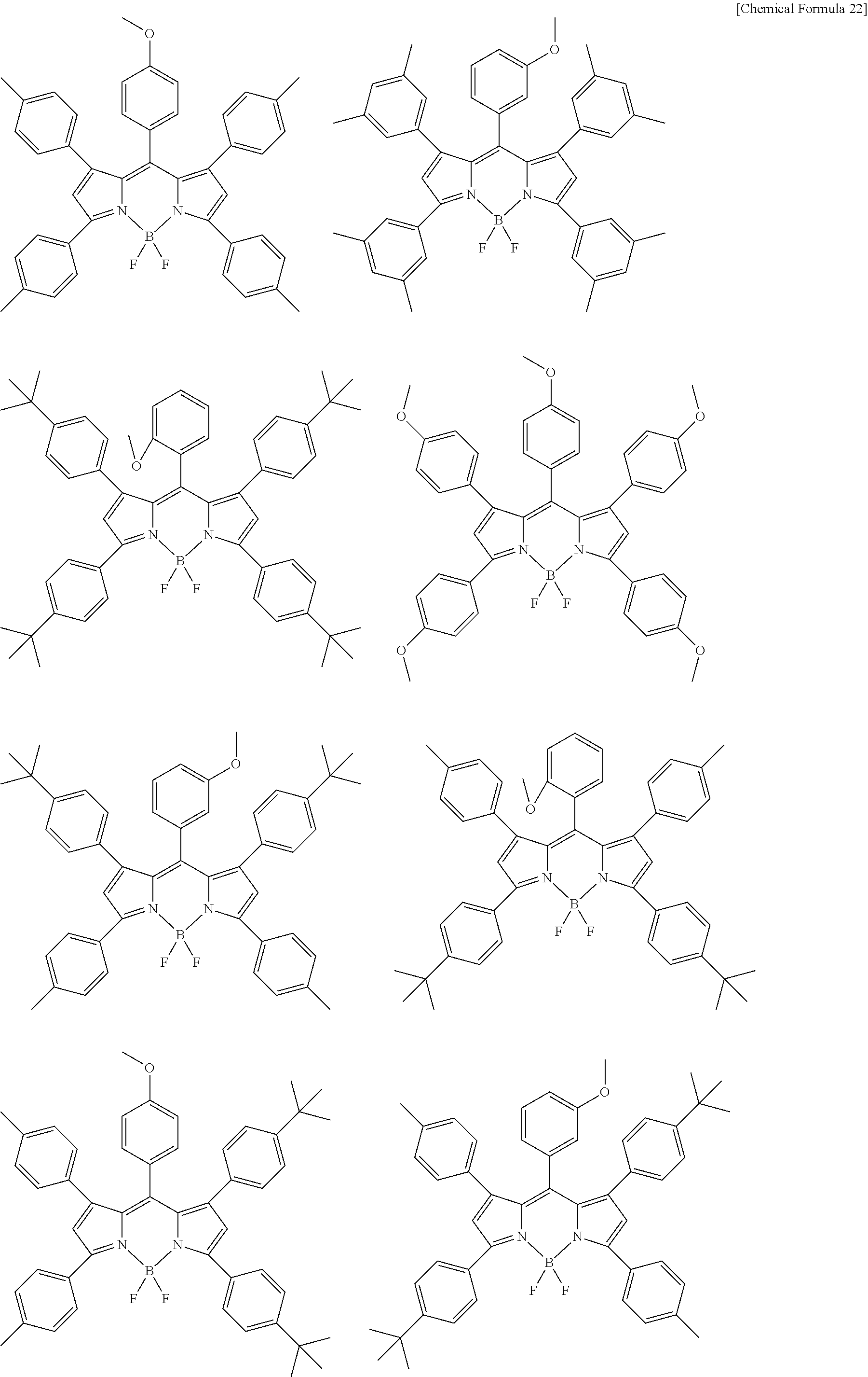

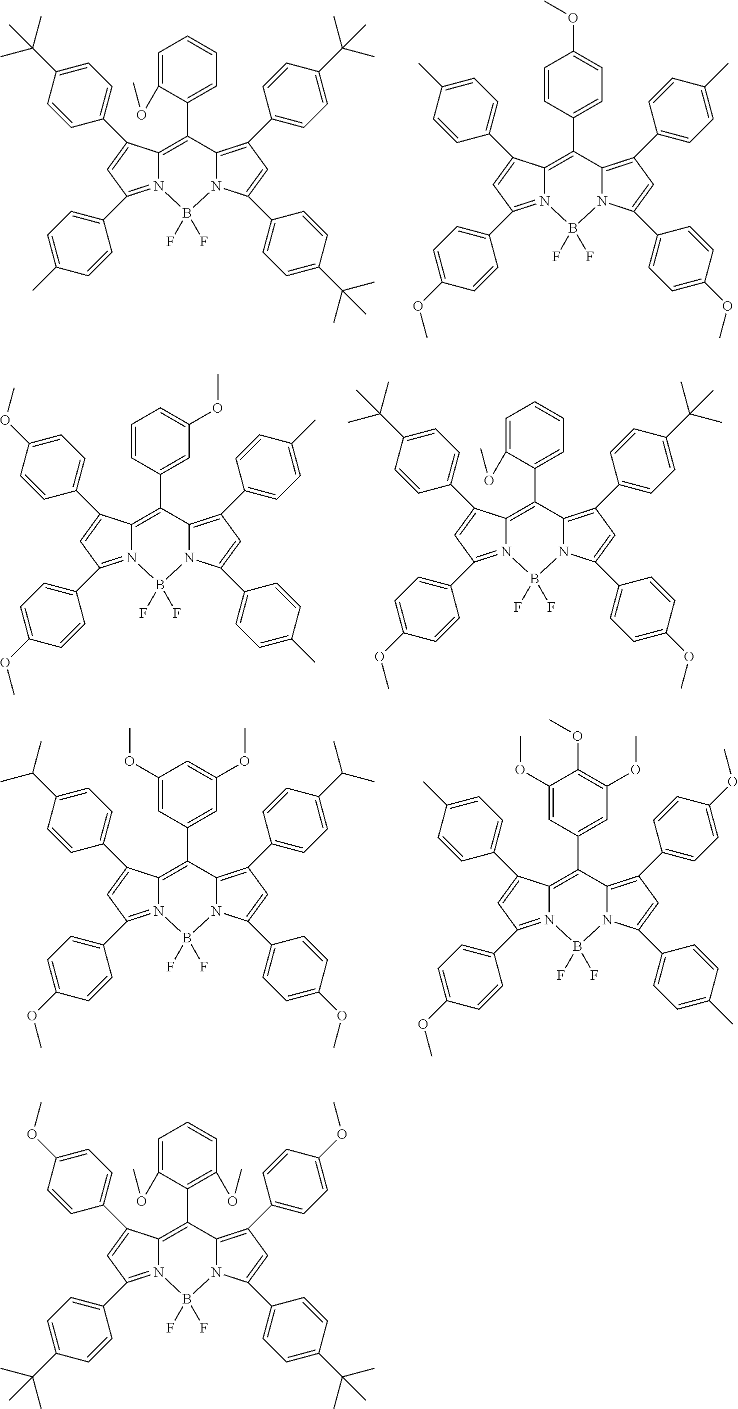

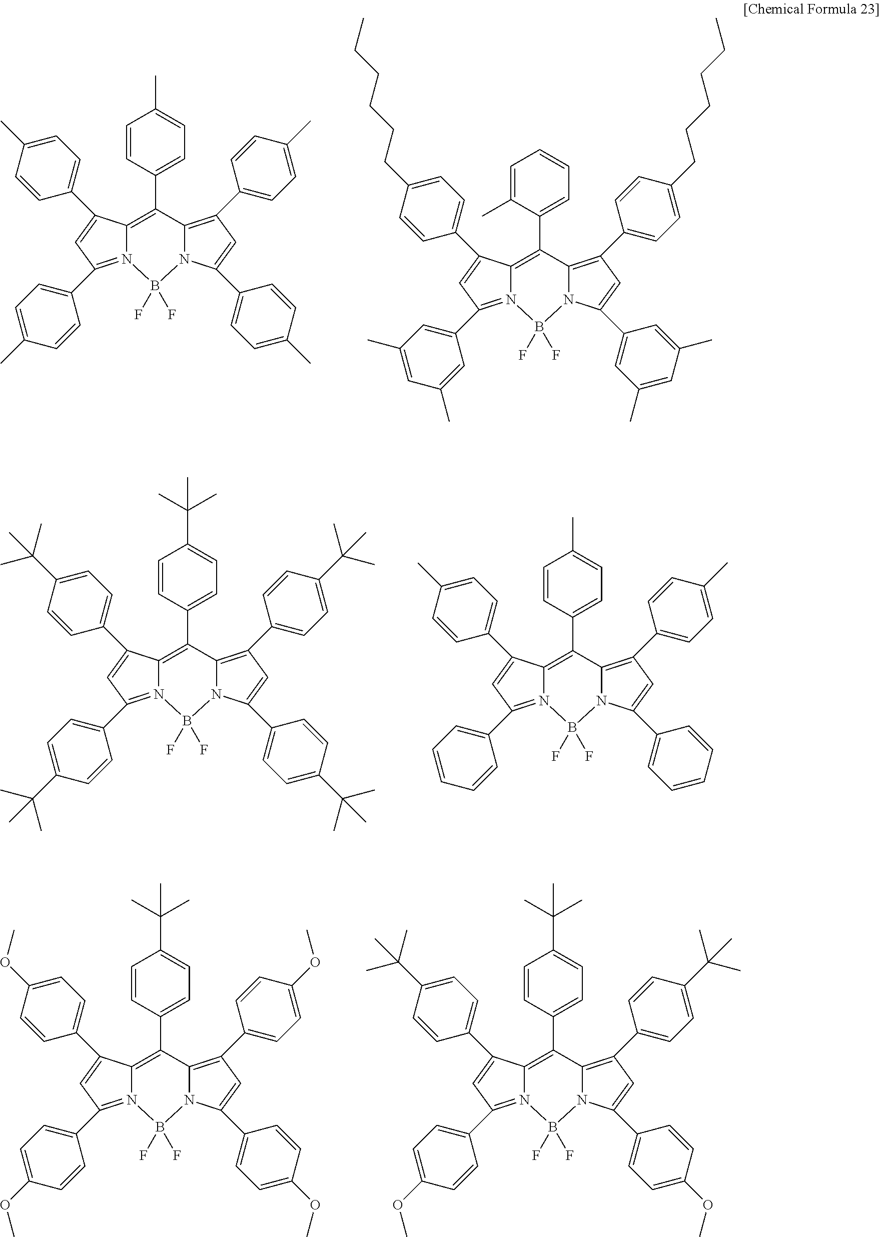

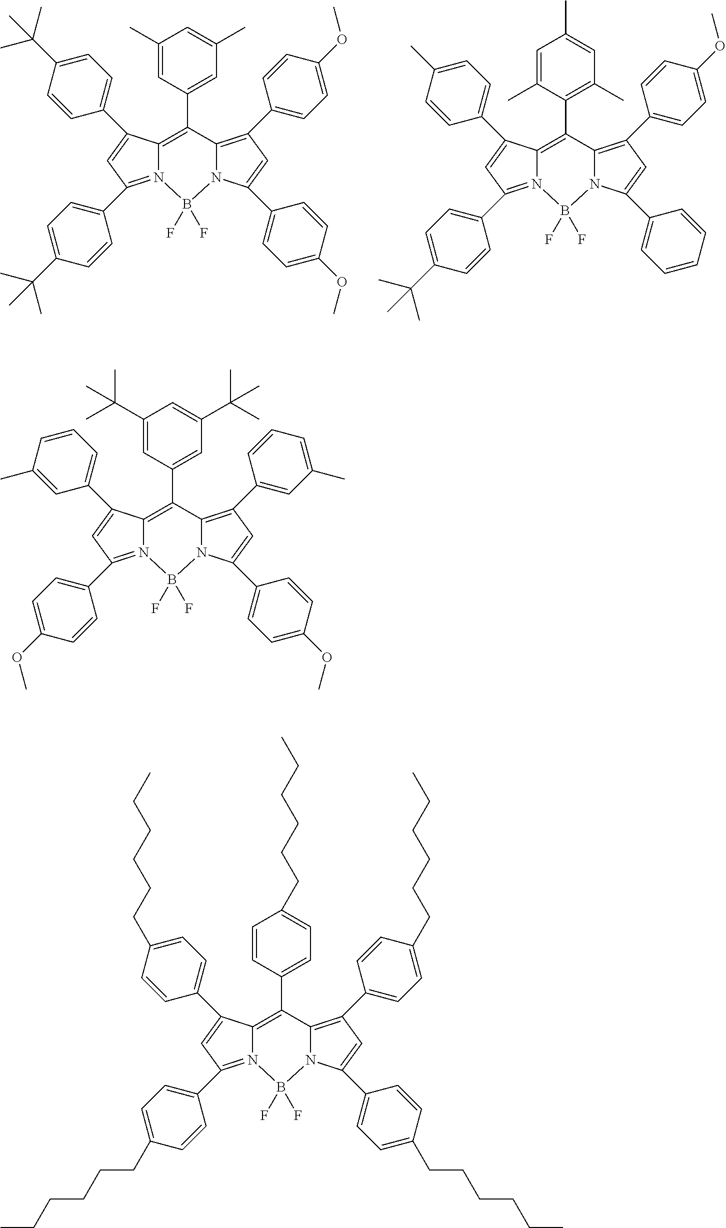

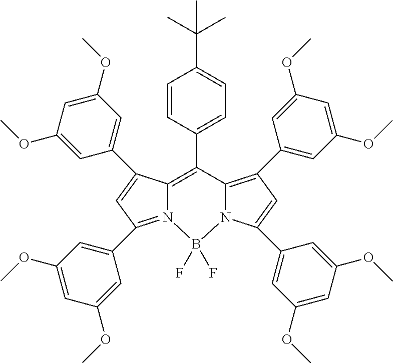

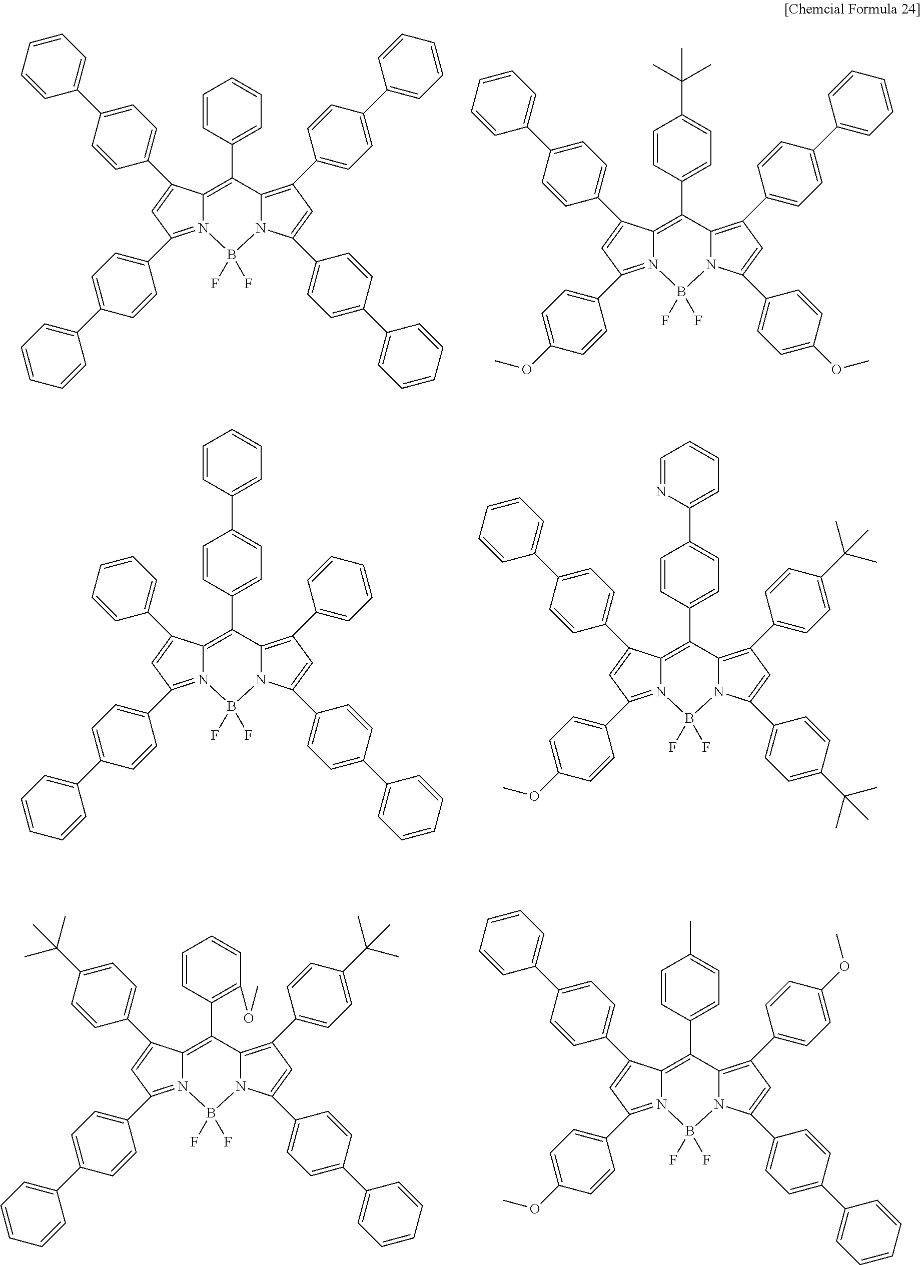

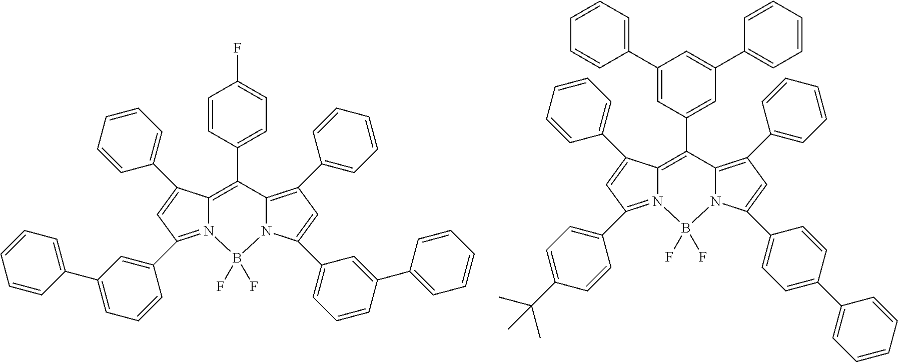

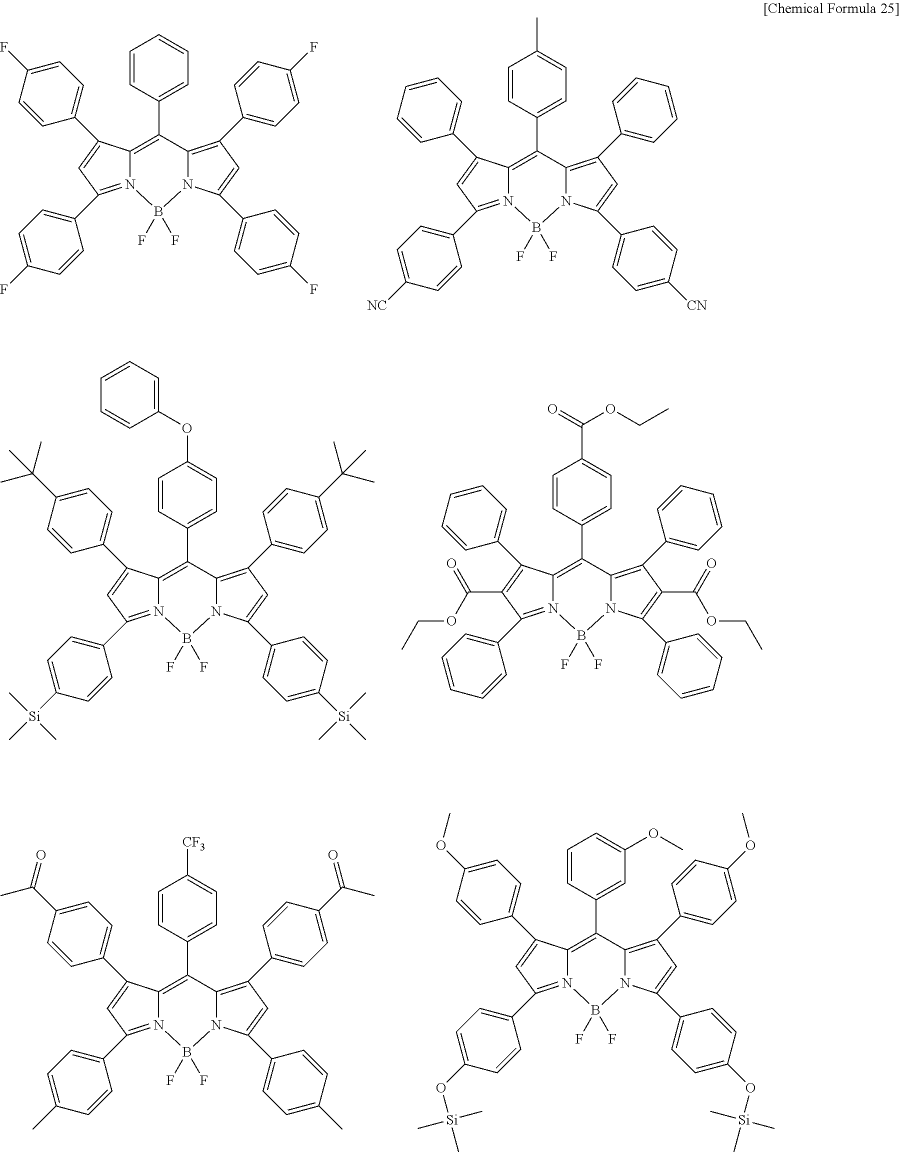

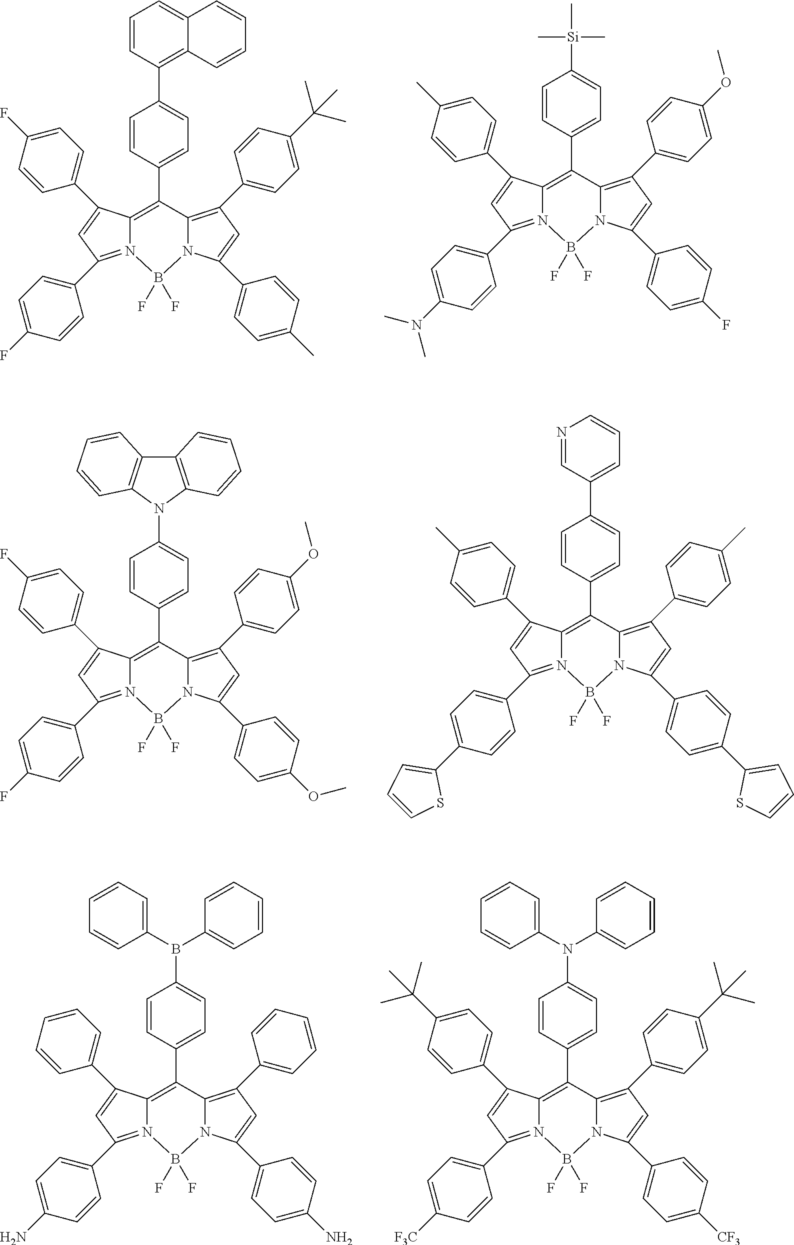

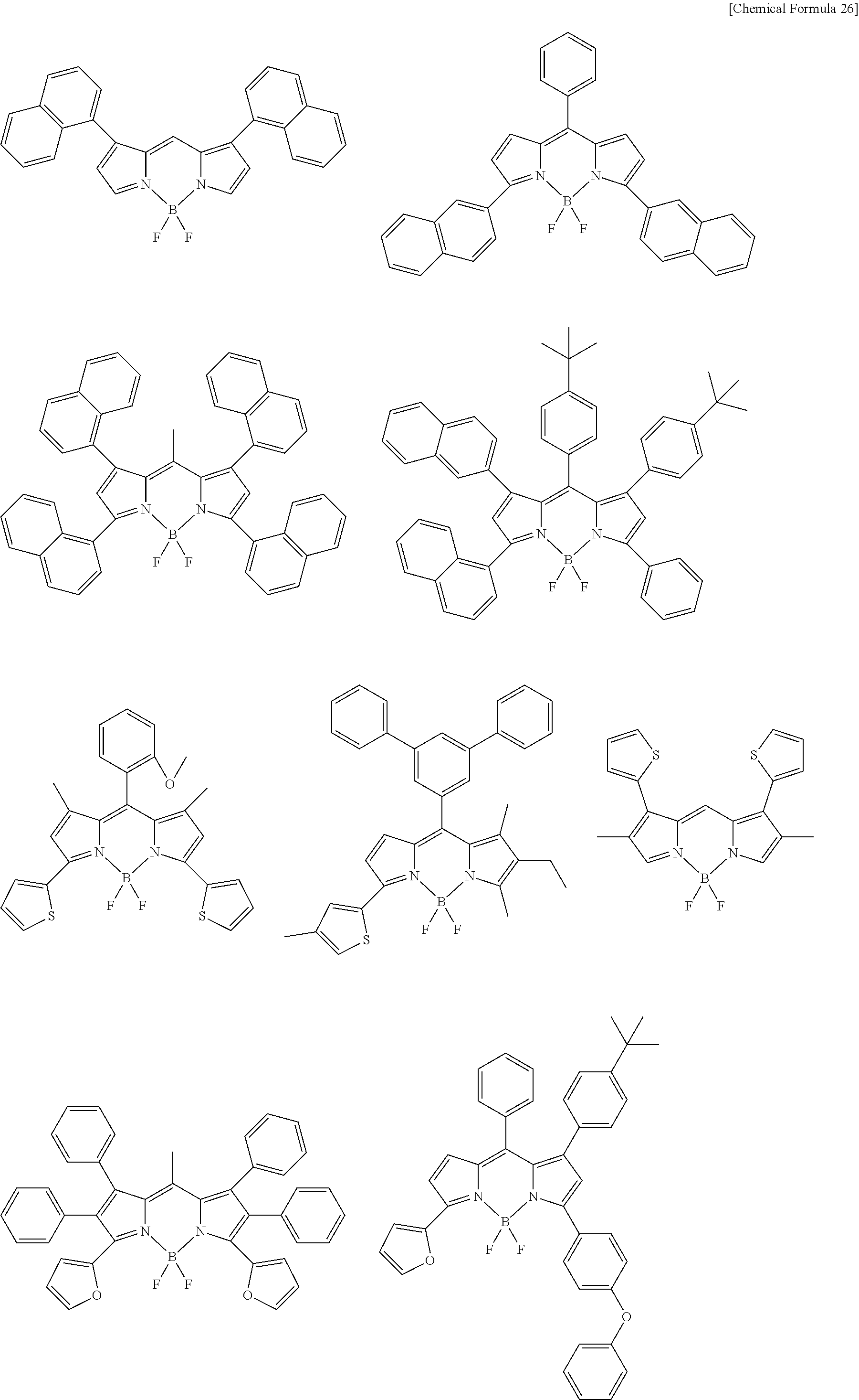

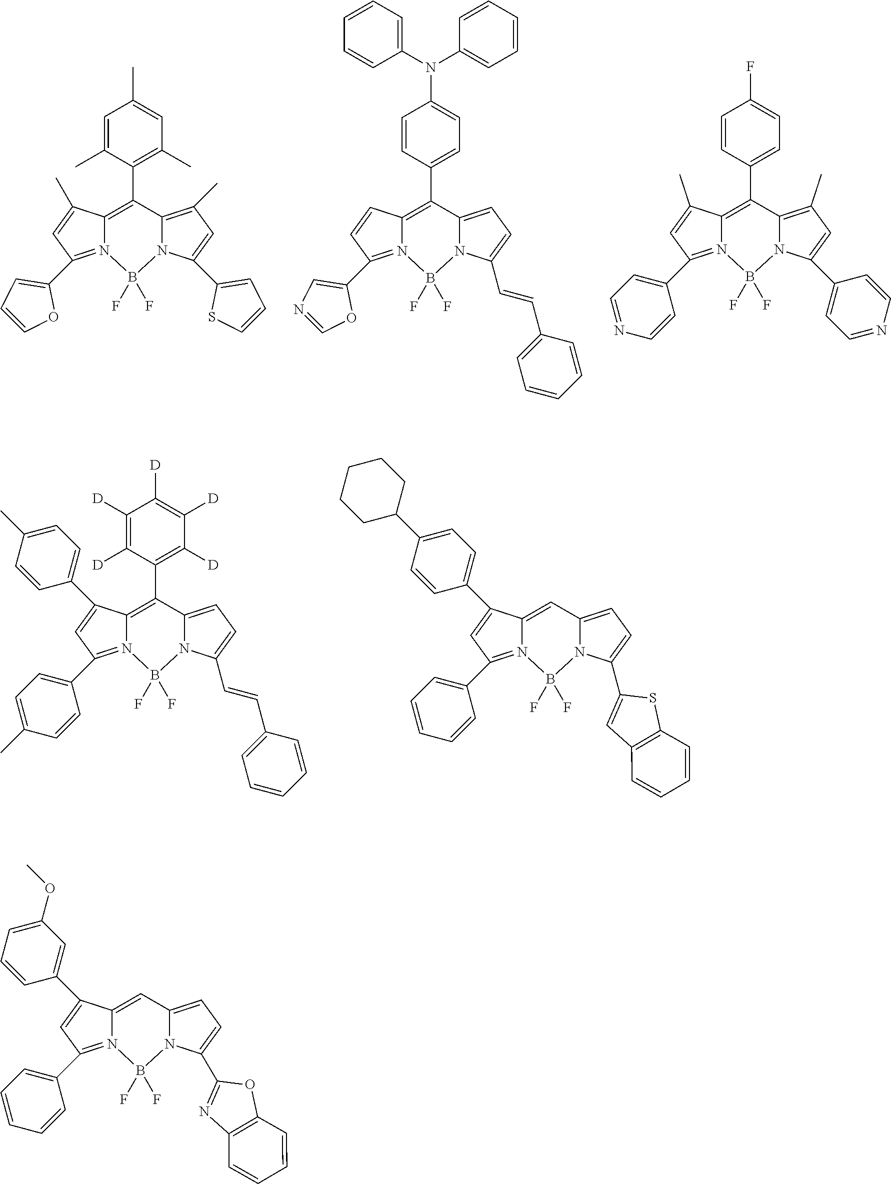

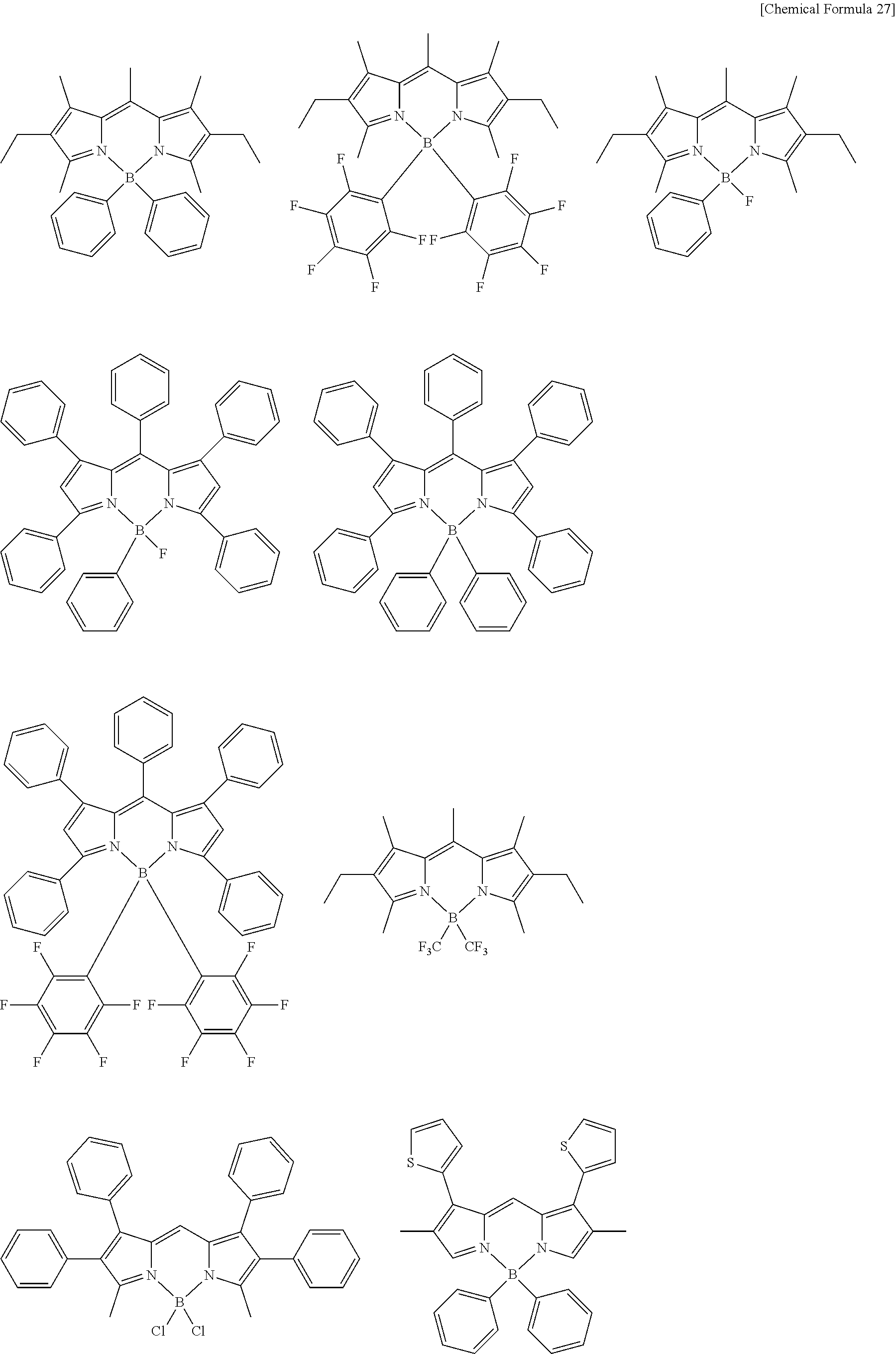

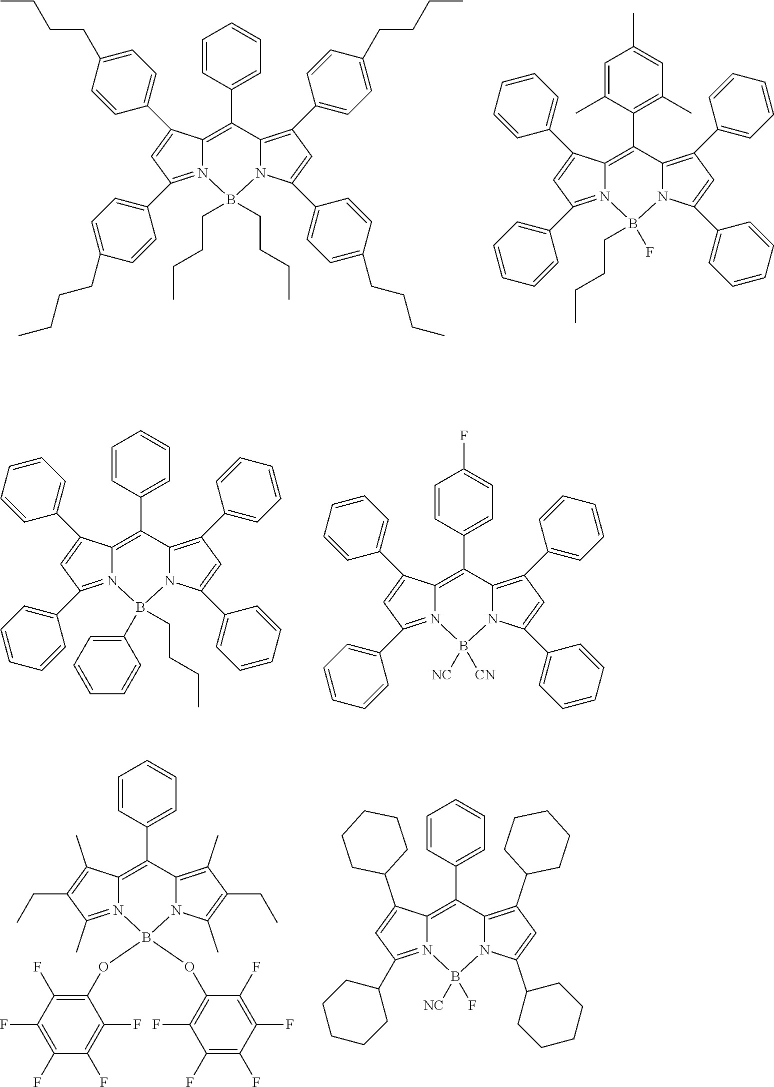

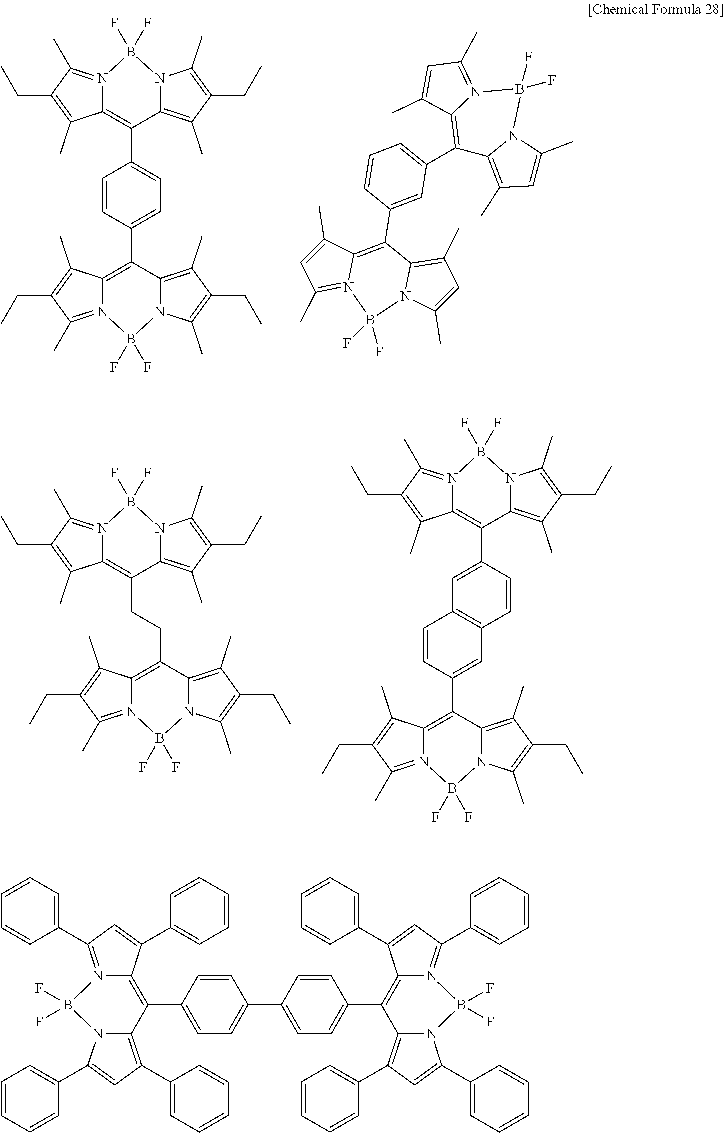

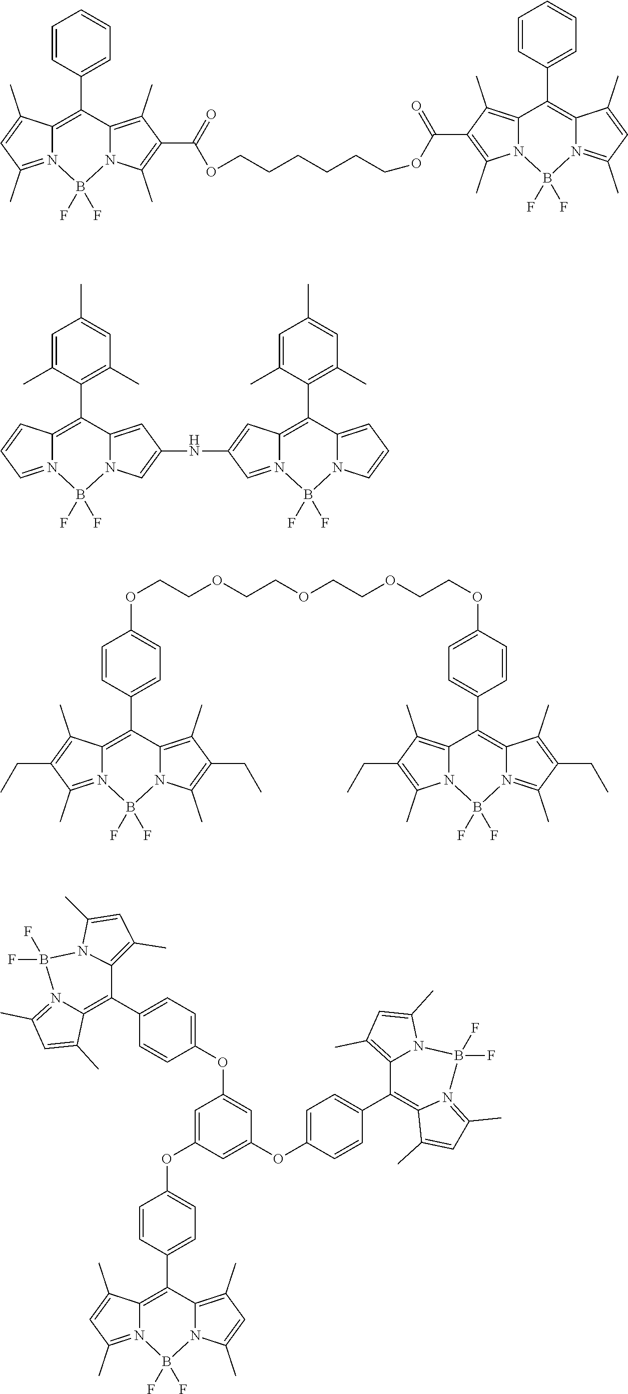

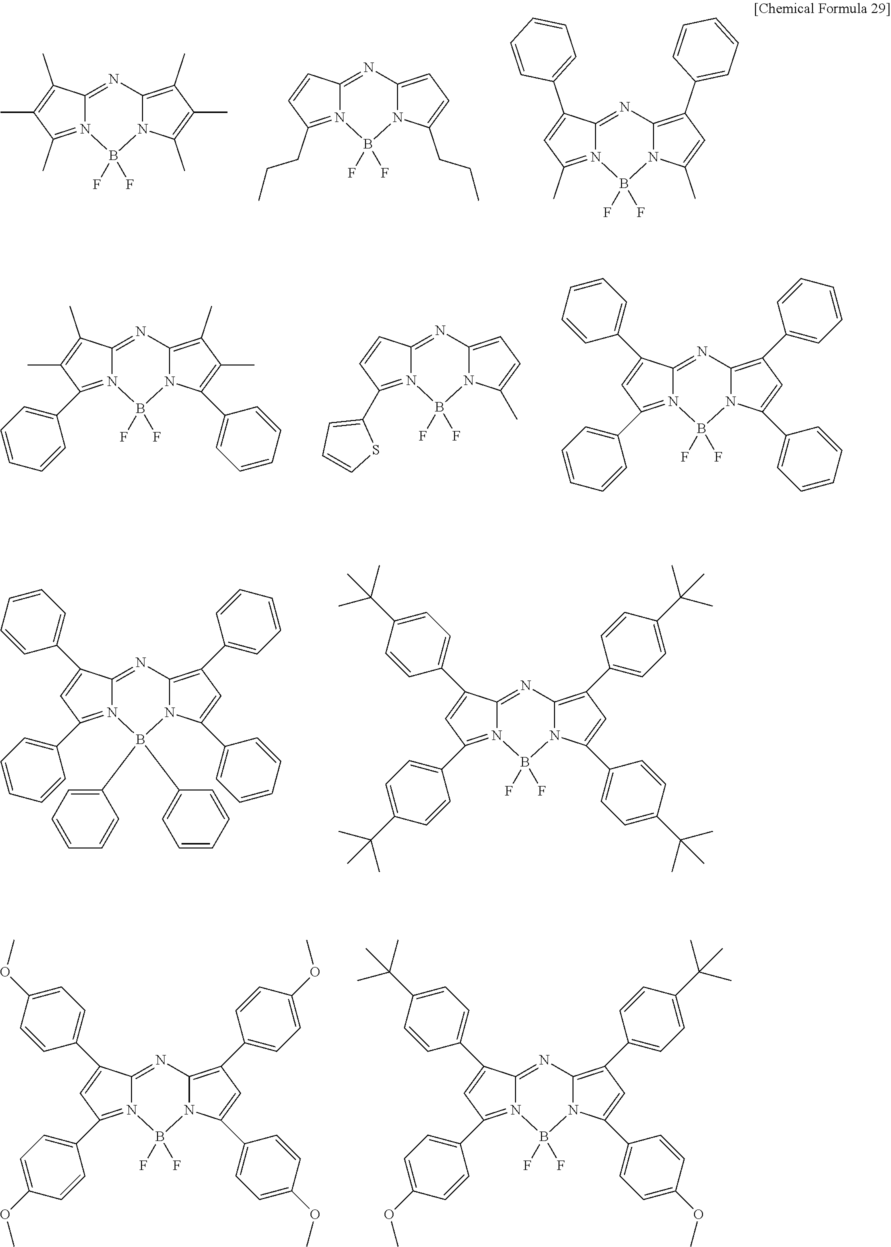

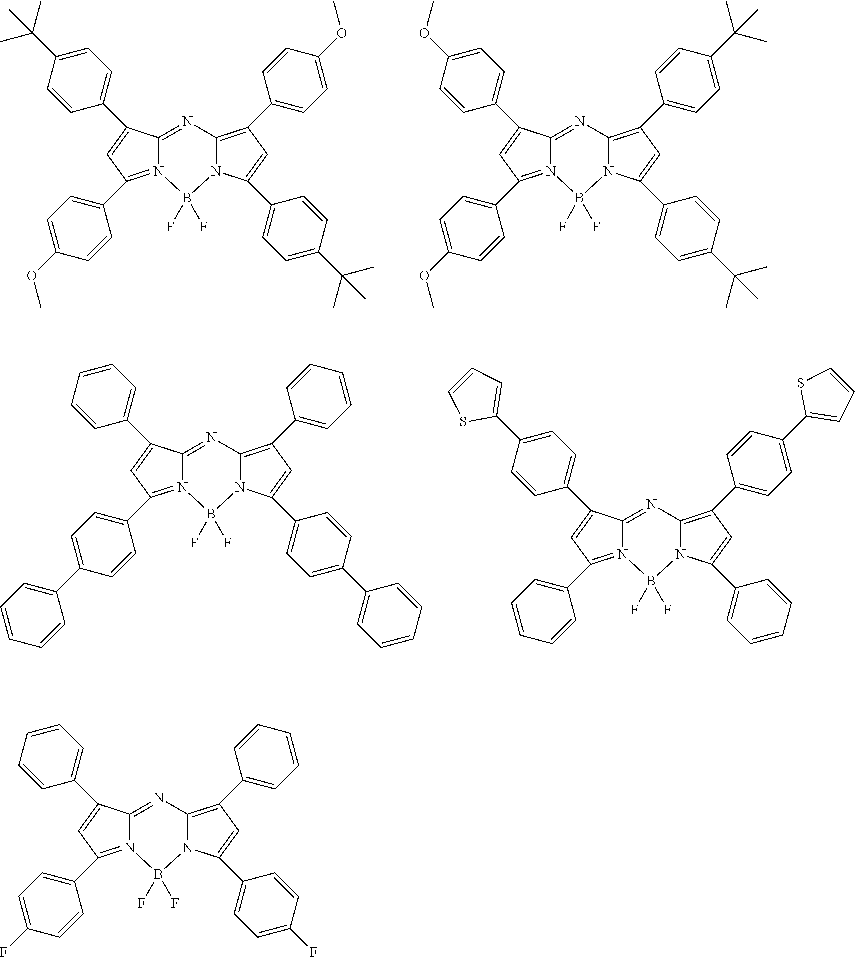

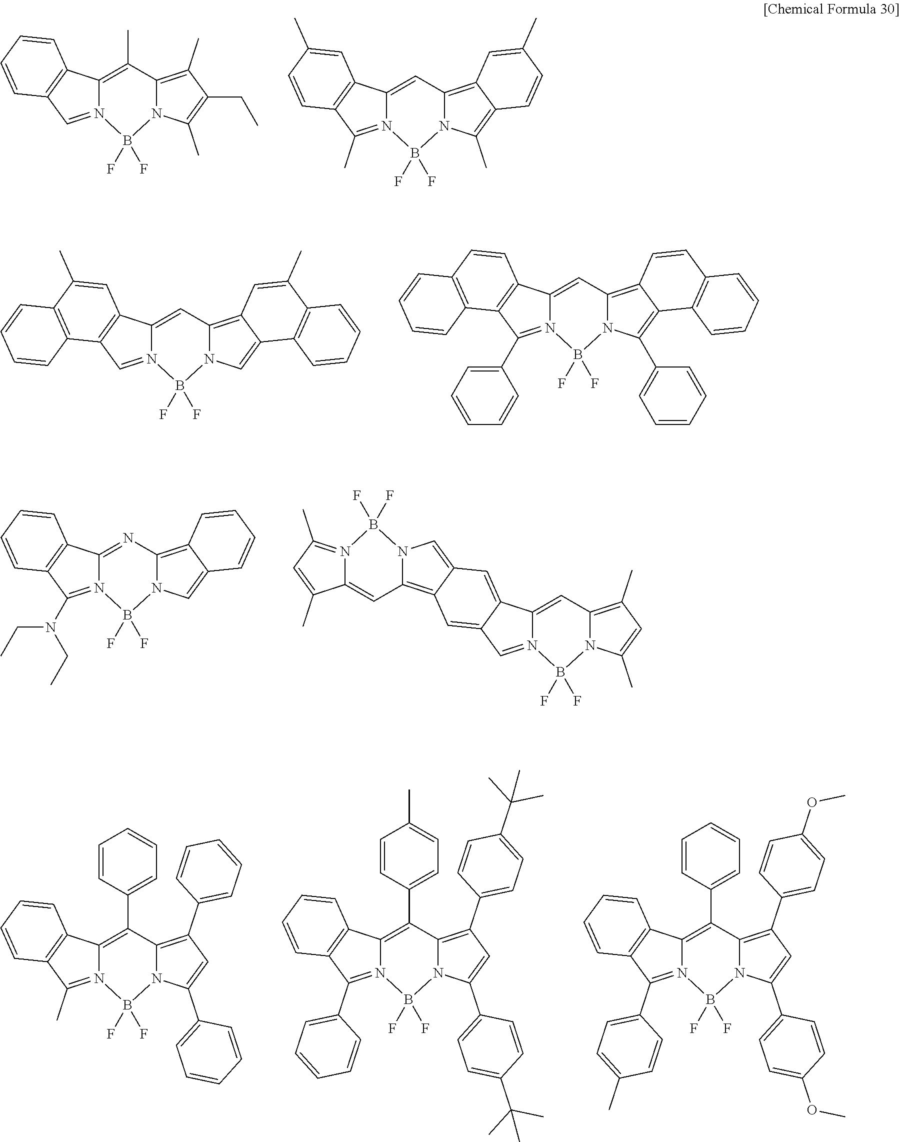

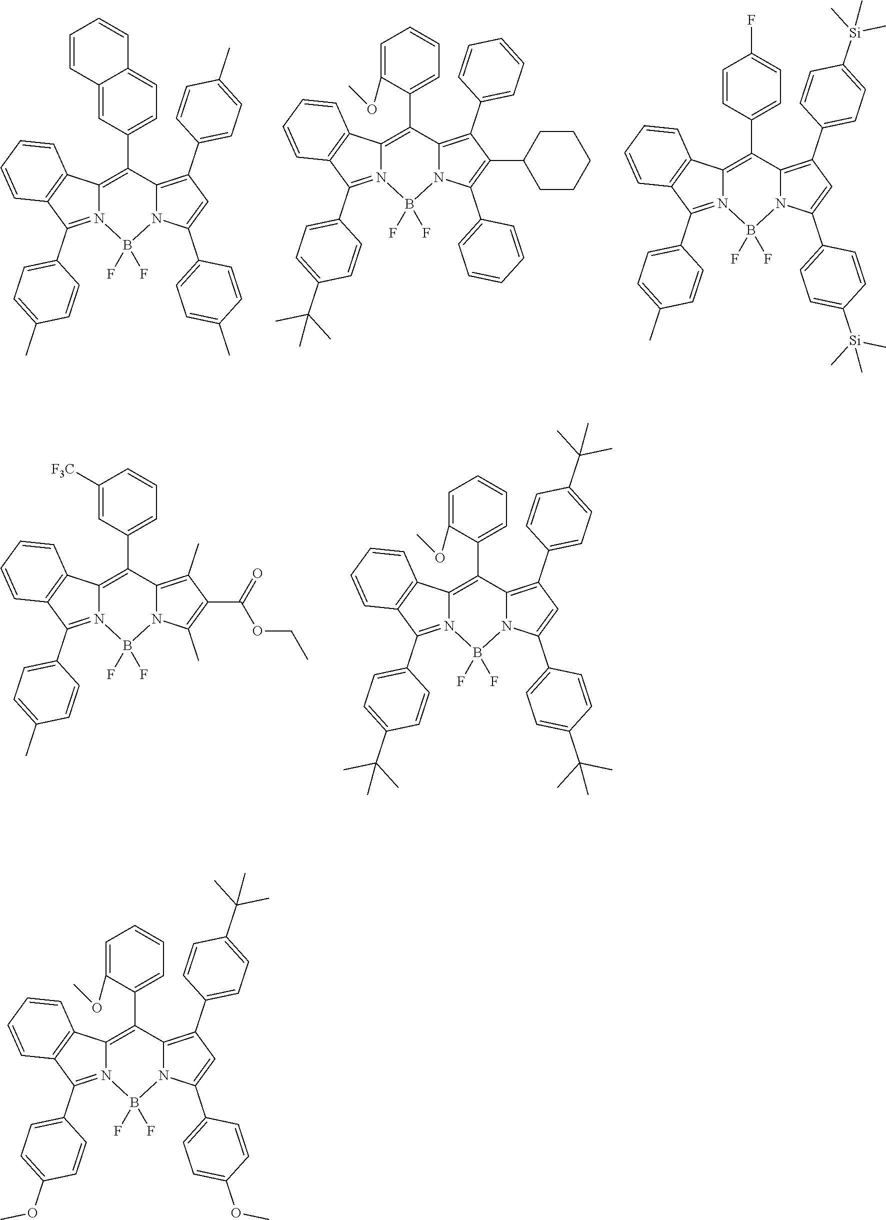

The compound represented by general formula (1) can be synthesized by a method described in Japanese Translation of PCT Application No. H08-509471 or Japanese Patent Application Laid-open No. 2000-208262, for example. That is, a pyrromethene compound and a metal salt are reacted with each other in the presence of a base to obtain a target pyrromethene-based metal complex.

For the synthesis of a pyrromethene-boron fluoride complex, methods described in J. Org. Chem., vol. 64, No. 21, pp. 7813-7819 (1999), and Angew. Chem., Int. Ed. Engl., vol. 36, pp. 1333-1335 (1997) and the like are referred to, whereby the compound represented by general formula (1) can be synthesized. Examples of the methods include a method which heats a compound represented by general formula (9) and a compound represented by general formula (10) in 1,2-dichloroethane in the presence of phosphoryl chloride and reacts them with a compound represented by general formula (11) in 1,2-dichloroethane in the presence of triethylamine, thereby obtaining the compound represented by general formula (1). However, the present invention is not limited thereto. Here, R¹ to R⁹ are similar to those described above J represents a halogen.

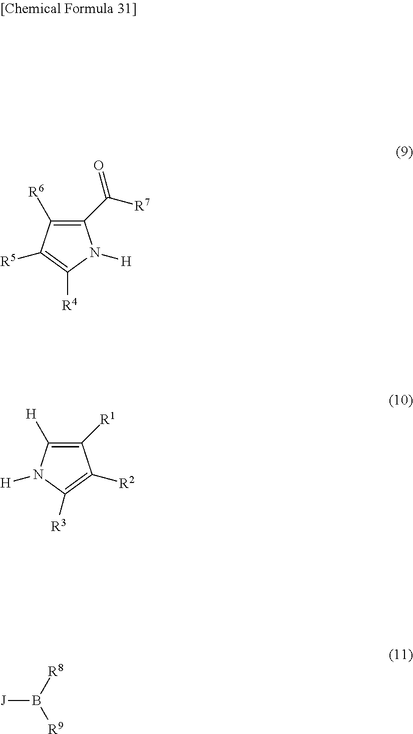

<Delayed Fluorescent Compound>

Delayed fluorescence is a phenomenon in which energy is once held in a metastable state, and the released energy is then released as light. Examples thereof include a phenomenon in which transition to a state having a different spin multiplicity occurs once after excitation and a light emission process is provided therefrom. In the case of a thermally activated delayed fluorescence (TADF) phenomenon, after excitation, reverse intersystem crossing from triplet excitons to singlet excitons occurs, which causes light emission to occur from the singlet level.

The compound represented by general formula (1) is suitable as a dopant for the emissive layer since it exhibits a high quantum efficiency and a narrow half-value width. However, since the compound is fluorescent, the triplet excitons among the excitons generated by the recombination of electrons and holes cannot be directly utilized as energy of light emission. However, by using a delayed fluorescent compound capable of converting triplet excitons into singlet excitons together with the compound represented by general formula (1), the triplet excitons generated by the recombination of electrons and holes can be converted into the singlet excitons capable of utilizing the compound represented by general formula (1). This makes it possible to efficiently utilize the excitons generated by the recombination of electrons and holes as light emission.

Preferred examples of the delayed fluorescent compound to be combined with the compound represented by general formula (1) include a compound represented by general formula (2).

In the following description, unless otherwise specified, the contents of substituents are the same as those shown in the description regarding the compound represented by general formula (1).

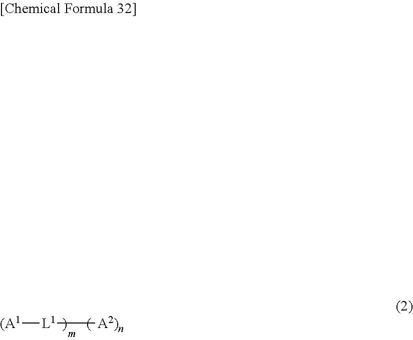

A¹ is an electron-donating moiety, and A² is an electron-accepting moiety. L¹s each are a linking group, are the same or different from each other, and each represent a single bond or a phenylene group. The symbols m and n each are a natural number of 1 or more and 10 or less. When m is 2 or more, a plurality of A¹s and L¹s are the same or different from each other. When n is 2 or more, a plurality of A²s are the same or different from each other. From the viewpoints of heat resistance and film formability, m and n each are preferably 6 or less, and particularly preferably 4 or less.

The electron-donating moiety as A¹ represents a moiety having relatively more electrons than those in an adjacent moiety. This generally represents a moiety having an unshared electron pair such as a nitrogen atom, an oxygen atom, a sulfur atom, or a silicon atom. Specific examples of the electron-donating moiety include a moiety including a structure such as a primary amine, a secondary amine, a tertiary amine, a pyrrole skeleton, ether, a furan skeleton, thiol, a thiophene skeleton, silane, a silole skeleton, or siloxane.

A¹ is preferably a group containing an electron-donating nitrogen atom, and preferably a group containing a tertiary amine or a heteroaryl group containing electron-donating nitrogen. Among these, a group containing a tertiary amine substituted with a substituted or unsubstituted aryl group or a substituted or unsubstituted heteroaryl group, or a heteroaryl group containing a carbazole skeleton is more preferable.

A¹ is preferably selected from groups represented by general formula (3) or (4), and more preferably a group represented by general formula (3).

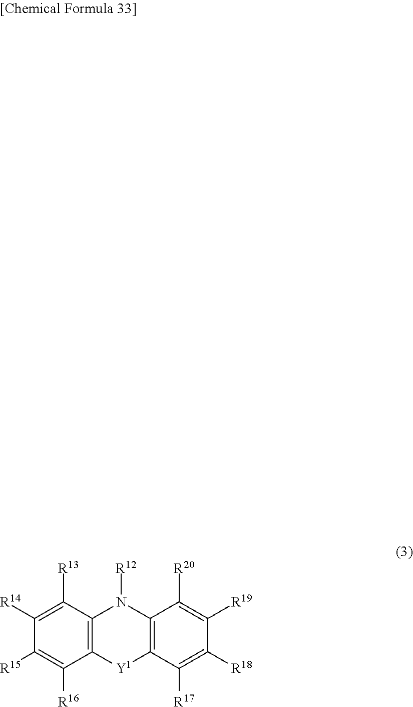

Y¹ is selected from a single bond, CR²¹R²², NR²³, O, or S. Among these, preferred is a single bond, CR²¹R²², or O, more preferred is a single bond or O, and particularly preferred is a single bond. By forming a carbazole skeleton or a cyclic tertiary amine skeleton, the electron-donating property of electron-donating nitrogen is improved, whereby charge transfer in the molecule is promoted, which is preferable.

R¹² to R²³ are the same or different from each other, and each are selected from a hydrogen atom, an alkyl group, a cycloalkyl group, a heterocyclic group, an alkenyl group, a cycloalkenyl group, an alkynyl group, a hydroxyl group, a thiol group, an alkoxy group, an alkylthio group, an aryl ether group, an aryl thioether group, an aryl group, a heteroaryl group, a halogen, a cyano group, an aldehyde group, a carbonyl group, a carboxyl group, an ester group, a carbamoyl group, an amino group, a nitro group, a silyl group, a siloxanyl group, a boryl group, —P(═O)R¹⁰R¹¹, and a fused ring and an aliphatic ring formed with an adjacent substituent. L¹ is bonded to at least one position of R¹² to R²³. R¹⁰ and R¹¹ each are an aryl group or a heteroaryl group.

L¹ bonded to at least one position of R¹² to R²³ means that L¹ is directly connected to a carbon atom or a nitrogen atom which corresponds to the root of each R.

R¹² to R²³ each are preferably an aryl group or a heteroaryl group, more preferably a phenyl group, a naphthalenyl group, a carbazolyl group, or a dibenzofuranyl group, and particularly preferably a phenyl group.

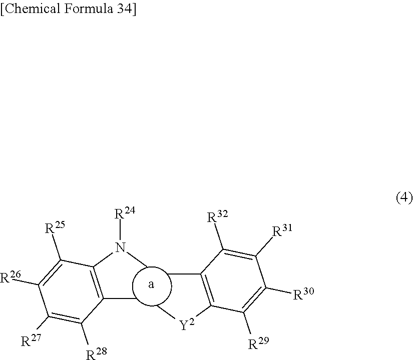

Ring a is a benzene ring or a naphthalene ring. A fused ring fused via the ring a has a relatively wide n-conjugated plane, and thus exhibits excellent carrier transport properties. Meanwhile, a too wide n-conjugated plane causes an excessive intermolecular interaction, which causes deteriorated thin film stability. From the viewpoint of the balance between the carrier transport properties and the thin film stability, a benzene ring is more preferred.

Y² is selected from CR³³R³⁴, NR³⁵, O, or S. Among these, Y² is preferably CR³³R³⁴, NR³⁵, or O, more preferably NR³⁵ or O, and particularly preferably NR³⁵.

R²⁴ to R³⁵ are the same or different from each other, and each are selected from a hydrogen atom, an alkyl group, a cycloalkyl group, a heterocyclic group, an alkenyl group, a cycloalkenyl group, an alkynyl group, a hydroxyl group, a thiol group, an alkoxy group, an alkylthio group, an aryl ether group, an aryl thioether group, an aryl group, a heteroaryl group, a halogen, a cyano group, an aldehyde group, a carbonyl group, a carboxyl group, an ester group, a carbamoyl group, an amino group, a nitro group, a silyl group, a siloxanyl group, a boryl group, —P(═O)R¹⁰R¹¹, and a fused ring and an aliphatic ring formed with an adjacent substituent. L¹ is bonded to at least one position of R²¹ to R³⁵. R¹⁰ and R¹¹ each are an aryl group or a heteroaryl group.

R²⁴ to R³⁵ each are preferably a phenyl group, a biphenyl group, a naphthalenyl group, a carbazolyl group, or a dibenzofuranyl group, and more preferably a phenyl group or a biphenyl group.

The fused structure represented by general formula (4) is not particularly limited, but specific examples thereof include the following. However, the following structure represents a basic skeleton, and may be substituted.

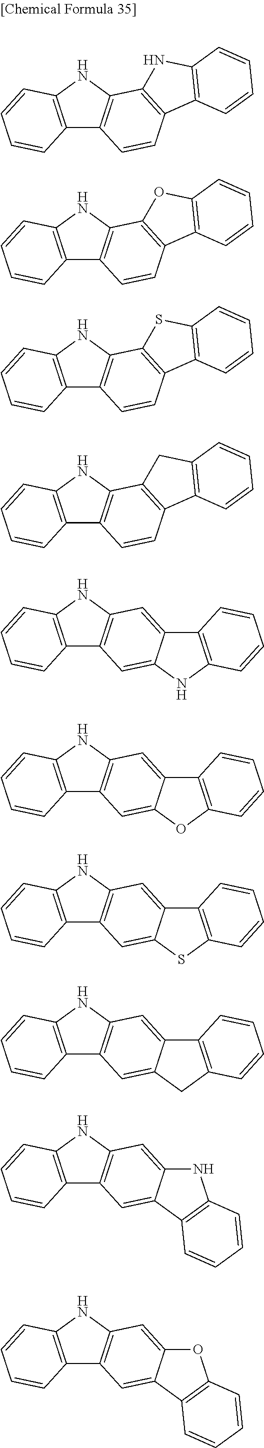

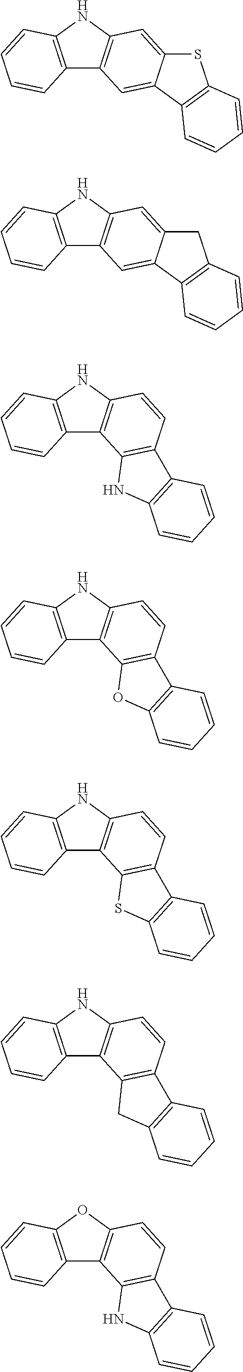

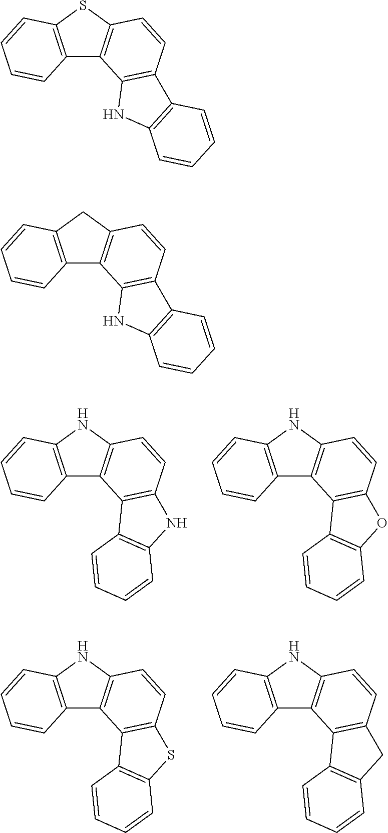

The electron-accepting moiety as A² represents a moiety having relatively less electrons than those in an adjacent moiety. Examples thereof generally include a moiety where a hetero atom forms a multiple bond with an adjacent atom. Specific examples of the electron-accepting moiety include a moiety containing electron-accepting nitrogen. Another examples thereof include electron-withdrawing substituents such as a cyano group, an aldehyde group, a carbonyl group, a carboxyl group, an ester group, a carbamoyl group, a nitro group, and —P(═O)R¹⁰R¹¹. Still other examples thereof include a moiety substituted with these substituents. R¹⁰ and R¹¹ each are an aryl group or a heteroaryl group.

A² is preferably a heteroaryl group containing electron-accepting nitrogen, and more preferably a group represented by general formula (5).

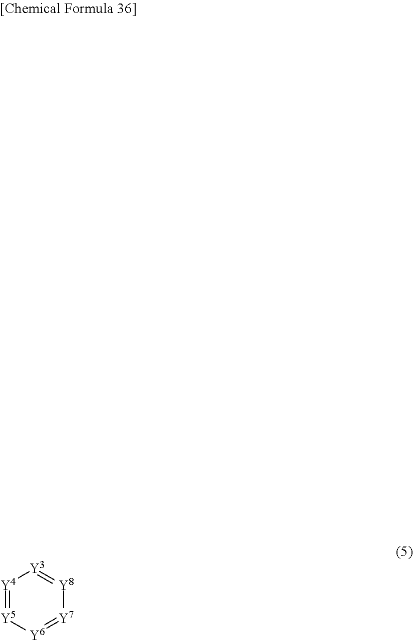

Y³ to Y⁸ are the same or different from each other, and each are selected from CR³⁶ or N. At least one of Y³ to Y⁸ is N, and all of Y³ to Y⁸ are not N. If the number of Ns is too large, the heat resistance is deteriorated, whereby the number of Ns is preferably 3 or less. R³⁶s are the same or different from each other, and each are selected from the group consisting of a hydrogen atom, an aryl group, a heteroaryl group, and a fused ring and an aliphatic ring formed with an adjacent substituent. L¹ is bonded to at least one position of Y³ to Y⁸.

The aryl group of R³⁶ is preferably a phenyl group, a biphenyl group, and a naphthalenyl group, and more preferably a phenyl group and a biphenyl group. The heteroaryl group of R³⁶ is preferably a heteroaryl group containing electron-accepting nitrogen. In particular, the heteroaryl group is preferably a pyridyl group and a quinolinyl group, and more preferably a pyridyl group.

L² bonded to at least one position of Y³ to Y⁸ means, for example, that Y³ is a carbon atom, and the carbon atom and L² are directly bonded to each other when L¹ is bonded to the position of Y³.

A² is preferably selected from groups represented by general formula (6) or (7), and more preferably a group represented by general formula (6).

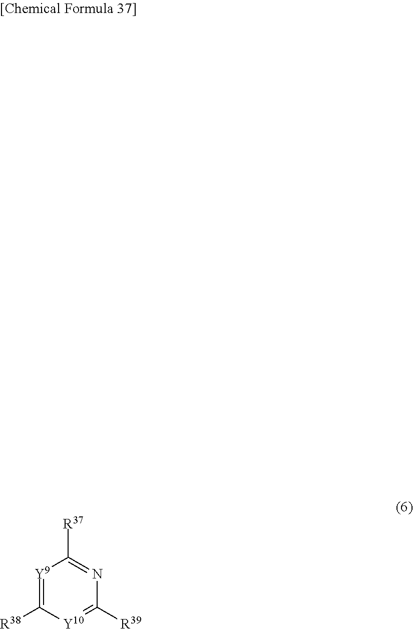

Y⁹ and Y¹⁰ are the same or different from each other, and each are selected from CR⁴⁰ or N. However, at least one of Y⁹ and Y¹⁰ is N. The nitrogen atoms are not adjacent to each other, and this provides improved heat resistance.

R³⁷ to R⁴⁰ are the same or different from each other, and each are selected from a hydrogen atom, an aryl group, or a heteroaryl group. L¹ is bonded to at least one position of R³⁷ to R⁴⁰.

The aryl group of R³⁷ to R⁴⁰ is preferably a phenyl group, a biphenyl group, and a naphthalenyl group, and more preferably a phenyl group and a biphenyl group. The heteroaryl group of R³⁷ to R⁴⁰ is preferably a heteroaryl group containing electron-accepting nitrogen. In particular, the heteroaryl group is preferably a pyridyl group and a quinolinyl group, and more preferably a pyridyl group.

L¹ bonded to any one position of R³⁷ to R⁴⁰ means, for example, that the carbon atom at the root of R³⁷ and L¹ are directly bonded to each other when L¹ is bonded to the position of R³⁷.

The group represented by general formula (6) is not particularly limited, but specific examples thereof include the following. However, the phenyl group in the following structure may be a biphenyl group, a naphthalenyl group, a pyridyl group, or a quinolinyl group, and may be further substituted.

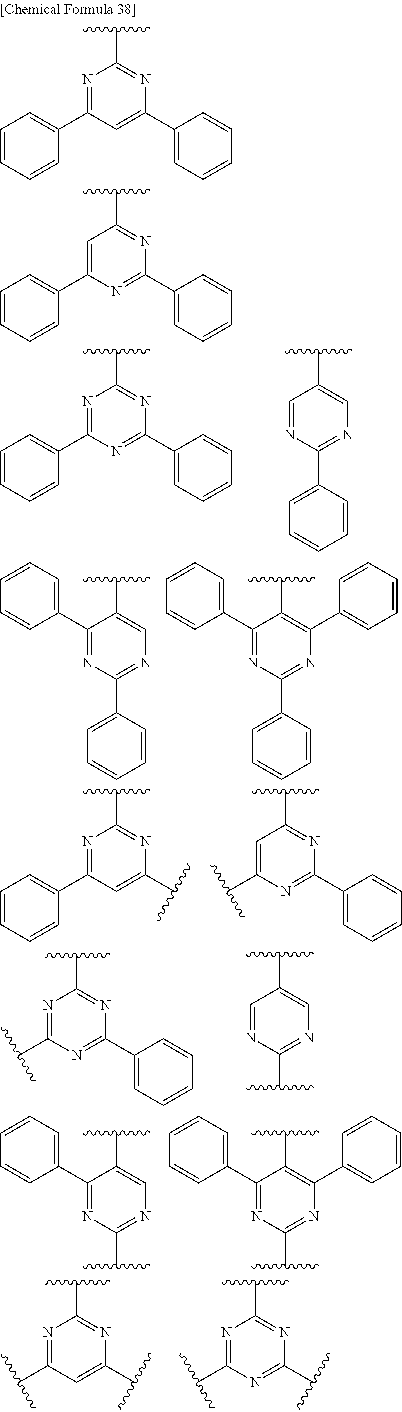

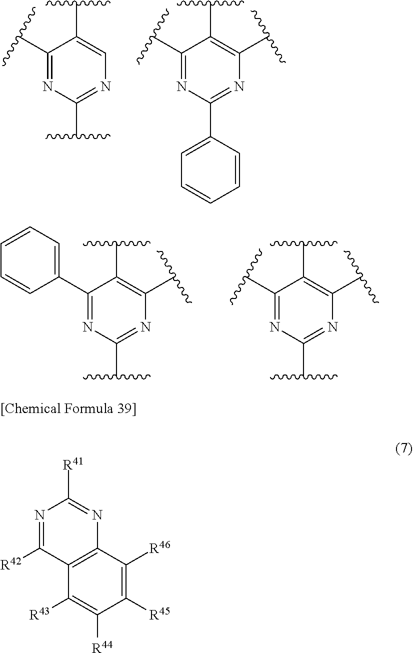

R⁴¹ to R⁴⁶ are the same or different from each other, and each are selected from a hydrogen atom, an aryl group, or a heteroaryl group. L¹ is bonded to at least one position of R⁴¹ or R⁴².

The aryl group of R⁴¹ to R⁴⁶ is preferably a phenyl group, a biphenyl group, and a naphthalenyl group, and more preferably a phenyl group and a biphenyl group. The heteroaryl group of R⁴′ to R⁴⁶ is preferably a heteroaryl group containing electron-accepting nitrogen. In particular, the heteroaryl group is preferably a pyridyl group and a quinolinyl group, and more preferably a pyridyl group.

When A² is a group represented by general formula (7), the energy difference between HOMO and LUMO is small. At this time, the compound represented by general formula (2) can be suitably combined with a compound which is represented by general formula (1) and exhibits light emission having a longer wavelength.

When A² is a group represented by general formula (6), the energy difference between HOMO and LUMO is moderate. At this time, the compound represented by general formula (2) can be suitably combined with more compounds represented by general formula (1), which is particularly preferable.

The molecular weight of the compound represented by general formula (2) is not particularly limited, but is preferably 900 or less, and more preferably 800 or less from the viewpoints of heat resistance and film formability. The molecular weight of the compound is still more preferably 700 or less, and particularly preferably 650 or less. Generally, as the molecular weight becomes larger, the glass transition temperature tends to increase, and as the glass transition temperature increases, thin film stability is improved. Thus, the molecular weight is preferably 400 or more, and more preferably 450 or more. The molecular weight is still more preferably 500 or more.

In the compound represented by general formula (2), an electron-donating moiety and an electron-accepting moiety exist in the same molecule. Such a compound is apt to have a small energy difference (ΔST) between a singlet level and a triplet level and to exhibit TADF properties. However, when the combination of the electron-donating moiety with the electron-accepting moiety is not appropriate, ΔST is not sufficiently small, so that a highly efficient TADF phenomenon cannot be exhibited.

The compound represented by general formula (2) is preferably formed by combining a specific electron-donating moiety represented by general formula (3) or (4) with a specific electron-accepting moiety represented by general formula (5). This is because the highly efficient TADF phenomenon is exhibited.

The electron-donating moieties represented by general formulae (3) and (4) have electron-donating nitrogen. Meanwhile, the electron-accepting moiety represented by general formula (5) has electron-accepting nitrogen. Electron distribution change efficiently occurs between the moiety having electron-donating nitrogen and the moiety having electron-accepting nitrogen. The electron-donating moieties represented by general formulae (3) and (4) have a relatively wide conjugated system, while the conjugated system of the specific electron-accepting moiety represented by general formula (5) is relatively narrow. Therefore, molecular distribution bias from the electron-donating moieties represented by general formulae (3) and (4) to the specific electron-accepting moiety represented by general formula (5) is apt to occur. In the compound represented by general formula (2), the electron orbitals of LUMO and HOMO are localized without overlapping. Further, dipoles formed in an excited state interact with each other, so that exchange interaction energy is apt to be small, which can provide sufficiently small ΔST.

Since the moieties represented by general formulae (3) and (4) have electron-donating nitrogen, they exhibit hole transporting properties. Meanwhile, since the moiety represented by general formula (5) has electron-accepting nitrogen, it exhibits electron transporting properties. That is, since the compound represented by general formula (2) has both a hole transporting moiety and an electron transporting moiety, it has bipolar characteristics capable of transporting both holes and electrons. Therefore, in the emissive layer, the localization of the recombination region is suppressed, which allows the lifetime of the element to be extended.

Further, since the compound represented by general formula (2) has an appropriate singlet level and triplet level, the transfer of the singlet energy to the compound represented by general formula (1) (as described later) efficiently occurs.

The compound represented by general formula (2) is not particularly limited, but specific examples thereof include the following.

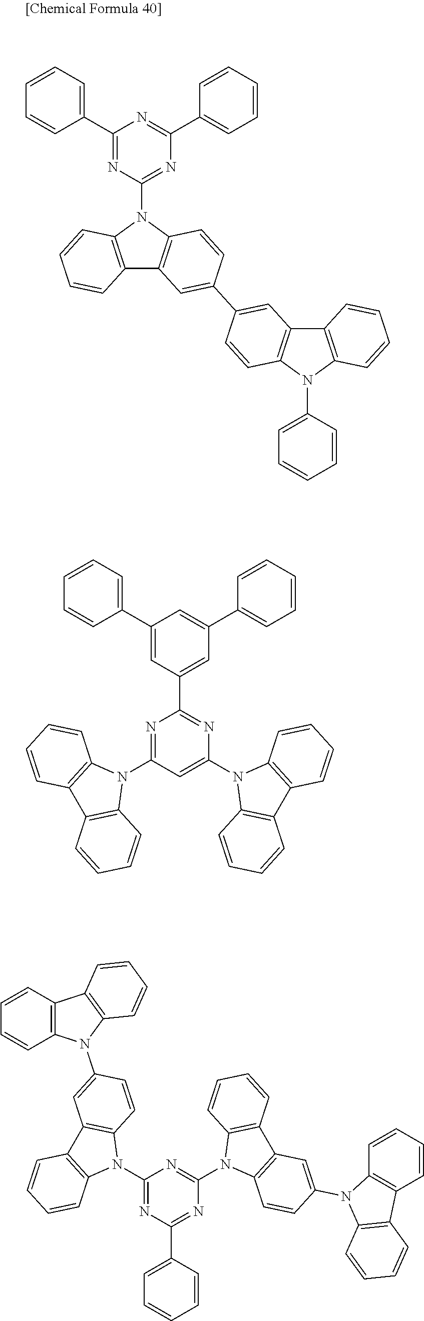

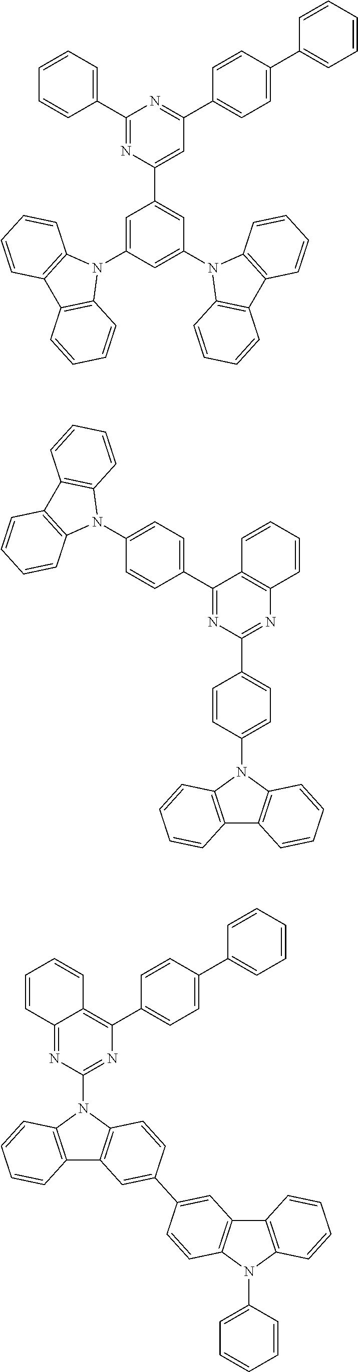

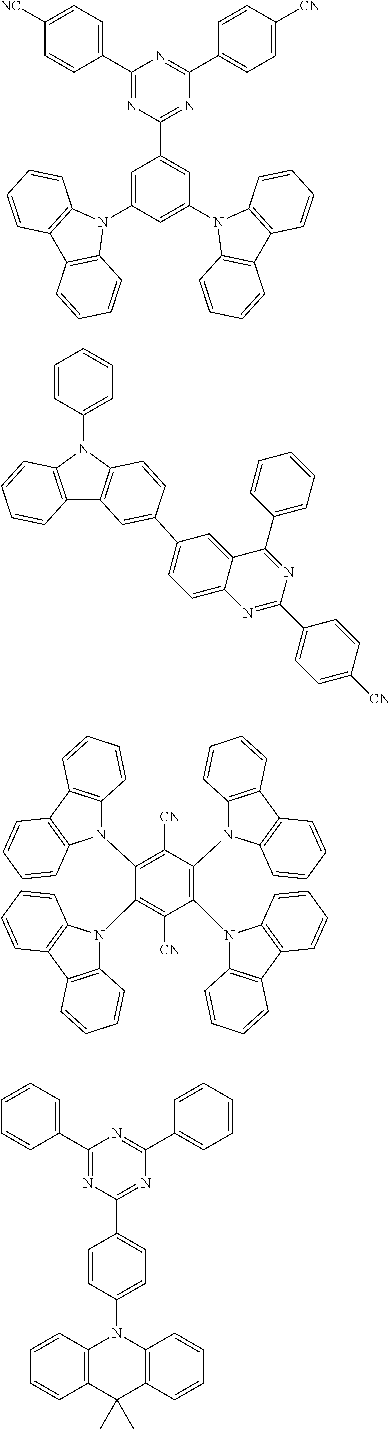

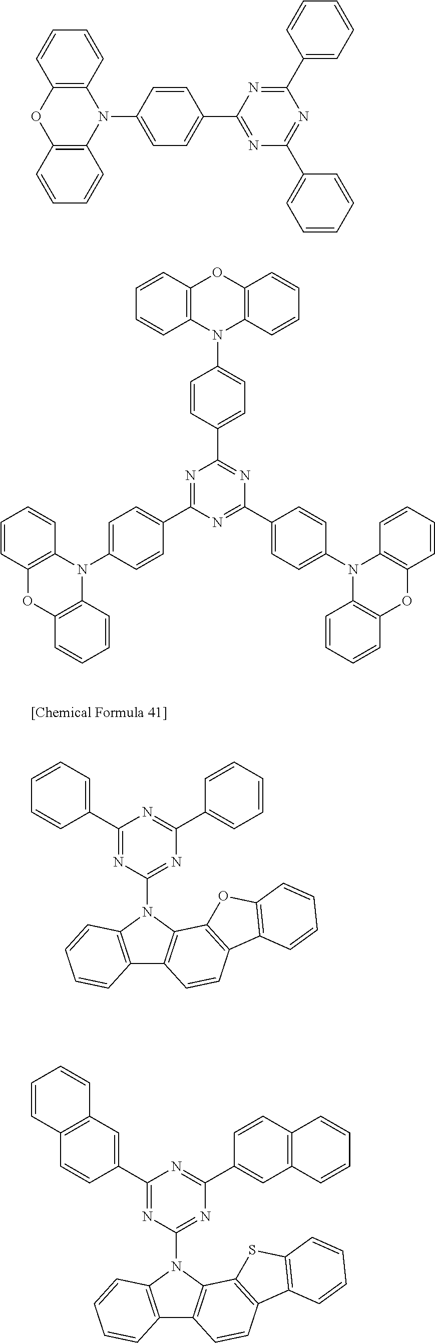

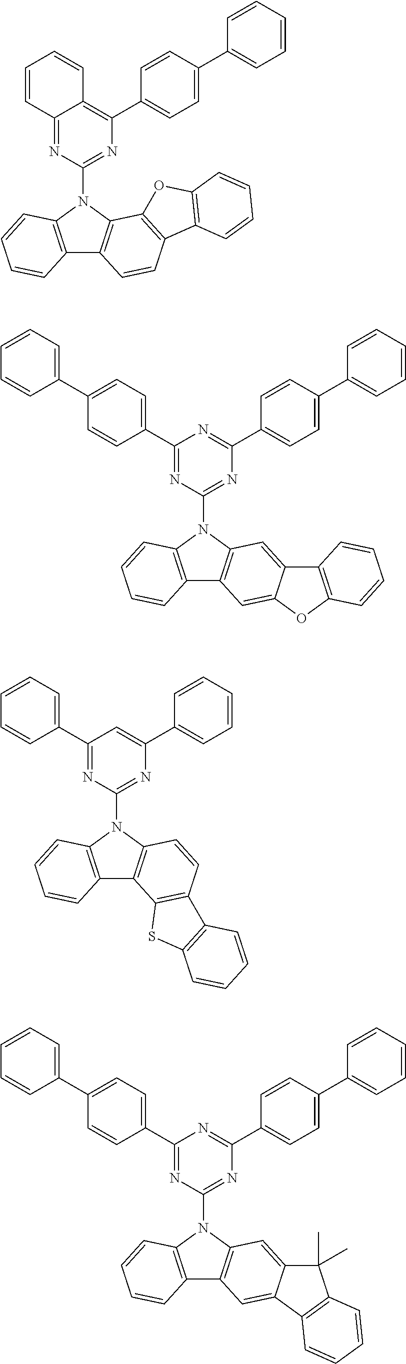

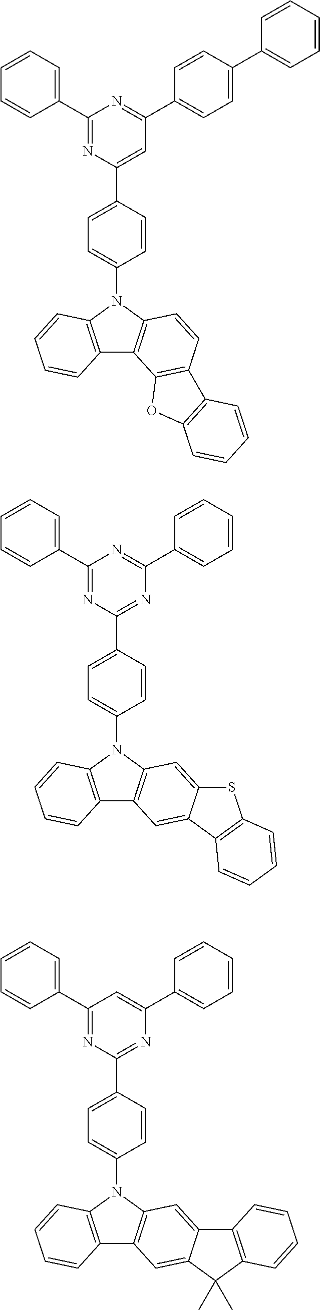

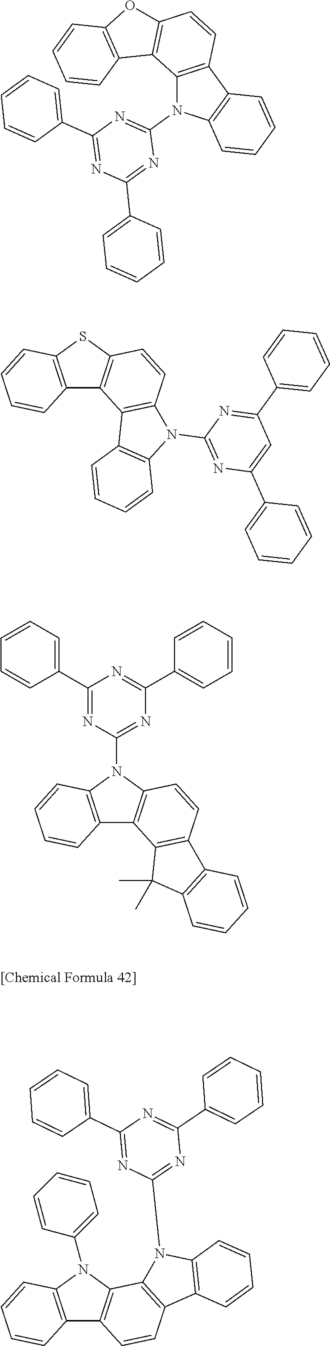

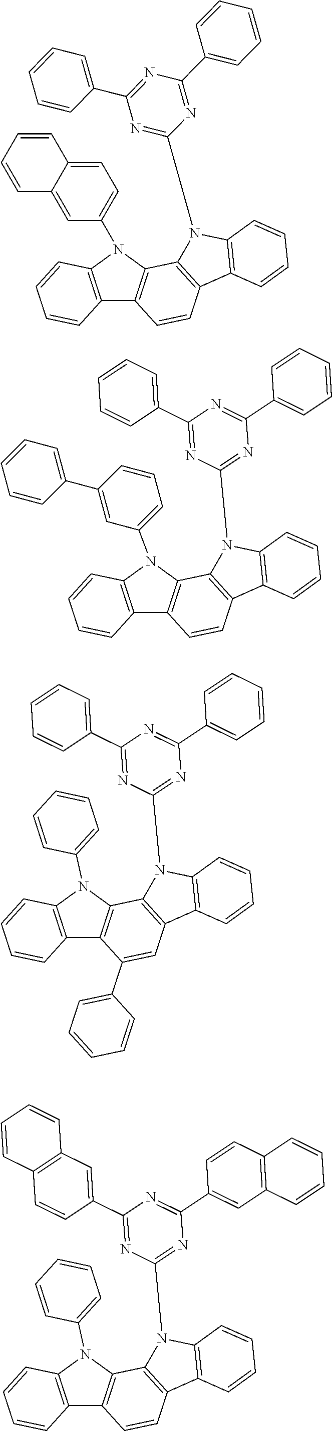

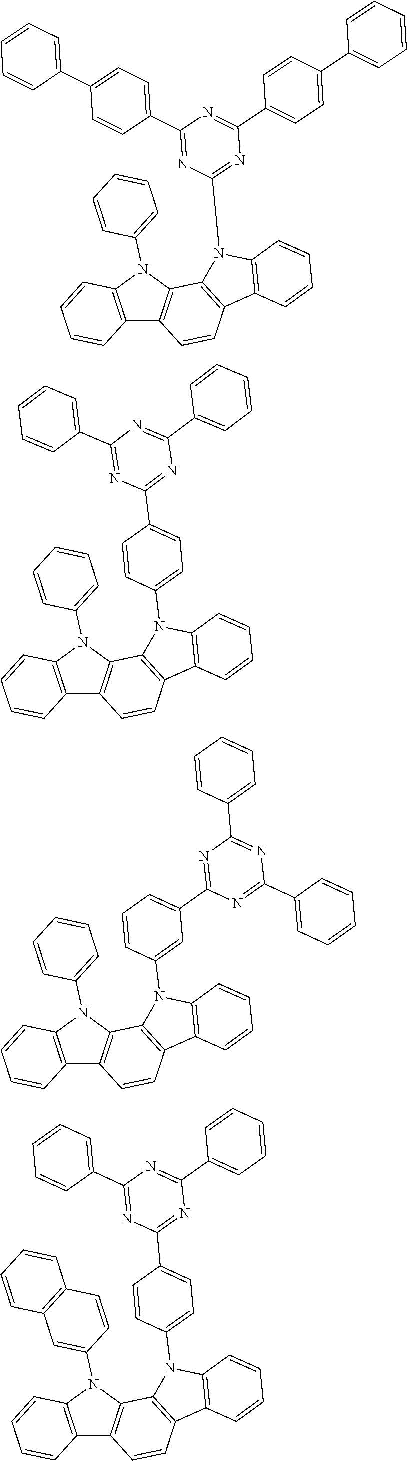

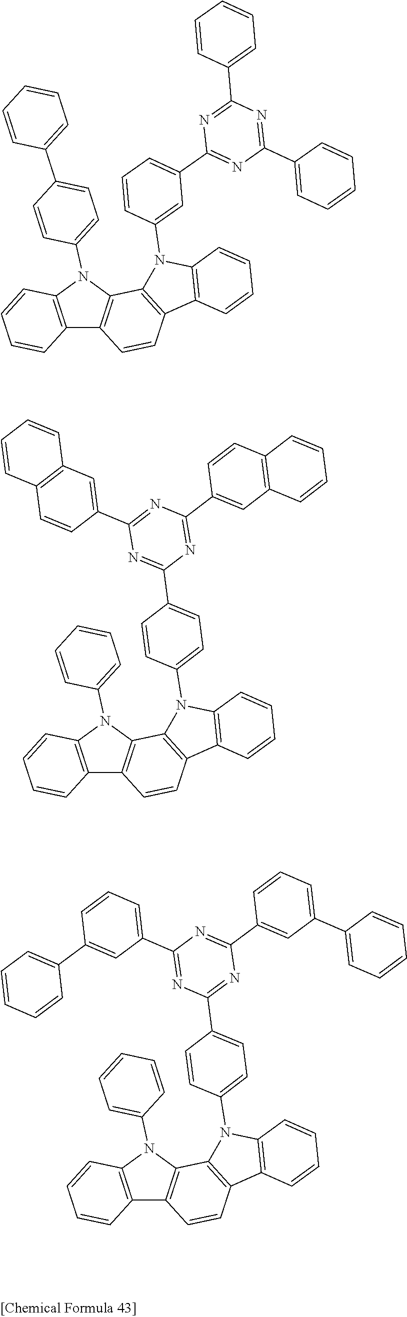

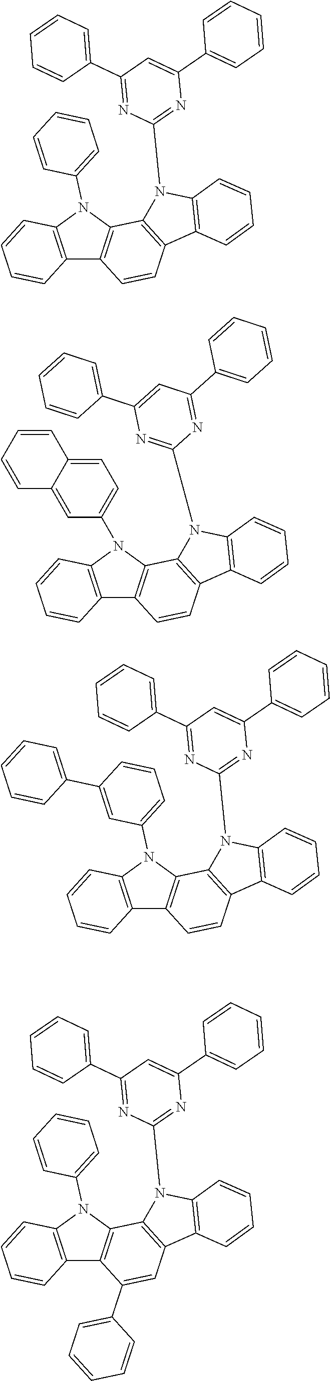

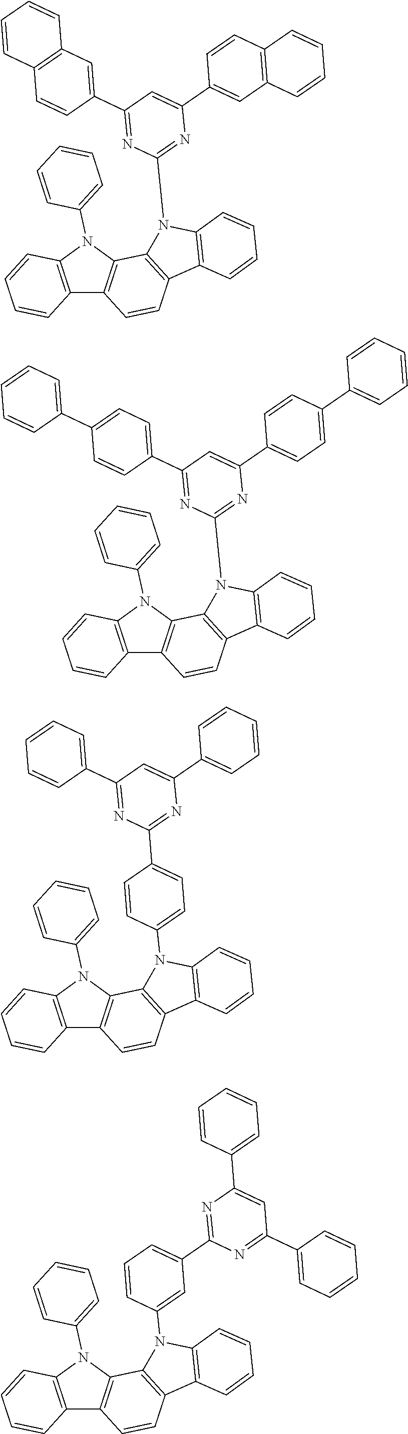

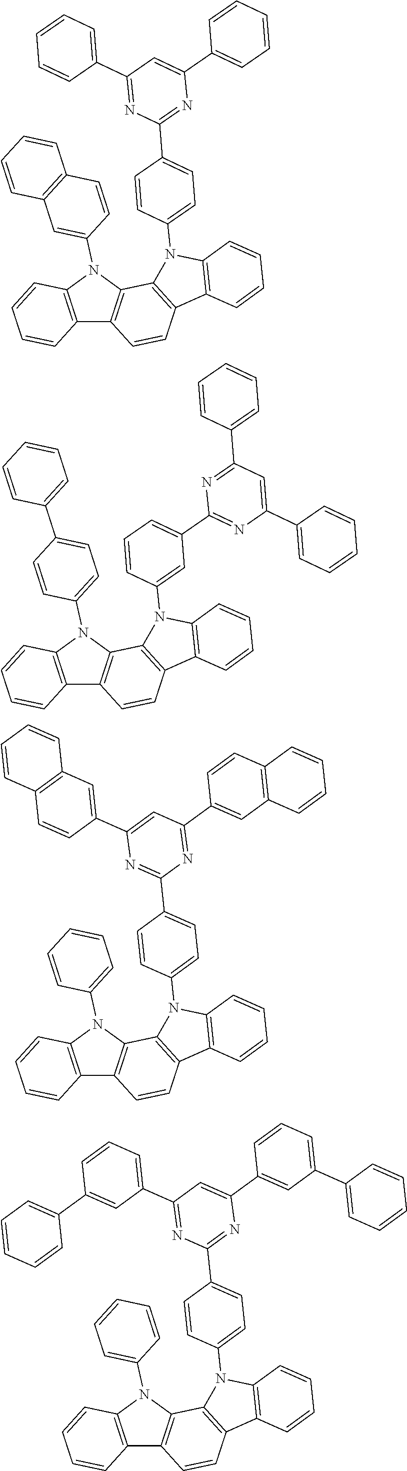

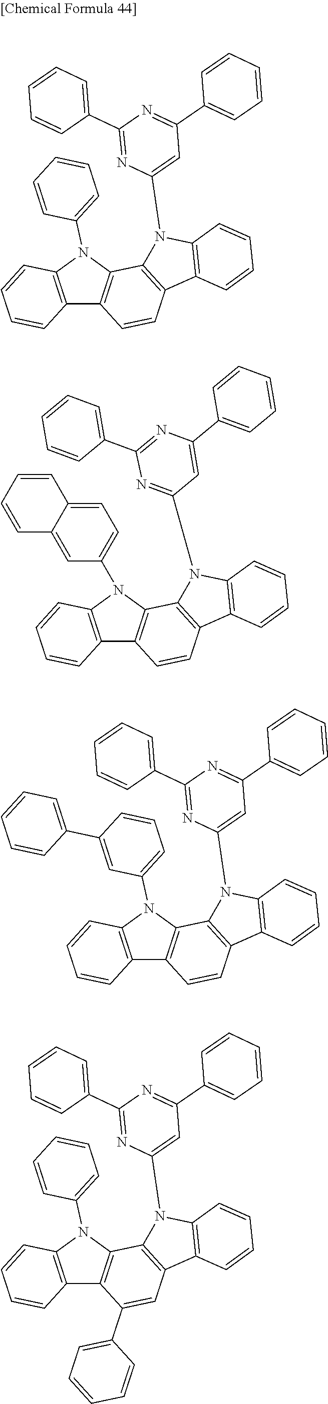

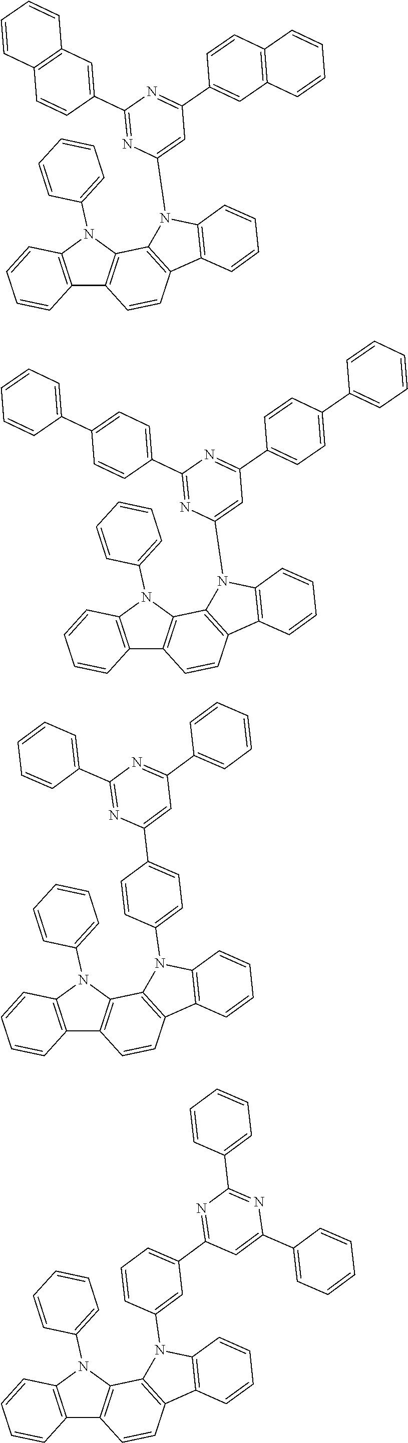

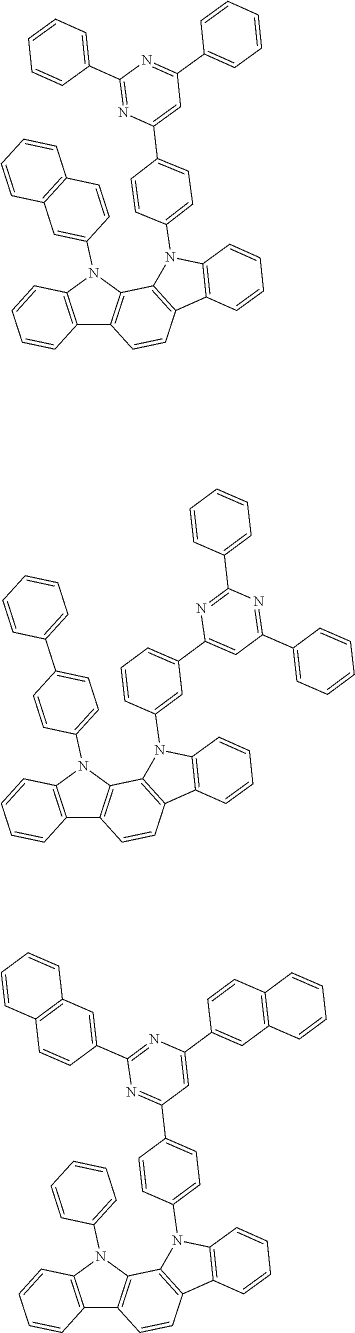

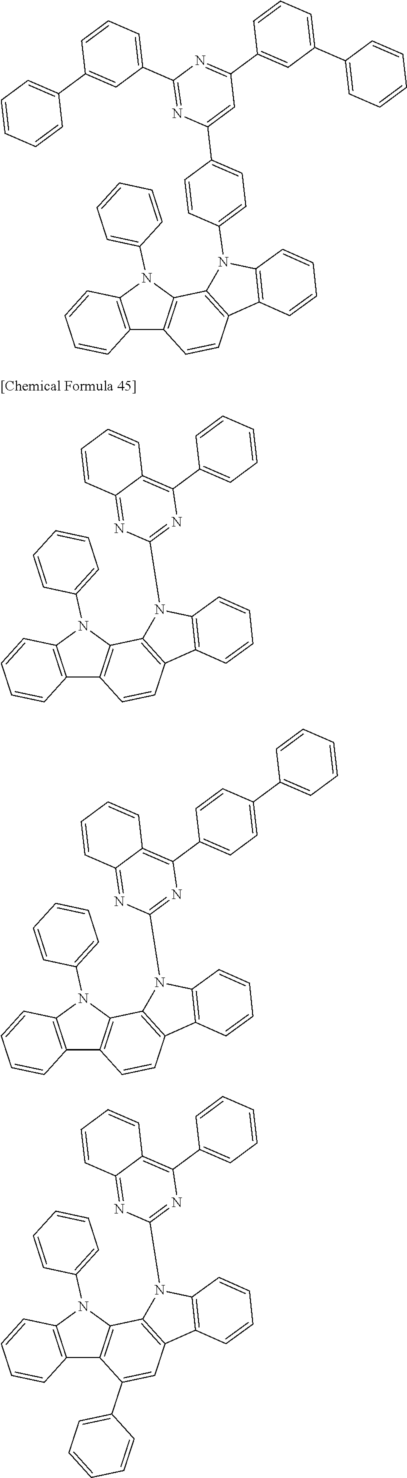

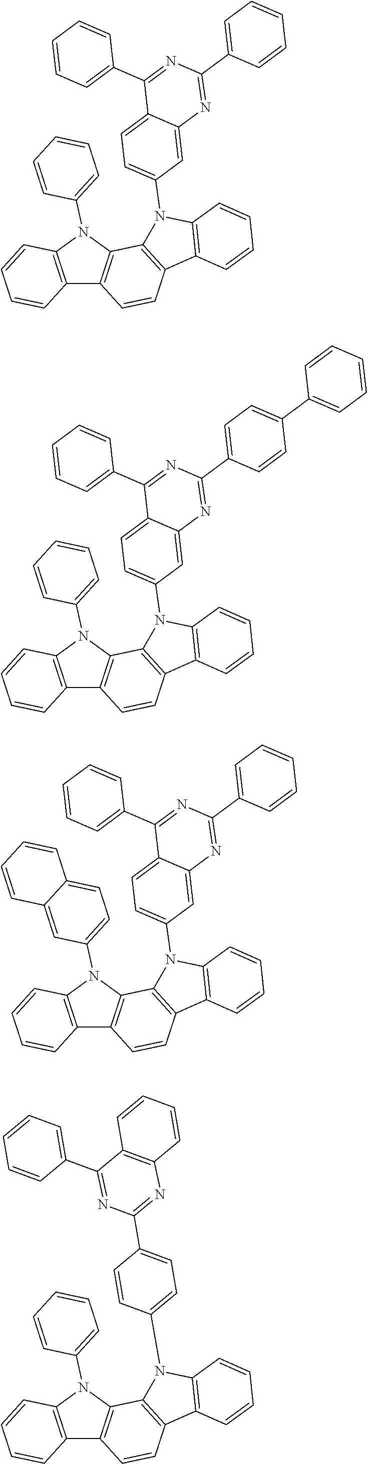

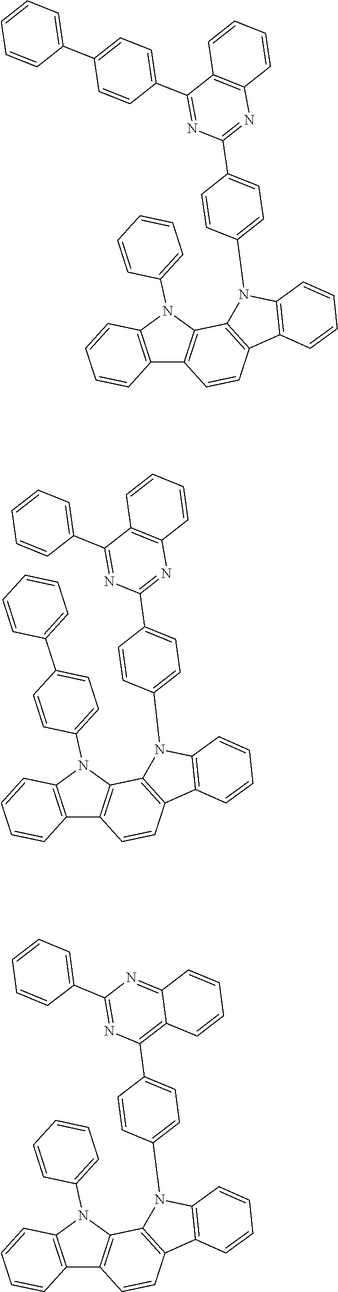

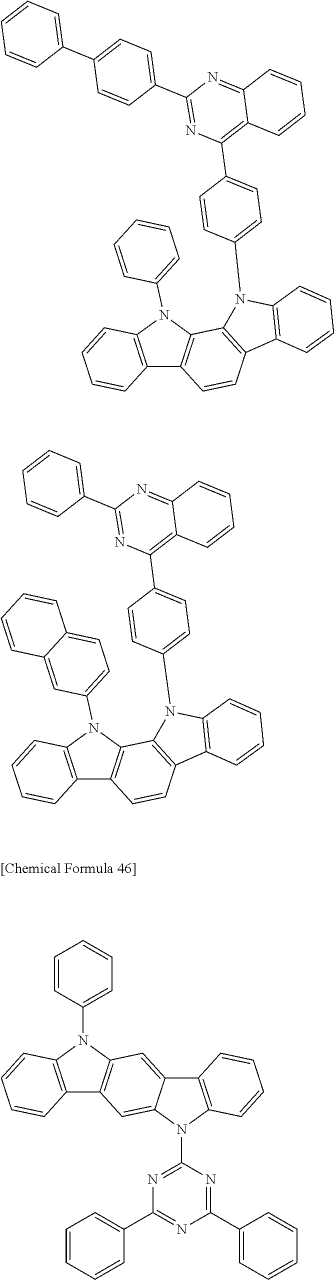

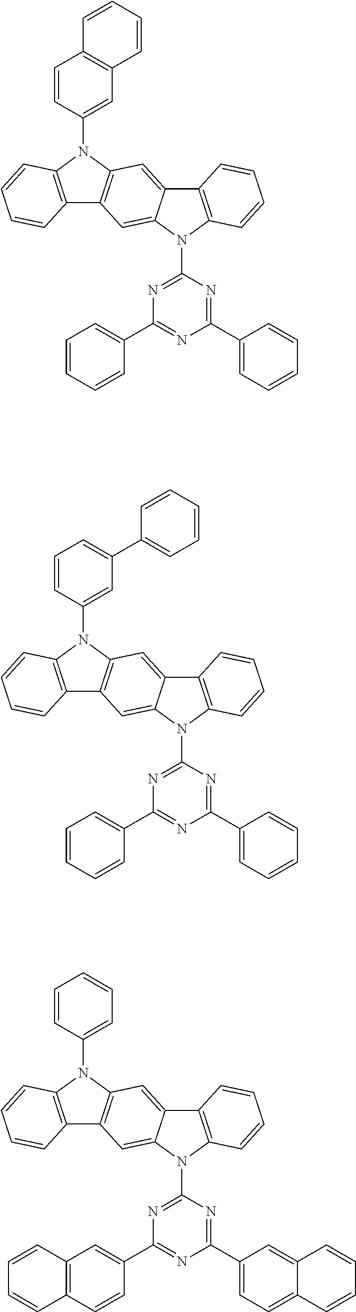

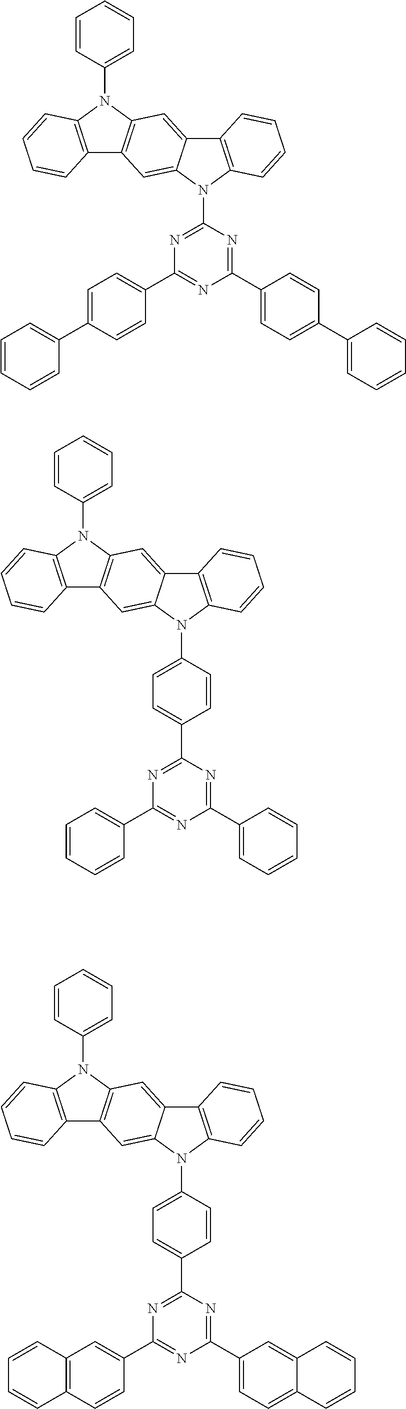

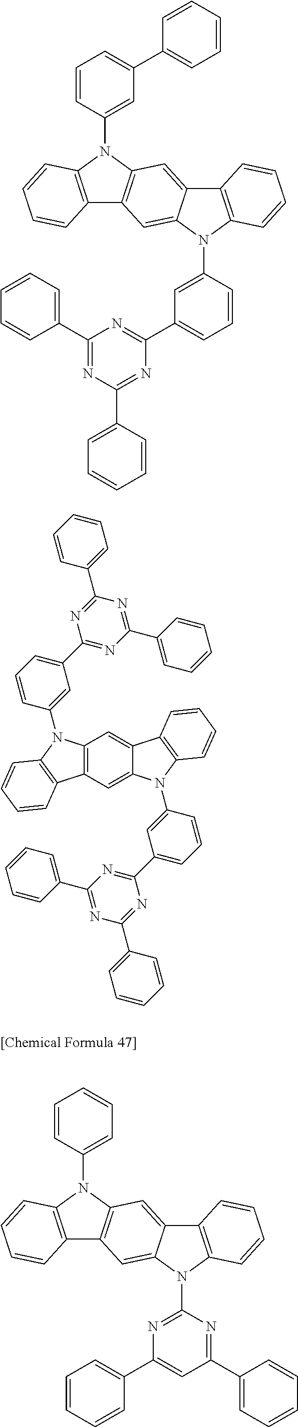

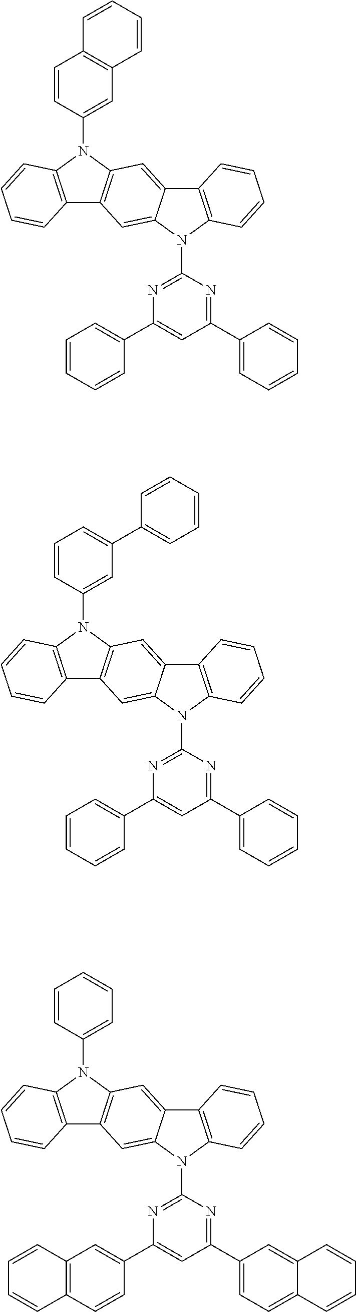

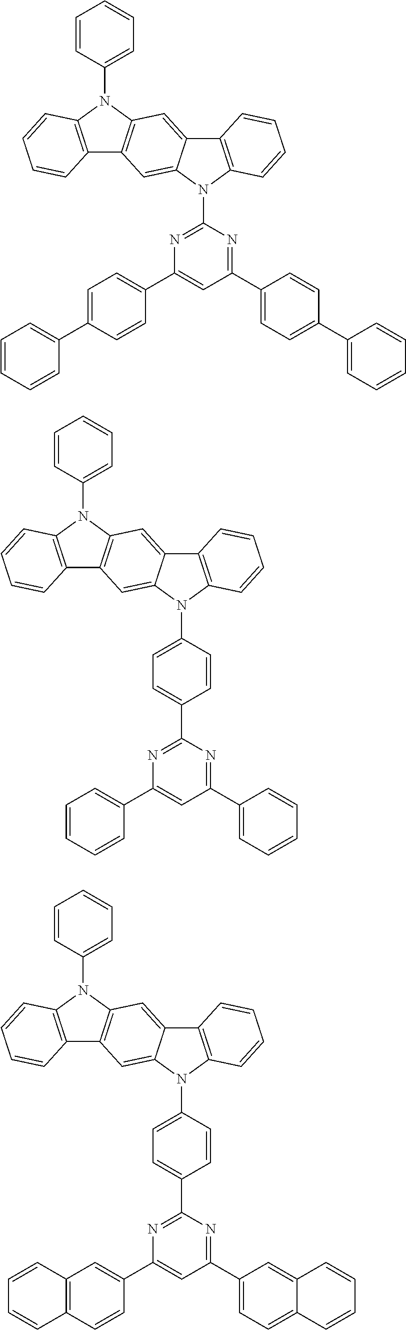

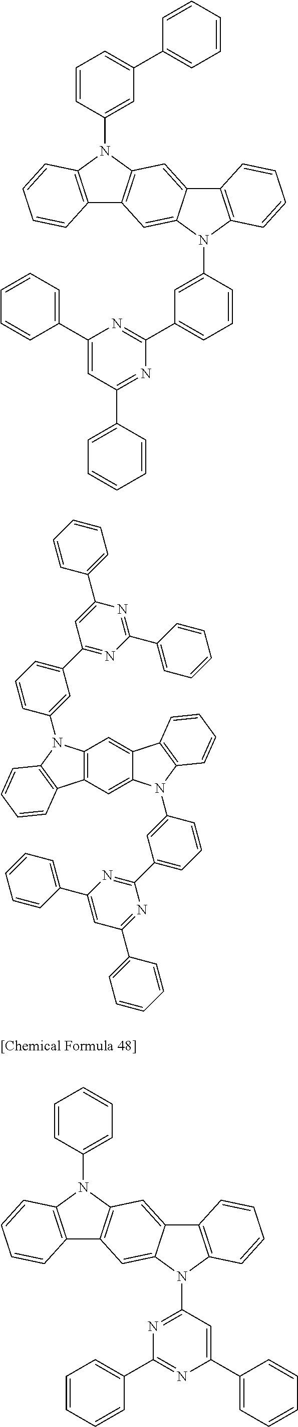

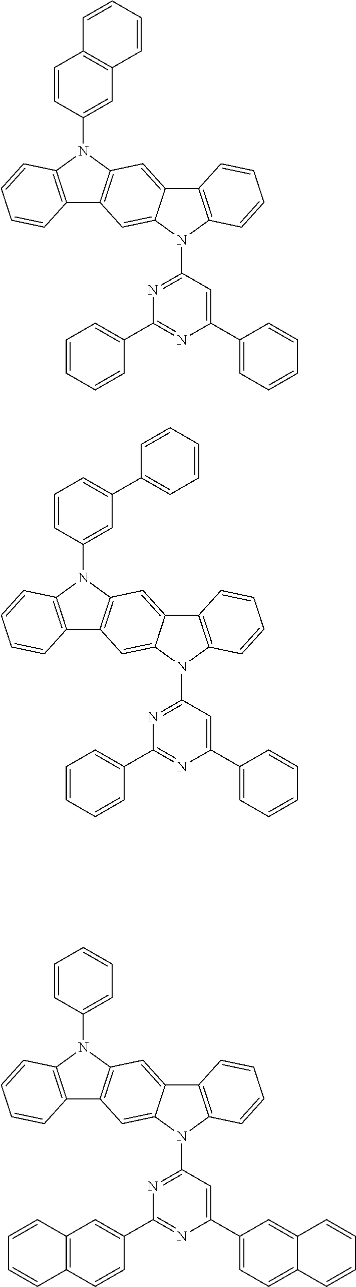

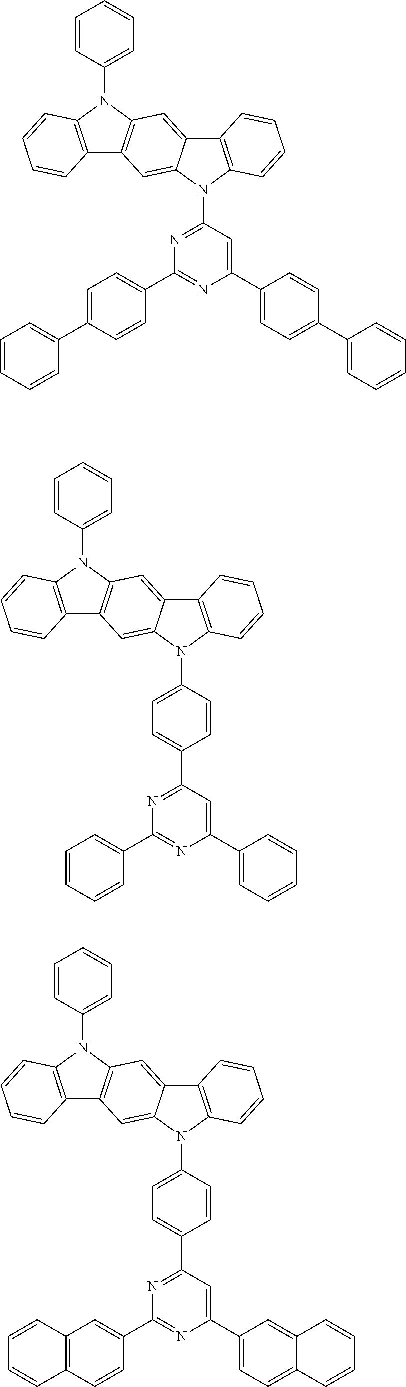

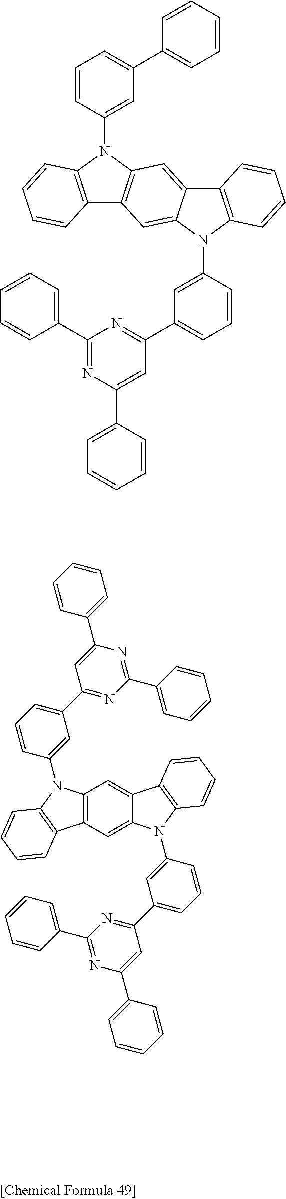

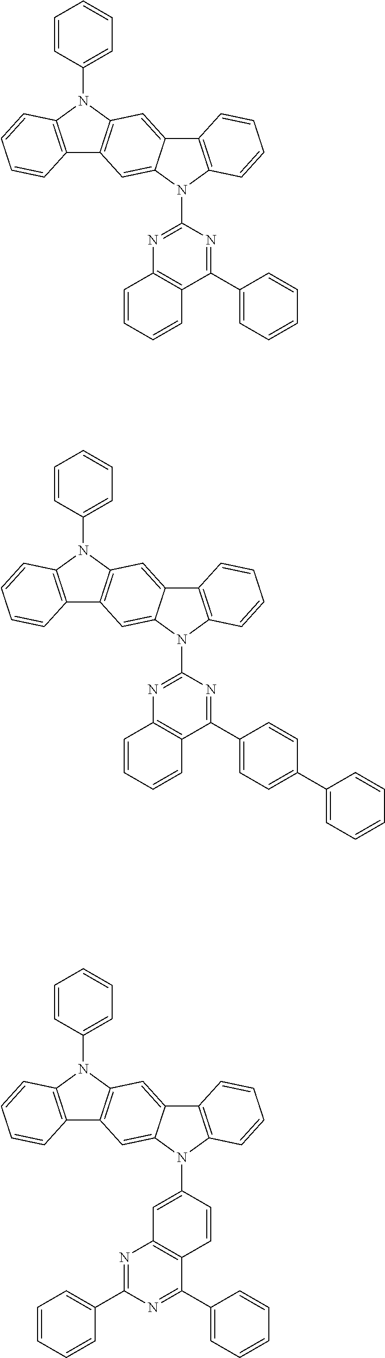

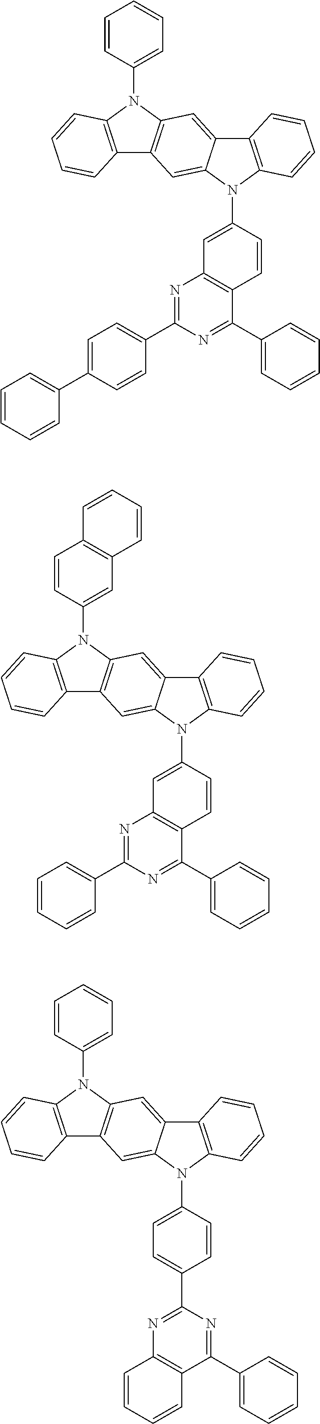

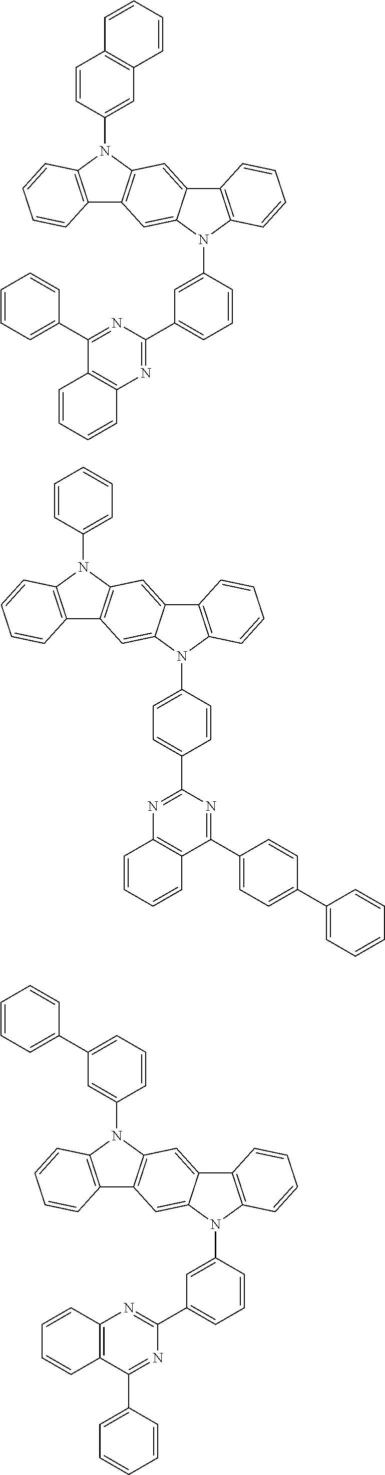

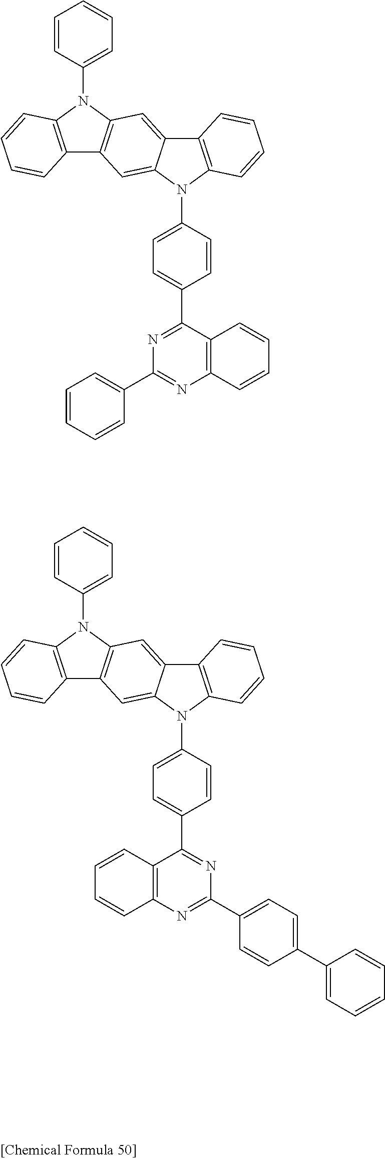

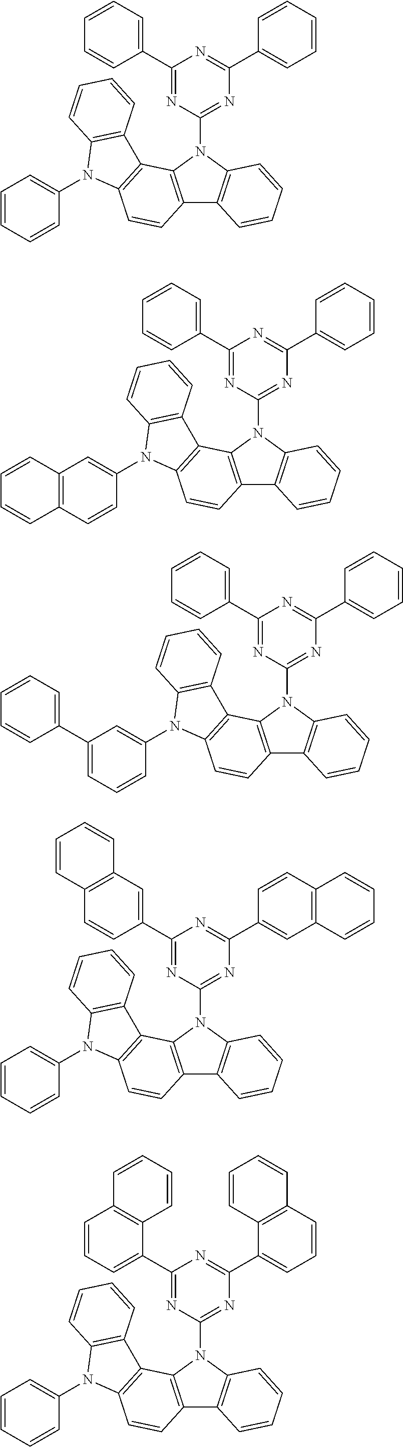

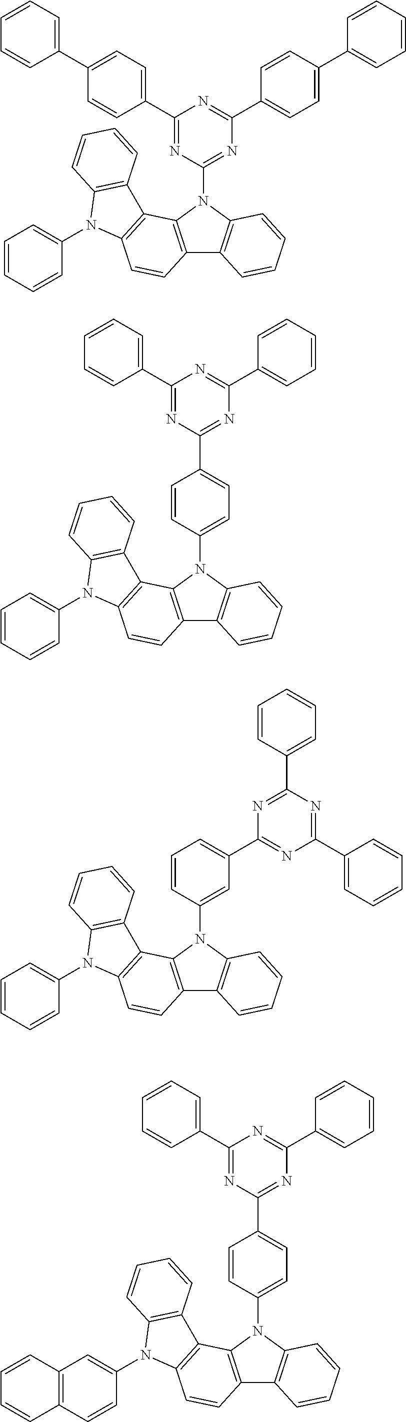

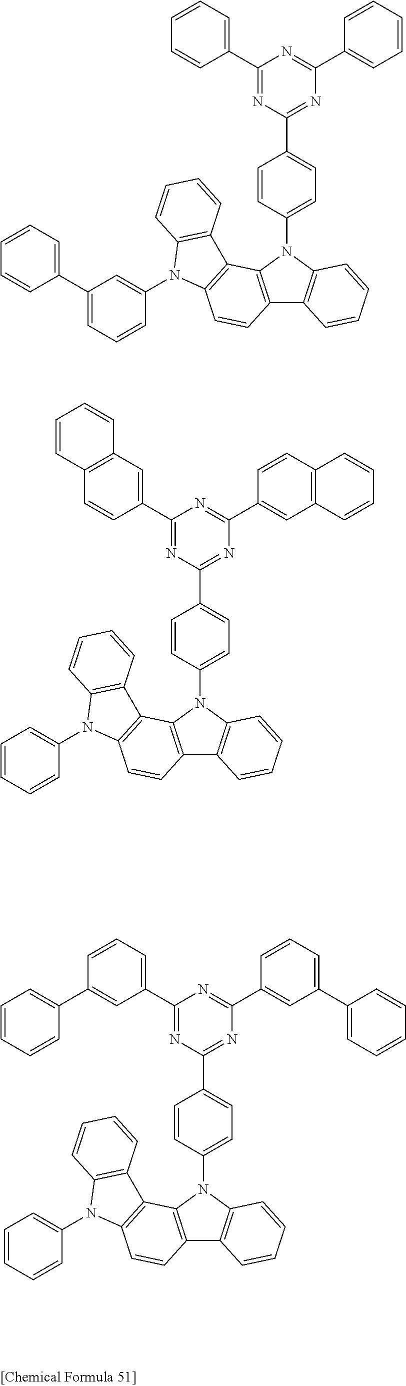

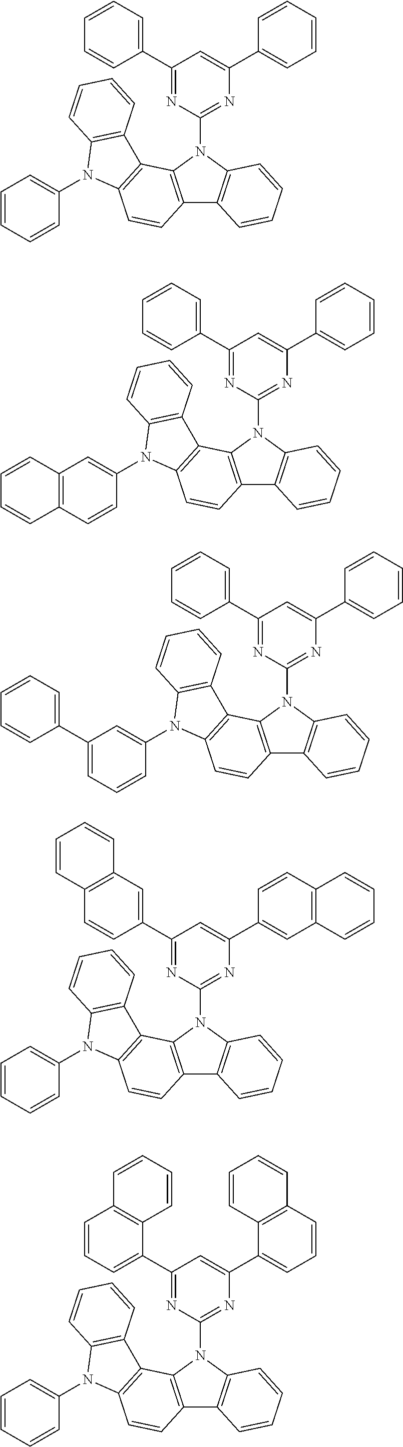

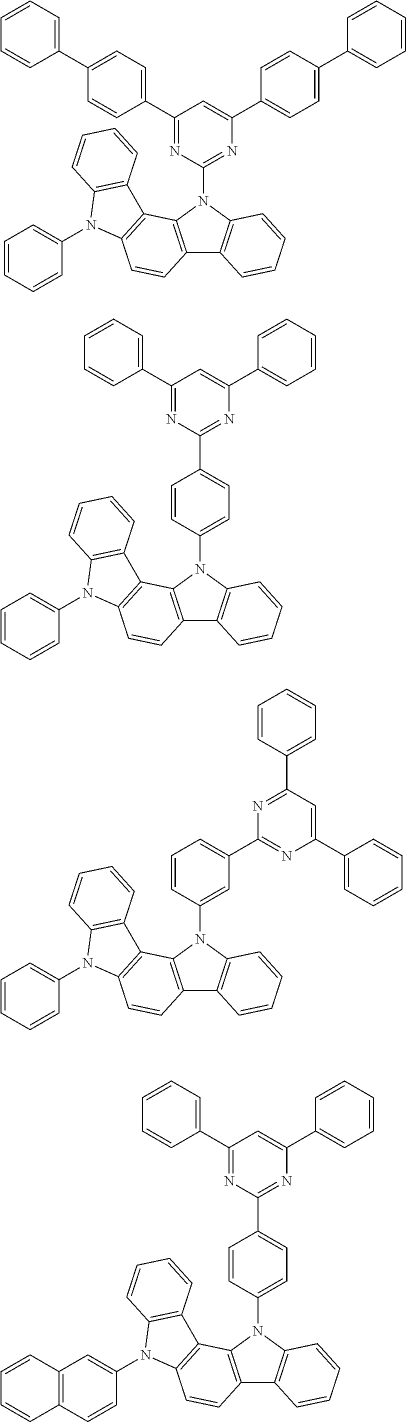

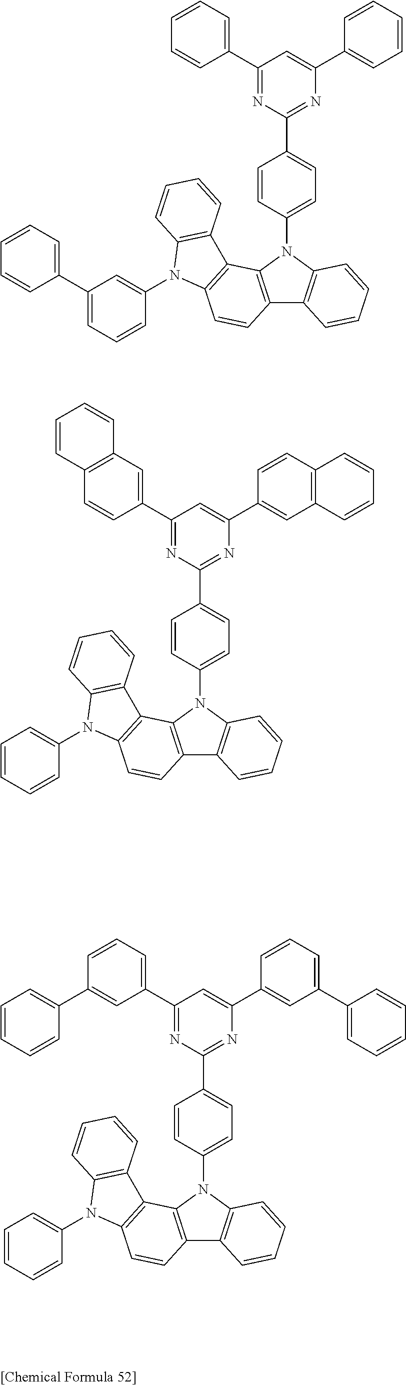

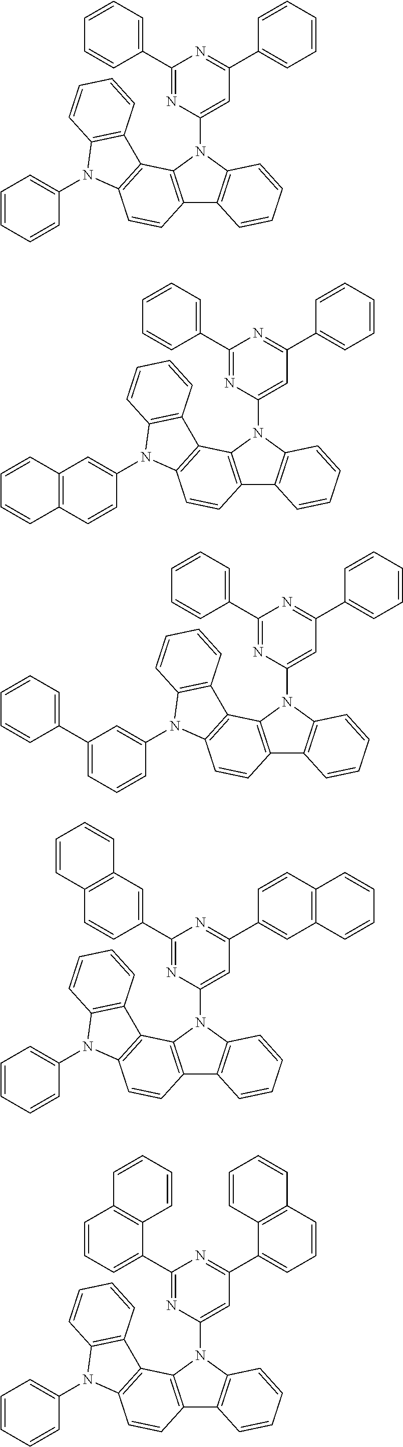

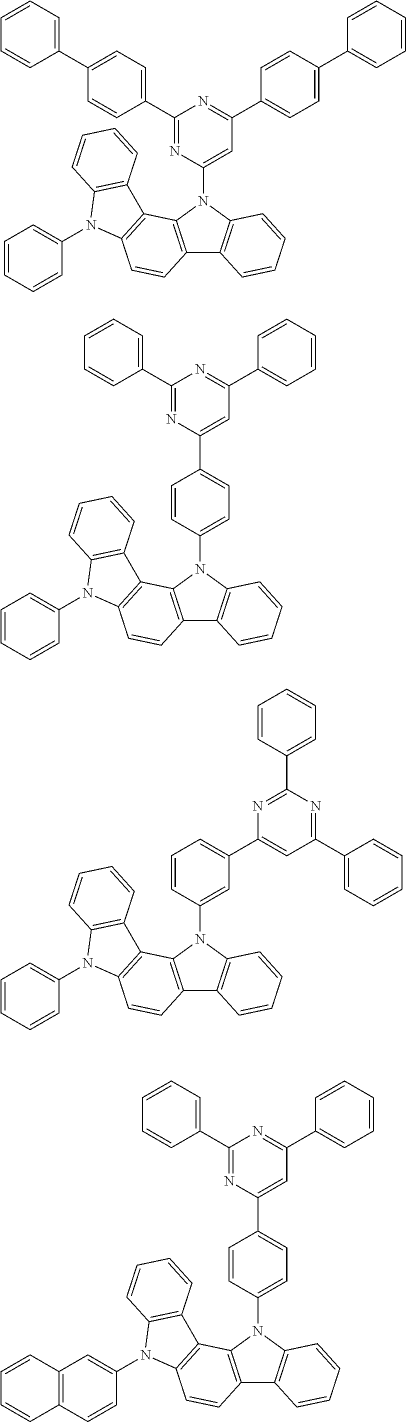

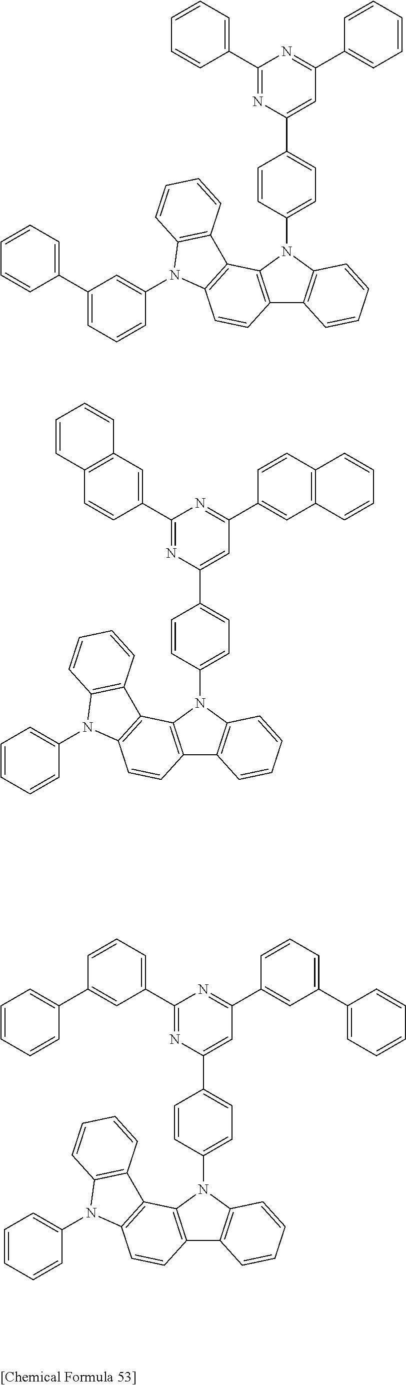

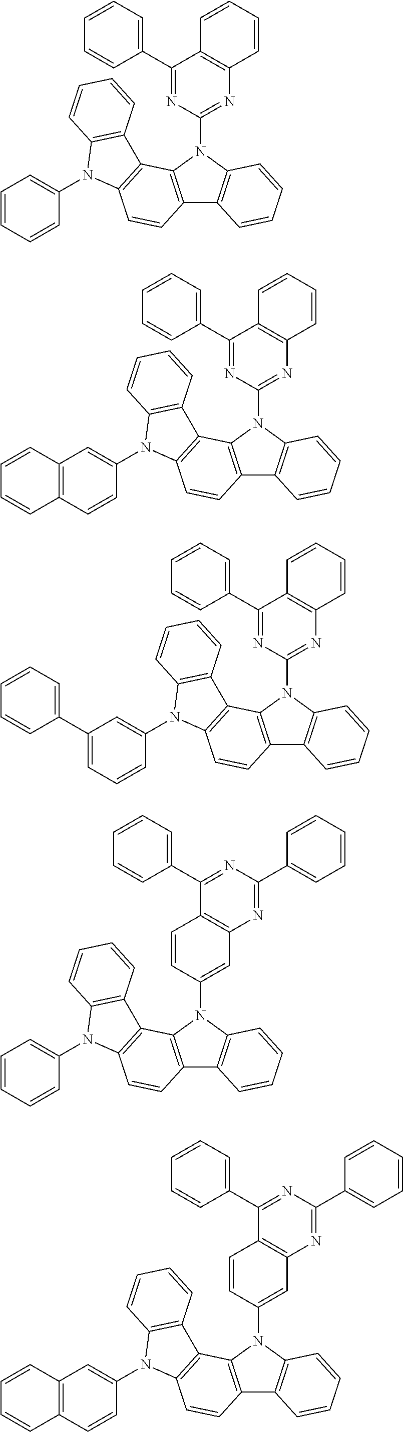

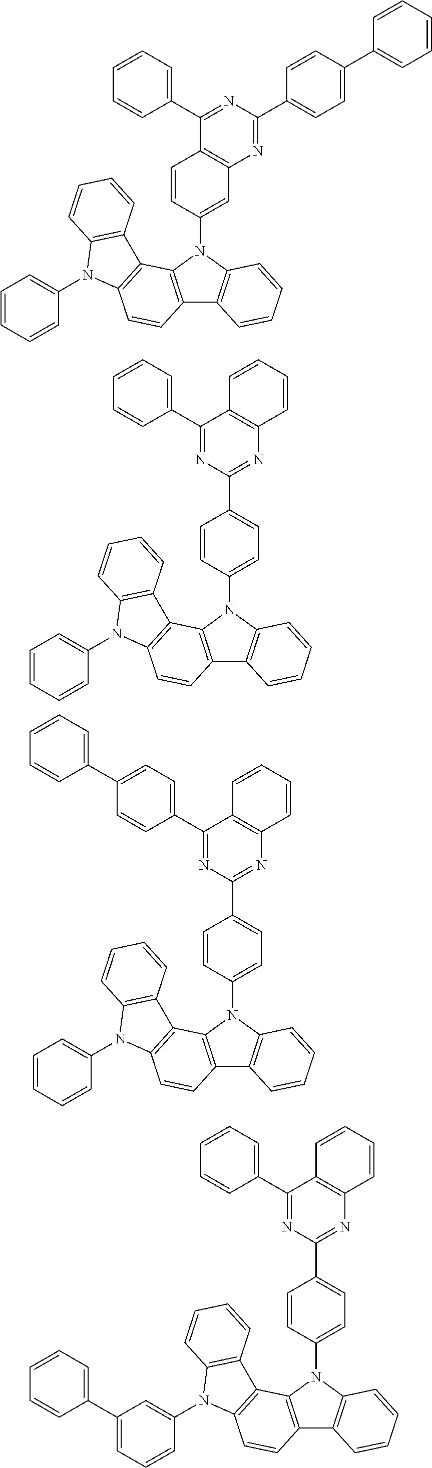

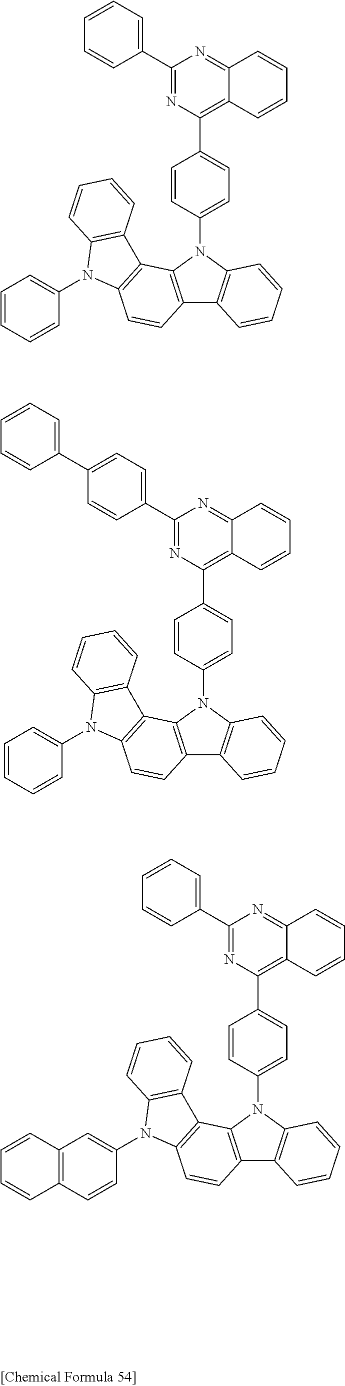

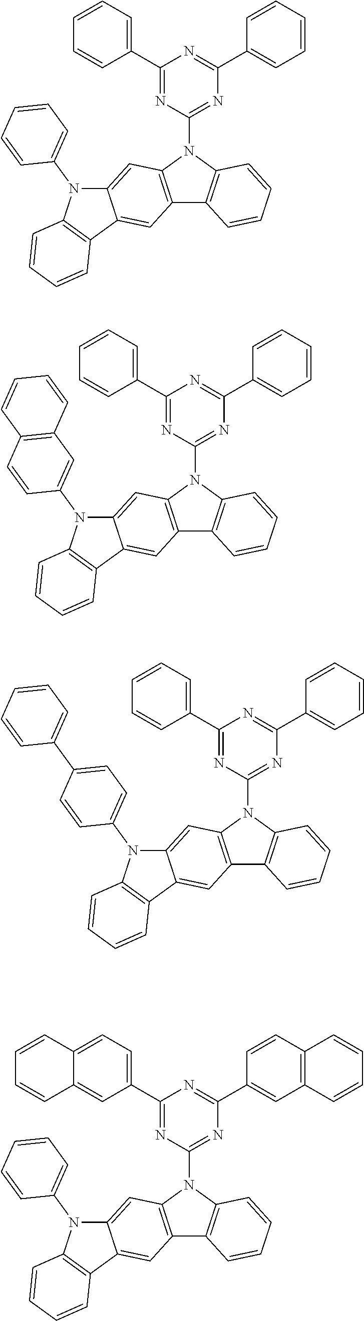

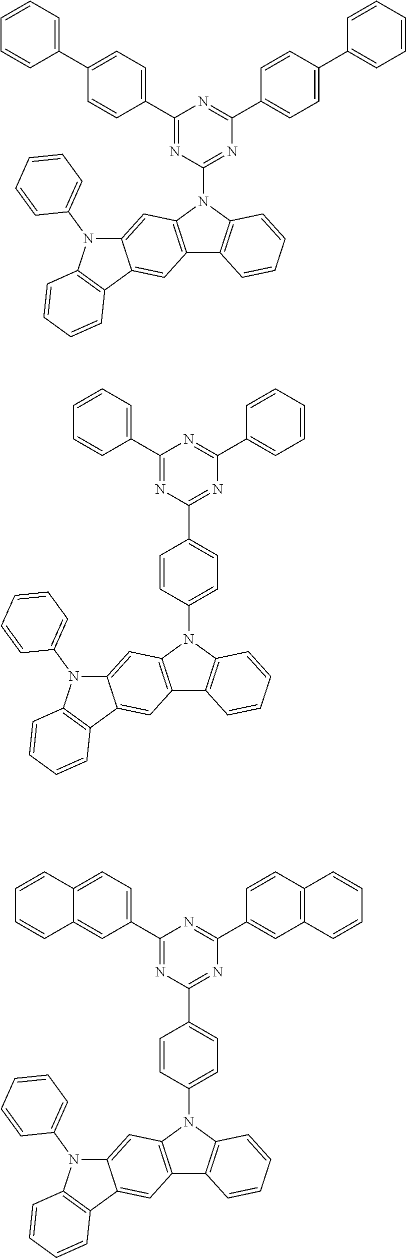

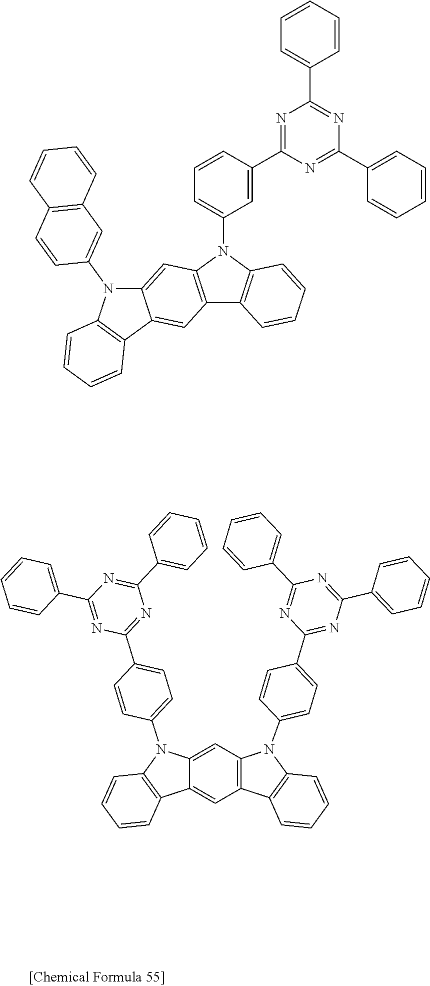

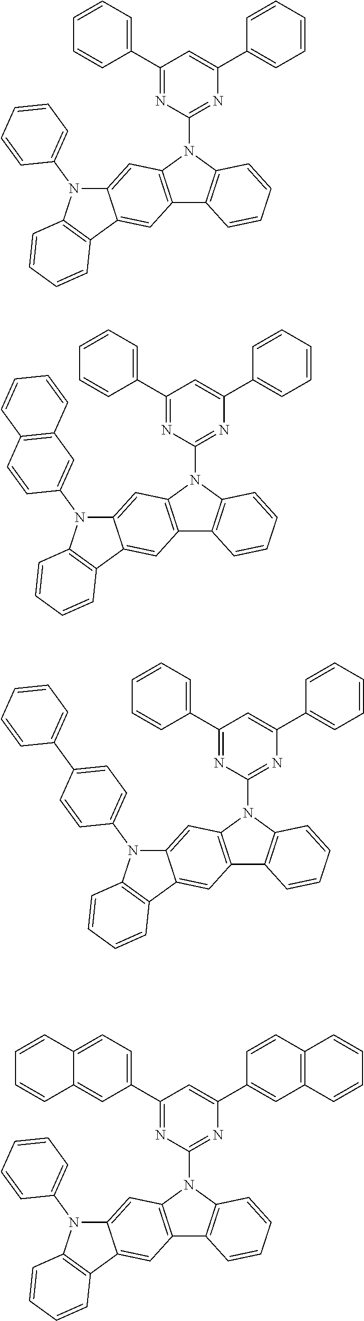

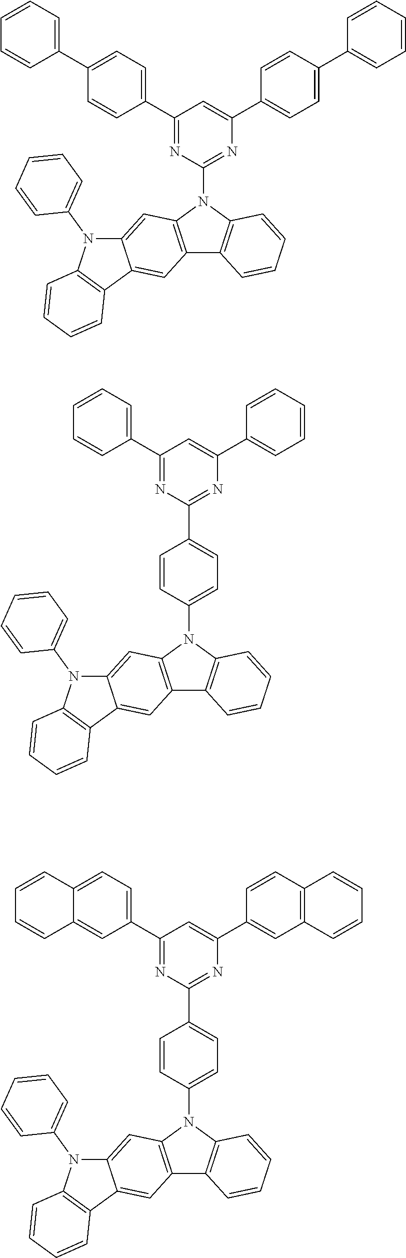

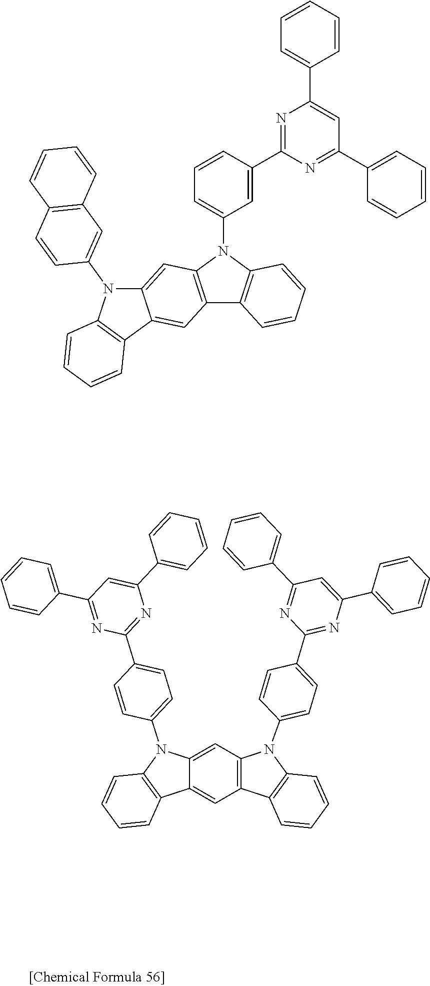

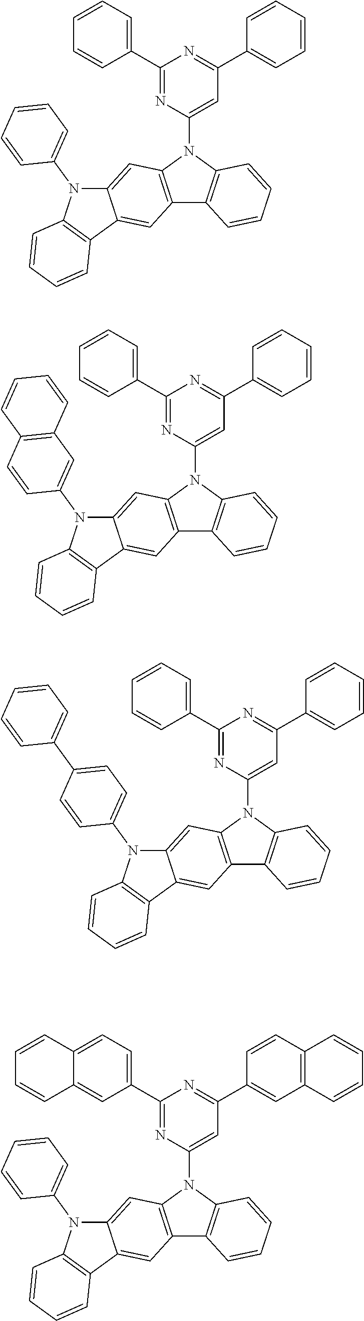

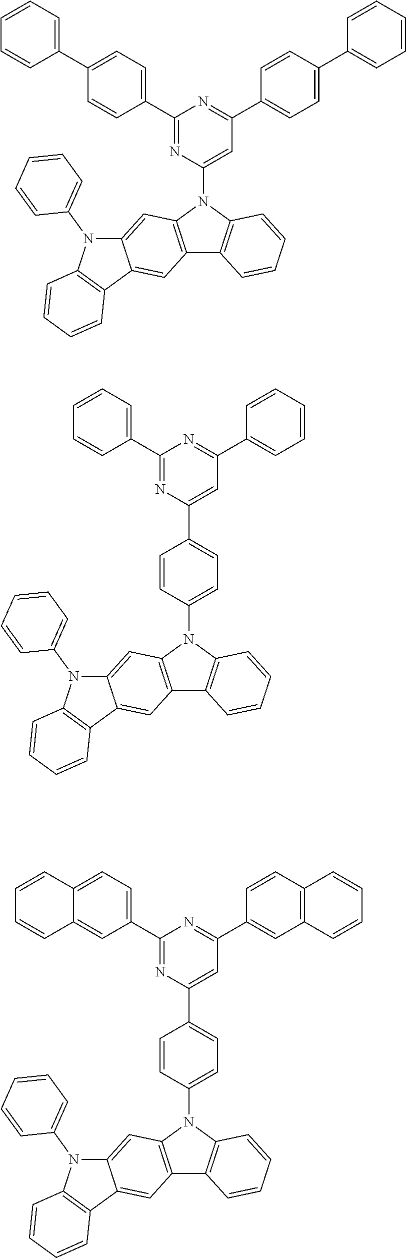

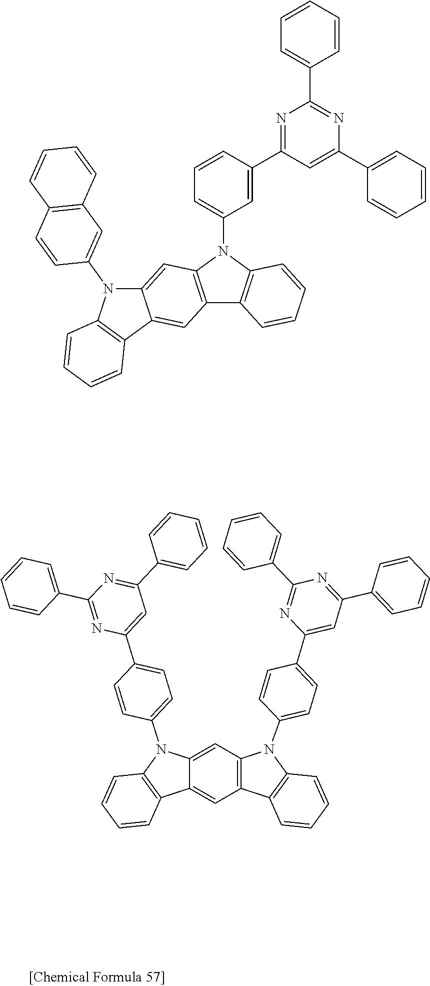

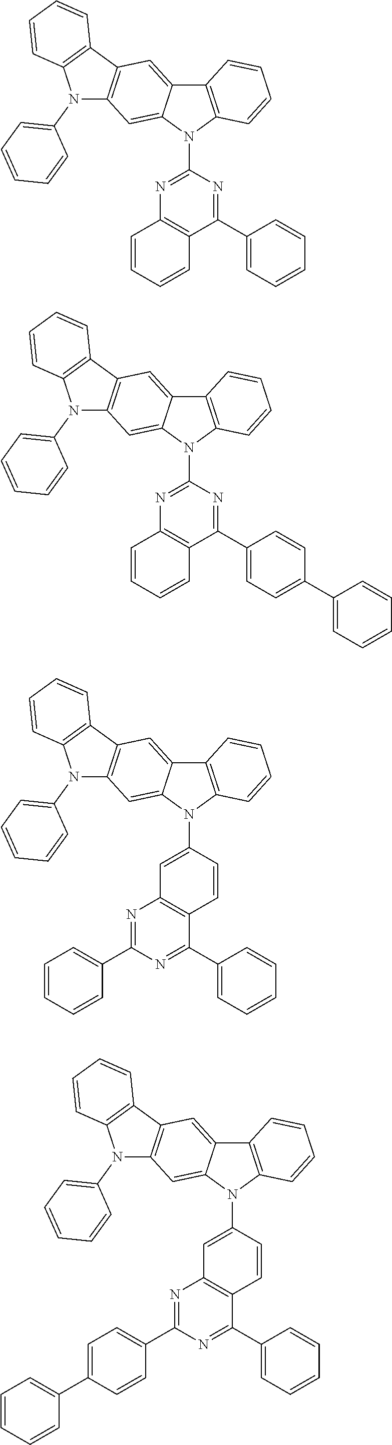

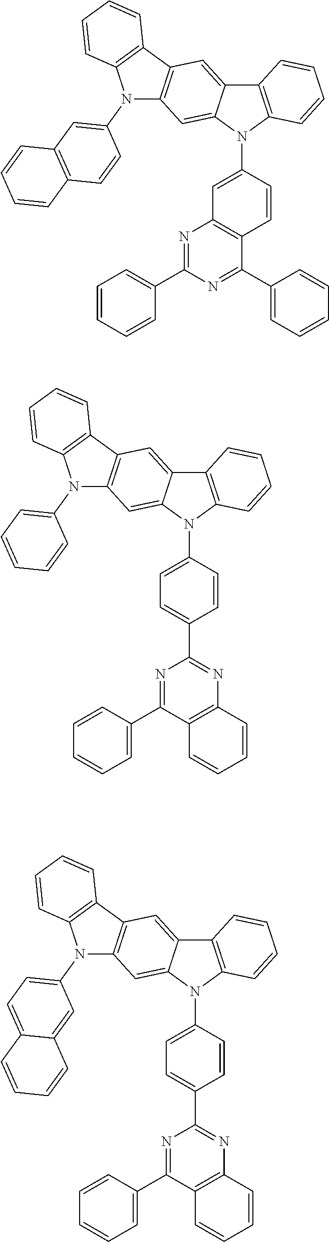

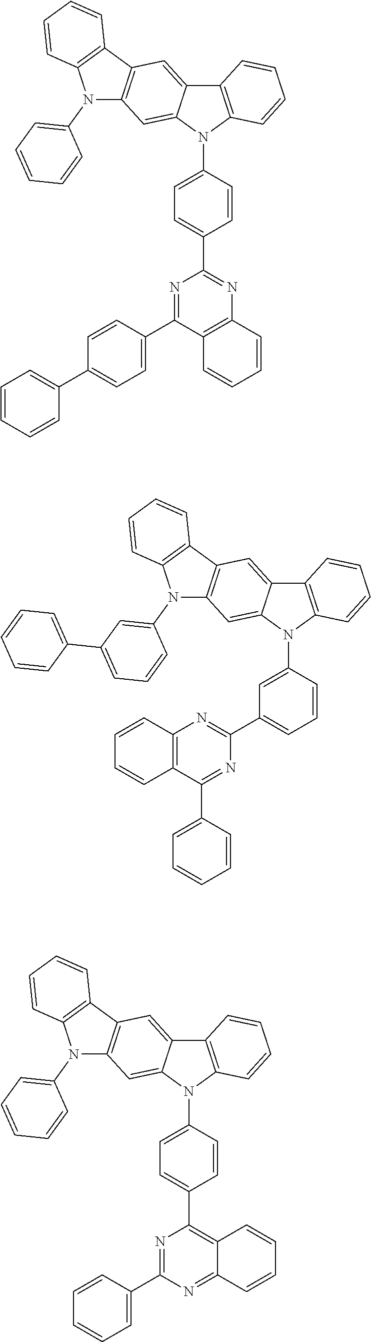

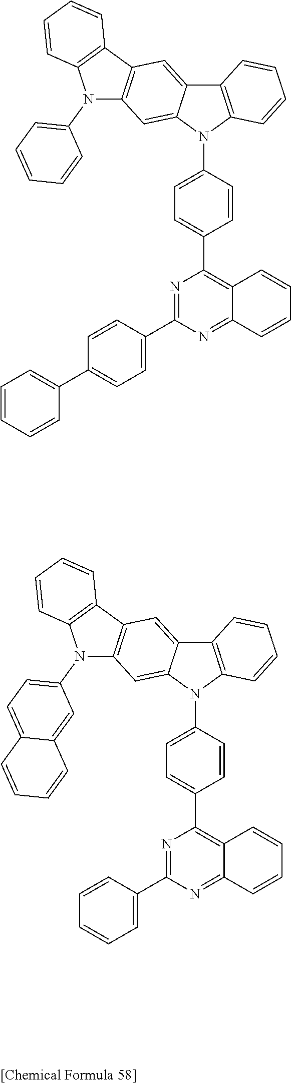

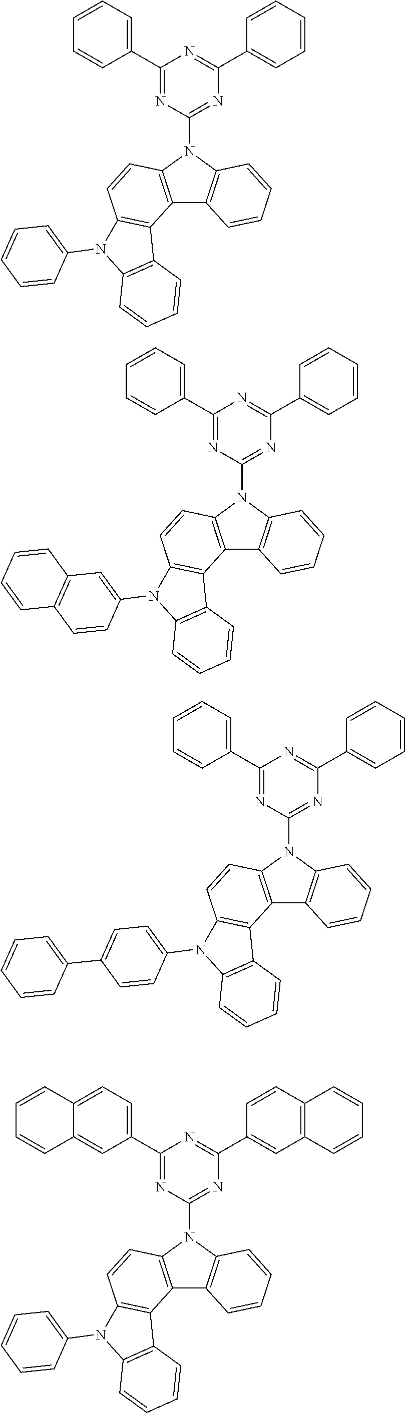

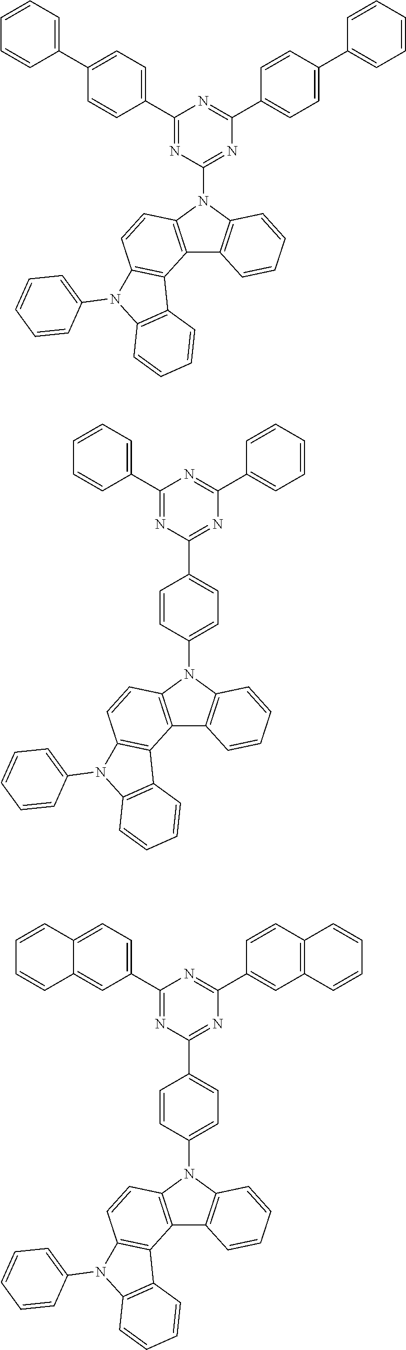

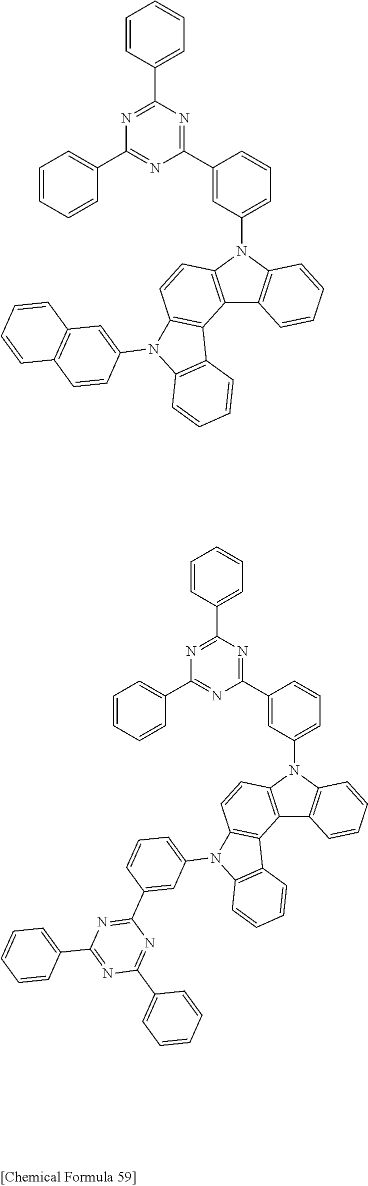

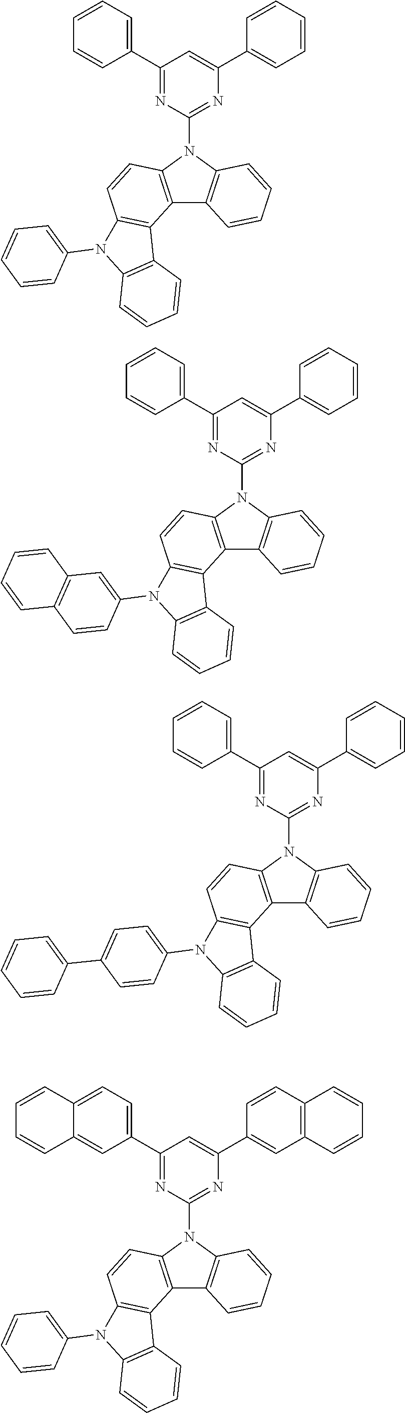

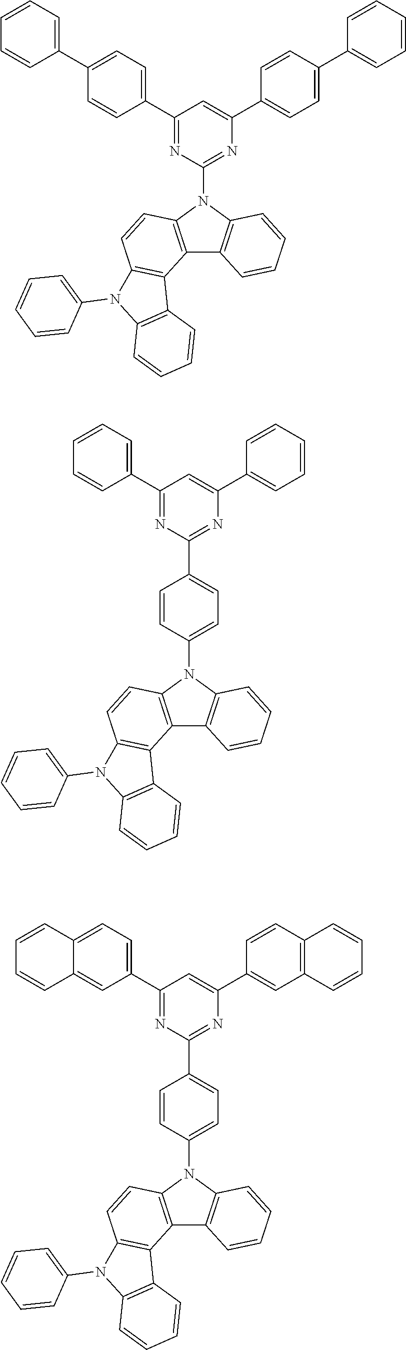

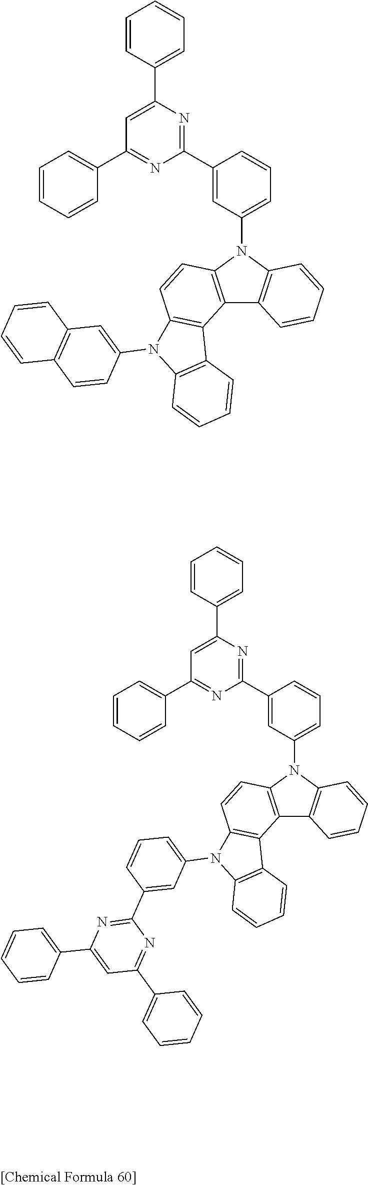

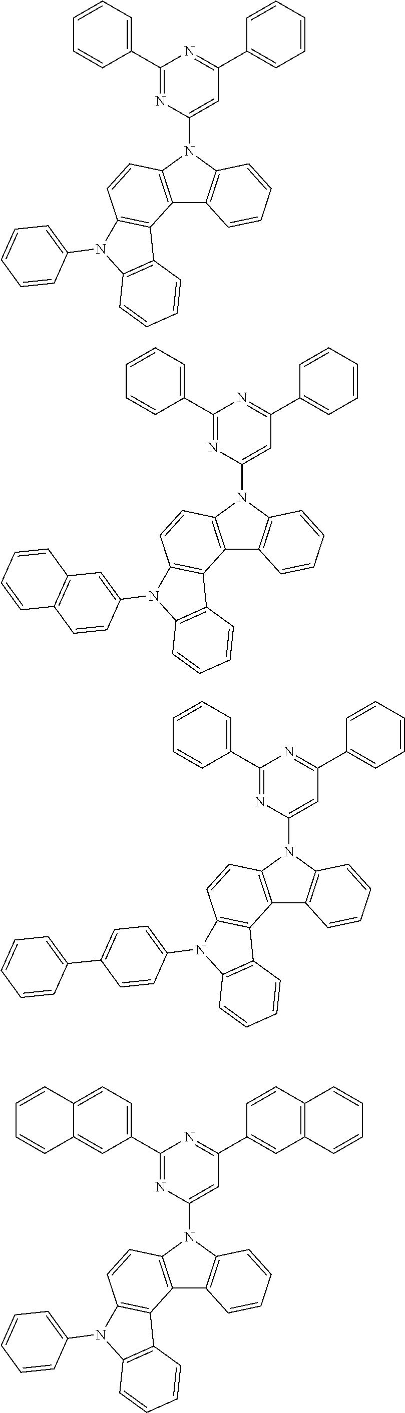

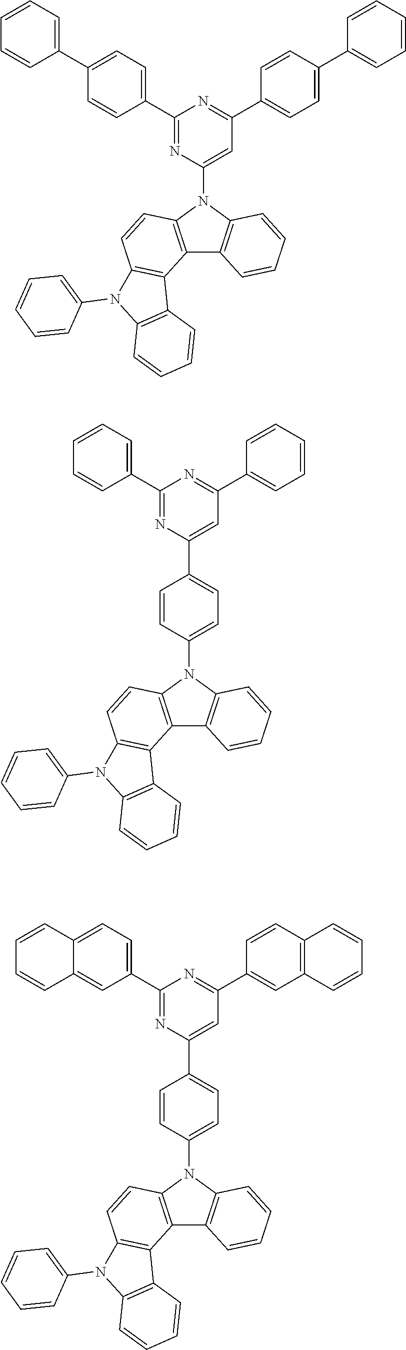

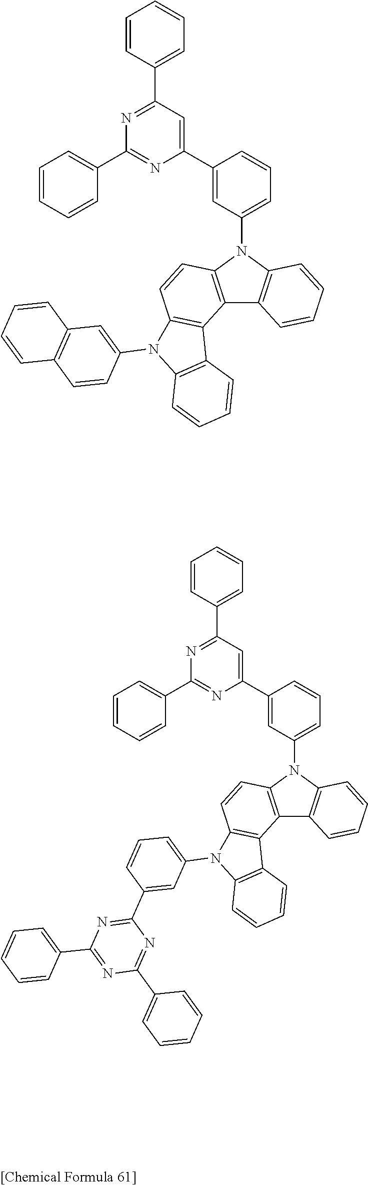

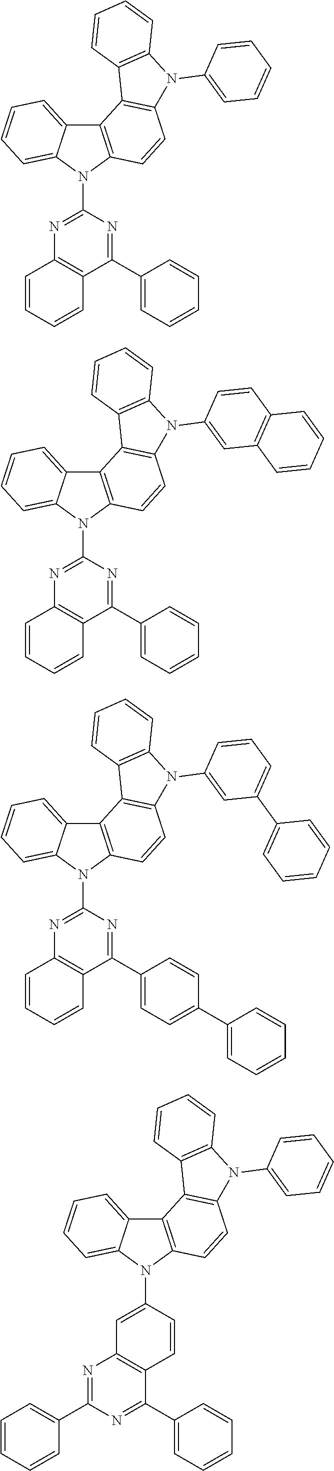

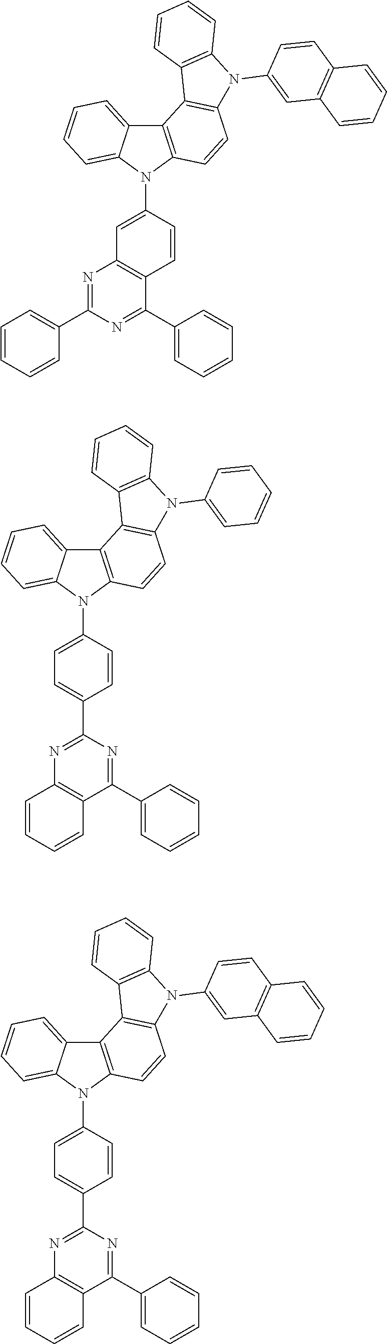

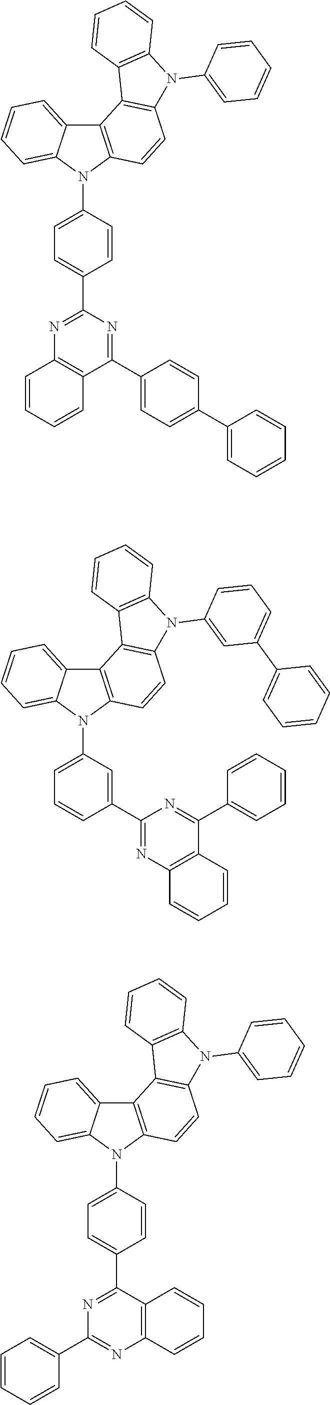

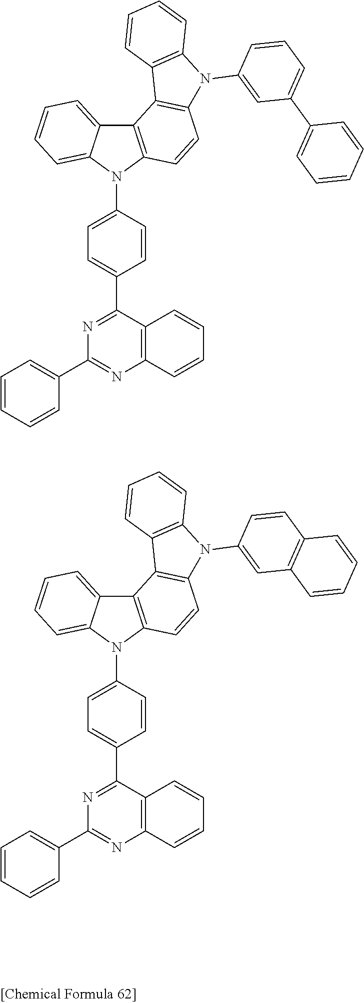

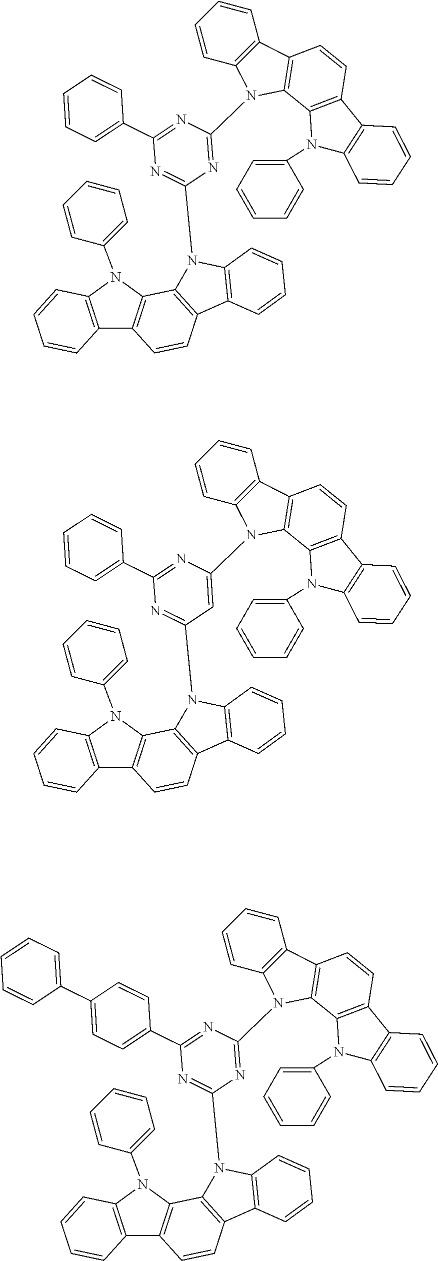

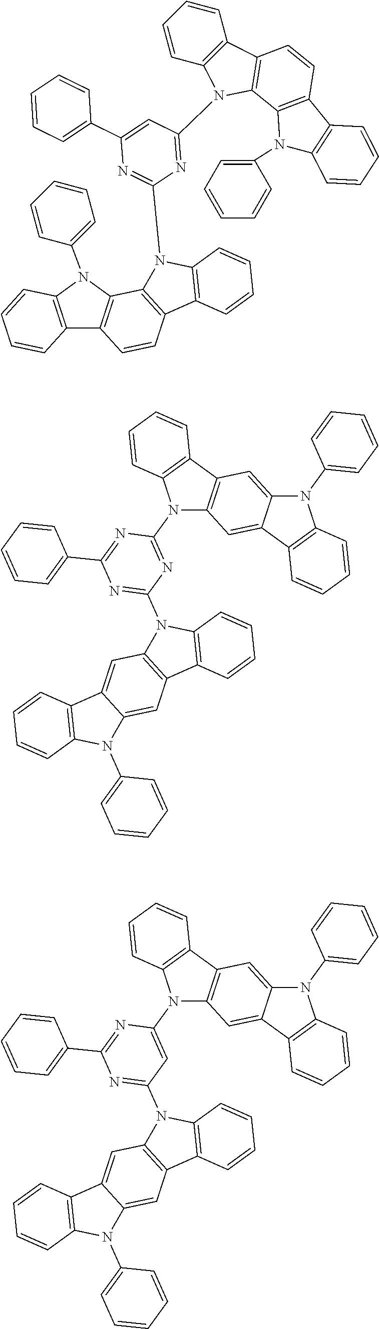

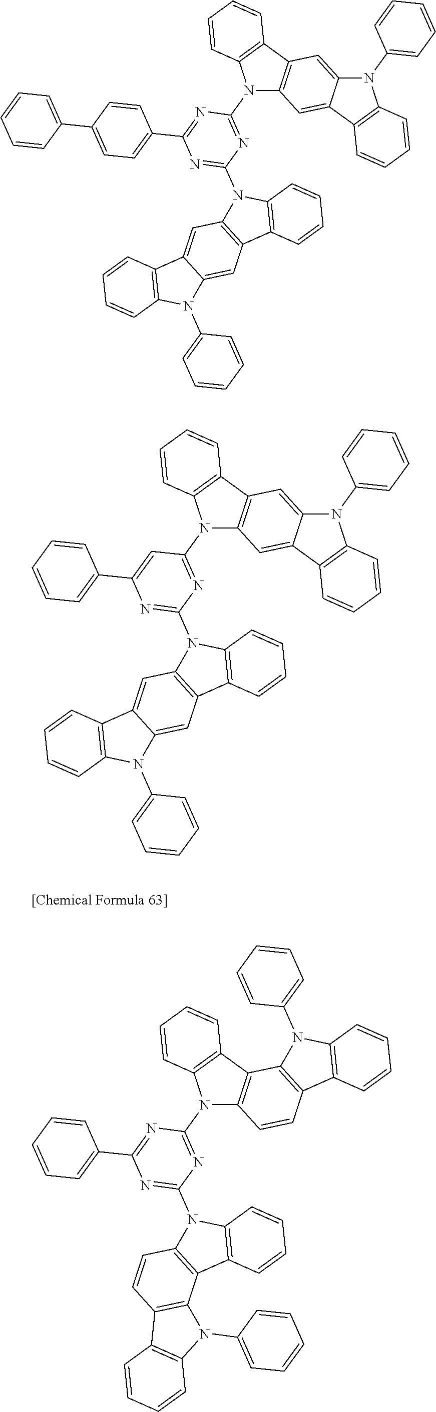

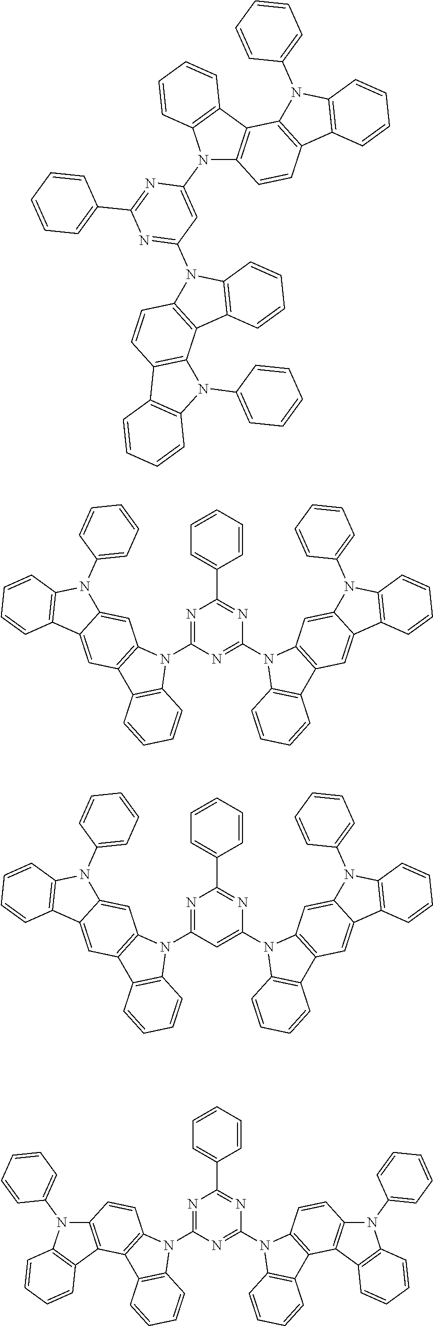

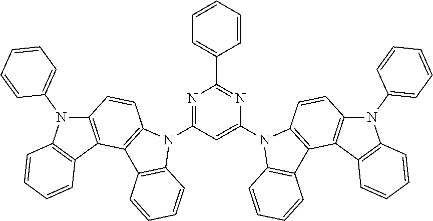

Known methods can be used for synthesizing the compound represented by general formula (2). Examples of a method in which an aryl group or a heteroaryl group is introduced into a moiety P include, but are not limited to, a method in which a carbon-carbon bond is generated by using a coupling reaction between a halogenated derivative of the moiety P and boronic acid or an esterified derivative of boronic acid of aryl or heteroaryl. Similarly, examples of a method in which an amino group or a carbazolyl group is introduced into a moiety Q include, but are not limited to, a method in which a carbon-nitrogen bond is generated by using a coupling reaction between a halogenated derivative of a moiety P and an amine or a carbazole derivative under the presence of a metal catalyst such as palladium.

### <Light-Emitting Element>

The light-emitting element according to the embodiment of the present invention includes an anode, a cathode, and an organic layer interposed between the anode and the cathode, and the organic layer emits light by means of electrical energy.

Examples of the laminated configuration of the organic layer include, besides a configuration made up of only an emissive layer, laminated configurations such as 1) hole transporting layer/emissive layer, 2) emissive layer/electron transporting layer, 3) hole transporting layer/emissive layer/electron transporting layer, 4) hole transporting layer/emissive layer/electron transporting layer/electron injection layer, and 5) hole injection layer/hole transporting layer/emissive layer/electron transporting layer/electron injection layer. Each of the layers may be in the form of a single layer or a plurality of layers. The light-emitting element may be a laminated-type light-emitting element including a plurality of phosphorescence emissive layers and fluorescence emissive layers, or a light-emitting element in which a phosphorescence emissive layer and a fluorescence emissive layer are combined with each other. Emissive layers exhibiting mutually different emitted colors can be laminated.

The light-emitting element may be a tandem-type light-emitting element in which a plurality of element configurations as described above are laminated with intermediate layers interposed therebetween. Generally, the intermediate layer is also called an intermediate electrode, an intermediate electroconductive layer, a charge generation layer, an electron draw-out layer, a connection layer, or an intermediate insulating layer, and for the intermediate layer, a known material configuration can be used. Specific examples of the tandem-type include laminated configurations which include a charge generation layer as an intermediate layer between an anode and a cathode, such as 4) hole transporting layer/emissive layer/electrode transporting layer/charge generation layer/hole transporting layer/emissive layer/electron transporting layer, and 5) hole injection layer/hole transporting layer/emissive layer/electron transporting layer/electron injection layer/charge generation layer/hole injection layer/hole transporting layer/emissive layer/electron transporting layer/electron injection layer.

The light-emitting element according to the embodiment of the present invention may have an element structure (top emission type) which takes out light from a cathode side or an element structure (bottom emission type) which takes out light from an anode side. However, the top emission type is more preferable in that an aperture ratio (the ratio of a light emission area to a pixel area) can be increased to provide increased luminance.

Further, in the top emission type, the color purity of light emission can be improved by using a microcavity structure in combination. The microcavity structure utilizes a resonance action with respect to a light emission wavelength. The top emission type is more preferable also in that it can further improve light emission having high color purity exhibited by the compound represented by general formula (1).

(Emissive Layer)

In the light-emitting element according to the embodiment of the present invention, at least one emissive layer contains a compound represented by general formula (1) and a compound represented by general formula (2). Although not particularly limited, it is preferred to use the compound represented by general formula (1) as a dopant and the compound represented by general formula (2) as a host material.

In a preferred embodiment of the present invention, the compound represented by general formula (2) exhibits TADF properties, and triplet excitation energy generated by the recombination of holes and electrons is converted into singlet excitation energy by the compound represented by general formula (2). Thereafter, the singlet excitation energy is transferred to the compound represented by general Formula (1), thereby emitting light.

The dopant material maybe only the compound represented by general formula (1), or may be a combination of a plurality of compounds. From the viewpoint of obtaining light emission having high color purity, only the compound represented by general formula (1) is preferred. From the viewpoint of color purity, the compound represented by general formula (1) is preferably dispersed in the emissive layer.

If the ratio of the compound represented by general formula (1) in the emissive layer is too large, a concentration quenching phenomenon occurs. Therefore, the ratio of the compound is preferably 5 wt % or less, more preferably 2 wt % or less, and still more preferably 1 wt % or less. The compound represented by general formula (1) has very high fluorescence quantum yield, and the singlet excitation energy received from general formula (2) is efficiently released as fluorescence. This can provide efficient light emission even at a low concentration.

The host material may be only the compound represented by general formula (2), or may be a combination of a plurality of compounds, but is preferably a combination of a plurality of compounds. When the compound represented by general formula (2) is combined with another host material, the content of the compound represented by general formula (2) in the emissive layer is decreased, whereby direct energy transfer from the triplet level of the compound represented by general formula (2) to the triplet level of the compound represented by general formula (1) can be suppressed by a Dexter mechanism. As a result, the efficiency of energy transfer through the TADF phenomenon is improved, whereby a high luminous efficiency can be expected.

The ratio of the compound represented by general formula (2) in the emissive layer is preferably less than 70 wt %, and more preferably less than 50 wt %.

The host material combined with the compound represented by general formula (2) is preferably a material having a triplet level higher than the singlet level of the compound represented by general formula (2). By suppressing energy transfer from the singlet level and triplet level of the compound represented by general formula (2) to the triplet level or singlet level of another host material, the excitation energy generated by the recombination of holes and electrons can be confined. Examples of the host material include, but are not limited to, fused aromatic ring derivatives such as anthracene and pyrene, fluorene derivatives, dibenzofuran derivatives, carbazole derivatives, and indolocarbazole derivatives. Among these, carbazole derivatives such as 4,4′-bis (carbazol-9-yl)biphenyl (CBP), 1,3-bis(carbazol-9-yl)benzene, and a carbazole multimer have a higher triplet level, which are preferable.

Further, a carbazole multimer is preferred in view of excellent carrier transport properties, and a bis(N-arylcarbazole) derivative represented by general formula (14) is more preferable.

In the following description, unless otherwise specified, the contents of substituents are the same as those shown in the description regarding the compound represented by general formula (1).

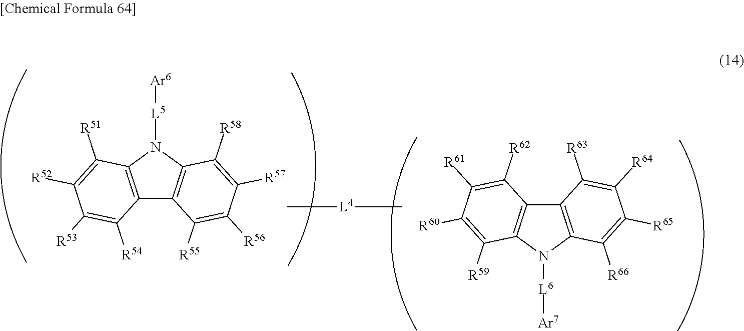

R⁵¹ to R⁶⁶ are the same or different from each other, and each are selected from a hydrogen atom, an alkyl group, a cycloalkyl group, a heterocyclic group, an alkenyl group, a cycloalkenyl group, an alkynyl group, a hydroxyl group, a thiol group, an alkoxy group, an alkylthio group, an aryl ether group, an aryl thioether group, an aryl group, a heteroaryl group, a halogen, a cyano group, an aldehyde group, a carbonyl group, a carboxyl group, an ester group, a carbamoyl group, an amino group, a nitro group, a silyl group, a siloxanyl group, a boryl group, —P(═O)R¹⁰R¹¹, and a fused ring and an aliphatic ring formed with an adjacent substituent. L⁴ is connected to one position of R⁵¹ to R⁵⁸ and one position of R⁵⁹ to R⁶⁶. R¹⁰ and R¹¹ each are an aryl group or a heteroaryl group.

L⁴ to L⁶ each are a single bond or a phenylene group. L⁴ is connected to one position of R⁵¹ to R⁵⁸ and one position of R⁵⁹ to R⁶⁶.

Ar⁶ and Ar⁷ are the same or different from each other, and each represent a substituted or unsubstituted aryl group.

In general formula (14), L⁴ is preferably connected to one position of R⁵⁶ and R⁵⁷ and one position of R⁶⁰ and R⁶¹. This is because the hole transport properties of the compound represented by general formula (14) are improved, to provide an improved carrier balance when the compound is combined with the compound represented by general formula (2). Further, it is more preferred that L⁴ is connected to the positions of R⁵⁶ and R⁶¹, or L⁴ is connected to the positions of R⁵⁷ and R⁶⁰, and it is particularly preferred that L⁴ is connected to the positions of R⁵⁶ and R⁶¹.

When L⁴ is a single bond, the triplet level becomes high, which is more preferable.

The aryl groups of Ar⁶ and Ar⁷ are the same or different from each other, and each are preferably a phenyl group, a biphenyl group, a terphenyl group, a naphthyl group, a fluorenyl group, a phenanthryl group, an anthracenyl group, a pyrenyl group, a fluoranthenyl group, or triphenylenyl group, and more preferably a phenyl group, a biphenyl group, a terphenyl group, a naphthyl group, a fluorenyl group, a phenanthryl group, or a triphenylenyl group since the conjugation is not excessively wide and the triplet level is not excessively low. Still more preferred are a phenyl group, a biphenyl group, a terphenyl group, a naphthyl group, and a fluorenyl group. When these groups are substituted, the substituent is preferably selected from an alkyl group, a cycloalkyl group, an alkoxy group, an aryl ether group, a halogen, a cyano group, an amino group, a nitro group, a silyl group, a phenyl group, and a naphthyl group.

Among these, Ar⁶ and Ar⁷ are the same or different from each other, and each are a substituted or unsubstituted phenyl group, a substituted or unsubstituted biphenyl group, a substituted or unsubstituted terphenyl group, or a substituted or unsubstituted 2-fluorenyl group, to provide a high triplet level, which is preferable. When these groups are substituted, the substituent is preferably selected from an alkyl group, a cycloalkyl group, an alkoxy group, an aryl ether group, a halogen, a cyano group, an amino group, a nitro group, a silyl group, and a phenyl group.

Preferred examples of Ar⁶ and Ar⁷ are not particularly limited, but specific examples thereof include the following.

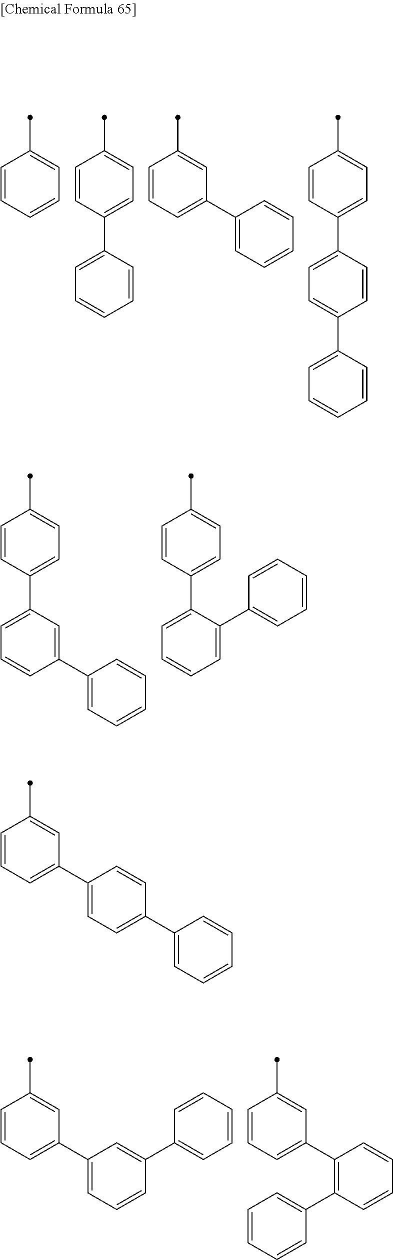

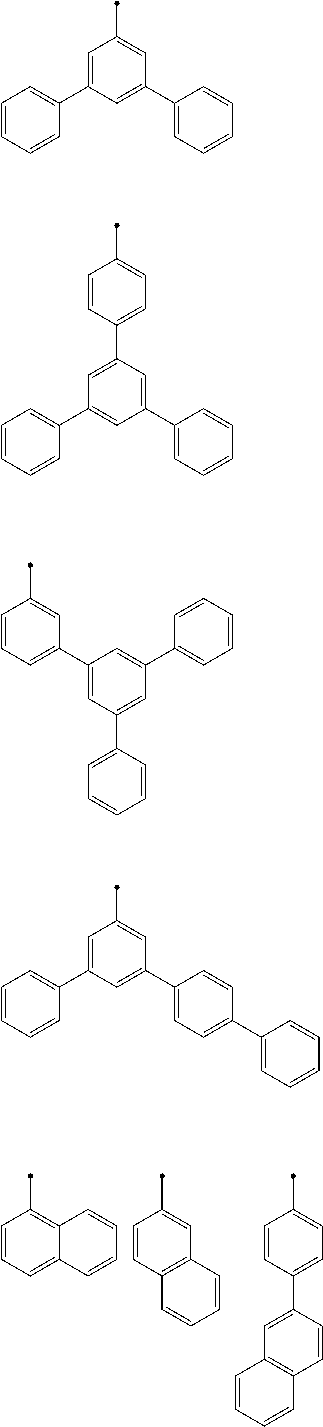

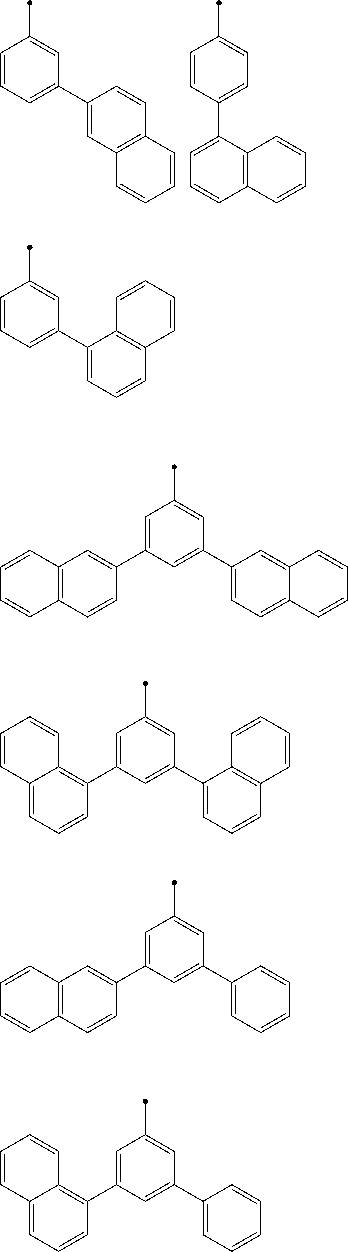

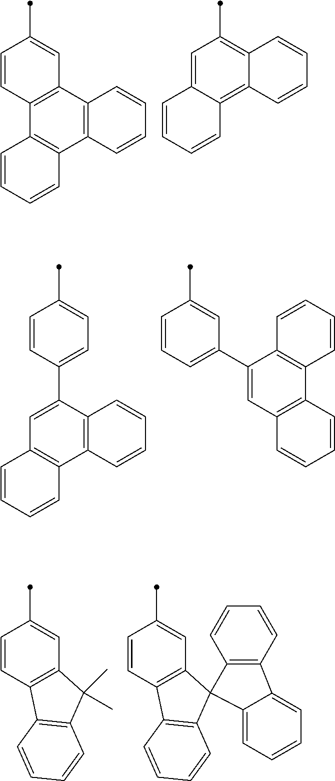

When Ar⁶ and Ar⁷ are different from each other, the compound represented by general formula (14) has an asymmetric structure, whereby an interaction between carbazole skeletons is suppressed to allow a stable thin film to be formed, which is preferable.

When, as one aspect of the compound represented by general formula (14), R⁶⁴ is an aryl group, the hole transport properties of the compound represented by general formula (14) are improved. When the compound is combined with the compound represented by general formula (2), a carrier balance is improved, which is preferable.

When R⁶⁴ is an aryl group, the aryl groups are the same or different from each other from the viewpoint that the conjugation is not excessively wide and the triplet level is not excessively low. The aryl group is preferably a substituted or unsubstituted phenyl group, biphenyl group, terphenyl group, naphthyl group, fluorenyl group, phenanthryl group, anthracenyl group, pyrenyl group, fluoranthenyl group, and triphenylenyl group, and more preferably a substituted or unsubstituted phenyl group, biphenyl group, terphenyl group, naphthyl group, fluorenyl group, phenanthryl group, or triphenylenyl group.

Among these, R⁶⁴ is a substituted or unsubstituted phenyl group, a substituted or unsubstituted biphenyl group, a substituted or unsubstituted 2-fluorenyl group, a substituted or unsubstituted terphenyl group, and a substituted or unsubstituted naphthyl group, whereby the triplet level is high, which is preferable. Particularly preferred are a substituted or unsubstituted phenyl group, a substituted or unsubstituted biphenyl group, a substituted or unsubstituted 2-fluorenyl group, and a substituted or unsubstituted terphenyl group. When these groups are substituted, the substituent is preferably selected from an alkyl group, a cycloalkyl group, an alkoxy group, an aryl ether group, a halogen, a cyano group, an amino group, a nitro group, and a silyl group.

The compound represented by general formula (14) is not particularly limited, but specific examples thereof include the following.

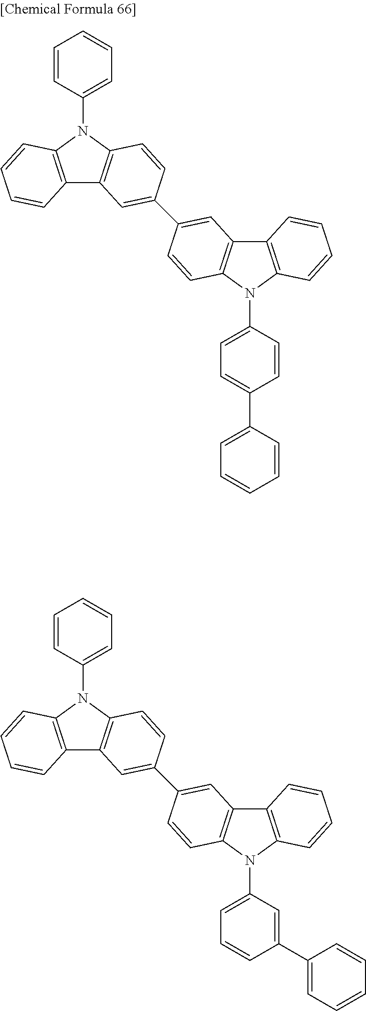

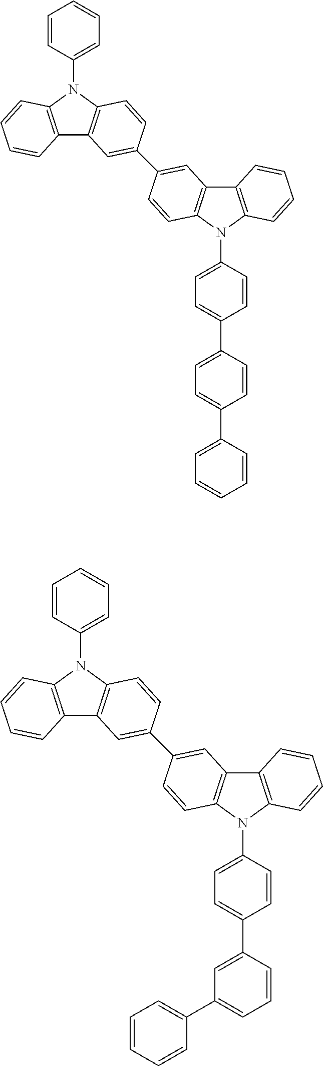

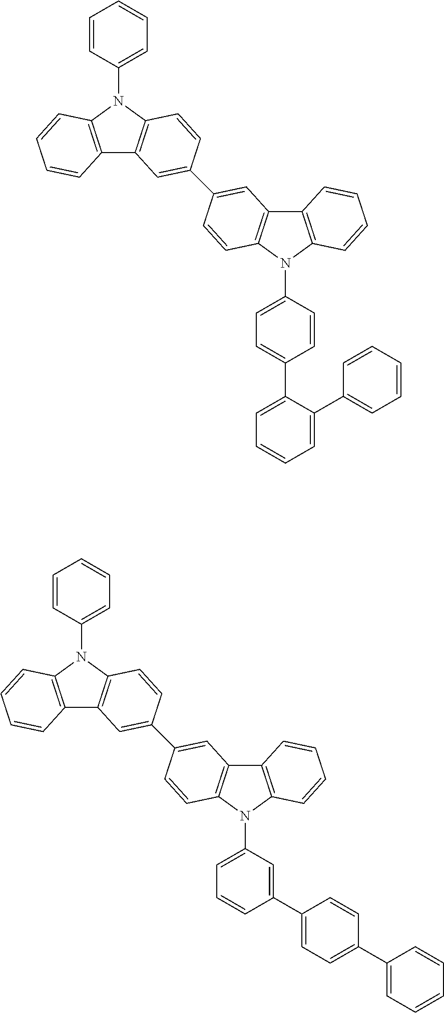

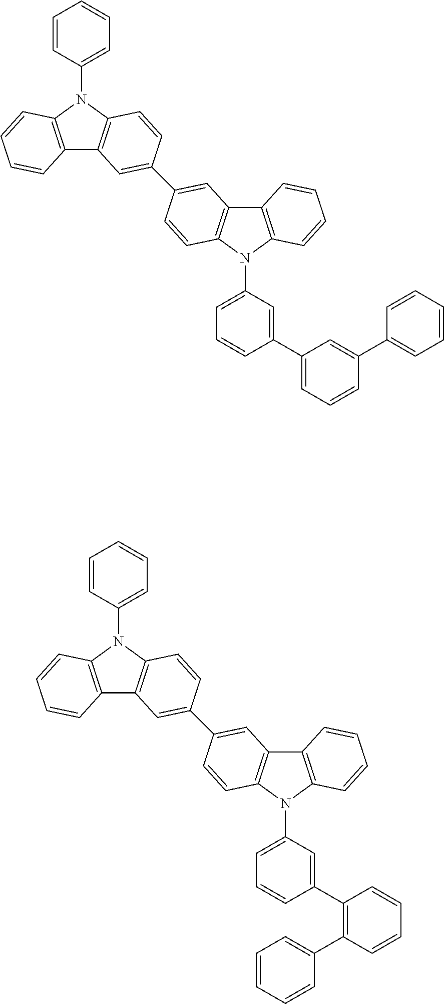

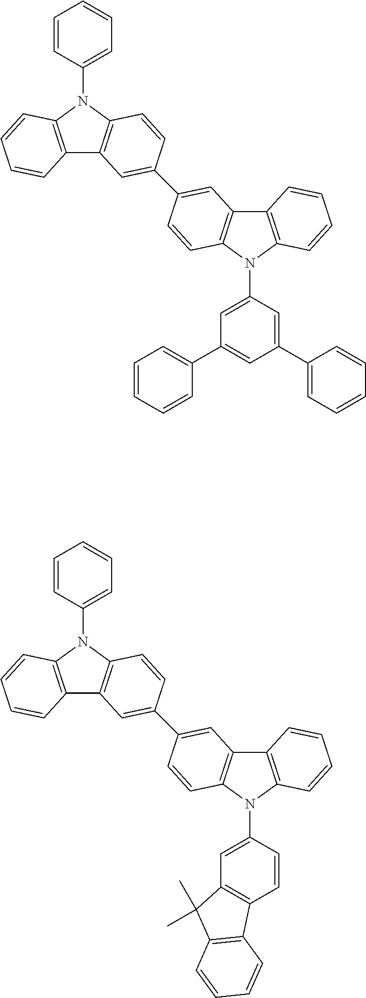

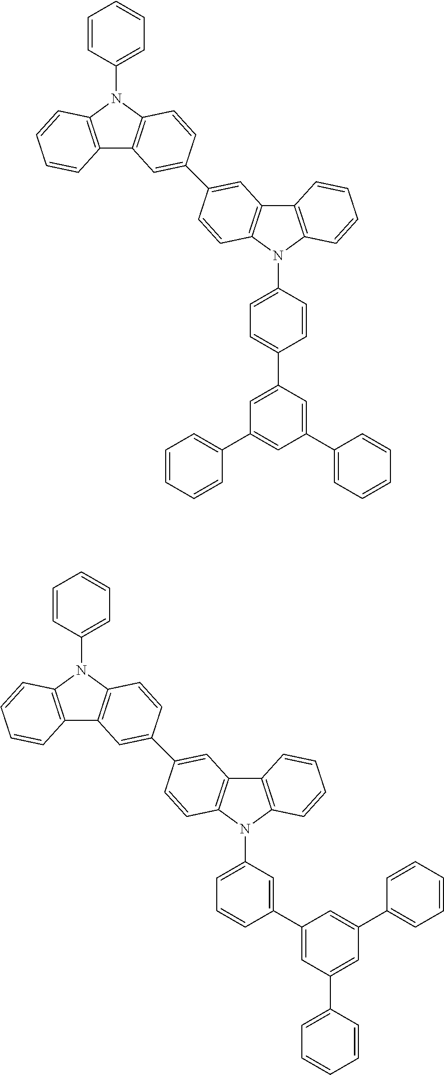

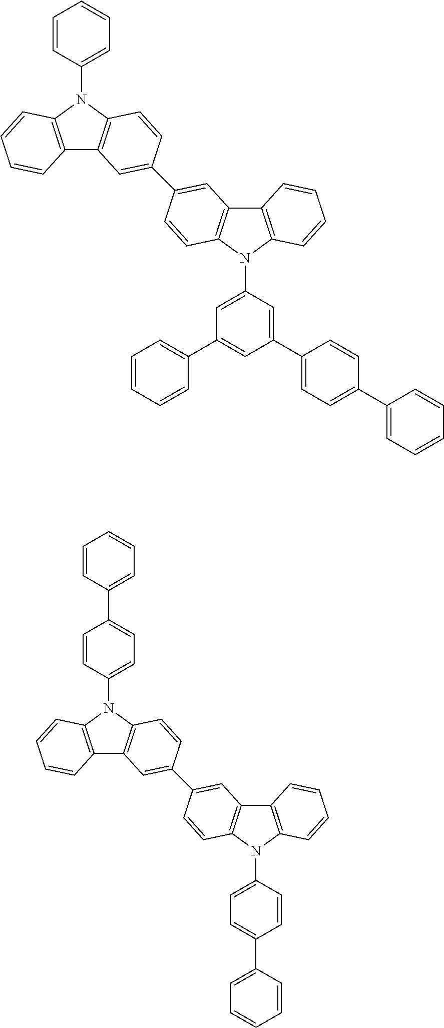

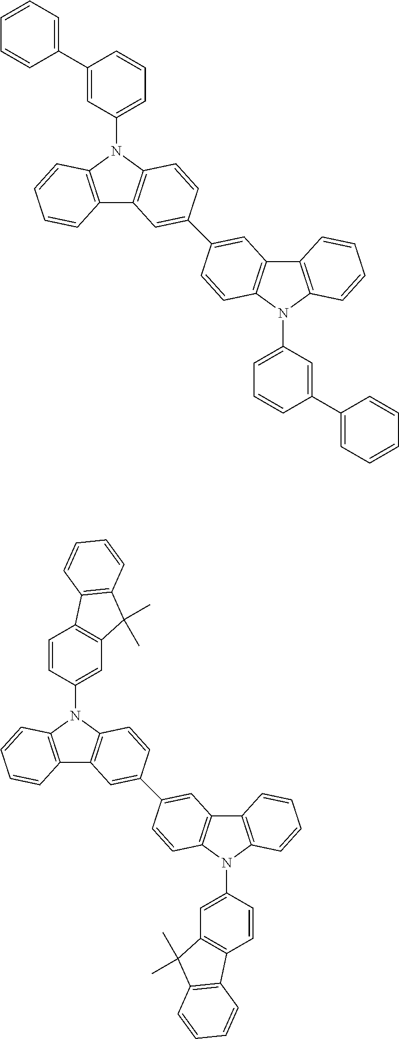

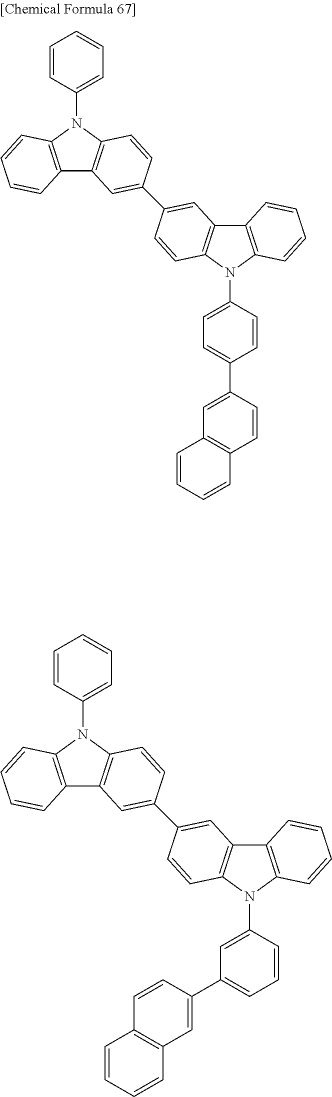

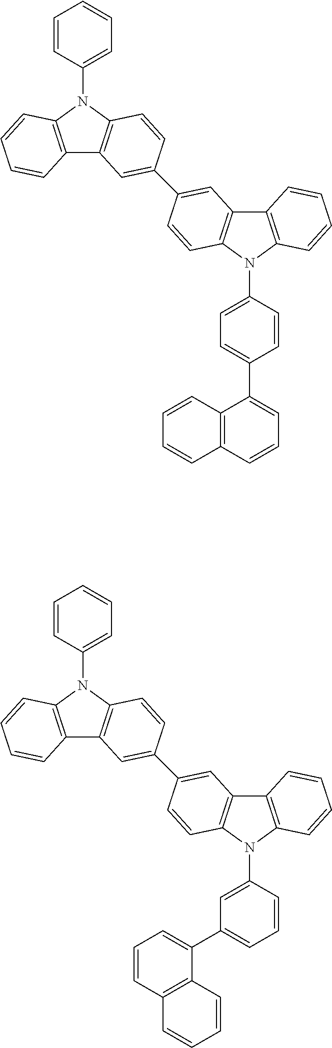

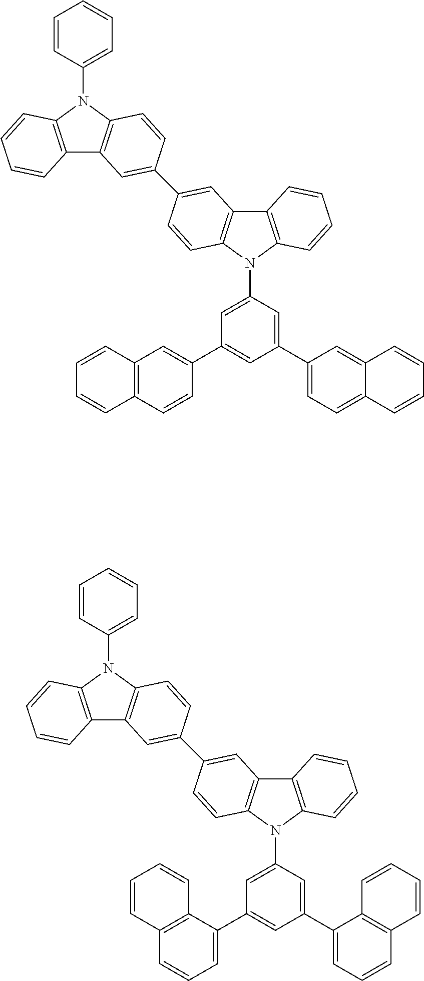

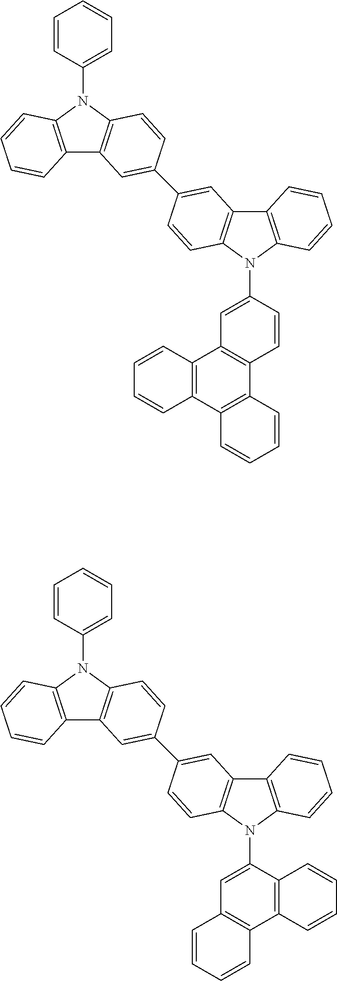

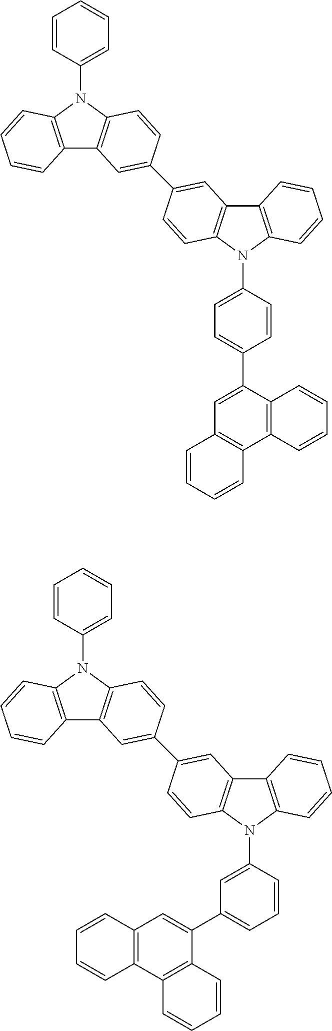

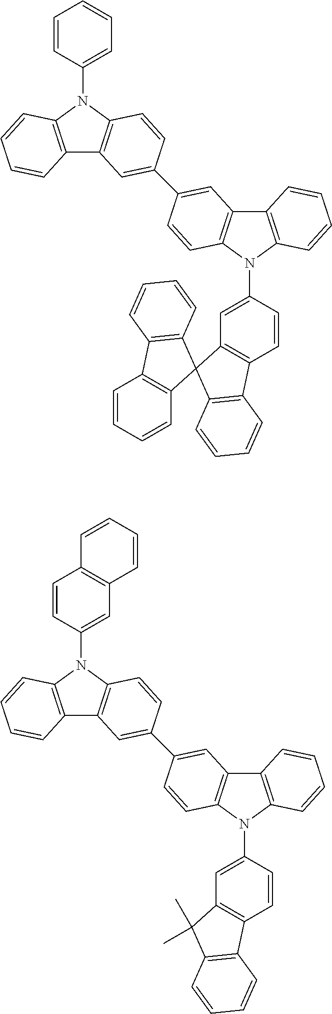

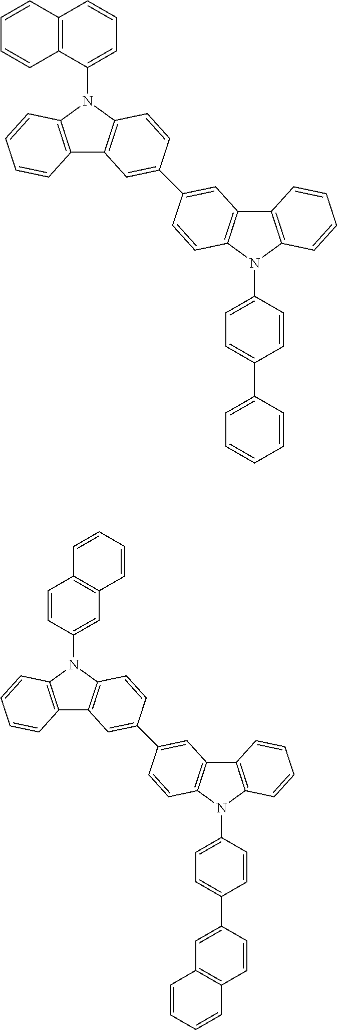

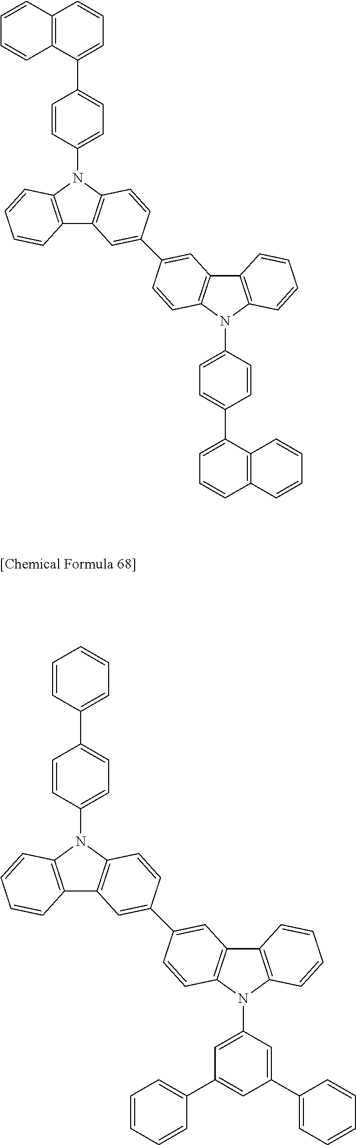

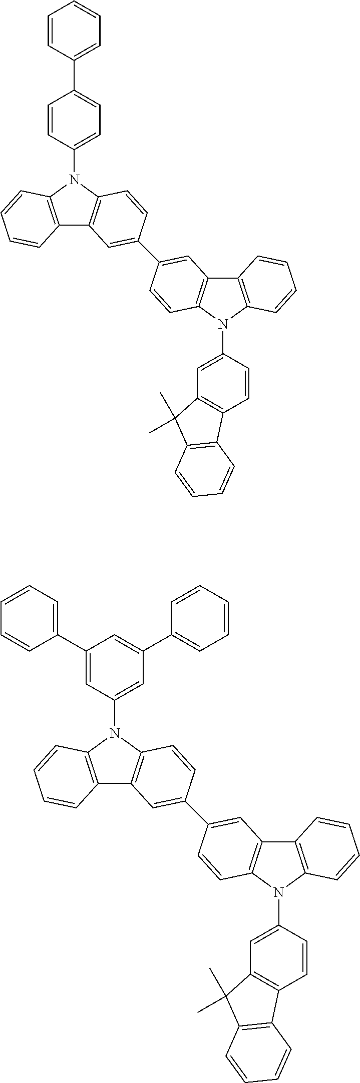

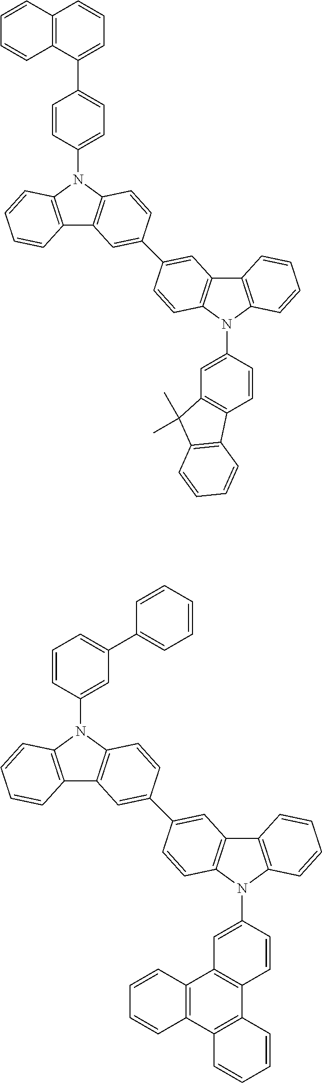

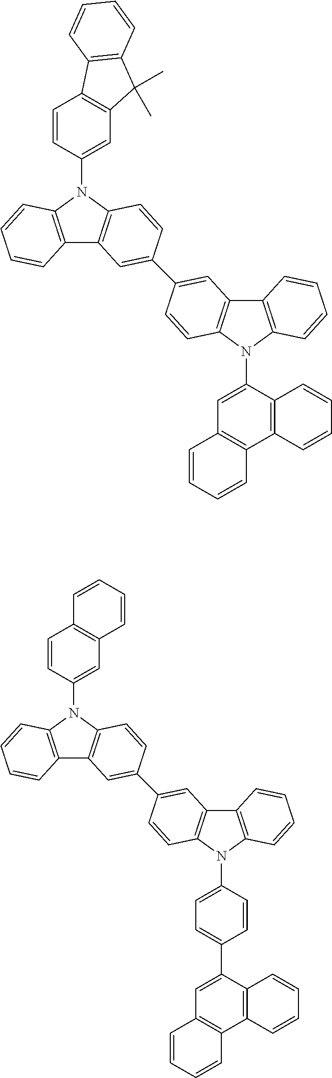

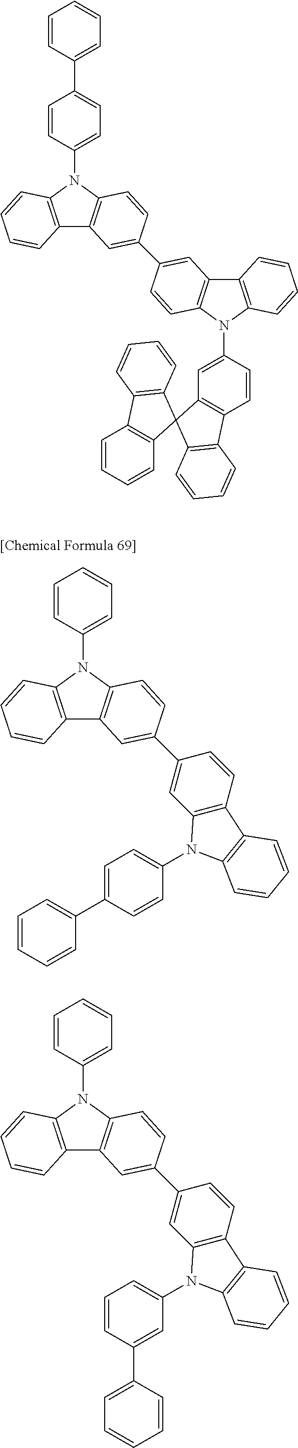

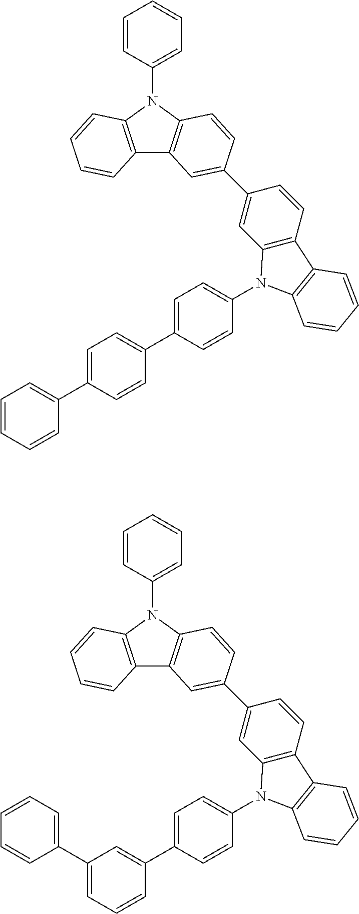

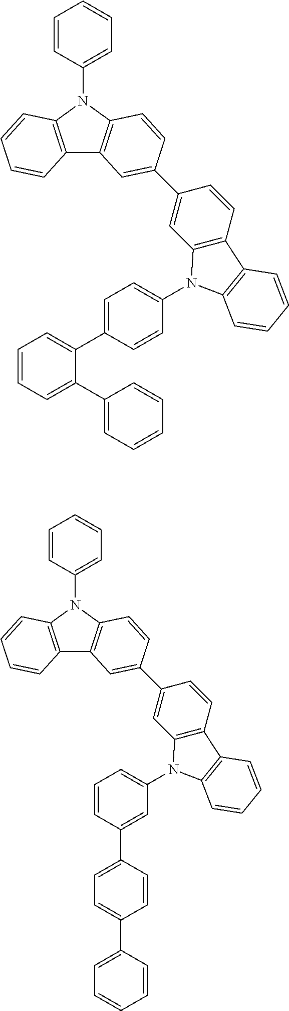

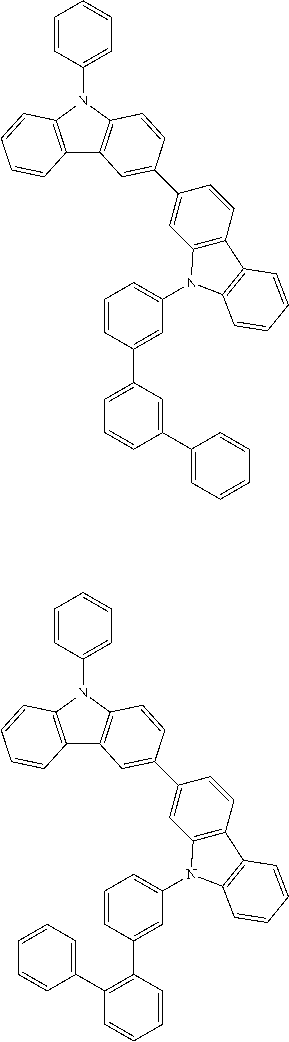

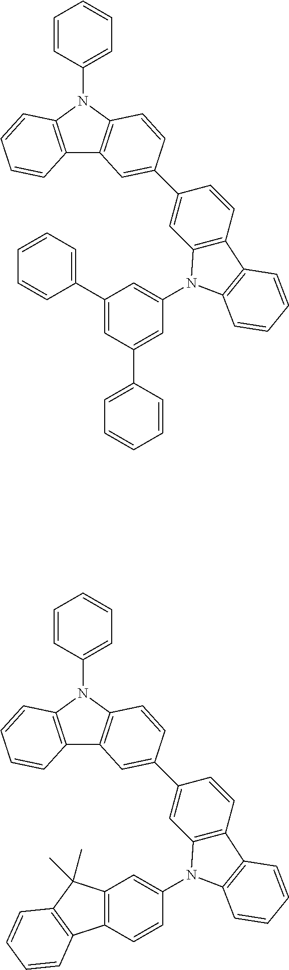

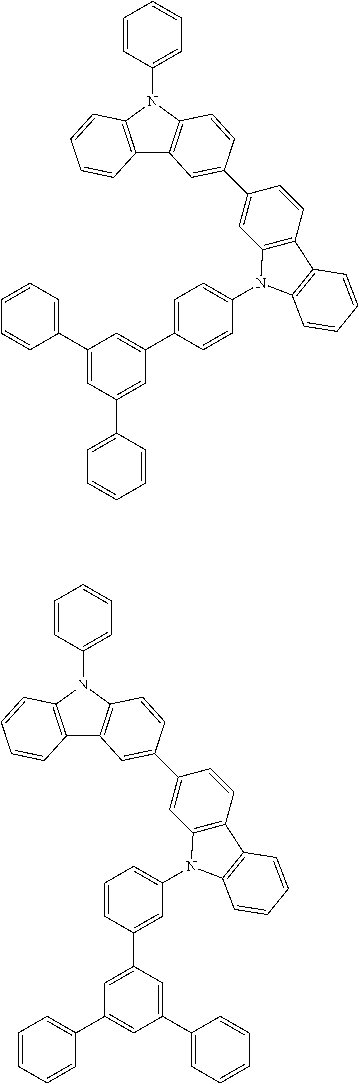

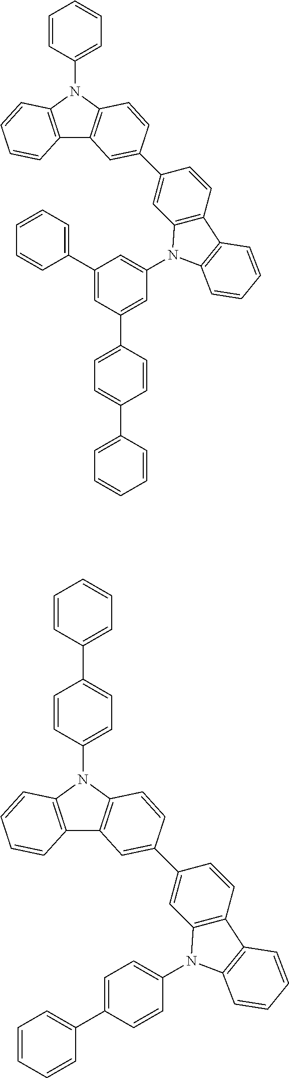

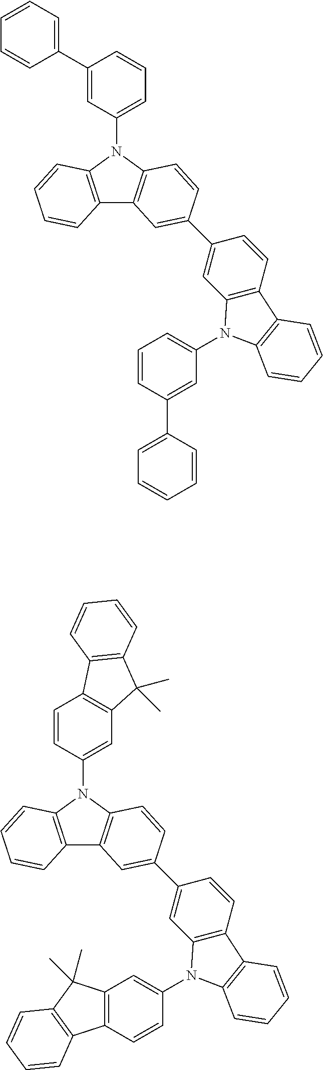

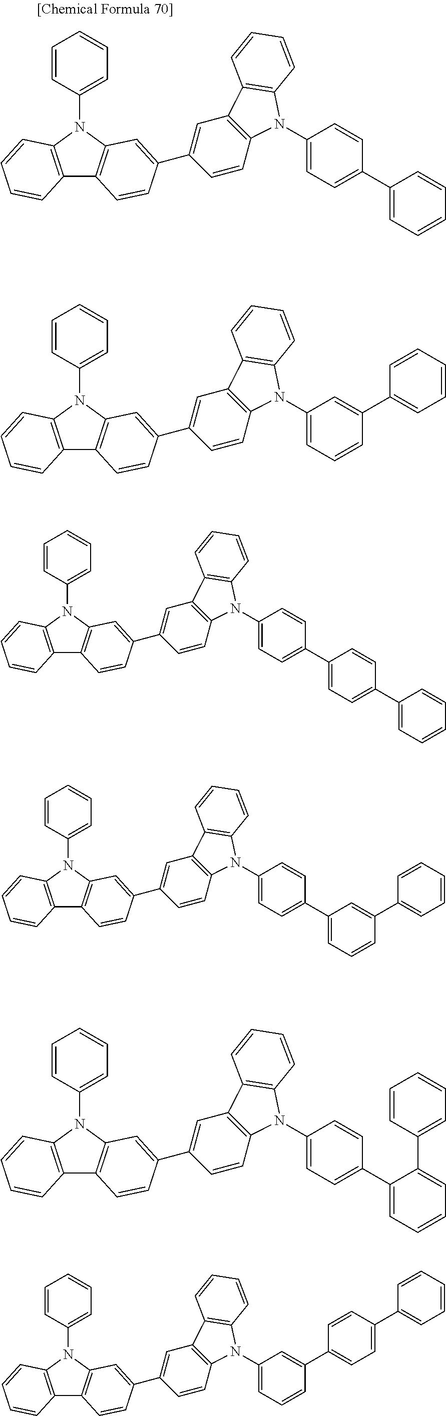

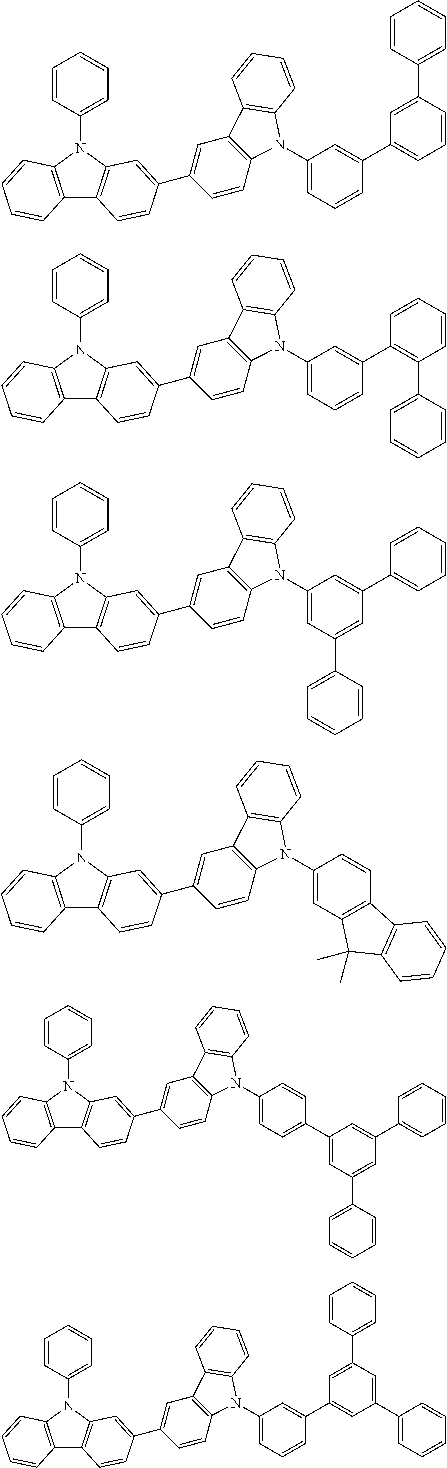

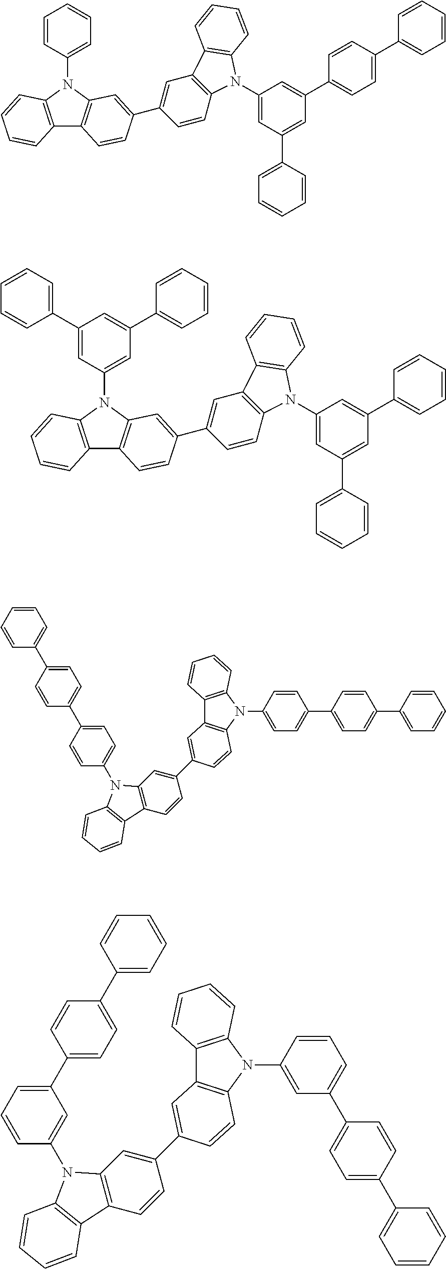

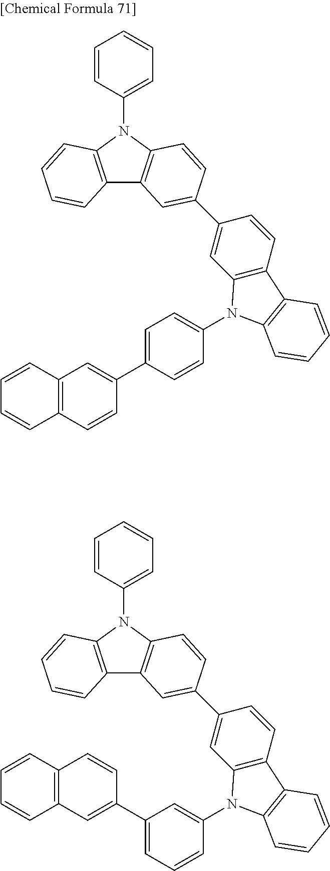

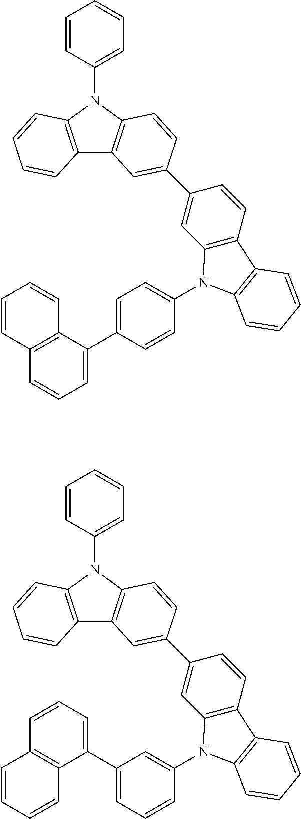

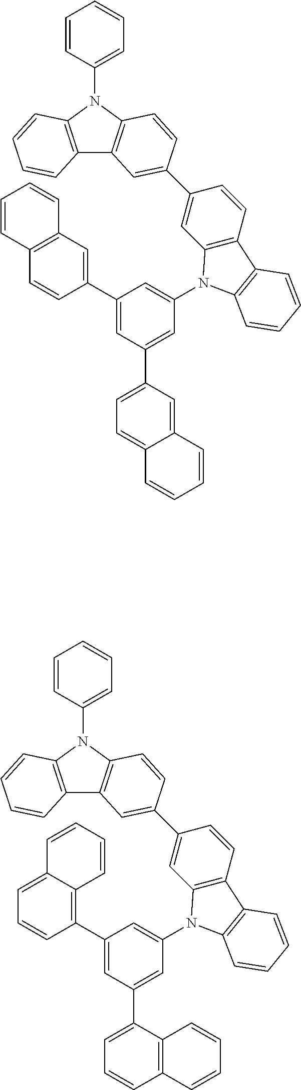

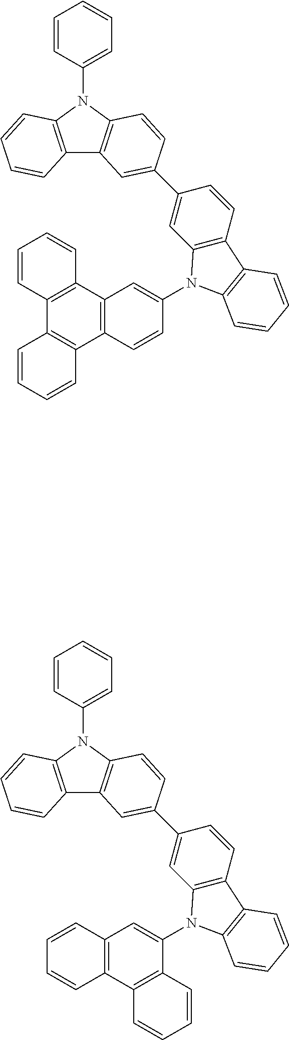

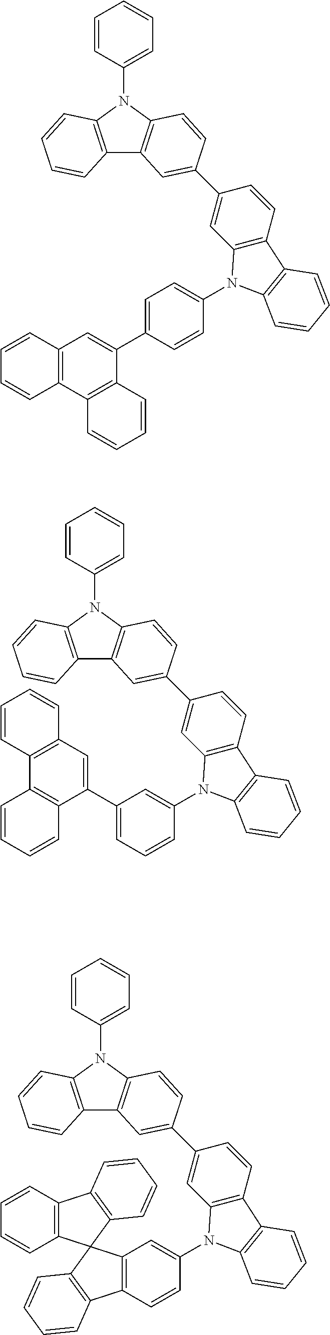

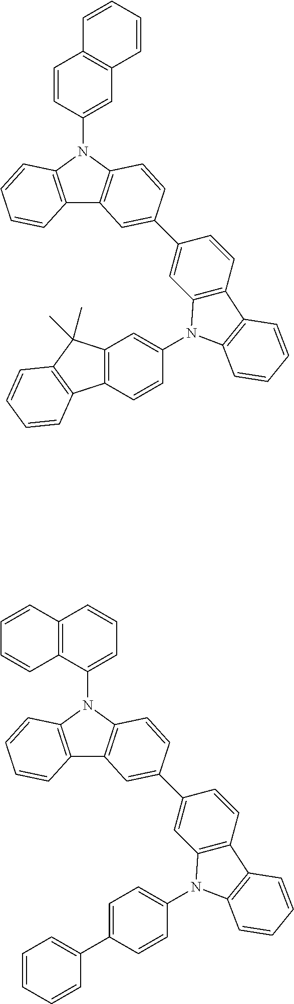

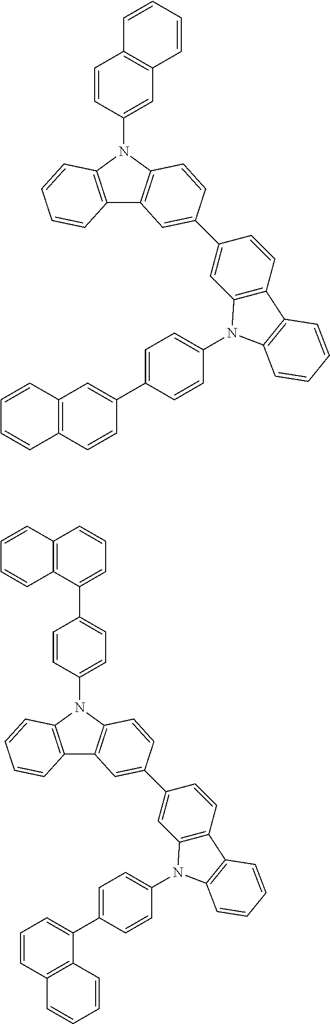

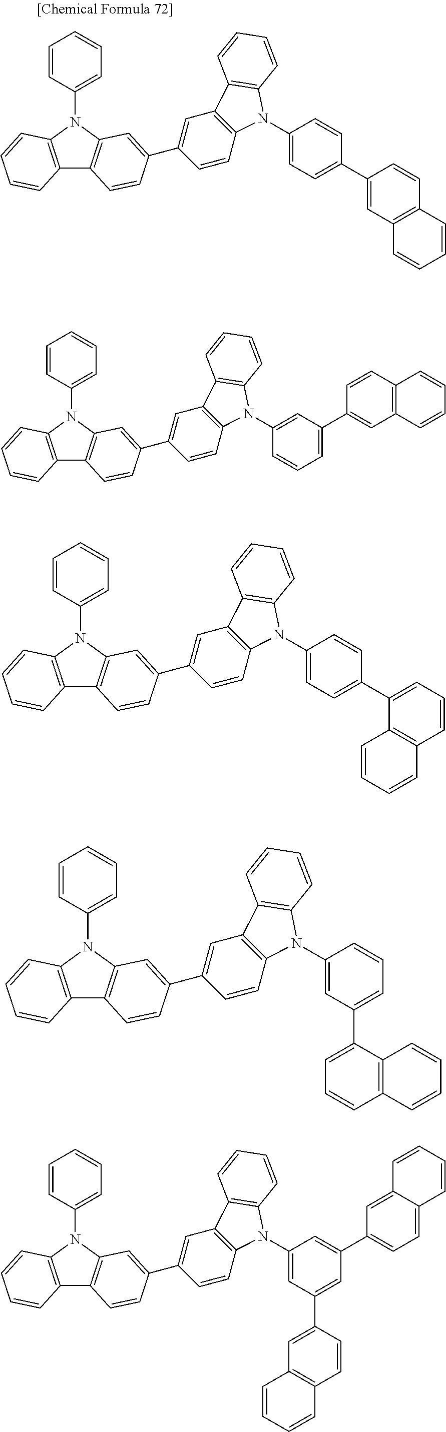

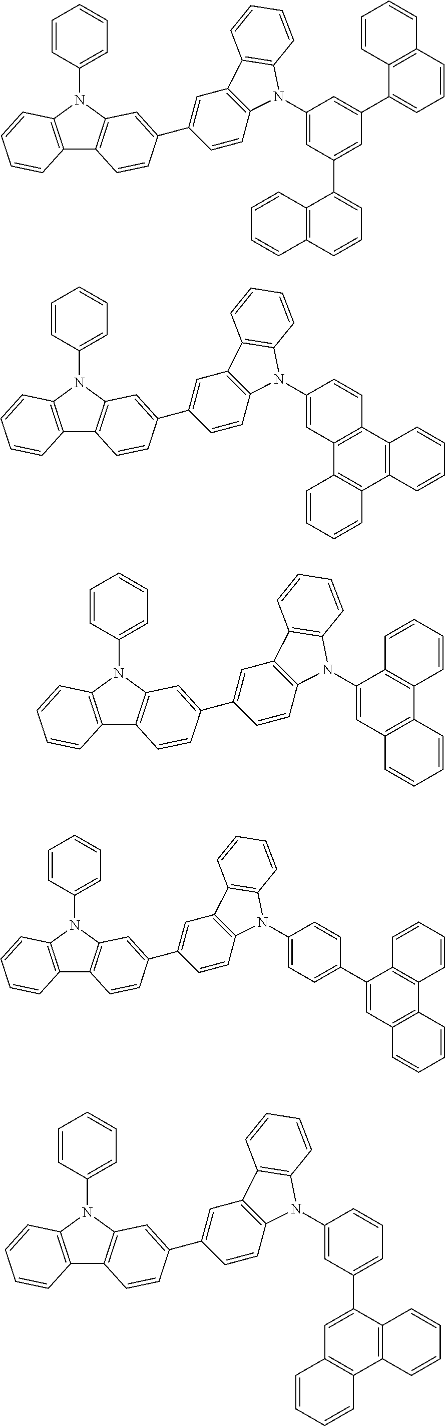

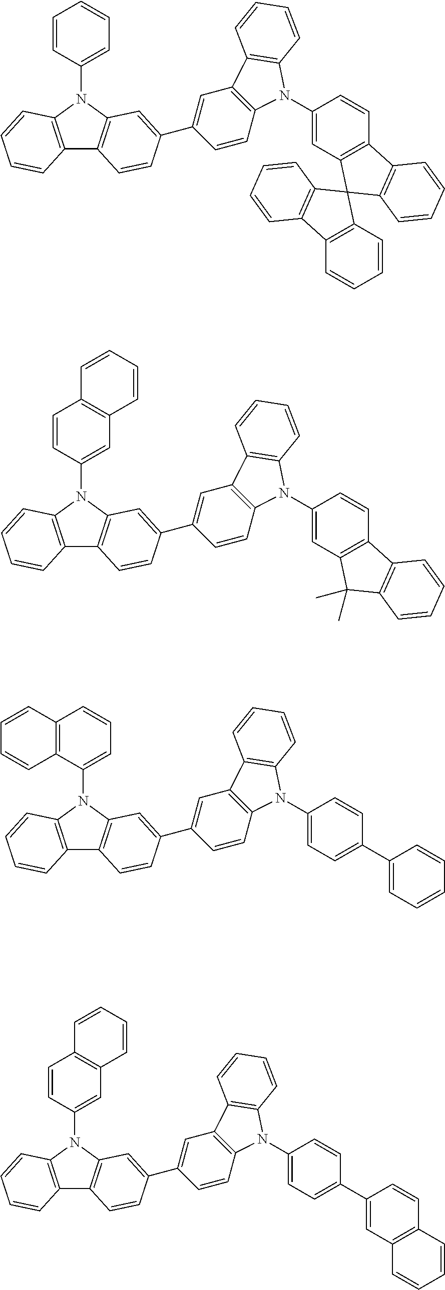

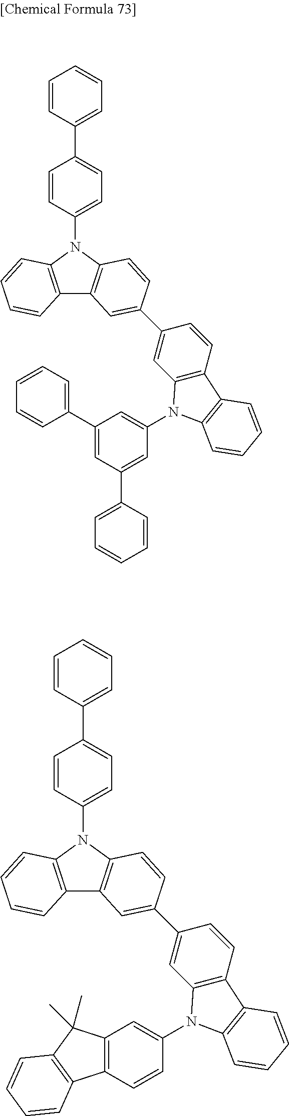

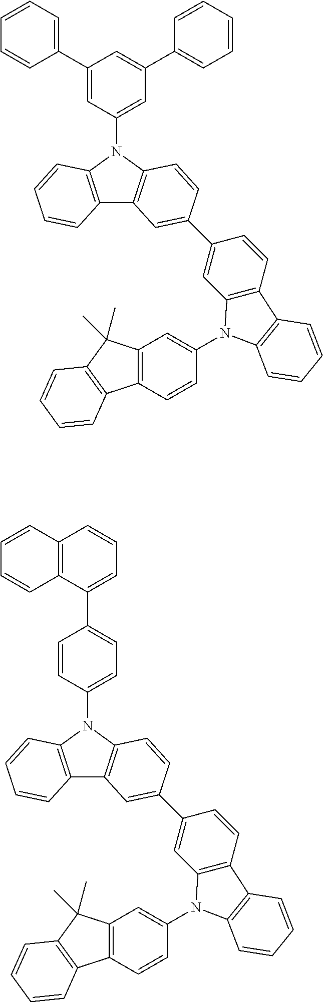

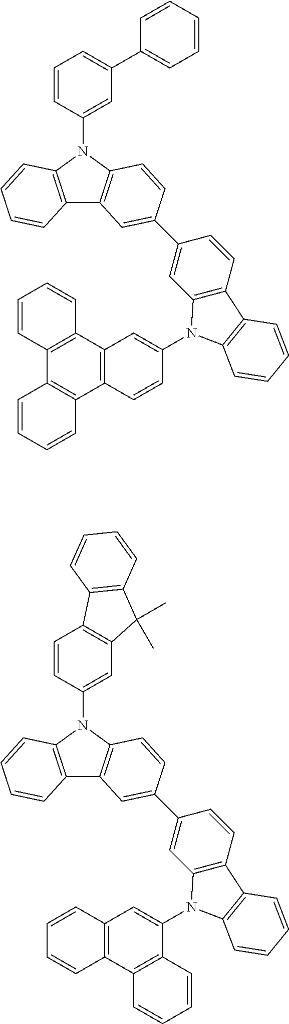

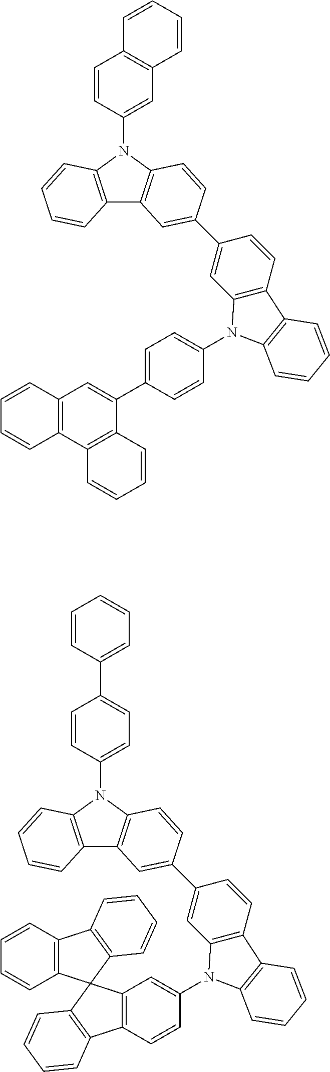

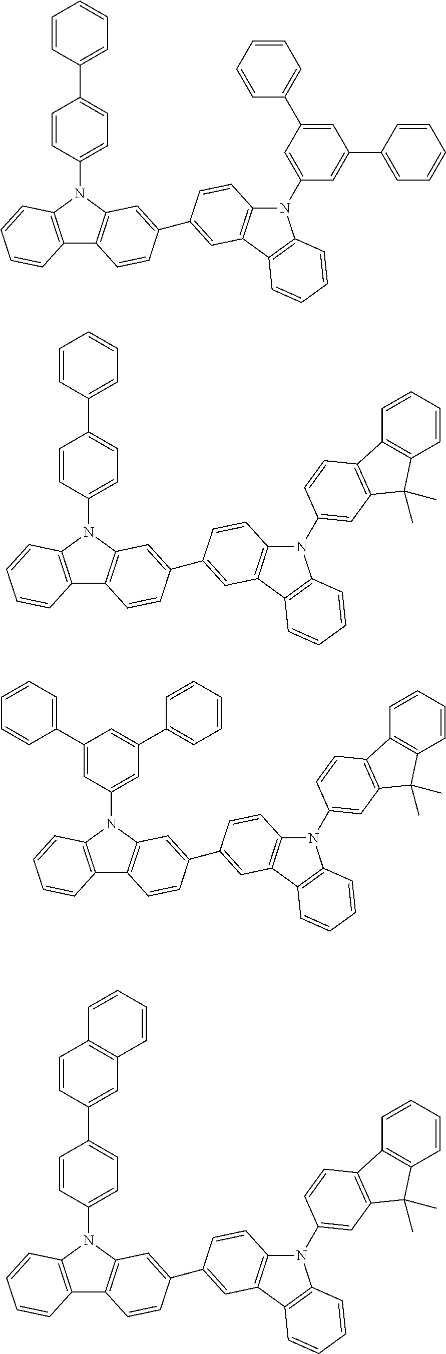

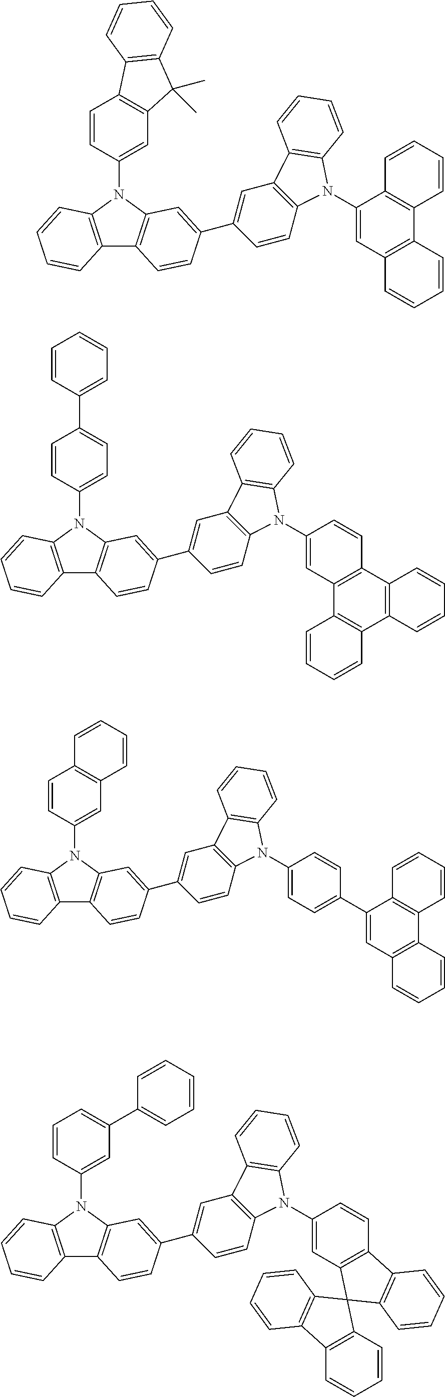

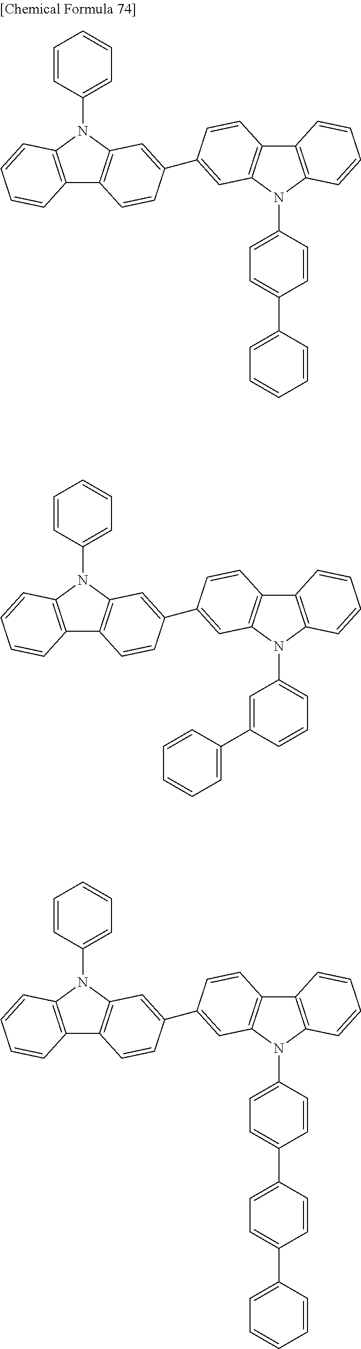

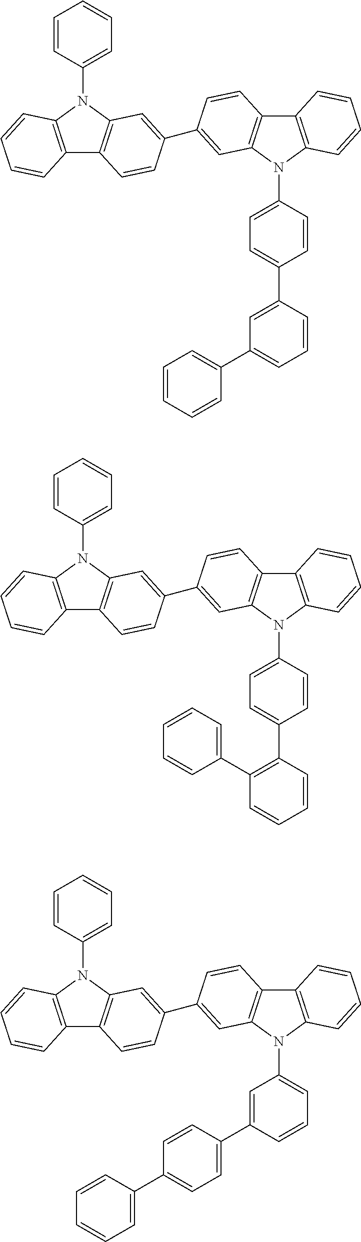

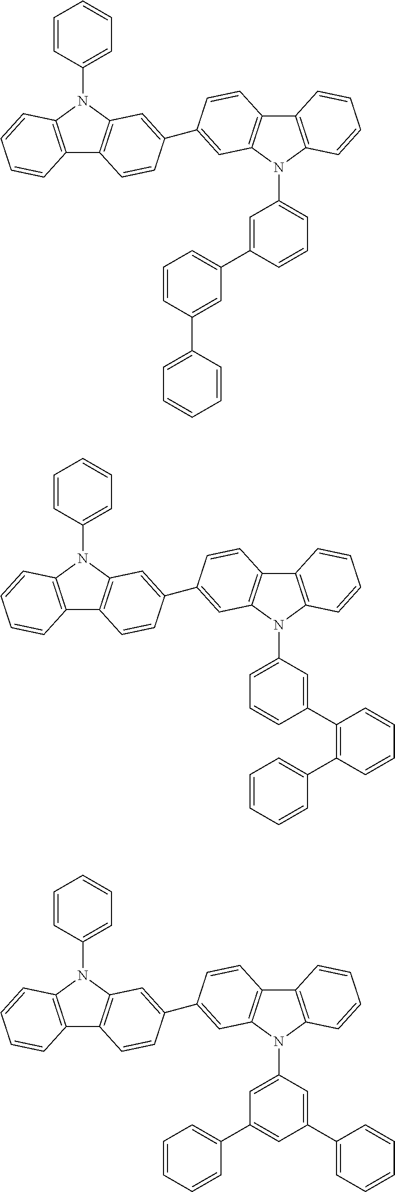

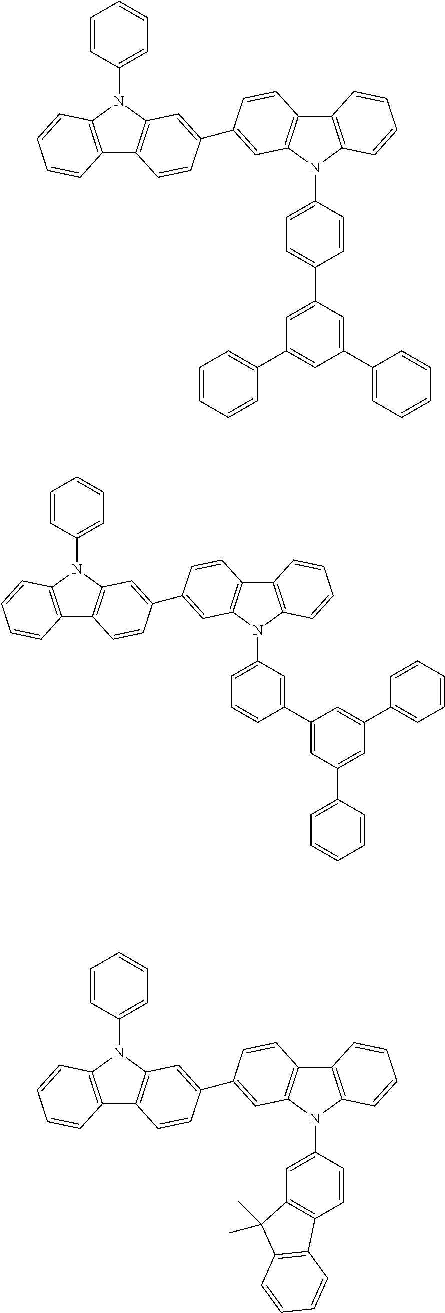

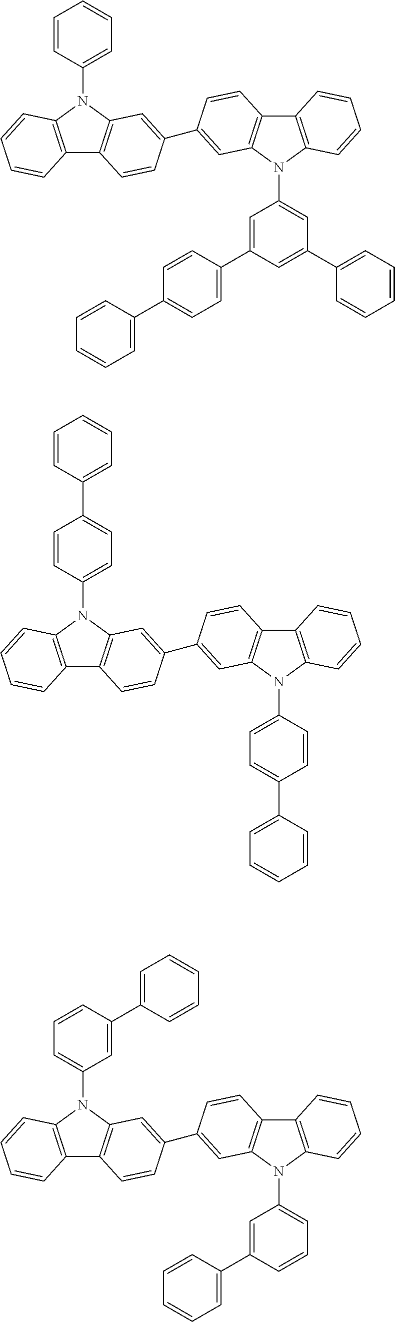

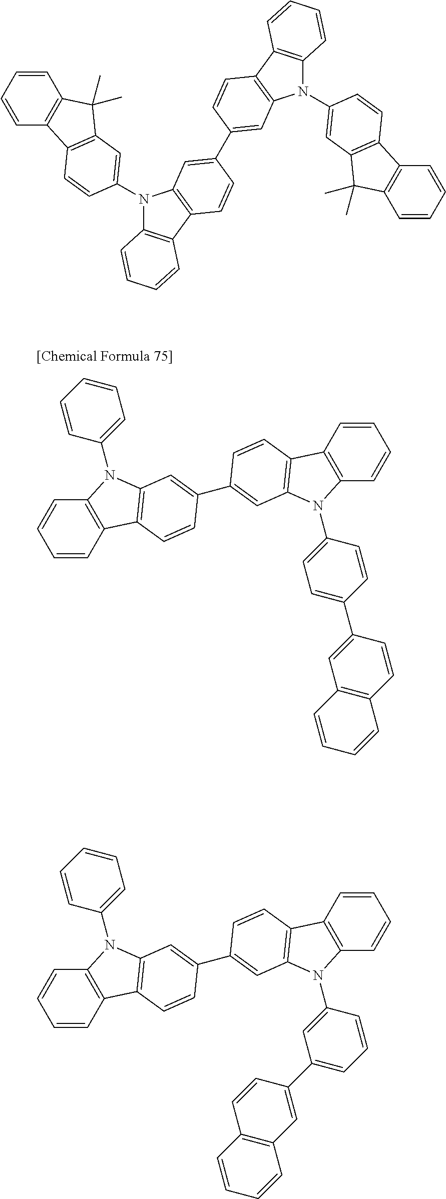

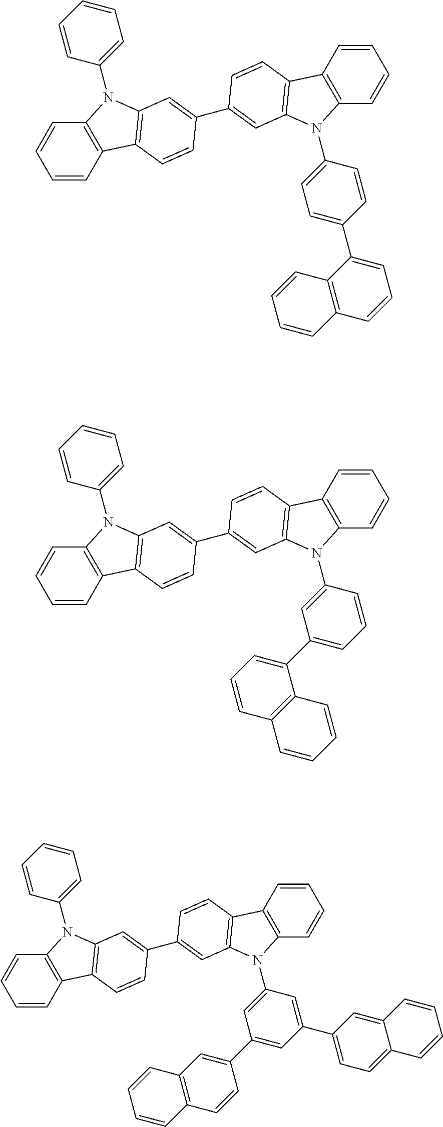

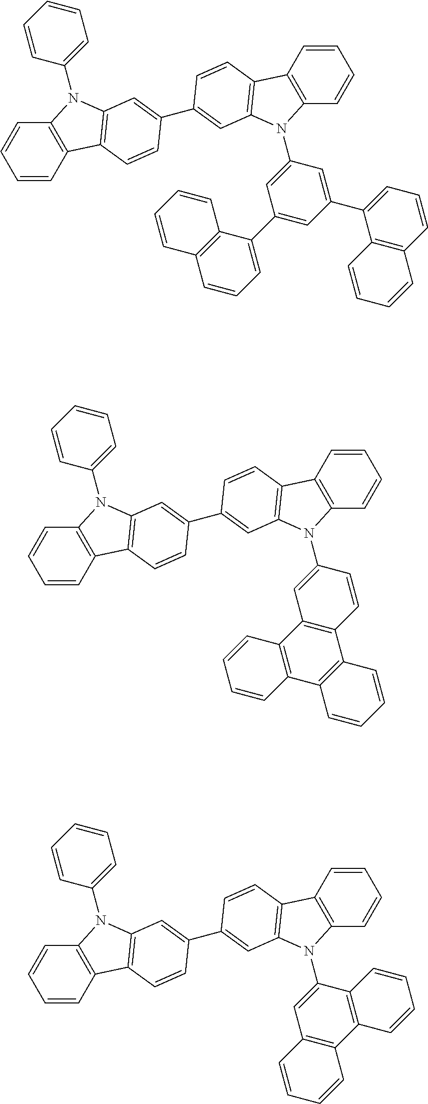

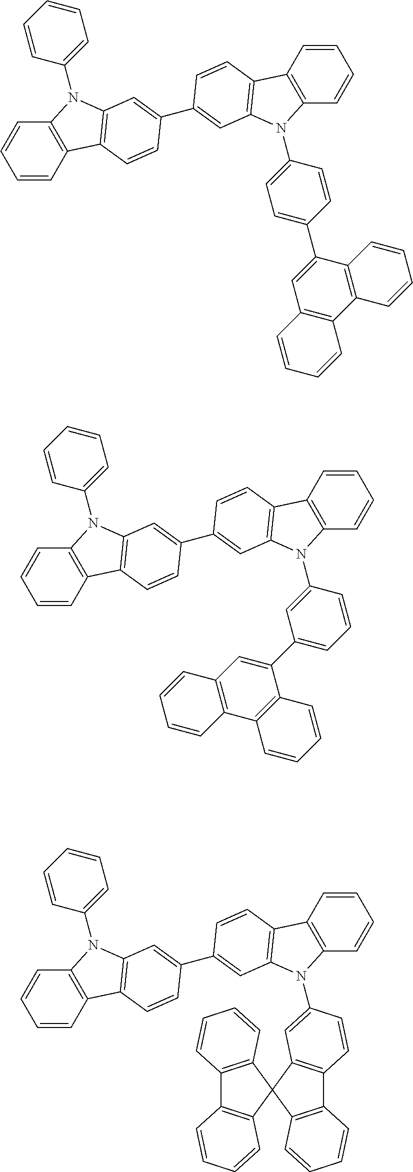

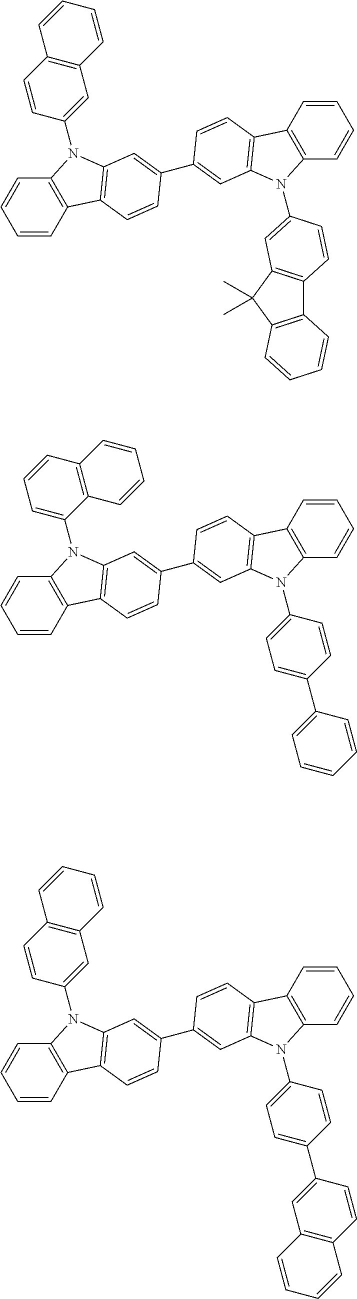

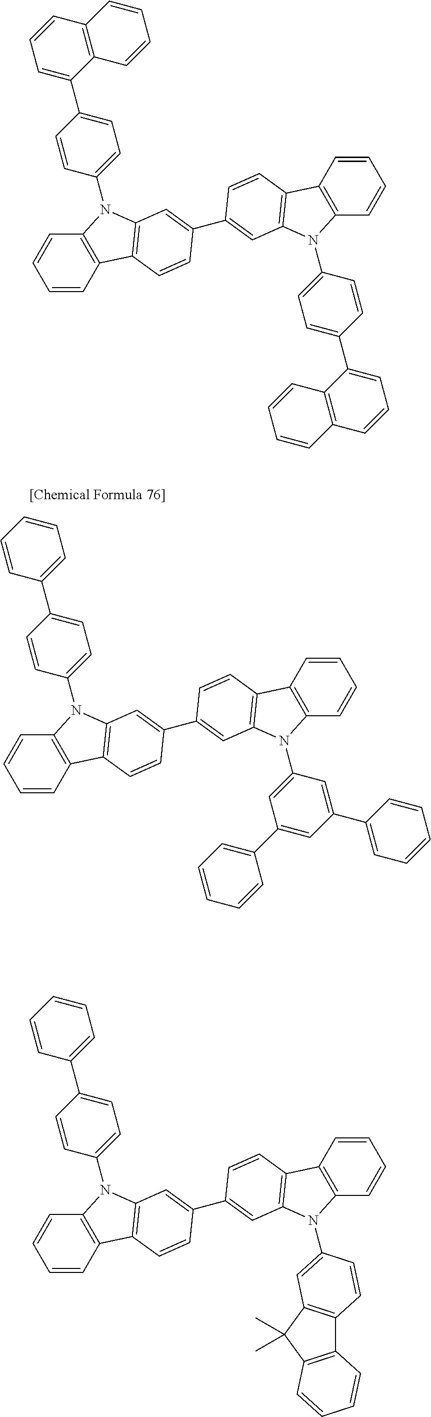

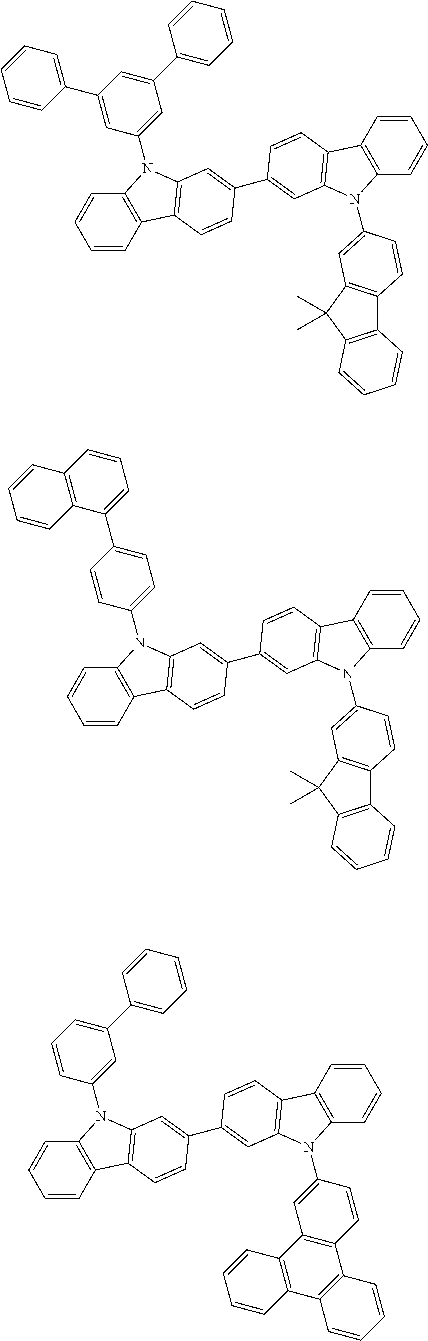

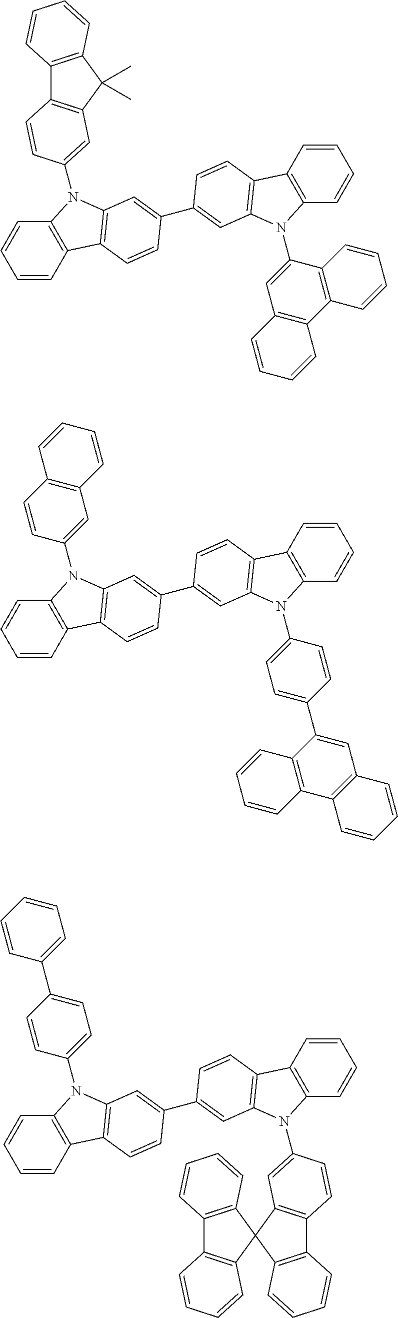

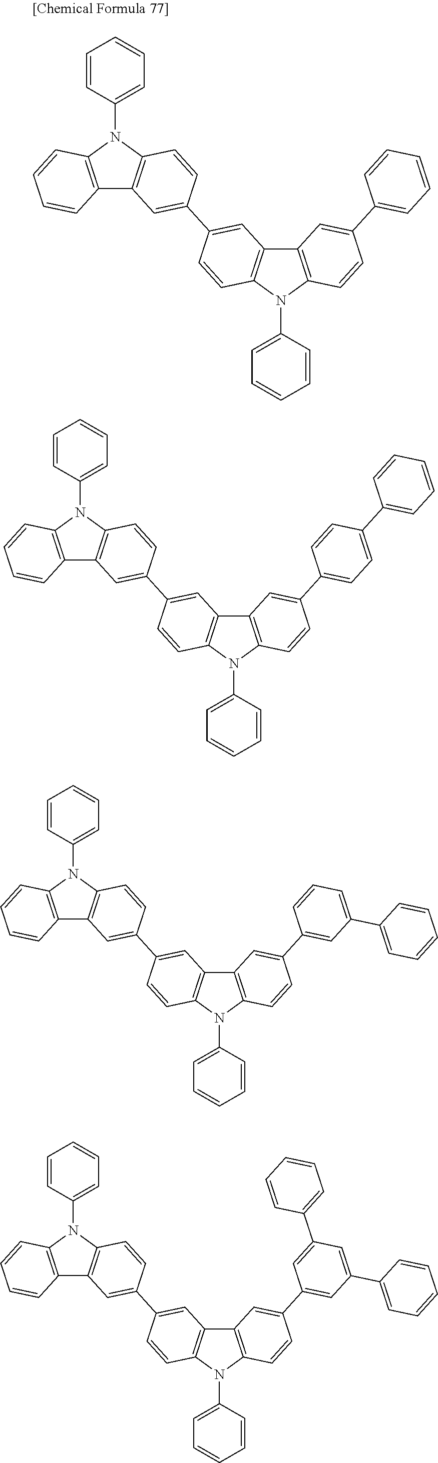

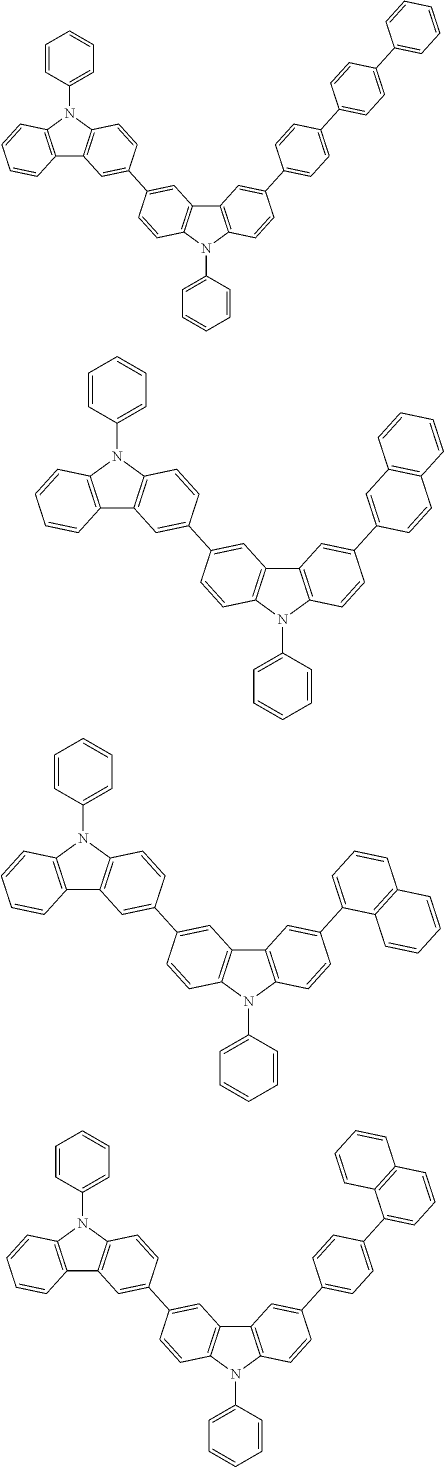

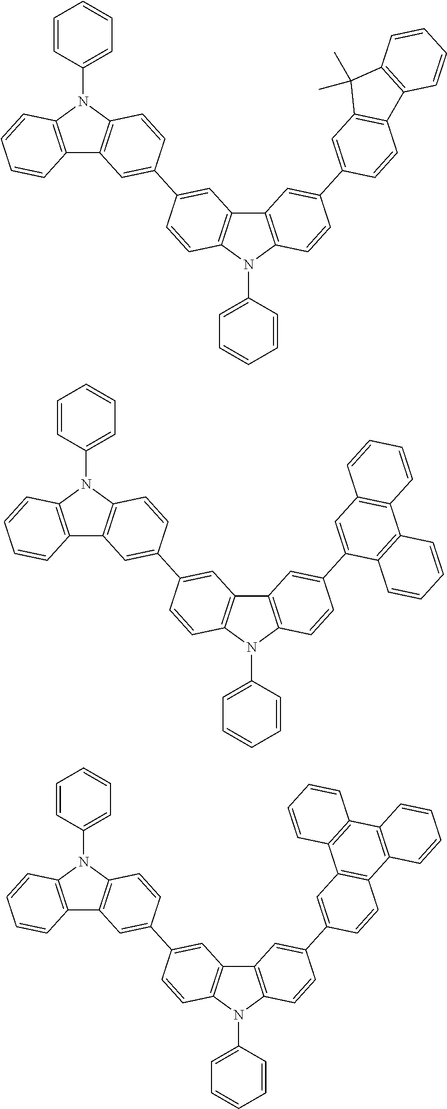

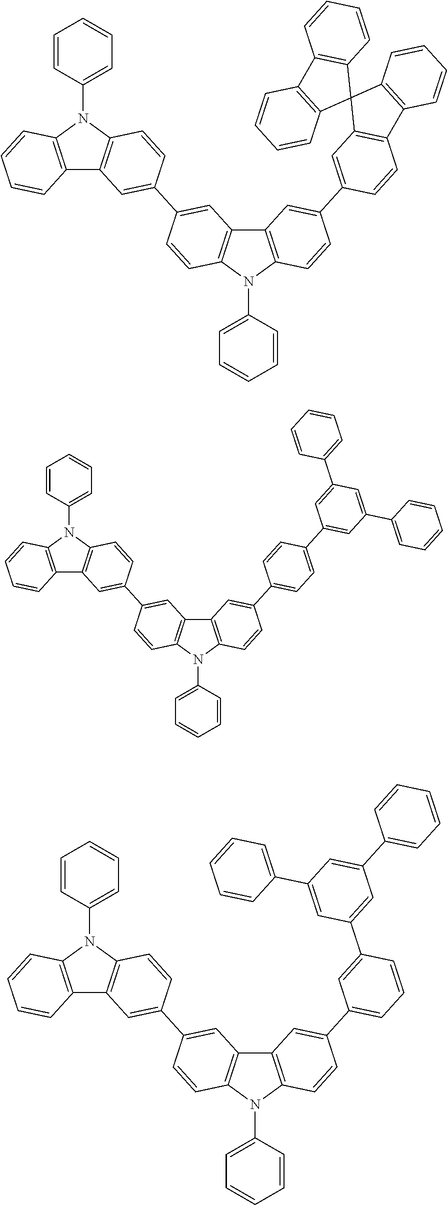

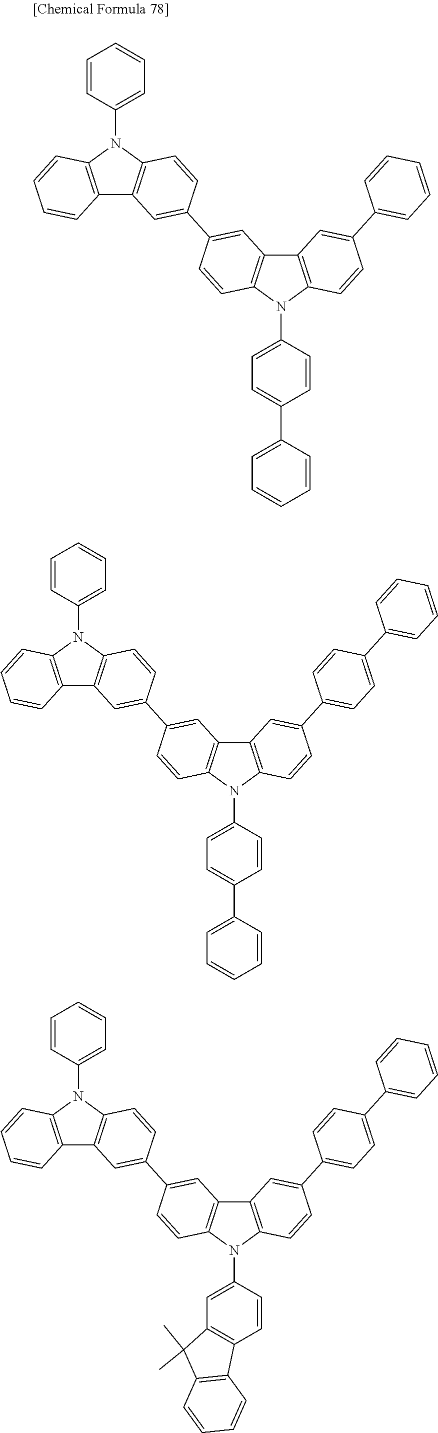

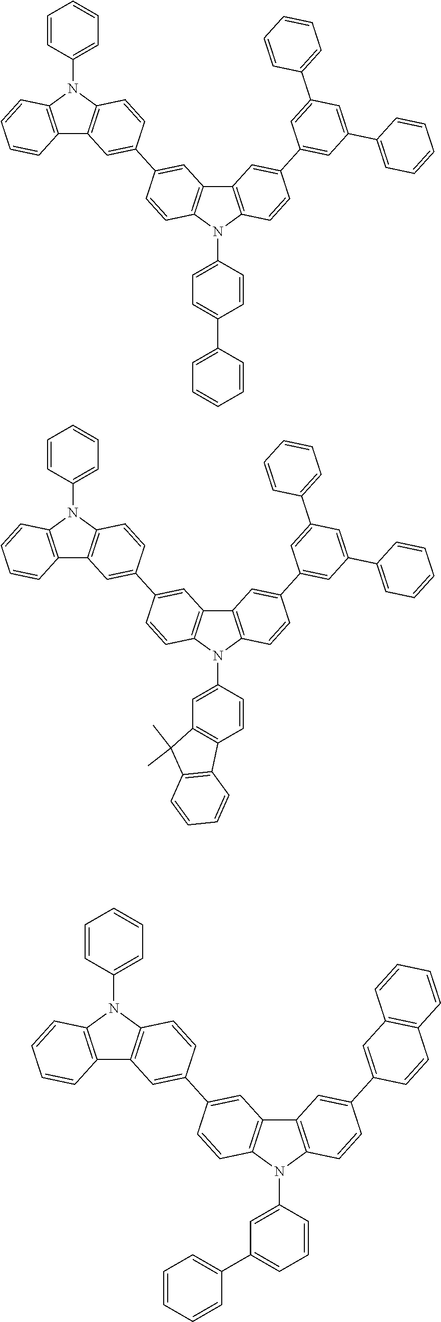

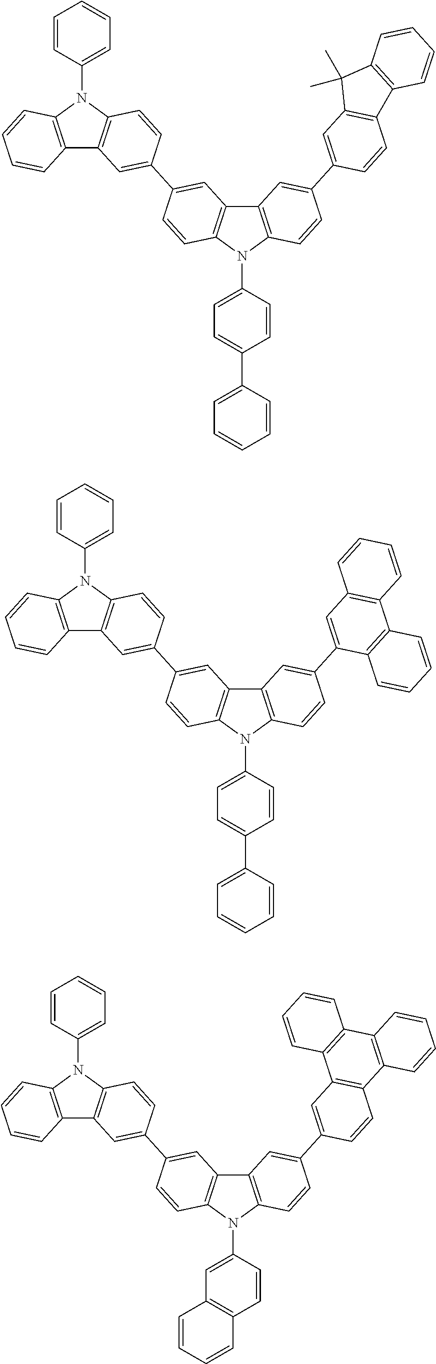

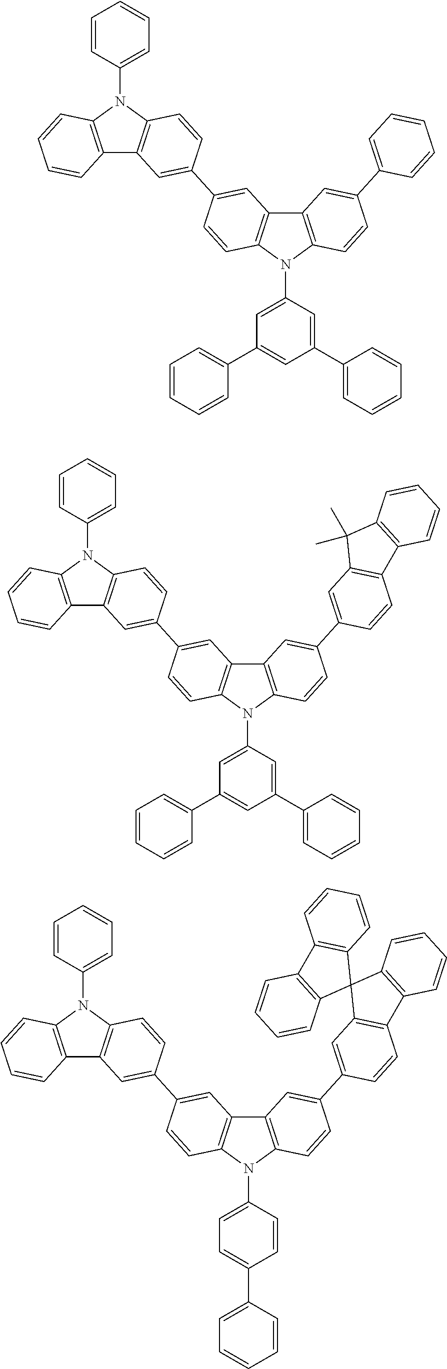

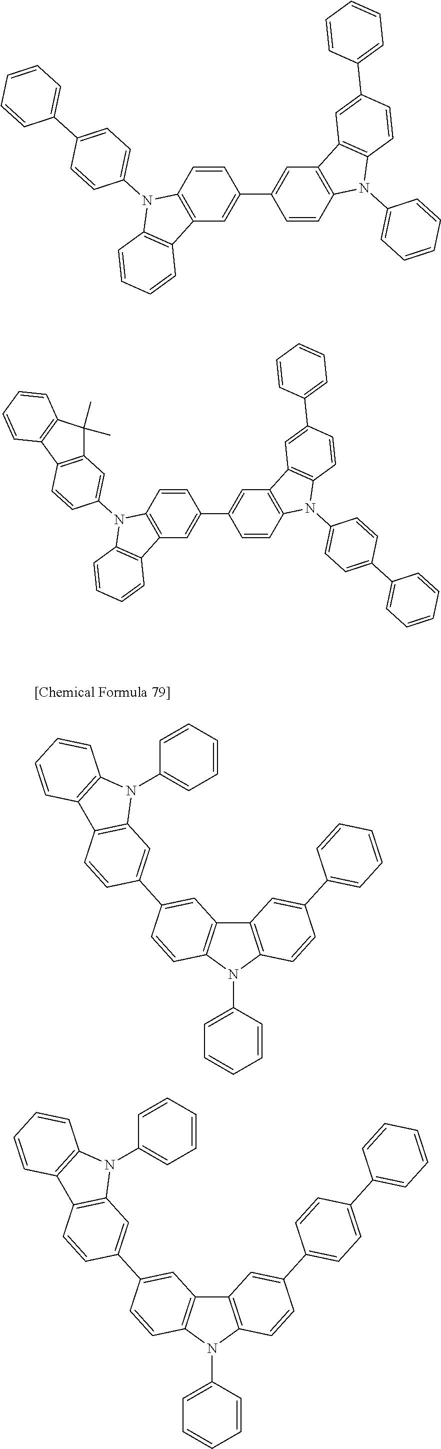

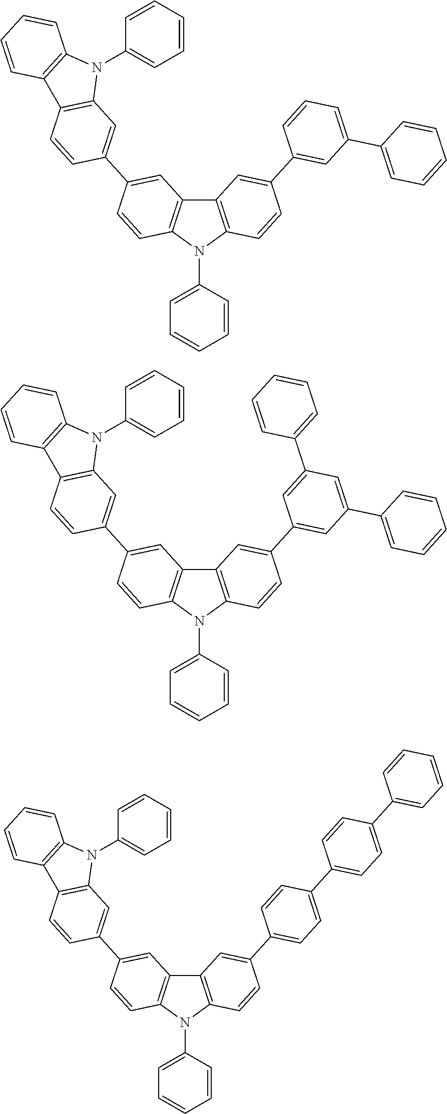

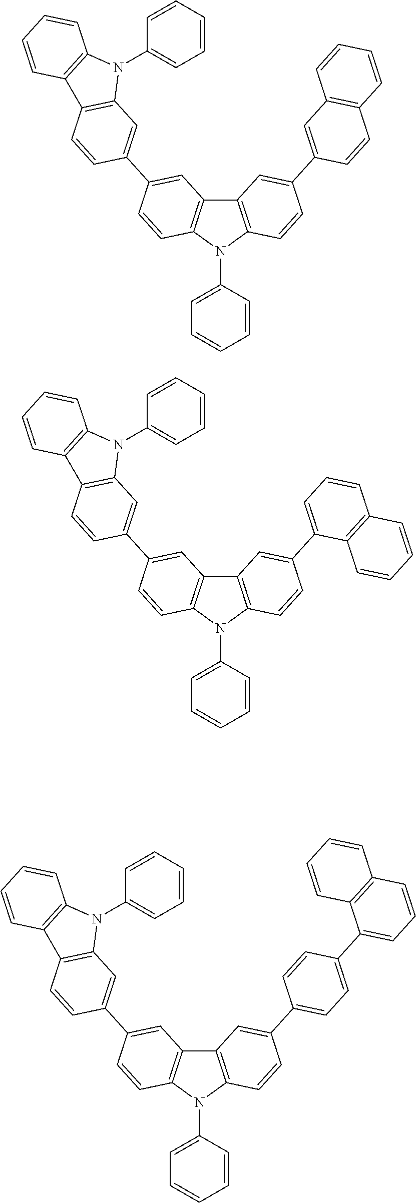

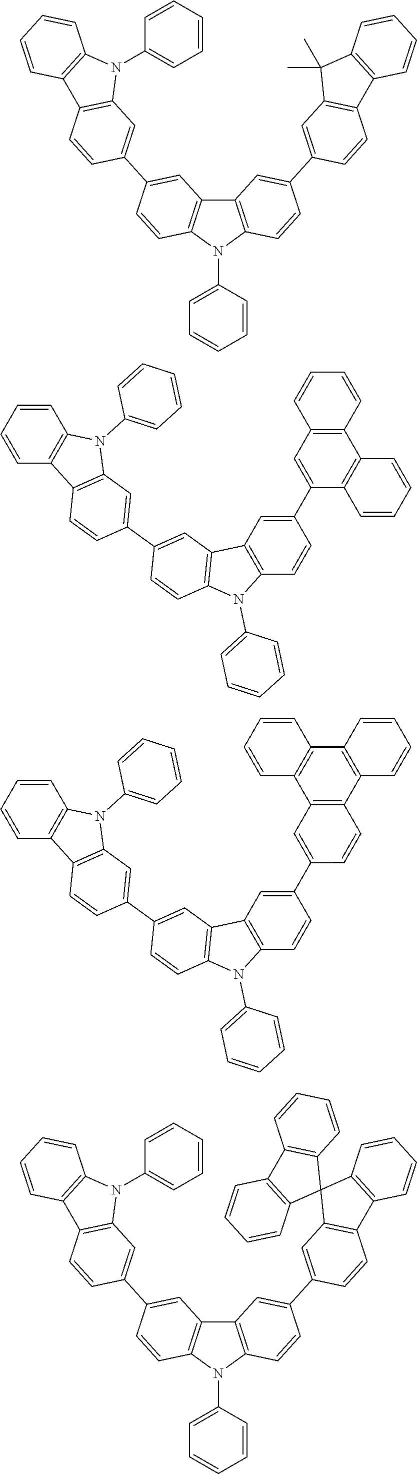

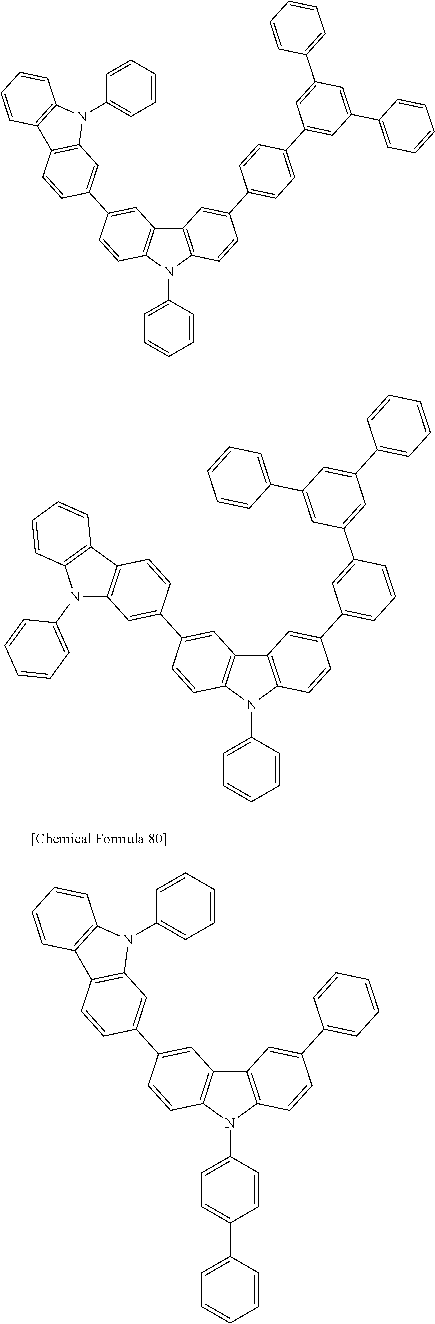

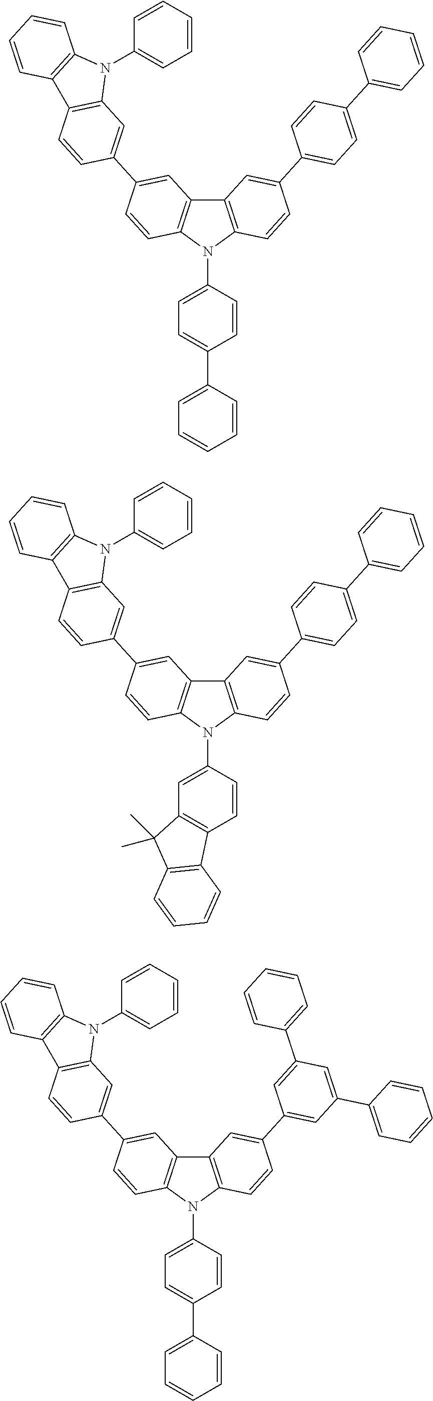

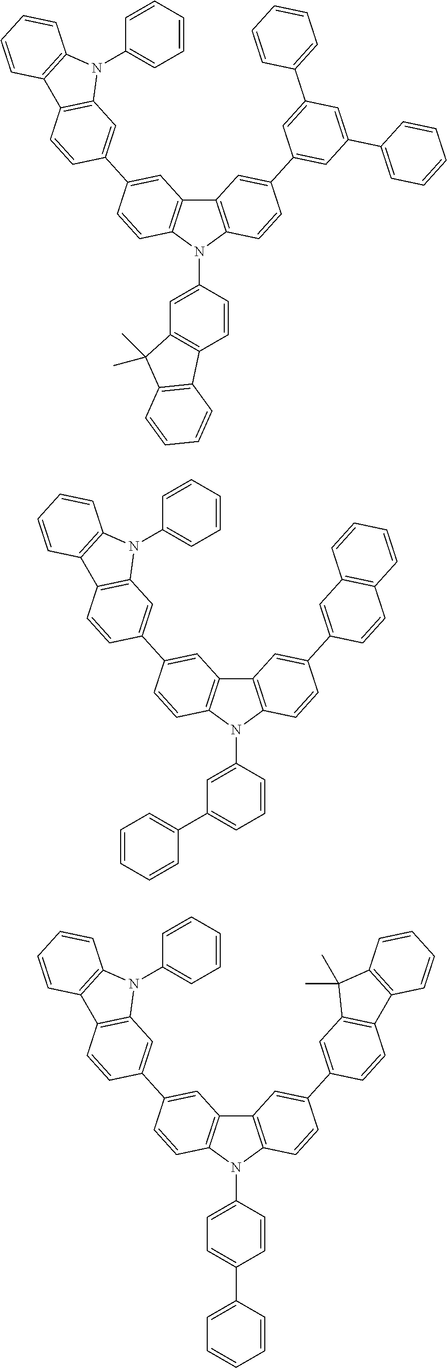

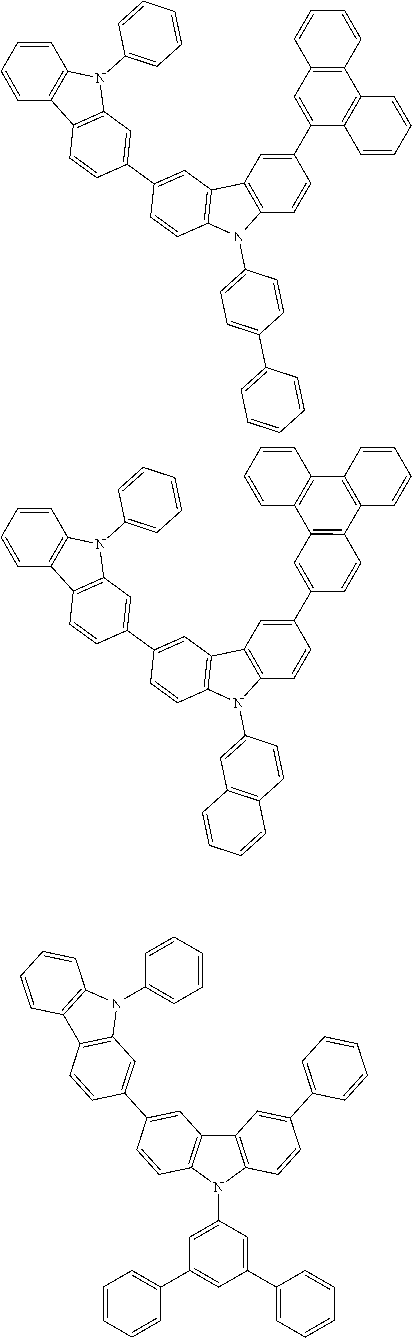

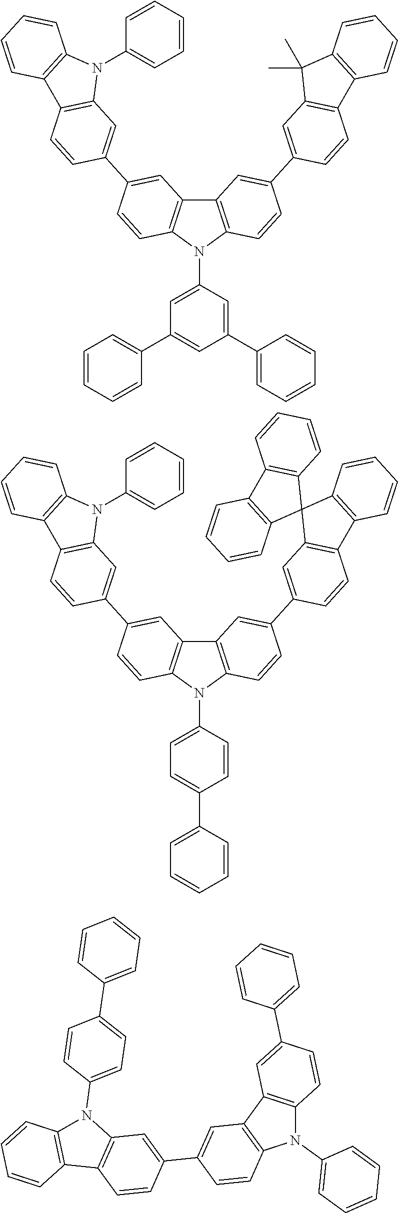

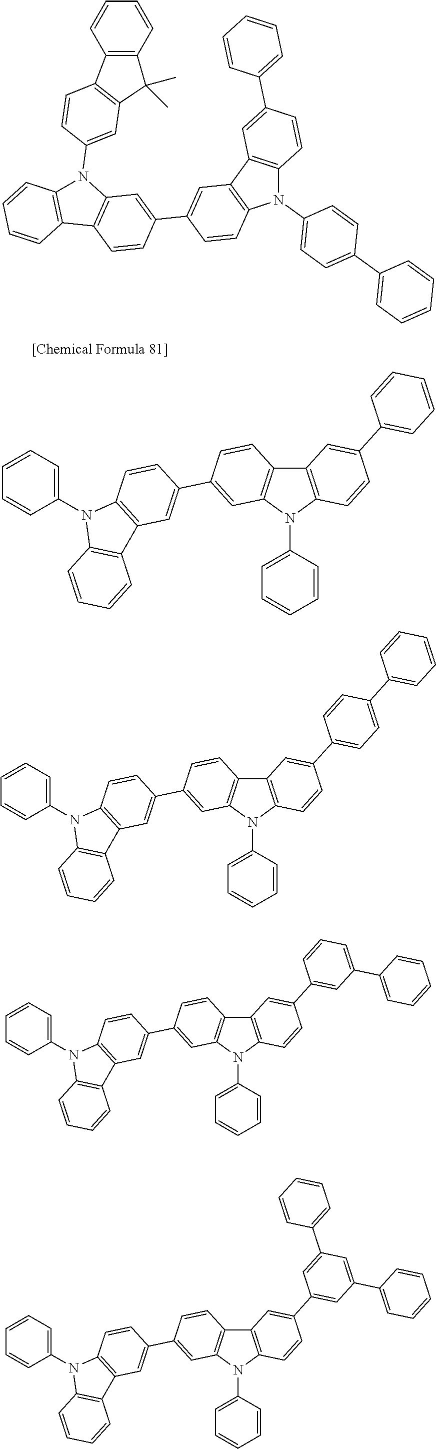

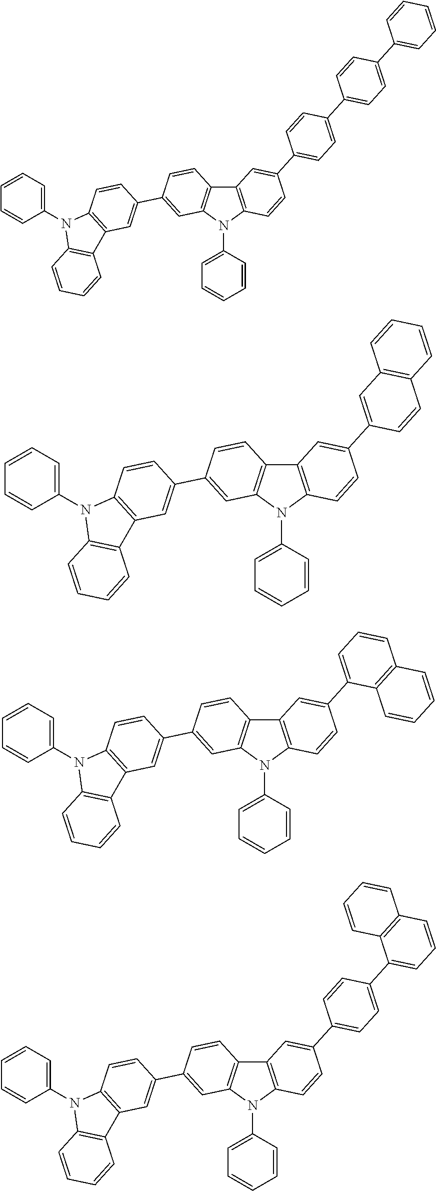

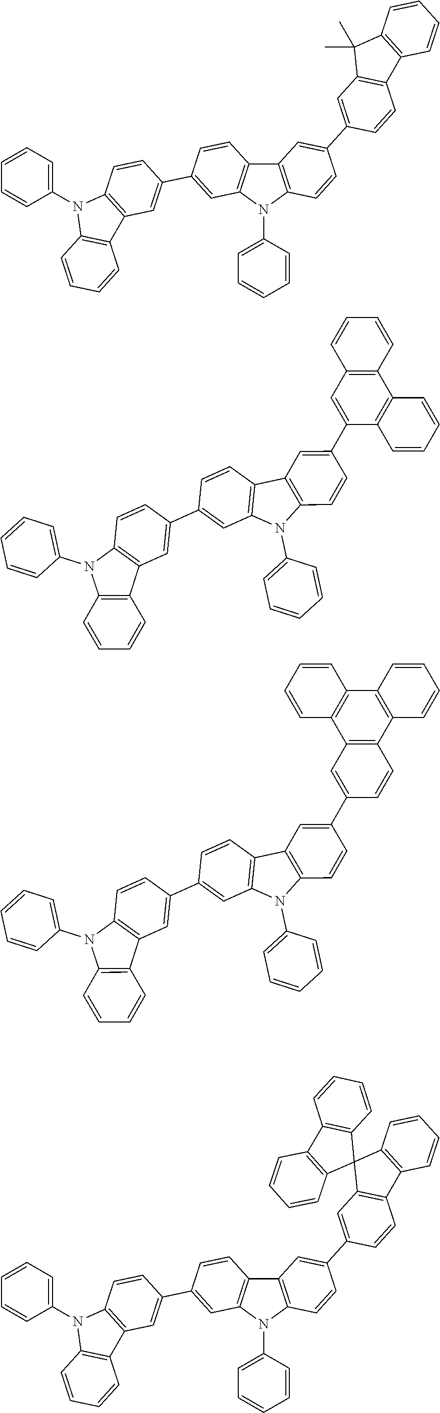

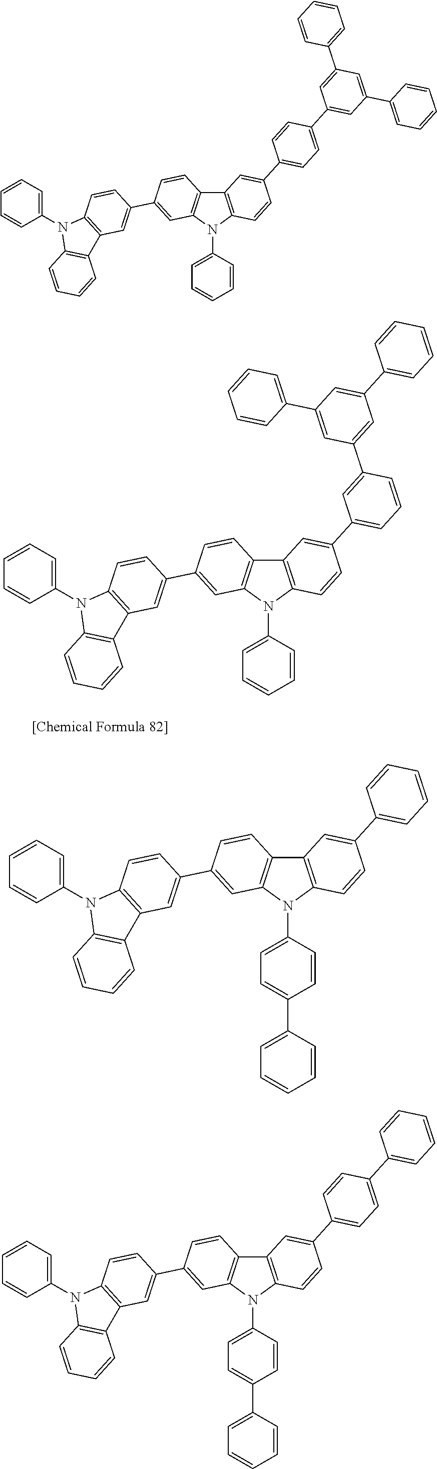

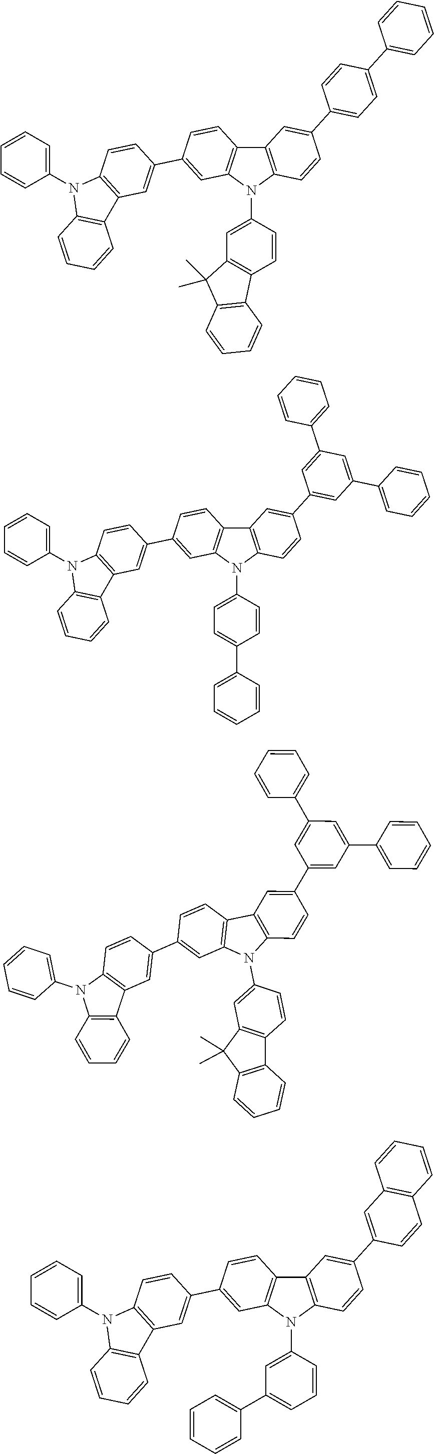

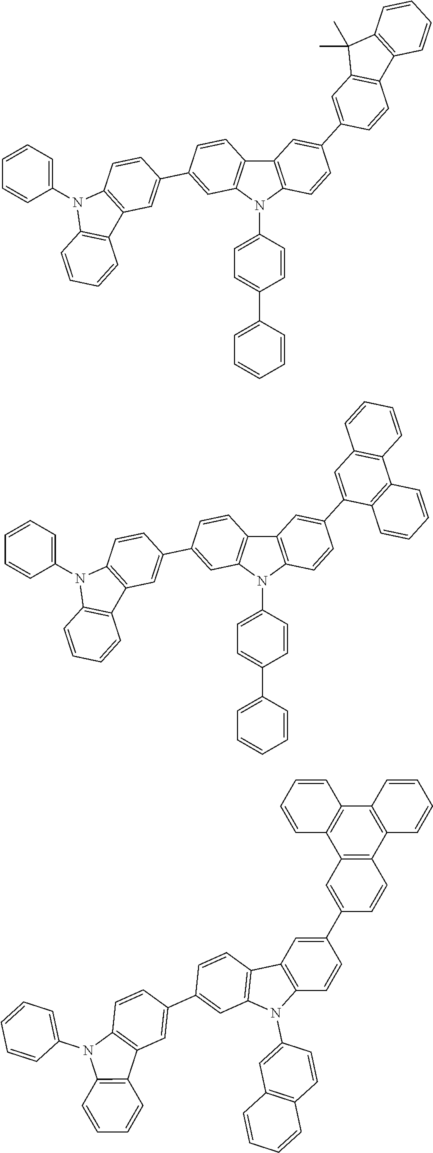

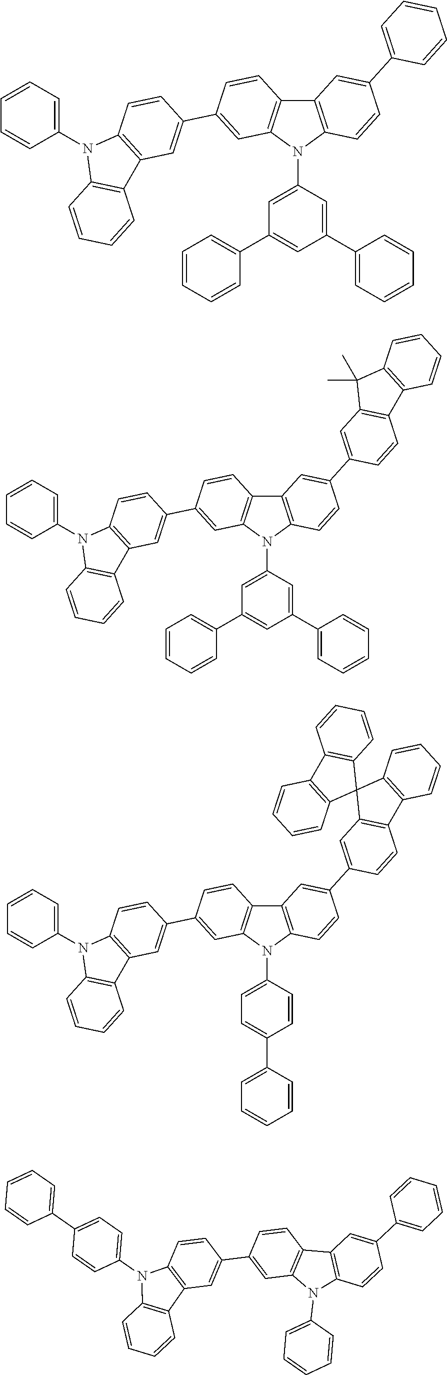

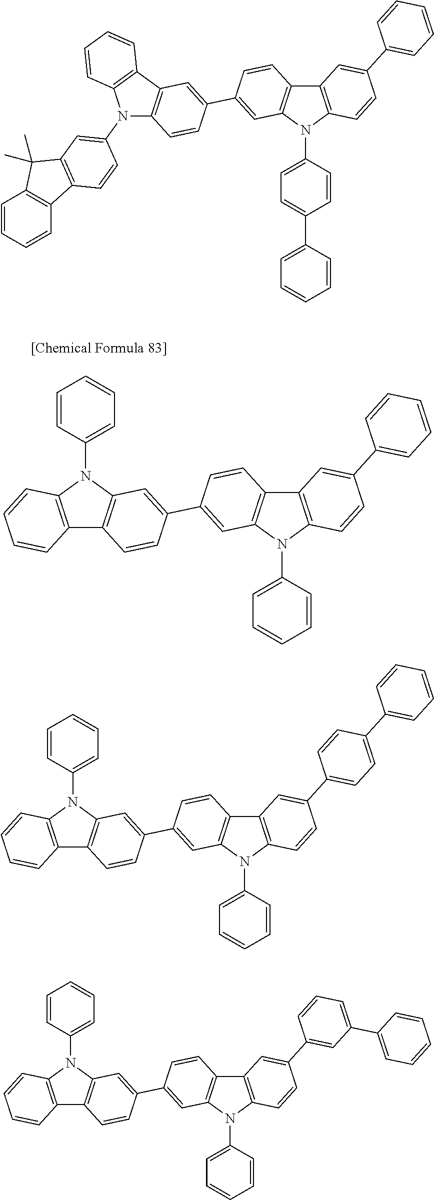

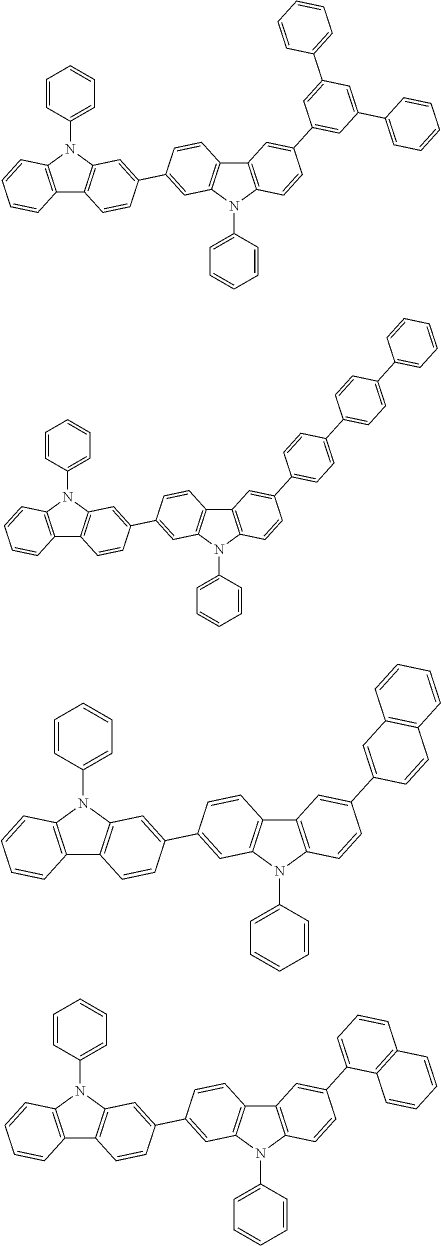

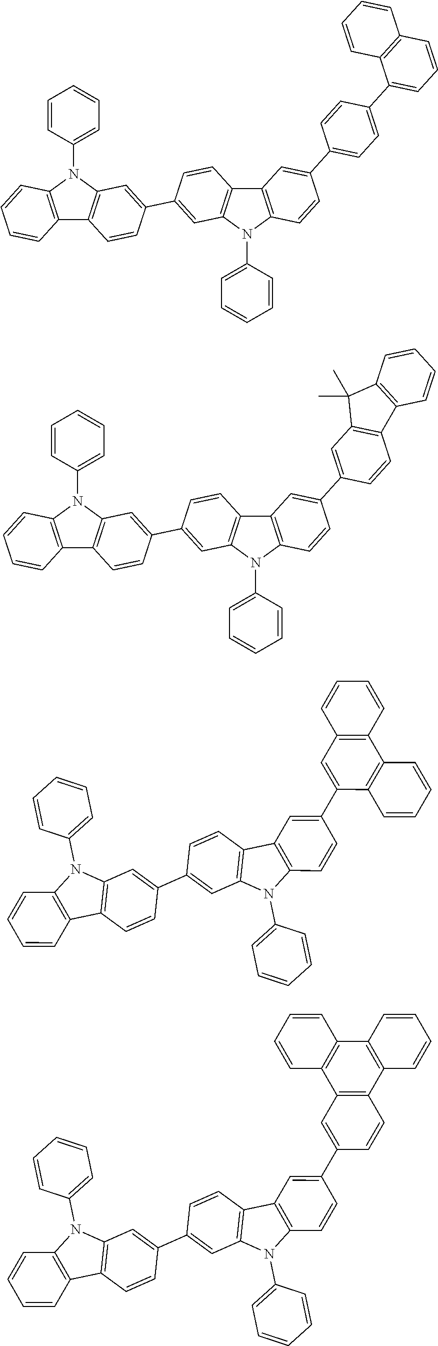

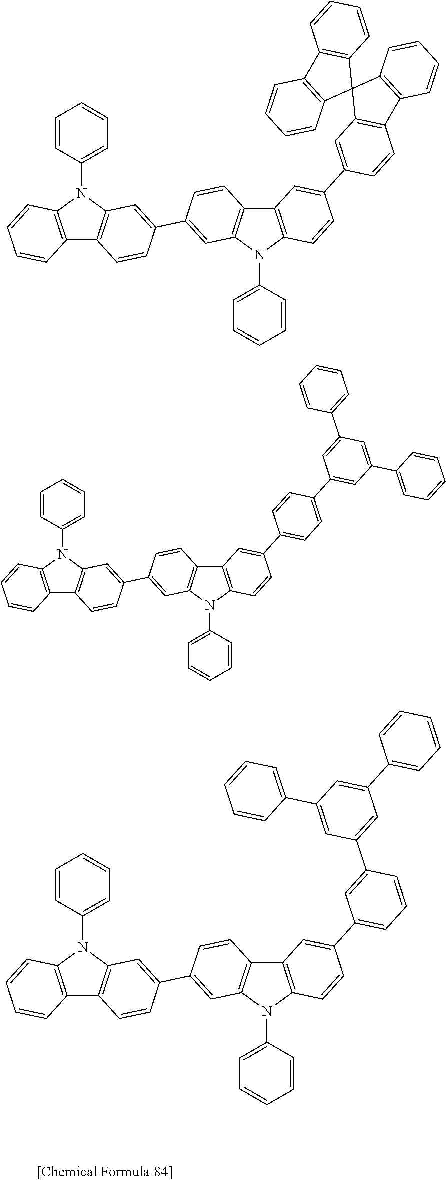

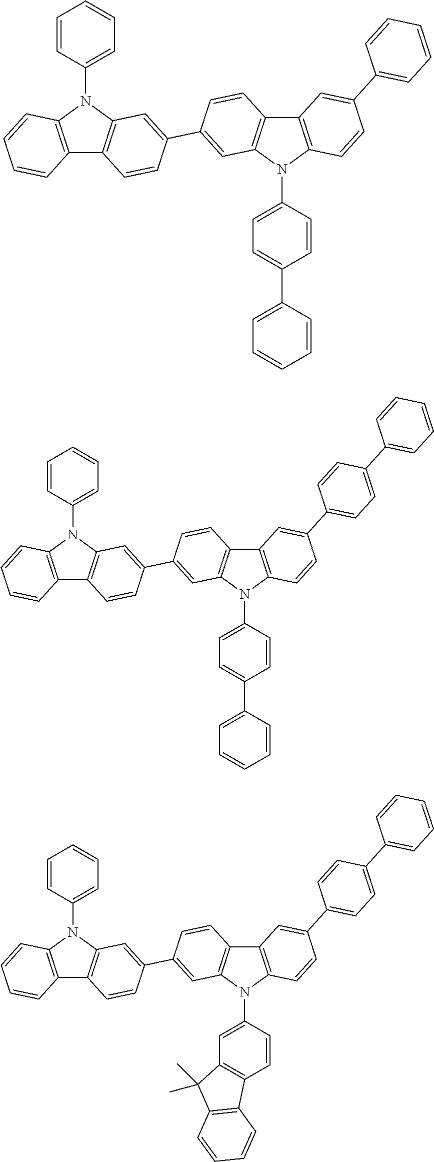

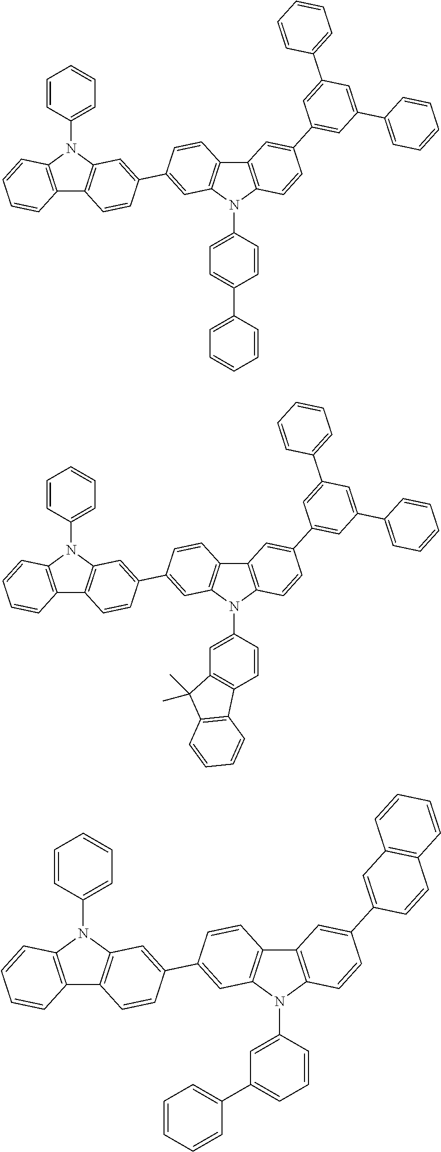

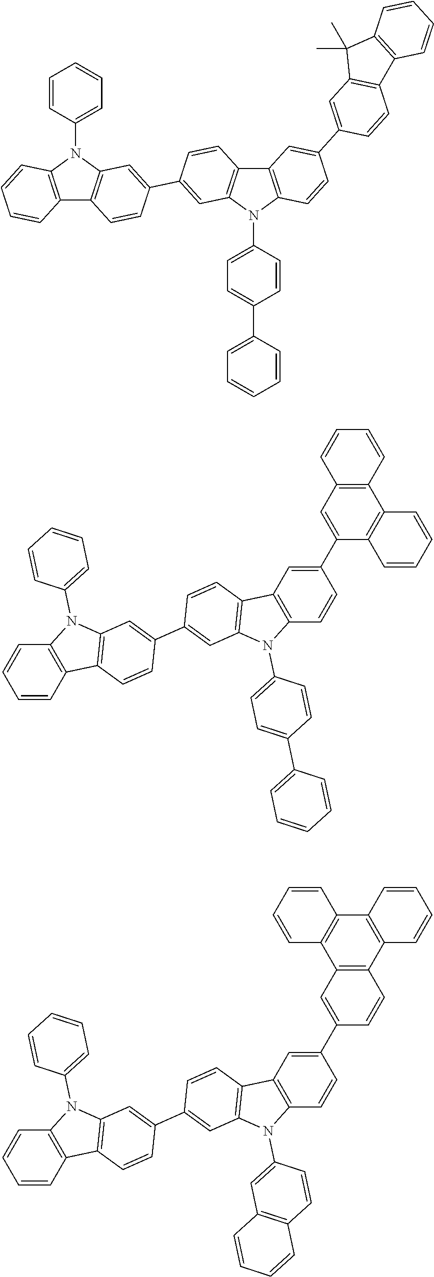

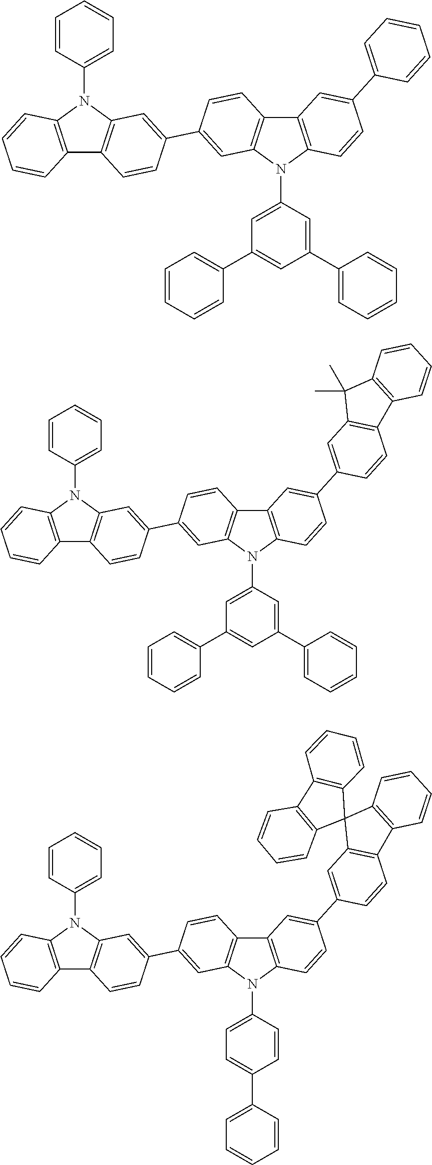

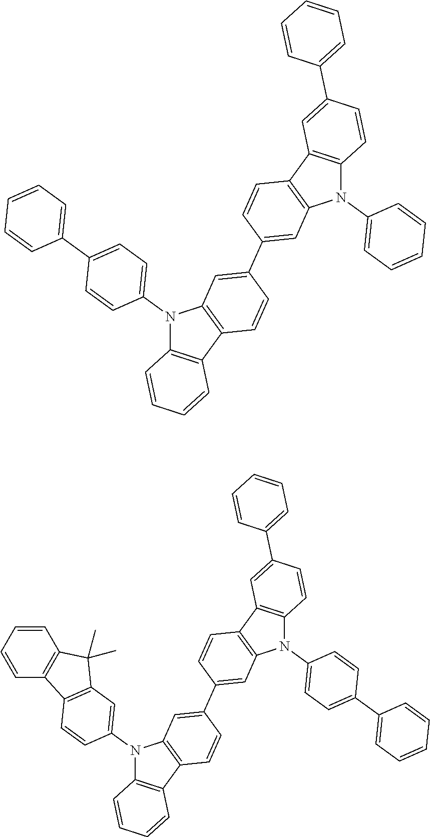

In order to achieve a high luminous efficiency, the efficiency of energy transfer from the compound represented by general formula (2) to the compound represented by general formula (1) is required to be improved. Further, when the transfer of the singlet excitation energy is not efficiently performed, the mixture of light emission derived from the compound represented by general formula (2) causes deteriorated color purity.

Examples of a mechanism by which the singlet excitation energy converted by the compound represented by general formula (2) is transferred to the compound represented by general formula (1) include a Forster mechanism. In the Forster mechanism, as the overlap integral between the light emission spectrum of an energy donor and the absorption spectrum of an energy acceptor increases, a Forster distance increases, whereby the energy transfer is likely to occur. Therefore, as the overlap between the fluorescence spectrum of the compound represented by general formula (2) as the donor and the absorption spectrum of the compound represented by general formula (1) as the acceptor increases, the transfer of the singlet excitation energy efficiently occurs.

As a result of the consideration, efficient energy transfer was confirmed to occur when the following numerical expression (i-1) is satisfied.

|λ1 (abs)−λ2 (FL)|≤50 (i-1)

λ1 (abs) represents a peak wavelength (nm) of a longest wavelength side peak in an absorption spectrum of the compound represented by general formula (1) at a wavelength of 400 nm or more and 900 nm or less. λ2 (FL) represents a peak wavelength (nm) of a longest wavelength side peak in a fluorescence spectrum of the compound represented by general formula (2) at a wavelength of 400 nm or more and 900 nm or less.

Here, the peak is a maximum portion of the spectrum, and the peak wavelength represents a wavelength at a maximum value. When the “longest wavelength side peak” is referred to, comparison is performed using main peaks excluding excessively small peaks such as noises. For example, small peaks having a half-value width of less than 10 nm are excluded.

When numerical expression (i-1) is satisfied, the overlap between the fluorescence spectrum of the compound represented by general formula (2) and the absorption spectrum of the compound represented by general formula (1) sufficiently increases, whereby energy transfer from the compound represented by general formula (2) to the compound represented by general formula (1) efficiently proceeds. Therefore, light emission derived from the compound represented by general formula (2) is suppressed, which mainly provides light emission derived from the compound represented by general formula (1). The light emission spectrum of the emissive layer represents a single peak. That is, it is possible to efficiently utilize the excitation energy and simultaneously achieve light emission having high color purity. In this case, the characteristics of light emission having a small half-value width and a high color purity as the characteristics of the compound represented by general formula (1) can be sufficiently utilized.

More preferably, the following numerical expression (i-2) is satisfied. λ1 (abs) and λ2 (FL) are the same as those in numerical expression (i-1).

|λ1 (abs)−λ2 (FL)|≤30 (i-2)

When numerical expression (i-2) is satisfied, the overlap between the fluorescence spectrum of the compound represented by general formula (2) and the absorption spectrum of the compound represented by general formula (1) further increases, whereby energy transfer from the compound represented by general formula (2) to the compound represented by general formula (1) particularly efficiently proceeds. Therefore, the light emission derived from the compound represented by general formula (2) is sufficiently suppressed, whereby more efficient utilization of excitation energy and light emission having higher color purity can be achieved.

Since the compound represented by general formula (1) has high fluorescence quantum yield, the singlet excitation energy transferred from the compound represented by general formula (2) can be smoothly converted into fluorescence. This makes it possible to suppress the singlet excitation energy from remaining in the compound represented by general formula (2), to suppress the light emission derived from the compound represented by general formula (2). The compound represented by general formula (2) does not necessarily have higher fluorescence quantum yield than that of the compound represented by general formula (1). Therefore, when the singlet excitation energy remains in the compound represented by general formula (2), energy loss due to non-radiative deactivation and the like occurs if only the compound represented by general formula (2) exists therein. However, the loss can be suppressed by combining the compound represented by general formula (2) with the compound represented by general formula (1).

Thus, by suitably combining the specific compound represented by general formula (1) with the specific compound represented by general formula (2), the light emission of a single peak in a wavelength range of 400 nm or more and 900 nm or less can be achieved. The half-value width of the single peak is preferably 60 nm or less, and more preferably 50 nm or less.

The light-emitting element according to the embodiment of the present invention may include an emissive layer (hereinafter referred to as “other emissive layer” as appropriate) in addition to the emissive layer containing the compound represented by general formula (1) and the compound represented by general formula (2). In that case, in addition to the compound represented by general formula (1) and the compound represented by general formula (2), an emissive material which is generally used can be used.

The other emissive layer may be in the form of a single layer or a plurality of layers, and is formed of an emissive material (host material, dopant material). The other emissive layer may be composed of a mixture of a host material and a dopant material, or may be composed of a host material alone. That is, only the host material or the dopant material may emit light, or both the host material and the dopant material may emit light, in each emissive layer. From the viewpoint that electrical energy is efficiently utilized to provide light emission having high color purity, it is preferred that the other emissive layer includes a mixture of the host material and the dopant material.

Each of the host material and the dopant material may be one kind or a combination of a plurality of kinds. The dopant material may be contained in a whole host material, or may be partially contained therein. The dopant material may be laminated, or may be dispersed.

The dopant material can control an emitted color. When the amount of the dopant material is too large, concentration quenching occurs, and therefore the dopant material is preferably used in an amount of 20% by weight or less, and more preferably 10% by weight or less based on the host material. Examples of a doping method include a method in which a host material and a doping material are co-evaporated, and a method in which a host material and a doping material are mixed in advance, and simultaneously evaporated.

The host material contained in the emissive material is not particularly limited. Examples of the host material which can be used include, but are not particularly limited to, compounds having a fused aryl ring such as naphthalene, anthracene, phenanthrene, pyrene, chrysene, naphthacene, triphenylene, perylene, fluoranthene, fluorene and indene, and derivatives thereof, aromatic amine derivatives such as N,N′-dinaphthyl-N,N′-diphenyl-4,4′-diphenyl-1,1′-diamine, metal chelated oxinoid compounds including tris(8-quinolinato)aluminum (III), bisstyryl derivatives such as distyrylbenzene derivatives, tetraphenylbutadiene derivatives, indene derivatives, coumarin derivatives, oxadiazole derivatives, pyrrolopyridine derivatives, perinone derivatives, cyclopentadiene derivatives, pyrrolopyrrole derivatives, thiadiazolopyridine derivatives, dibenzofuran derivatives, carbazole derivatives, indolocarbazole derivatives and triazine derivatives and, as a polymer series, polyphenylenevinylene derivatives, polyparaphenylene derivatives, polyfluorene derivatives, polyvinylcarbazole derivatives, and polythiophene derivatives.

The dopant material is not particularly limited, but examples of the dopant material which can be used include compounds having a fused aryl ring such as naphthalene, anthracene, phenanthrene, pyrene, chrysene, triphenylene, perylene, fluoranthene, fluorene and indene, and derivatives thereof (e.g., 2-(benzothiazol-2-yl)-9,10-diphenylanthracene and 5,6,11,12-tetraphenylnaphthacene); compounds having a heteroaryl ring such as furan, pyrrole, thiophene, silole, 9-silafluorene, 9,9′-spirobisilafluorene, benzothiophene, benzofuran, indole, dibenzothiophene, dibenzofuran, imidazopyridine, phenanthroline, pyridine, pyrazine, naphthyridine, quinoxaline, pyrrolopyridine and thioxanthene, and derivatives thereof; borane derivatives; distyrylbenzene derivatives; aminostyryl derivatives such as 4,4′-bis(2-(4-diphenylaminophenyl)ethenyl)biphenyl and 4,4′-bis(N-(stilben-4-yl)-N-phenylamino)stilbene; aromatic acetylene derivatives; tetraphenylbutadiene derivatives; stilbene derivatives; aldazine derivatives; pyrromethene derivatives; diketopyrrolo[3,4-c]pyrrole derivatives; coumarin derivatives such as 2,3,5,6-1H,4H-tetrahydro-9-(2′-benzothiazolyl)quinolizino[9,9a,1-gh]coumarin; azole derivatives such as imidazole, triazole, thiadiazole, carbazole, oxazole, oxadiazole and triazole, and metal complexes thereof; and aromatic amine derivatives typified by N,N′-diphenyl-N,N′-di(3-methylphenyl)-4,4′-diphenyl-1,1′-diamine.

The other emissive layer may contain a phosphorescence emitting material. The phosphorescence emitting material is a material which emits phosphorescence at room temperature. When a phosphorescence emitting material is used as a dopant, the phosphorescence emitting material is not particularly limited, and is preferably an organic metal complex compound containing at least one metal selected from the group consisting of iridium (Ir), ruthenium (Ru), rhodium (Rh), palladium (Pd), platinum (Pt), osmium (Os), and rhenium (Re). Among these, an organic metal complex having iridium or platinum is more preferred from the viewpoint that it has high phosphorescence light emission yield at room temperature.

As the host to be used in combination with a phosphorescence emitting dopant, preferred are aromatic hydrocarbon compound derivatives such as indole derivatives, carbazole derivatives, indolocarbazole derivatives, nitrogen-containing aromatic compound derivatives having a pyridine, pyrimidine or triazine skeleton, polyarylbenzene derivatives, spirofluorene derivatives, truxene derivatives and triphenylene derivatives; compounds containing a chalcogen element, such as dibenzofuran derivatives and dibenzothiophene derivatives; and organic metal complexes such as beryllium quinolinol complexes, and the like. The host is not limited thereto as long as basically it has higher triplet energy than a dopant used, and electrons and holes are smoothly injected and transported from the respective transporting layers. Two or more triplet emissive dopants may be contained in the other emissive layer, and two or more host materials maybe contained. Further, one or more triplet emissive dopants and one or more fluorescence emitting dopants may be contained in the other emissive layer.

The preferable phosphorescence emitting host or dopant is not particularly limited, but specific examples thereof include the following.

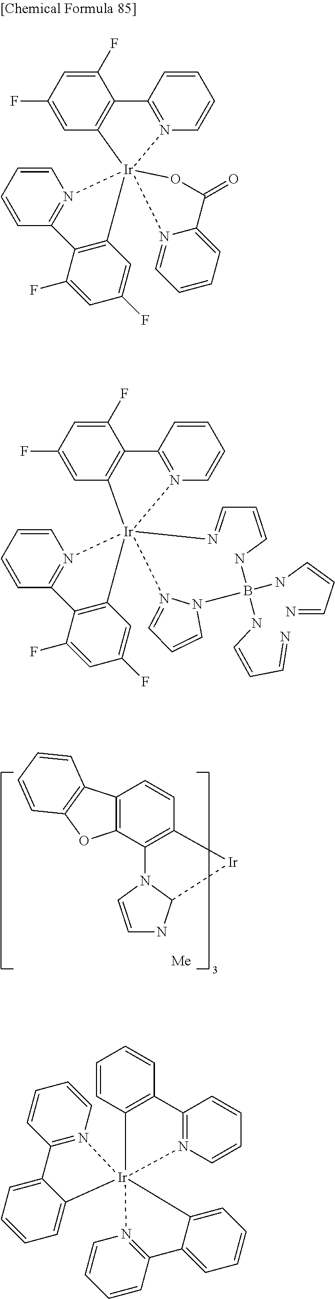

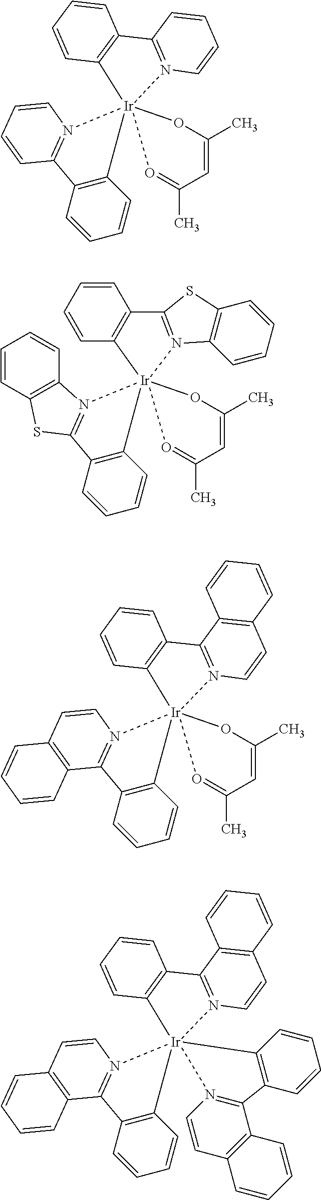

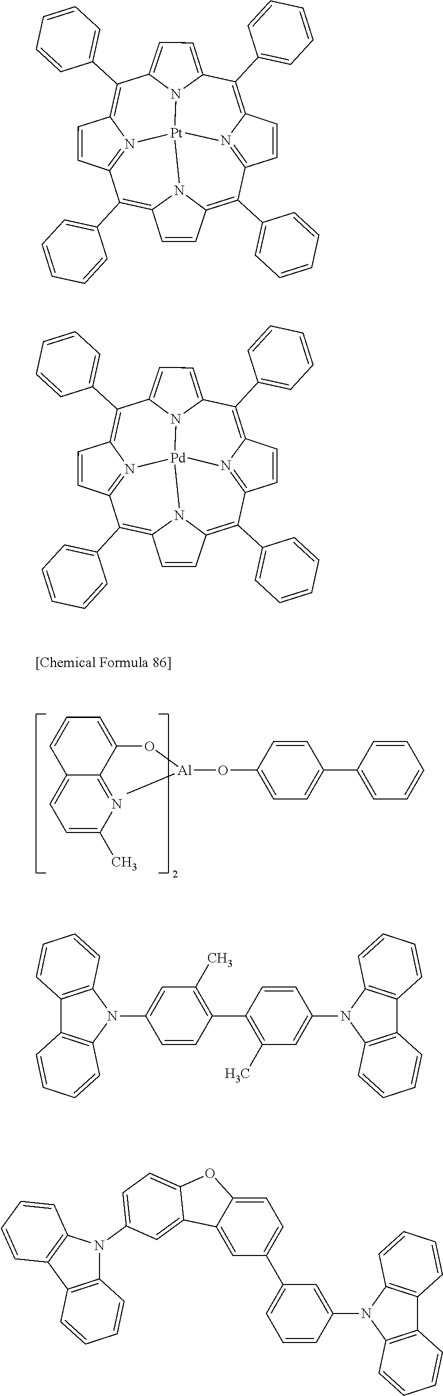

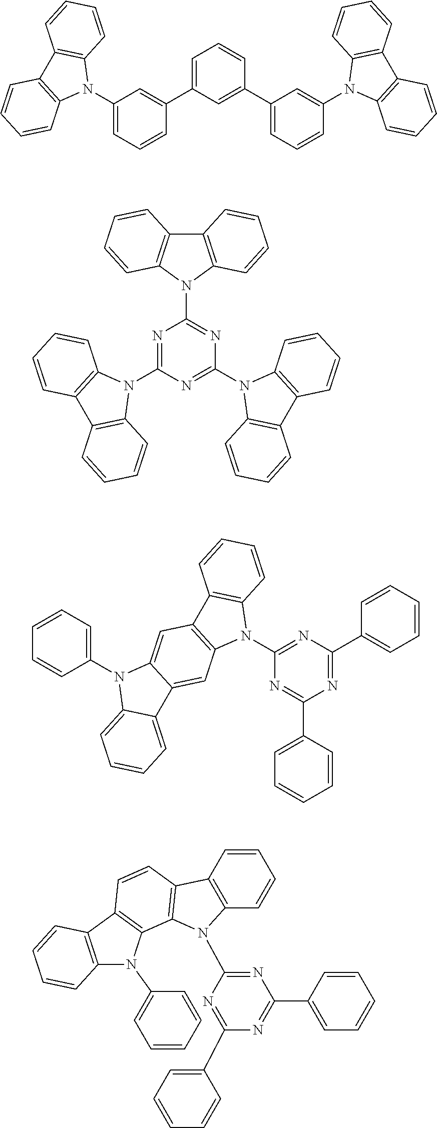 [SMILES: c1cc(-c2cccc(-n3c4ccccc4c4ccccc43)c2)cc(-c2cccc(-n3c4ccccc4c4ccccc43)c2)c1 ; c1ccc2c(c1)c1ccccc1n2-c1nc(-n2c3ccccc3c3ccccc32)nc(-n2c3ccccc3c3ccccc32)n1 ; c1ccc(-c2nc(-c3ccccc3)nc(-n3c4ccccc4c4cc5c(cc43)c3ccccc3n5-c3ccccc3)n2)cc1 ; c1ccc(-c2nc(-c3ccccc3)nc(-n3c4ccccc4c4ccc5c6ccccc6n(-c6ccccc6)c5c43)n2)cc1]

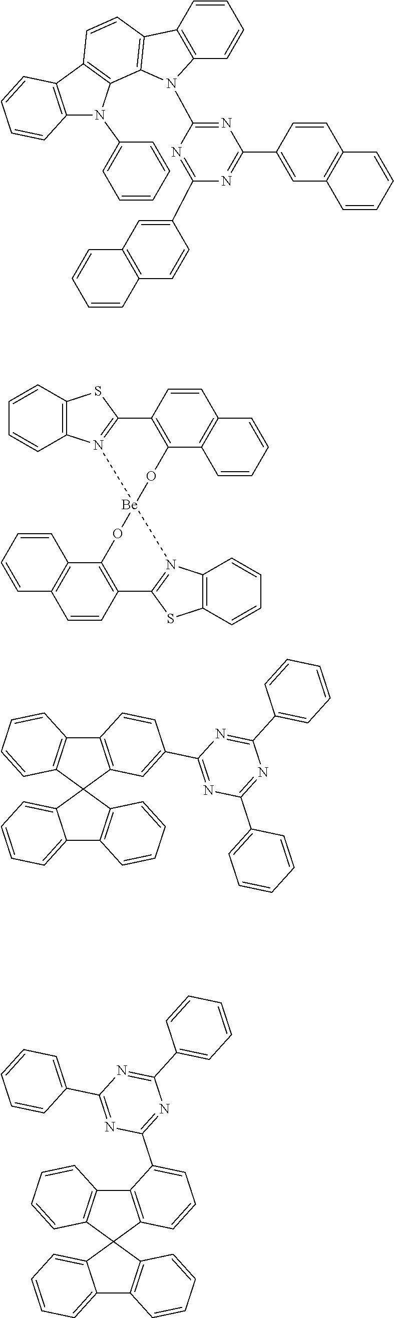

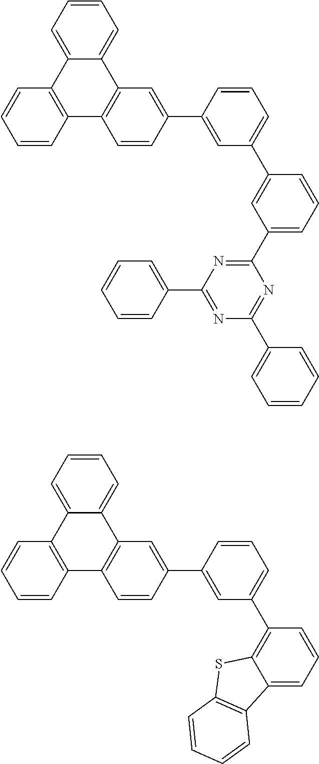 [SMILES: c1ccc(-c2nc(-c3ccccc3)nc(-c3cccc(-c4cccc(-c5ccc6c7ccccc7c7ccccc7c6c5)c4)c3)n2)cc1 ; c1cc(-c2ccc3c4ccccc4c4ccccc4c3c2)cc(-c2cccc3c2sc2ccccc23)c1]

The other emissive layer may contain a TADF material as a dopant. The TADF material may be a material which exhibits TADF provided by a single material, or may be a material which exhibits TADF provided by a plurality of materials. The TADF material to be used may include a single material or a plurality of materials, and for the TADF material, a known material can be used. Specific examples thereof include benzonitrile derivatives, triazine derivatives, disulfoxide derivatives, carbazole derivatives, indolocarbazole derivatives, dihydrophenazine derivatives, thiazole derivatives, and oxadiazole derivatives. The compound represented by general formula (2) of the present invention can also be suitably used as a TADF dopant.

(Anode and Cathode)

In the light-emitting element according to the embodiment of the present invention, the anode and the cathode have a role for supplying a sufficient current for light emission of the element. It is preferred that at least one of the anode and the cathode is transparent or translucent in order to take out light. Usually, the anode formed on a substrate is made to be a transparent electrode.

The material to be used for an anode is not particularly limited as long as being a material which is capable of efficiently injecting holes into an organic layer, and examples thereof include electroconductive metal oxides such as tin oxide, indium oxide, indium tin oxide (ITO), and indium zinc oxide (IZO), metals such as gold, silver, and chromium, inorganic electroconductive substances such as copper iodide and copper sulfide, or electroconductive polymers such as polythiophene, polypyrrole, andpolyaniline. Among these, ITO and tin oxide are preferred. These electrode materials may be used alone, ora plurality of materials maybe used in lamination or in admixture. As long as a sufficient current for light emission of the element can be supplied, the resistance of an anode is not limited, but from the viewpoint of the power consumption of the element, the element preferably has low resistance. For example, an ITO substrate having resistance of 300 Ω/□ or lower functions as an element electrode, but it is particularly preferable to use a substrate having low resistance of 20 Ω/□ or lower. The thickness of the anode can be optionally selected according to a resistance value, but is usually preferably 100 to 300 nm.

In addition, in order to retain the mechanical strength of the light-emitting element, it is preferred to form the light-emitting element on a substrate. As the substrate, a glass substrate composed of soda glass or alkali-free glass and the like is suitably used. Since it is favorable that the glass substrate has a sufficient thickness for retaining the mechanical strength, the thickness of 0.5 mm or more is sufficient. Regarding the material of glass, since it is preferred that the amount of ions eluted from glass is small, alkali-free glass is more preferable. Since soda lime glass provided with a barrier coating such as SiO₂ is also commercially available, it can also be used. Further, as long as the anode stably functions, it is not necessary that the substrate is composed of glass and, for example, the anode may be formed on a plastic substrate. Examples of a method for forming an anode include, but are not particularly limited to, an electron beam method, a sputtering method, and a chemical reaction method.

A material to be used in the cathode is not particularly limited, as long as it is a substance which can efficiently inject electrons into the emissive layer. Generally, metals such as platinum, gold, silver, copper, iron, tin, aluminum, and indium, or alloys or multilayer laminated body of these metals with metals having a low work function such as lithium, sodium, potassium, calcium and magnesium are preferred. Among these, as a main component of the cathode, aluminum, silver, and magnesium are preferred from the viewpoints of an electric resistance value, easiness of making a film, stability of a film, and a luminous efficiency and the like. In particular, the cathode is composed of magnesium and silver to provide easy electron injection into the electron transporting layer and the electron injection layer, thereby allowing low voltage driving, which is preferable.

Further, metals such as platinum, gold, silver, copper, iron, tin, aluminum, and indium, alloys using at least one or more of these metals, inorganic substances such as silica, titania, and silicon nitride, and an organic polymer compound such as polyvinyl alcohol, polyvinyl chloride, or a hydrocarbon-based polymer compound are preferably laminated as a protective film layer on the cathode in order to protect the cathode. However, in the case of an element structure for taking out light from the cathode side (top emission structure), the protective film layer is selected from materials having light permeability in a visible light region. Examples of a method for preparation of these cathodes include, but are not particularly limited to, resistance heating, electron beam, sputtering, ion plating and coating.

(Hole Transporting Layer)

The hole transporting layer is formed by a method in which one or two or more hole transporting materials are laminated or mixed, or a method using a mixture of a hole transporting material and a polymer binder. It is preferable that the hole transporting material efficiently transports holes from the positive electrode. Therefore, it is preferred that the hole transporting material has a high hole injection efficiency to efficiently transport injected holes.

The hole transporting material is not particularly limited, but example thereof include benzidine derivatives such as 4,4′-bis(N-(3-methylphenyl)-N-phenylamino)biphenyl (TPD), 4,4′-bis(N-(1-naphthyl)-N-phenylamino)biphenyl (NPD), 4,4′-bis(N,N-bis(4-biphenylyl)amino)biphenyl (TBDB) and bis(N,N′-diphenyl-4-aminophenyl)-N,N-diphenyl-4,4′-diamino-1,1′-biphenyl (TPD232); materials called starburst arylamines, such as 4,4′,4″-tris(3-methylphenyl(phenyl)amino)triphenylamine (m-MTDATA) and 4,4′,4″-tris(1-naphthyl(phenyl)amino)triphenylamine (1-TNATA); and materials having a carbazole skeleton.

Among these, carbazole multimers, specifically derivatives of carbazole dimers such as bis(N-arylcarbazole) or bis(N-alkylcarbazole), derivatives of carbazole trimers, and derivatives of carbazole tetramers are preferable, and derivatives of carbazole dimers and derivatives of carbazole trimers are more preferable. Furthermore, asymmetric bis(N-arylcarbazole) derivatives are particularly preferable. Since these carbazole multimers have both good electron blocking properties and hole injecting transporting properties, they can contribute to a further improvement in an efficiency of the light-emitting element.

Materials having one carbazole skeleton and one triarylamine skeleton are also preferable. Materials having an arylene group as a linking group between the nitrogen atom of amine and a carbazole skeleton are more preferable, and materials having skeletons represented by general formulae (12) and (13) are particularly preferable.

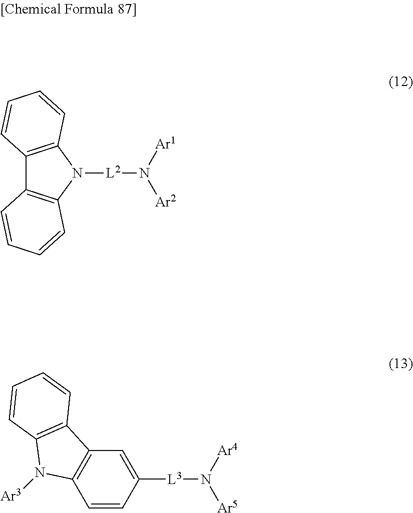

L² and L³ each are an arylene group, and Ar¹ to Ar⁵ each are an aryl group.

Besides the above-mentioned compounds, examples of hole transporting materials include heterocyclic compounds such as triphenylene compounds, pyrazoline derivatives, stilbene derivatives, hydrazone derivatives, benzofuran derivatives and thiophene derivatives, oxadiazole derivatives, phthalocyanine derivatives and porphyrin derivatives; and fullerene derivatives. Polymer-based compounds such as polycarbonate and styrene derivatives, each having the same structure as that of the hole transporting material on the side chain can be preferably used as the hole transporting material. In addition, polythiophene, polyaniline, polyfluorene, polyvinylcarbazole, and polysilane and the like can also be preferably used. Further, inorganic compounds such as p-type Si and p-type SiC can also be used.

Although the hole transporting layer may be composed of a plurality of layers, a monoamine compound having a spirofluorene skeleton is preferably used as the hole transporting layer which is directly in contact with the emissive layer of the present invention. Usually, electrons are injected into the emissive layer at the LUMO level of the host material. The delayed fluorescent compound exemplified by the compound represented by general formula (2) used in the emissive layer of the present invention has a strong electron-accepting property, in other words, a substituent having a high electron affinity, whereby the LUMO level of the delayed fluorescent compound is deeper than that of the host material. Therefore, the emissive layer containing the delayed fluorescent compound more easily receives electrons from the electron transporting layer than a general emissive layer receives. Further, when the compounds represented by general formula (1) are contained in the emissive layer, the compounds have a LUMO level deeper than that of the delayed fluorescent compound as well as the host material. Therefore, the emissive layer containing the delayed fluorescent compound and the compound represented by general formula (1) more easily receives electrons from the electron transporting layer, whereby the emissive layer of the present invention is apt to have excess electrons. For this reason, the electrons are apt to leak to the hole transporting layer side. In order to suppress the leakage, the electrons are required to be confined in the emissive layer using a hole transporting material having a low electron affinity, that is, a shallow LUMO level.

For such a problem, a monoamine compound having a spirofluorene skeleton is a material having a large steric hindrance. The planarity of molecules of such a material can be reduced to reduce the interaction between the molecules. The interaction between the molecules is reduced, so that the energy gap is larger to cause a shallower LUMO level. That is, the electron affinity is reduced, and the electron blocking property is increased, whereby the electrons can be confined in the emissive layer, to allow the luminous efficiency and the durability to be further improved. Further, the interaction between the molecules is reduced, whereby the fluorescence quantum yield in the amorphous state is improved. Therefore, in the organic thin-film light-emitting element, the decomposition of the material in an excited state can be suppressed, whereby an element having high durability is obtained.

Preferred examples of the remaining two substituents bonded to the nitrogen atom of the monoamine compound having a spirofluorene skeleton include an aryl group and a heteroaryl group. From the viewpoint of having a high triplet level to prevent the deepening of the LUMO level, the aryl group is more preferably a substituted or unsubstituted biphenyl group, a substituted or unsubstituted terphenyl group, a substituted or unsubstituted fluorenyl group, or a substituted or unsubstituted spirofluorenyl group, and still more preferably a substituted or unsubstituted biphenyl group or a substituted or unsubstituted fluorenyl group. From the viewpoint of having higher mobility to allow a driving voltage to be reduced, a substituted or unsubstituted p-biphenyl group, a substituted or unsubstituted p-terphenyl group, and a substituted or unsubstituted 2-fluorenyl group are most preferred.

For example, there is a concern that the LUMO level is deep when a group containing electron-accepting nitrogen such as a pyridyl group exists, whereby it is preferred that the heteroaryl group does not contain electron-accepting nitrogen. In particular, the heteroaryl group is more preferably a substituted or unsubstituted dibenzofuranyl group which has electron resistance and can be expected to have improved durability, or a group having a substituted or unsubstituted dibenzothiophenyl group, and still more preferably a substituted or unsubstituted dibenzofuranyl group. The preferred monoamine compound having a spirofluorene skeleton is not particularly limited, but specific examples thereof include the following.

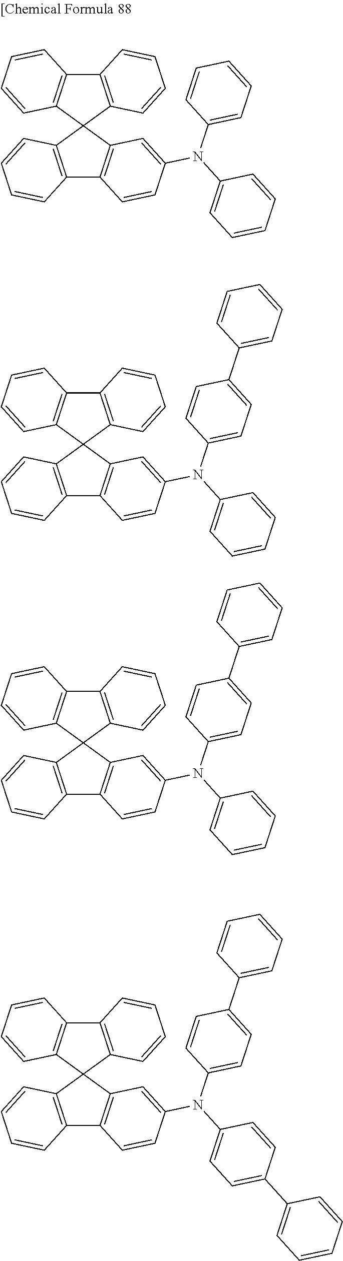

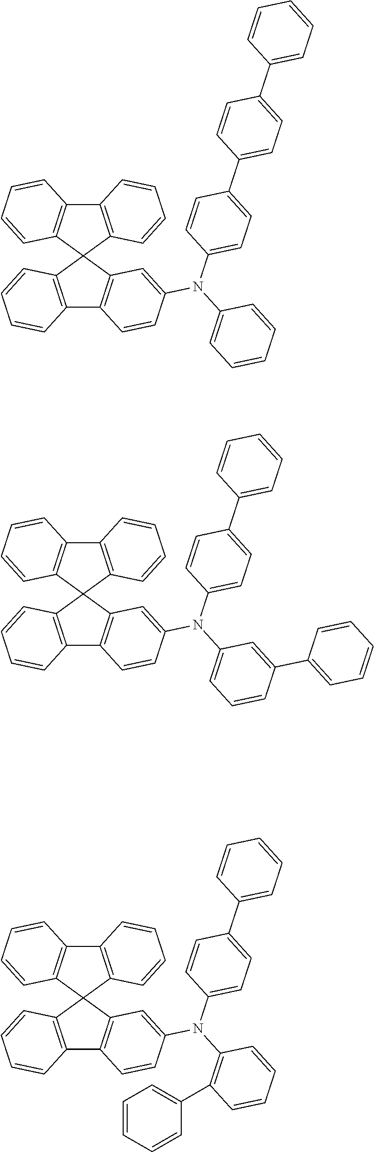 [SMILES: c1ccc(-c2ccc(-c3ccc(N(c4ccccc4)c4ccc5c(c4)C4(c6ccccc6-c6ccccc64)c4ccccc4-5)cc3)cc2)cc1 ; c1ccc(-c2ccc(N(c3cccc(-c4ccccc4)c3)c3ccc4c(c3)C3(c5ccccc5-c5ccccc53)c3ccccc3-4)cc2)cc1 ; c1ccc(-c2ccc(N(c3ccc4c(c3)C3(c5ccccc5-c5ccccc53)c3ccccc3-4)c3ccccc3-c3ccccc3)cc2)cc1]

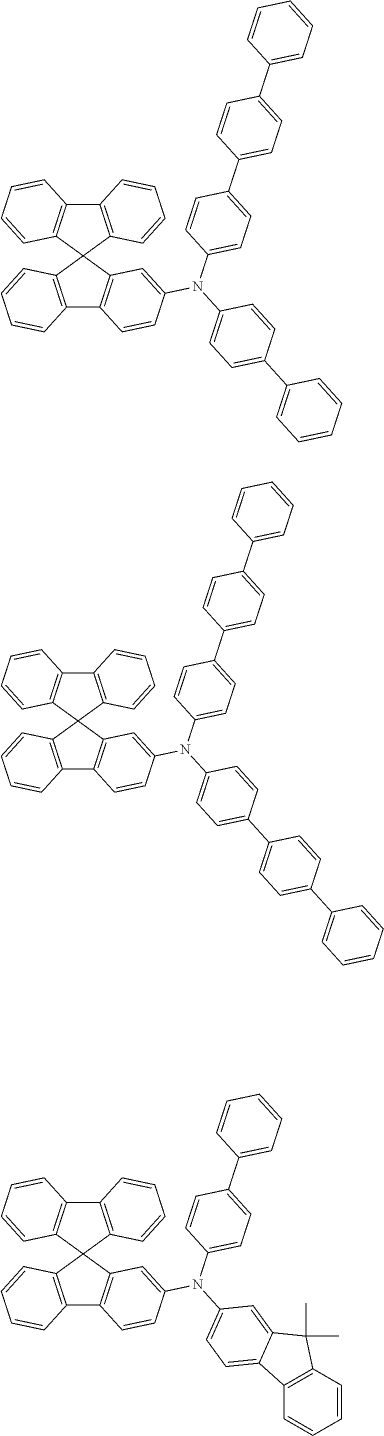 [SMILES: c1ccc(-c2ccc(-c3ccc(N(c4ccc(-c5ccccc5)cc4)c4ccc5c(c4)C4(c6ccccc6-c6ccccc64)c4ccccc4-5)cc3)cc2)cc1 ; c1ccc(-c2ccc(-c3ccc(N(c4ccc(-c5ccc(-c6ccccc6)cc5)cc4)c4ccc5c(c4)C4(c6ccccc6-c6ccccc64)c4ccccc4-5)cc3)cc2)cc1 ; CC1(C)c2ccccc2-c2ccc(N(c3ccc(-c4ccccc4)cc3)c3ccc4c(c3)C3(c5ccccc5-c5ccccc53)c3ccccc3-4)cc21]

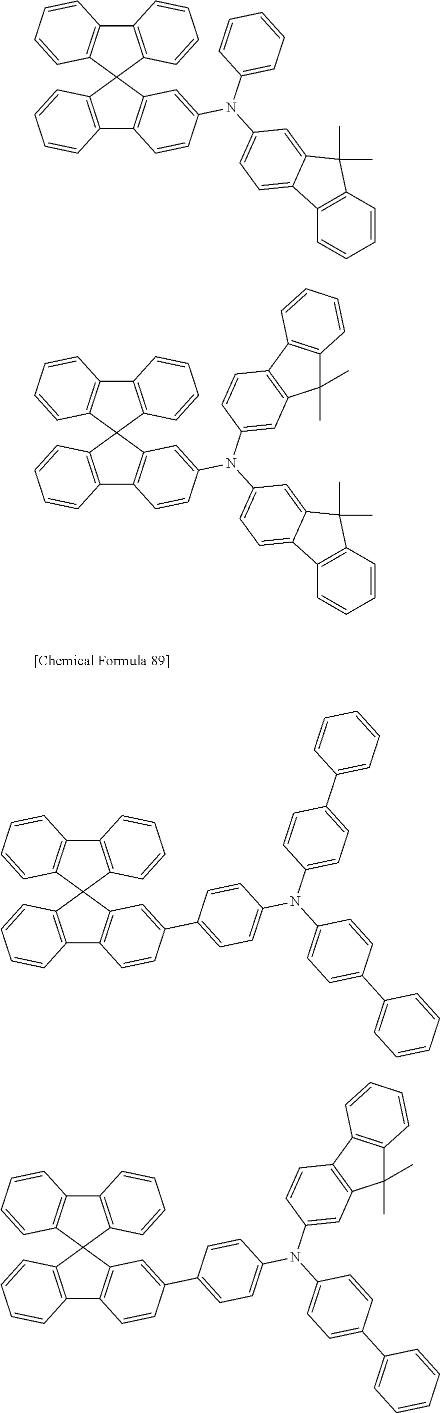

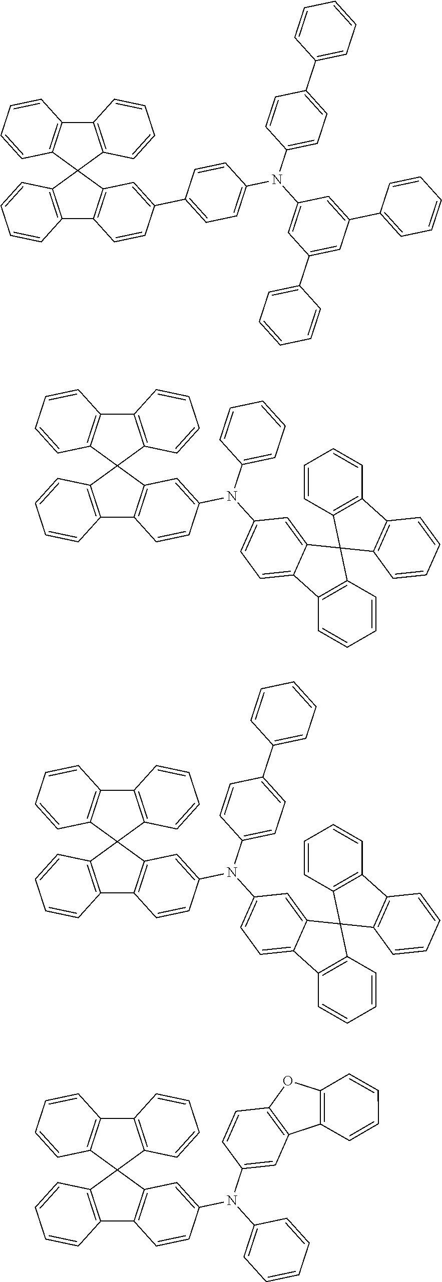 [SMILES: c1ccc(-c2ccc(N(c3ccc(-c4ccc5c(c4)C4(c6ccccc6-c6ccccc64)c4ccccc4-5)cc3)c3cc(-c4ccccc4)cc(-c4ccccc4)c3)cc2)cc1 ; c1ccc(N(c2ccc3c(c2)C2(c4ccccc4-c4ccccc42)c2ccccc2-3)c2ccc3c(c2)C2(c4ccccc4-c4ccccc42)c2ccccc2-3)cc1 ; c1ccc(-c2ccc(N(c3ccc4c(c3)C3(c5ccccc5-c5ccccc53)c3ccccc3-4)c3ccc4c(c3)C3(c5ccccc5-c5ccccc53)c3ccccc3-4)cc2)cc1 ; c1ccc(N(c2ccc3c(c2)C2(c4ccccc4-c4ccccc42)c2ccccc2-3)c2ccc3oc4ccccc4c3c2)cc1]

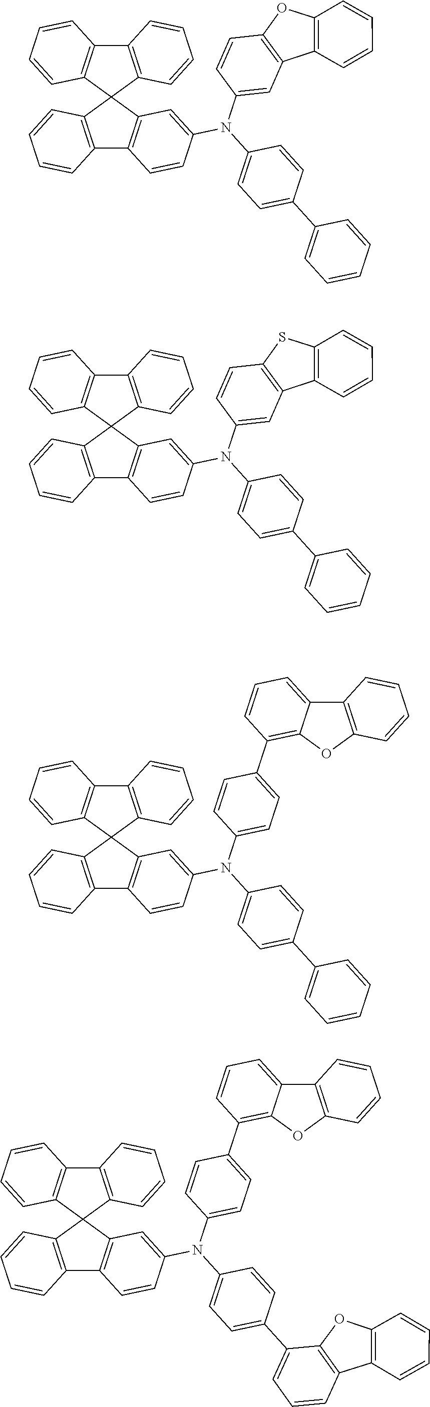

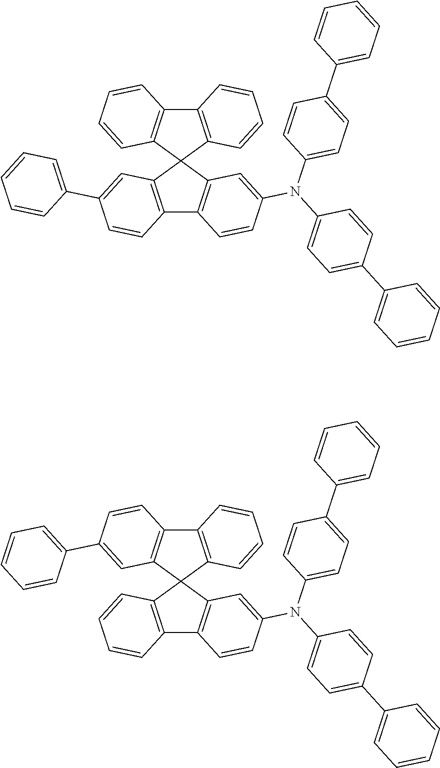 [SMILES: c1ccc(-c2ccc(N(c3ccc(-c4ccccc4)cc3)c3ccc4c(c3)C3(c5ccccc5-c5ccccc53)c3cc(-c5ccccc5)ccc3-4)cc2)cc1 ; c1ccc(-c2ccc(N(c3ccc(-c4ccccc4)cc3)c3ccc4c(c3)C3(c5ccccc5-c5ccc(-c6ccccc6)cc53)c3ccccc3-4)cc2)cc1]

(Hole Injection Layer)

In the light-emitting element according to the embodiment of the present invention, a hole injection layer may be provided between an anode and a hole transporting layer. When the hole injection layer is provided, the light-emitting element has a reduced driving voltage, and durable life is also improved.

Specific examples of the hole injection layer include benzidine derivatives such as TPD232, and starburst arylamine materials, and besides, phthalocyanine derivatives and the like can also be used.

It is preferred that the hole injection layer is formed of an acceptor compound alone, or used with another hole transporting material doped with an acceptor compound. Examples of the acceptor compound include, but are not particularly limited to, metal chlorides such as iron(III) chloride, aluminum chloride, gallium chloride, indium chloride, and antimony chloride, metal oxides such as molybdenum oxide, vanadium oxide, tungsten oxide, and ruthenium oxide, and charge transfer complexes such as tris(4-bromophenyl)aminium hexachloroantimonate (TBPAH). Organic compounds having a nitro group, a cyano group, a halogen, or a trifluoromethyl group in the molecule, quinone-based compounds, acid anhydride-based compounds, and fullerene and the like can also be suitably used.

Of these, metal oxides and cyano group-containing compounds are preferred. This is because these compounds are easily handled and deposited, and therefore the above-described effects are easily obtained. Specific examples of the cyano group-containing compound include the following compounds.

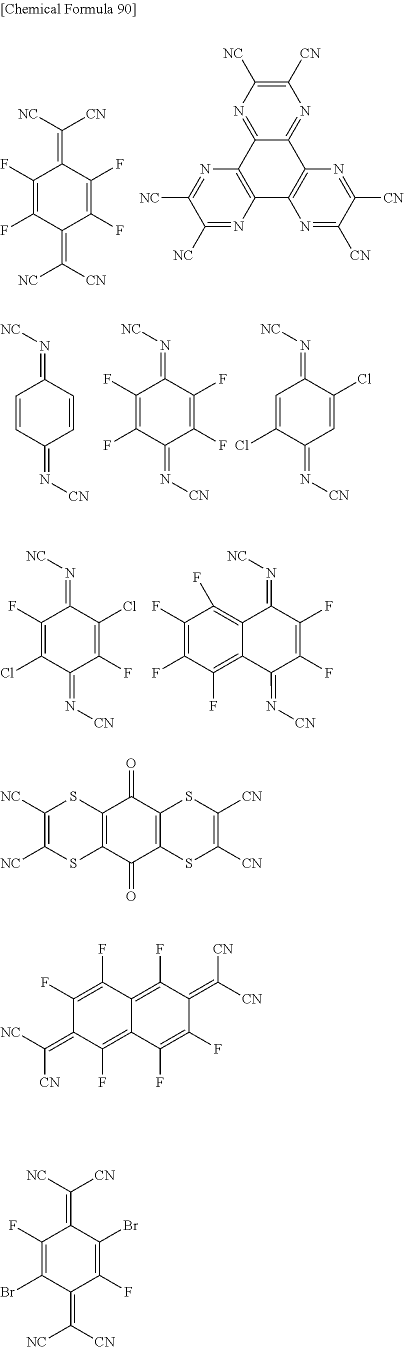

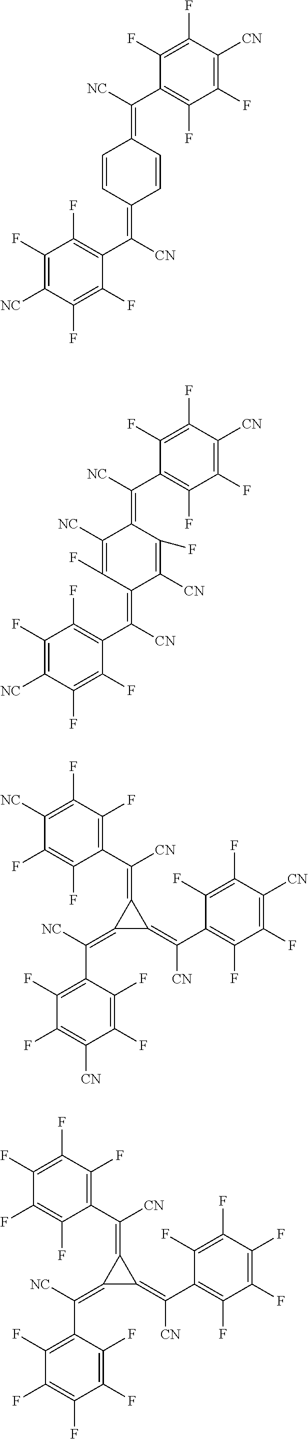

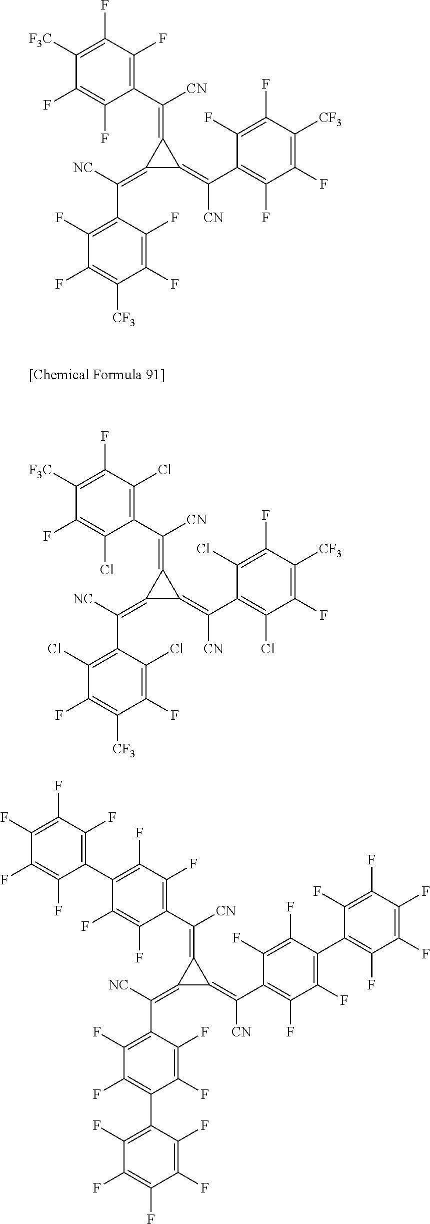 [SMILES: N#CC(=C1C(=C(C#N)c2c(F)c(F)c(C(F)(F)F)c(F)c2F)C1=C(C#N)c1c(F)c(F)c(C(F)(F)F)c(F)c1F)c1c(F)c(F)c(C(F)(F)F)c(F)c1F ; N#CC(=C1C(=C(C#N)c2c(Cl)c(F)c(C(F)(F)F)c(F)c2Cl)C1=C(C#N)c1c(Cl)c(F)c(C(F)(F)F)c(F)c1Cl)c1c(Cl)c(F)c(C(F)(F)F)c(F)c1Cl ; N#CC(=C1C(=C(C#N)c2c(F)c(F)c(-c3c(F)c(F)c(F)c(F)c3F)c(F)c2F)C1=C(C#N)c1c(F)c(F)c(-c2c(F)c(F)c(F)c(F)c2F)c(F)c1F)c1c(F)c(F)c(-c2c(F)c(F)c(F)c(F)c2F)c(F)c1F]

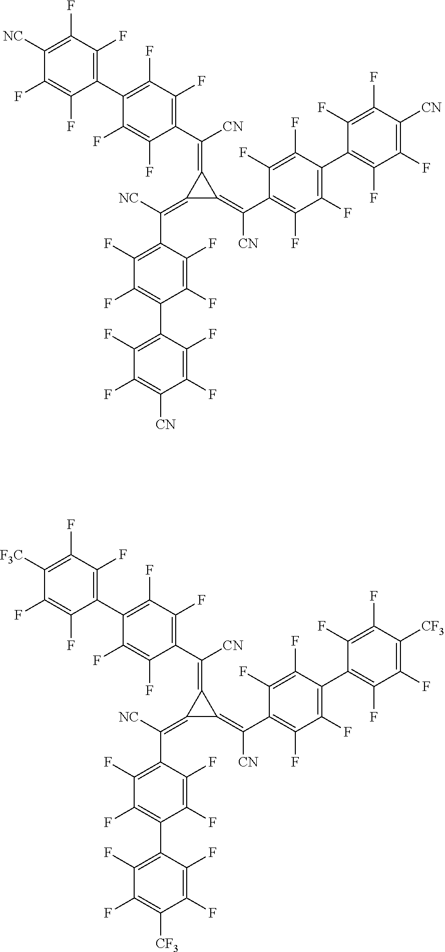 [SMILES: N#CC(=C1C(=C(C#N)c2c(F)c(F)c(-c3c(F)c(F)c(C#N)c(F)c3F)c(F)c2F)C1=C(C#N)c1c(F)c(F)c(-c2c(F)c(F)c(C#N)c(F)c2F)c(F)c1F)c1c(F)c(F)c(-c2c(F)c(F)c(C#N)c(F)c2F)c(F)c1F ; N#CC(=C1C(=C(C#N)c2c(F)c(F)c(-c3c(F)c(F)c(C(F)(F)F)c(F)c3F)c(F)c2F)C1=C(C#N)c1c(F)c(F)c(-c2c(F)c(F)c(C(F)(F)F)c(F)c2F)c(F)c1F)c1c(F)c(F)c(-c2c(F)c(F)c(C(F)(F)F)c(F)c2F)c(F)c1F]

In either of the case where a hole injection layer is formed of an acceptor compound alone or the case where a hole injection layer is doped with an acceptor compound, the hole injection layer may be a single layer or may be a laminate of a plurality of layers. The hole injection material to be used in combination when the hole injection layer is doped with an acceptor compound is preferably the same compound as the compound to be used for the hole transporting layer from the viewpoint that a barrier to injection of holes into the hole transporting layer can be mitigated.

(Electron Transporting Layer)

In the present invention, the electron transporting layer is a layer existing between a cathode and an emissive layer. The electron transporting layer may include a single layer, or a plurality of layers, and may or may not be in contact with a cathode or an emissive layer.

The electron transporting layer is desired to have a high electron injection efficiency from a cathode, efficiently transport injected electrons, and have a high electron injection efficiency to an emissive layer. Meanwhile, even if the electron transporting capability of the electron transporting layer is not so high, the electron transporting layer also desirably has a role of allowing efficient inhibition of holes flowing to the cathode side without the holes being recombined. Therefore, the electron transporting layer in the present invention also includes a hole inhibition layer which can efficiently inhibit the transfer of holes as the same meaning.

Examples of the electron transporting material to be used for the electron transporting layer include, but are not particularly limited to, fused polycyclic aromatic derivatives such as naphthalene and anthracene, styryl-based aromatic ring derivatives typified by 4,4′-bis(diphenylethenyl)biphenyl, quinone derivatives such as anthraquinone and diphenoquinone, phosphorus oxide derivatives, and various types of metal complexes such as quinolinol complexes, e.g., tris(8-quinolinolato)aluminum(III), benzoquinolinol complexes, hydroxyazole complexes, azomethine complexes, tropolone metal complexes, and flavonol metal complexes. It is also preferred to use a compound which includes an element selected from carbon, hydrogen, nitrogen, oxygen, silicon and phosphorus, and has an aromatic heterocyclic structure containing electron-accepting nitrogen.

Examples of the compound having an aromatic heterocyclic structure containing electron-accepting nitrogen include, but are not particularly limited to, pyrimidine derivatives, triazine derivatives, benzimidazole derivatives, benzoxazole derivatives, benzothiazole derivatives, oxadiazole derivatives, thiadiazole derivatives, triazole derivatives, pyrazine derivatives, phenanthroline derivatives, quinoline derivatives, benzoquinoline derivatives, oligopyridine derivatives such as bipyridine and terpyridine, quinoxaline derivatives and naphthyridine derivatives. Among these, triazine derivatives such as 2,4,6-tri([1,1′-biphenyl]-4-yl)-1,3,5-triazine; imidazole derivatives such as tris(N-phenylbenzimidazol-2-yl)benzene; oxadiazole derivatives such as 1,3-bis[(4-tert-butylphenyl)1,3,4-oxadiazolyl]phenylene; triazole derivatives such as N-naphthyl-2,5-diphenyl-1,3,4-triazole; phenanthroline derivatives such as bathocuproine and 1,3-bis(1,10-phenanthrolin-9-yl)benzene; benzoquinoline derivatives such as 2,2′-bis(benzo[h]quinolin-2-yl)-9,9′-spirobifluorene; bipyridine derivatives such as 2,5-bis(6′-(2′,2″-bipyridyl))-1,1-dimethyl-3,4-diphenylsilole; terpyridine derivatives such as 1,3-bis(4′-(2,2′:6′2″-terpyridinyl))benzene; and naphthyridine derivatives such as bis(1-naphthyl)-4-(1,8-naphthyridin-2-yl)phenylphosphine oxide are preferably used from the viewpoint of electron transporting ability.

Among these, particularly preferred examples of the electron transporting material include triazine derivatives and phenanthroline derivatives. The triazine derivative has high triplet energy, whereby triplet exciton energy generated in the emissive layer can be prevented from leaking to the electron transporting layer. Further, the TADF material used in the emissive layer has the LUMO energy level equivalent to that of the triazine derivative, whereby the use of the triazine derivative in the electron transporting layer can provide effective electron injection having a small barrier into the TADF material in the emissive layer to allow a low voltage, a high efficiency, and a long life to be achieved. Further, when the triazine derivative is the compound represented by general formula (15), the above-described effect is improved, which is more preferable.

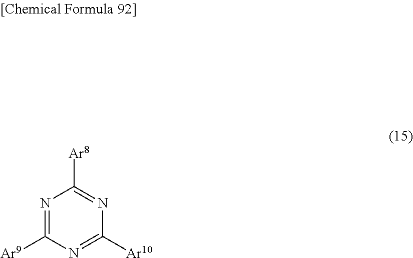

In general formula (15), Ar⁸ to Ar¹⁰ are the same or different from each other, and each are a substituted or unsubstituted aryl group or a substituted or unsubstituted heteroaryl group. The aryl group is preferably a phenyl group, a biphenyl group, a terphenyl group, a naphthyl group, a fluorenyl group, a spirofluorenyl group, a triphenylenyl group, or a phenanthrenyl group, and particularly preferably a phenyl group, a biphenyl group, a naphthyl group, or a fluorenyl group. The electron transporting layer may be formed of a plurality of layers. In that case, the triazine derivative is preferably used in a layer which is in direct contact with the emissive layer for the reason described above.

The phenanthroline derivative has a high electron mobility, and further has a property that electrons are easily injected from the cathode. For this reason, by using the phenanthroline derivative as the electron transporting layer, a significant voltage reduction and a high efficiency can be achieved. When the phenanthroline derivative is a phenanthroline multimer, the above-described effect is further improved, which is more preferable. Preferred examples of the phenanthroline derivative include a compound represented by general formula (16).

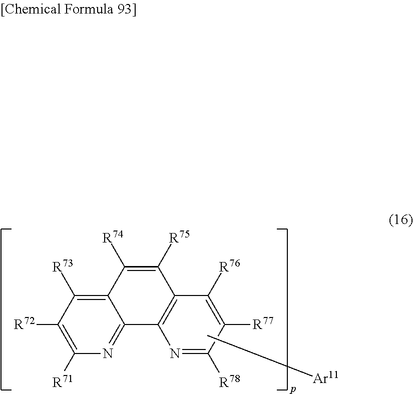

R⁷¹ to R⁷⁸ are the same or different from each other, and each are a hydrogen atom, a substituted or unsubstituted aryl group, or a substituted or unsubstituted heteroaryl group. Ar¹¹ is a substituted or unsubstituted aryl group. The symbol p is a natural number of 1 to 3. When the electron transporting layer is formed of a plurality of layers, the phenanthroline derivative is preferably used in the cathode or the layer which is in contact with the electron injection layer for the reason described above.

Preferable electron transporting materials are not particularly limited, but specific examples thereof include the following.

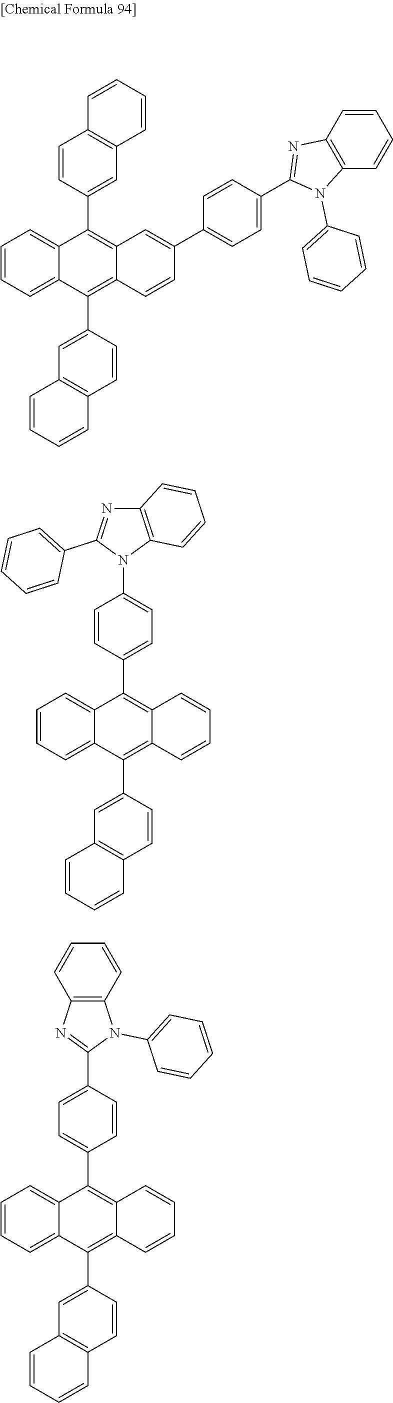

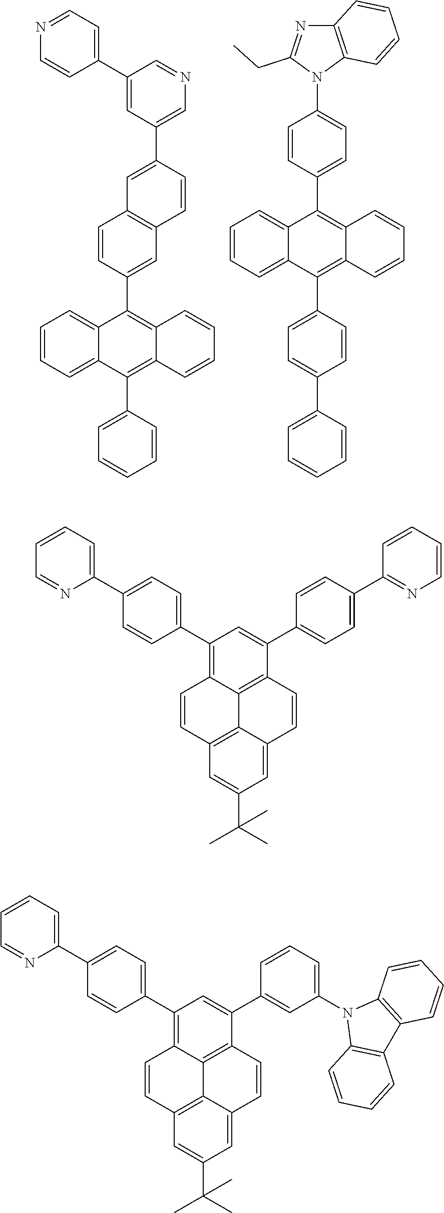 [SMILES: c1ccc(-c2c3ccccc3c(-c3ccc4cc(-c5cncc(-c6ccncc6)c5)ccc4c3)c3ccccc23)cc1 ; CCc1nc2ccccc2n1-c1ccc(-c2c3ccccc3c(-c3ccc(-c4ccccc4)cc3)c3ccccc23)cc1 ; CC(C)(C)c1cc2ccc3c(-c4ccc(-c5ccccn5)cc4)cc(-c4ccc(-c5ccccn5)cc4)c4ccc(c1)c2c34 ; CC(C)(C)c1cc2ccc3c(-c4ccc(-c5ccccn5)cc4)cc(-c4cccc(-n5c6ccccc6c6ccccc65)c4)c4ccc(c1)c2c34]

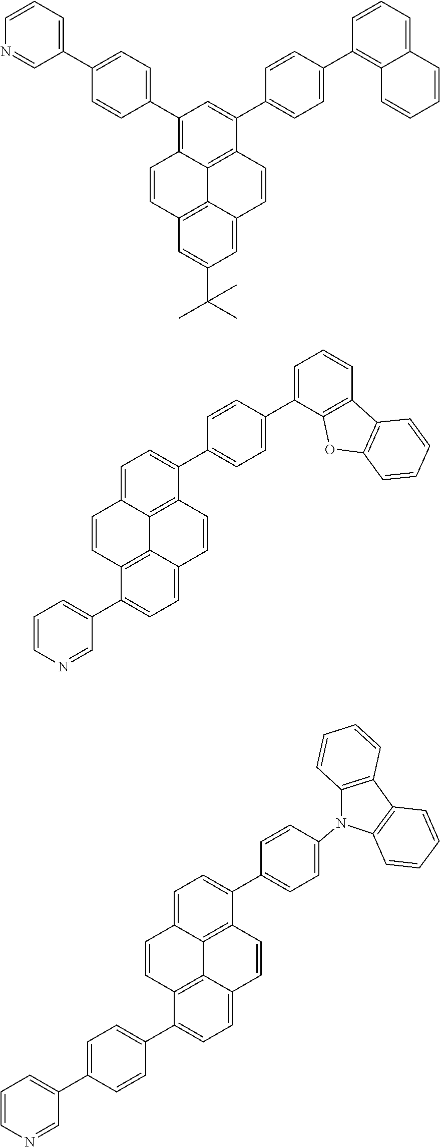 [SMILES: CC(C)(C)c1cc2ccc3c(-c4ccc(-c5cccnc5)cc4)cc(-c4ccc(-c5cccc6ccccc56)cc4)c4ccc(c1)c2c34 ; c1cncc(-c2ccc3ccc4c(-c5ccc(-c6cccc7c6oc6ccccc67)cc5)ccc5ccc2c3c54)c1 ; c1cncc(-c2ccc(-c3ccc4ccc5c(-c6ccc(-n7c8ccccc8c8ccccc87)cc6)ccc6ccc3c4c65)cc2)c1]

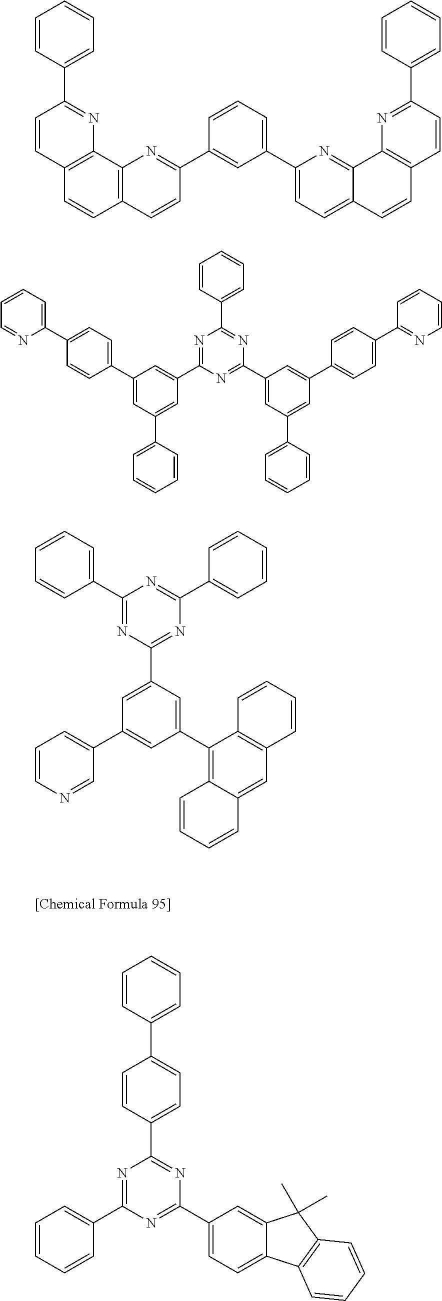 [SMILES: c1ccc(-c2ccc3ccc4ccc(-c5cccc(-c6ccc7ccc8ccc(-c9ccccc9)nc8c7n6)c5)nc4c3n2)cc1 ; c1ccc(-c2cc(-c3ccc(-c4ccccn4)cc3)cc(-c3nc(-c4ccccc4)nc(-c4cc(-c5ccccc5)cc(-c5ccc(-c6ccccn6)cc5)c4)n3)c2)cc1 ; c1ccc(-c2nc(-c3ccccc3)nc(-c3cc(-c4cccnc4)cc(-c4c5ccccc5cc5ccccc45)c3)n2)cc1 ; CC1(C)c2ccccc2-c2ccc(-c3nc(-c4ccccc4)nc(-c4ccc(-c5ccccc5)cc4)n3)cc21]

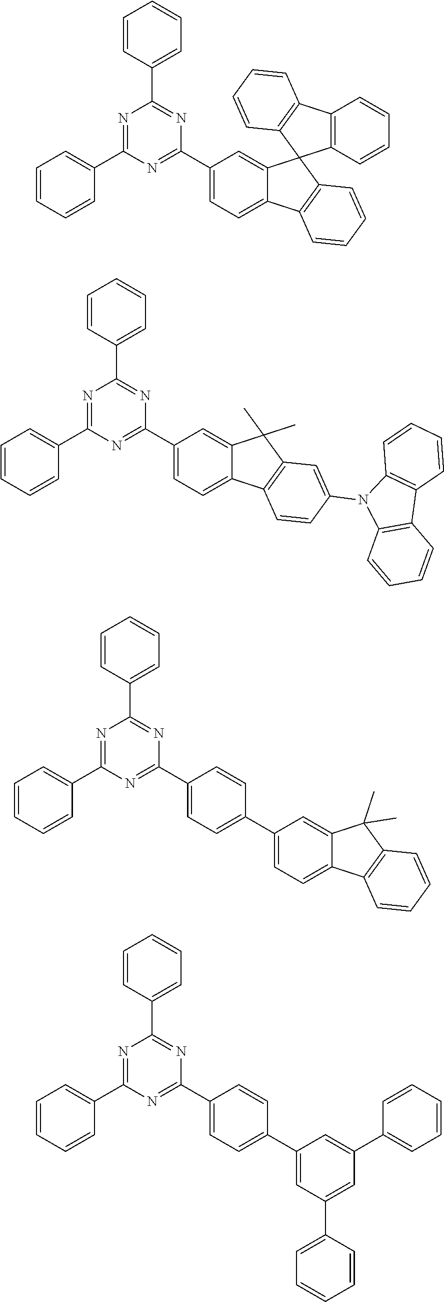 [SMILES: c1ccc(-c2nc(-c3ccccc3)nc(-c3ccc4c(c3)C3(c5ccccc5-c5ccccc53)c3ccccc3-4)n2)cc1 ; CC1(C)c2cc(-c3nc(-c4ccccc4)nc(-c4ccccc4)n3)ccc2-c2ccc(-n3c4ccccc4c4ccccc43)cc21 ; CC1(C)c2ccccc2-c2ccc(-c3ccc(-c4nc(-c5ccccc5)nc(-c5ccccc5)n4)cc3)cc21 ; c1ccc(-c2cc(-c3ccccc3)cc(-c3ccc(-c4nc(-c5ccccc5)nc(-c5ccccc5)n4)cc3)c2)cc1]

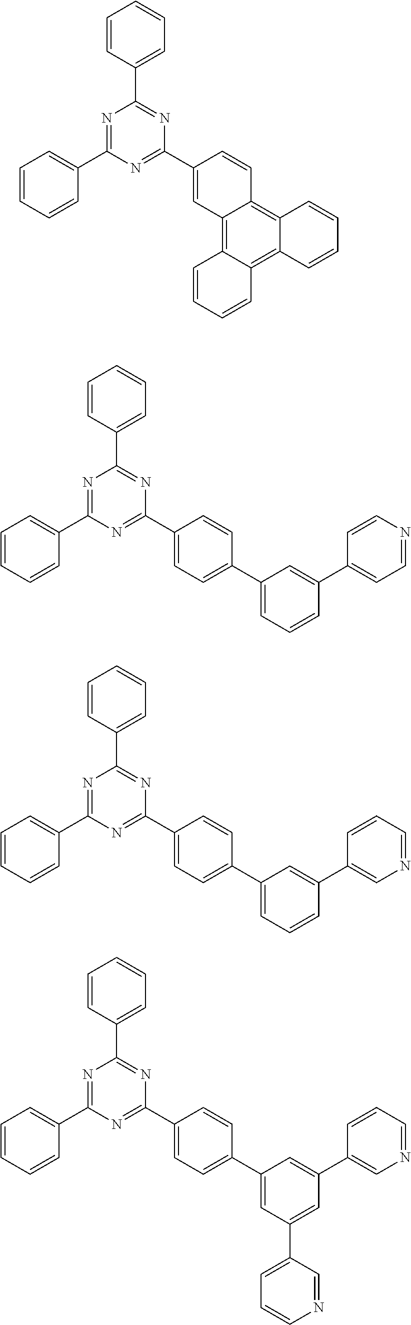 [SMILES: c1ccc(-c2nc(-c3ccccc3)nc(-c3ccc4c5ccccc5c5ccccc5c4c3)n2)cc1 ; c1ccc(-c2nc(-c3ccccc3)nc(-c3ccc(-c4cccc(-c5ccncc5)c4)cc3)n2)cc1 ; c1ccc(-c2nc(-c3ccccc3)nc(-c3ccc(-c4cccc(-c5cccnc5)c4)cc3)n2)cc1 ; c1ccc(-c2nc(-c3ccccc3)nc(-c3ccc(-c4cc(-c5cccnc5)cc(-c5cccnc5)c4)cc3)n2)cc1]

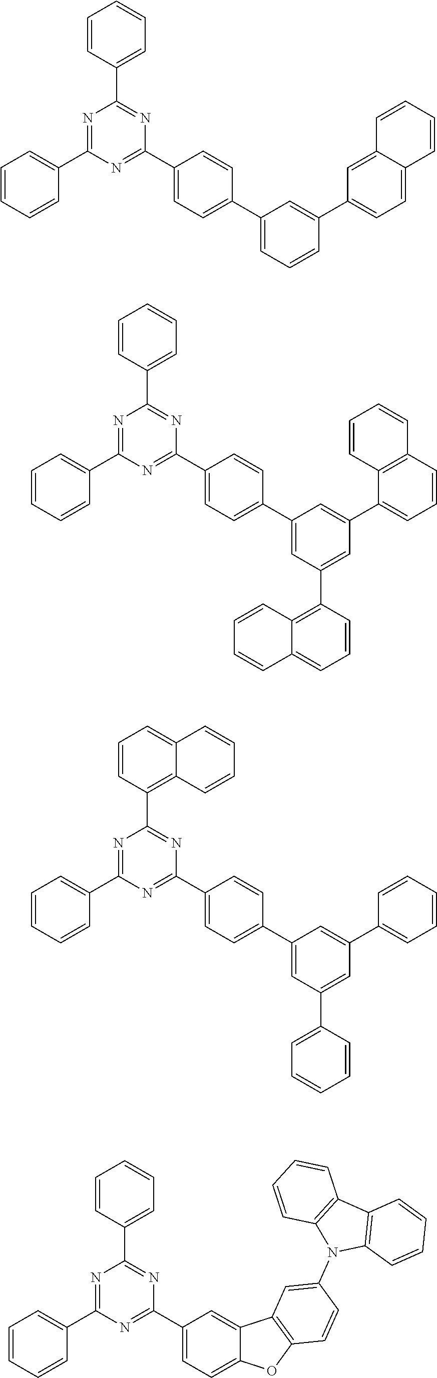 [SMILES: c1ccc(-c2nc(-c3ccccc3)nc(-c3ccc(-c4cccc(-c5ccc6ccccc6c5)c4)cc3)n2)cc1 ; c1ccc(-c2nc(-c3ccccc3)nc(-c3ccc(-c4cc(-c5cccc6ccccc56)cc(-c5cccc6ccccc56)c4)cc3)n2)cc1 ; c1ccc(-c2cc(-c3ccccc3)cc(-c3ccc(-c4nc(-c5ccccc5)nc(-c5cccc6ccccc56)n4)cc3)c2)cc1 ; c1ccc(-c2nc(-c3ccccc3)nc(-c3ccc4oc5ccc(-n6c7ccccc7c7ccccc76)cc5c4c3)n2)cc1]

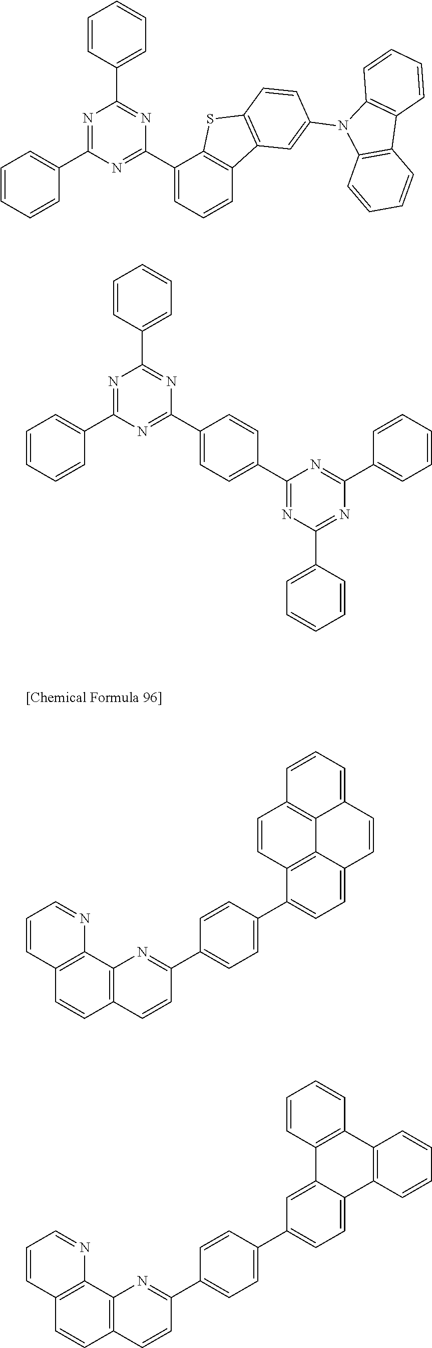 [SMILES: c1ccc(-c2nc(-c3ccccc3)nc(-c3cccc4c3sc3ccc(-n5c6ccccc6c6ccccc65)cc34)n2)cc1 ; c1ccc(-c2nc(-c3ccccc3)nc(-c3ccc(-c4nc(-c5ccccc5)nc(-c5ccccc5)n4)cc3)n2)cc1 ; c1cnc2c(c1)ccc1ccc(-c3ccc(-c4ccc5ccc6cccc7ccc4c5c67)cc3)nc12 ; c1cnc2c(c1)ccc1ccc(-c3ccc(-c4ccc5c6ccccc6c6ccccc6c5c4)cc3)nc12]

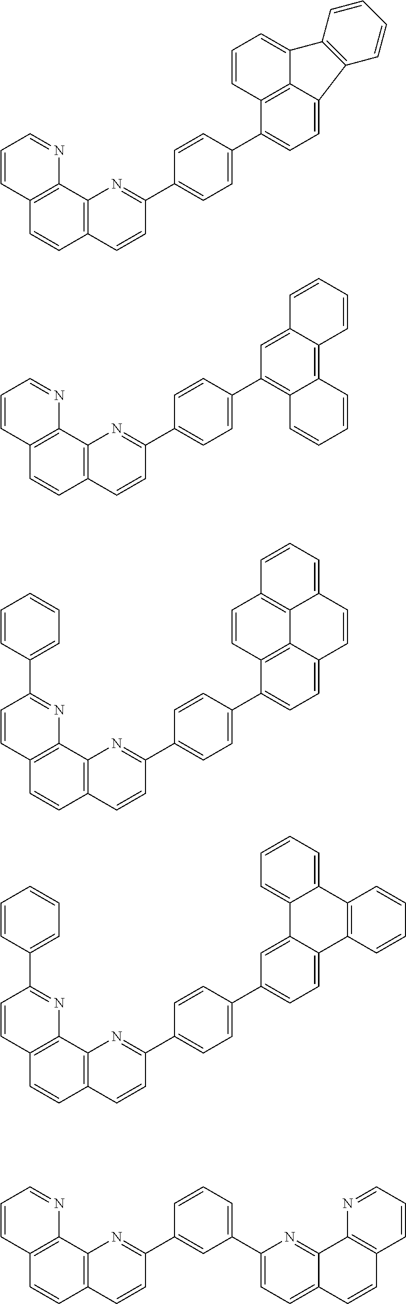 [SMILES: c1ccc2c(c1)-c1cccc3c(-c4ccc(-c5ccc6ccc7cccnc7c6n5)cc4)ccc-2c13 ; c1ccc2c(c1)cc(-c1ccc(-c3ccc4ccc5cccnc5c4n3)cc1)c1ccccc12 ; c1ccc(-c2ccc3ccc4ccc(-c5ccc(-c6ccc7ccc8cccc9ccc6c7c89)cc5)nc4c3n2)cc1 ; c1ccc(-c2ccc3ccc4ccc(-c5ccc(-c6ccc7c8ccccc8c8ccccc8c7c6)cc5)nc4c3n2)cc1 ; c1cc(-c2ccc3ccc4cccnc4c3n2)cc(-c2ccc3ccc4cccnc4c3n2)c1]

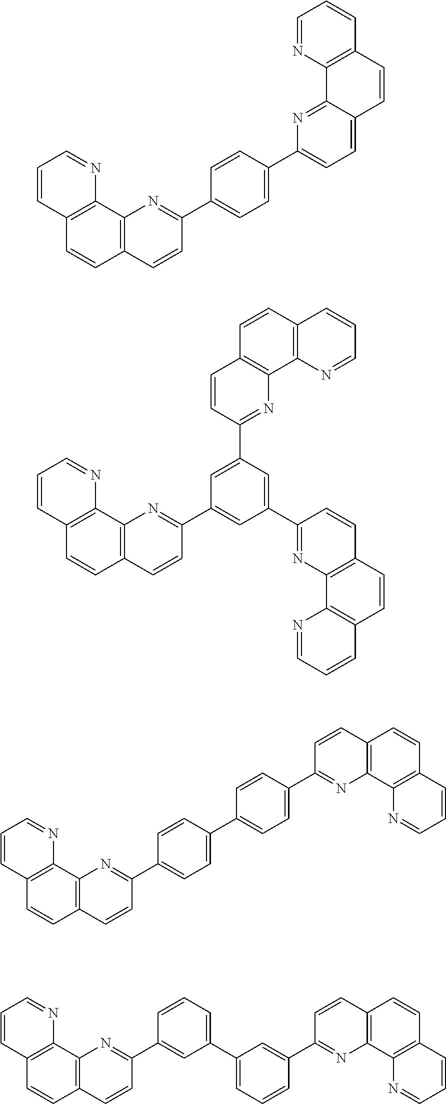 [SMILES: c1cnc2c(c1)ccc1ccc(-c3ccc(-c4ccc5ccc6cccnc6c5n4)cc3)nc12 ; c1cnc2c(c1)ccc1ccc(-c3cc(-c4ccc5ccc6cccnc6c5n4)cc(-c4ccc5ccc6cccnc6c5n4)c3)nc12 ; c1cnc2c(c1)ccc1ccc(-c3ccc(-c4ccc(-c5ccc6ccc7cccnc7c6n5)cc4)cc3)nc12 ; c1cc(-c2cccc(-c3ccc4ccc5cccnc5c4n3)c2)cc(-c2ccc3ccc4cccnc4c3n2)c1]

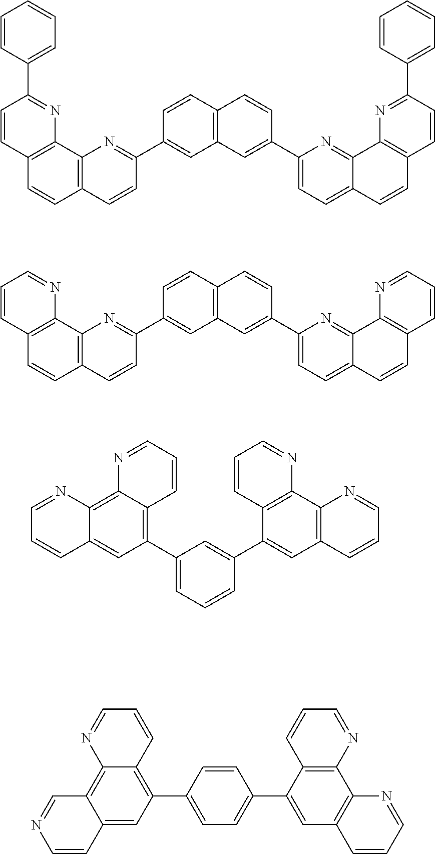 [SMILES: c1ccc(-c2ccc3ccc4ccc(-c5ccc6ccc(-c7ccc8ccc9ccc(-c%10ccccc%10)nc9c8n7)cc6c5)nc4c3n2)cc1 ; c1cnc2c(c1)ccc1ccc(-c3ccc4ccc(-c5ccc6ccc7cccnc7c6n5)cc4c3)nc12 ; c1cc(-c2cc3cccnc3c3ncccc23)cc(-c2cc3cccnc3c3ncccc23)c1 ; c1cnc2c(c1)cc(-c1ccc(-c3cc4ccncc4c4ncccc34)cc1)c1cccnc12]

Besides these electron transporting materials, those disclosed in WO 2004/63159, WO 2003/60956, Appl. Phys. Lett. 74, 865 (1999), Org. Electron. 4, 113 (2003), WO 2010/113743, WO 2010/1817 and WO 2016/121597 and the like can also be used.

While the electron transporting material may be used alone, two or more kinds of the electron transporting materials may be used in combination, or one or more kinds of other electron transporting materials may be used in a combination with the electron transporting material. The electron transporting layer may further contain a donor material. The donor material is a compound which makes easy electron injection into the electron transporting layer from the cathode or the electron injection layer and, moreover, improves the electric conductivity of the electron transporting layer, by improving an electron injection barrier.

Preferred examples of the donor material include an alkali metal, an inorganic salt containing an alkali metal, a complex of an alkali metal and an organic substance, an alkaline earth metal, an inorganic salt containing an alkaline earth metal, or a complex of an alkaline earth metal and an organic substance. Examples of the preferable kind of the alkali metal and the alkaline earth metal include alkali metals such as lithium, sodium and cesium, and alkaline earth metals such as magnesium and calcium which have a low work function and have a great effect of improving electron transporting ability.

(Electron Injection Layer)

In the light-emitting element according to the embodiment of the present invention, an electron injection layer may be provided between a cathode and an electron transporting layer. Generally, the electron injection layer is inserted for the purpose of helping injection of electrons from the cathode into the electron transporting layer. For the electron injection layer, a compound having a heteroaryl ring structure containing electron-accepting nitrogen may be used, or a layer containing the above-mentioned donor material may be used.

An inorganic substance such as an insulator or a semiconductor can also be used for the electron injection layer. The use of such a material can effectively prevent a short-circuit of the light-emitting element, and improve electron injection property.

It is preferred that at least one metal compound selected from the group consisting of an alkali metal chalcogenide, an alkaline earth metal chalcogenide, a halide of an alkali metal and a halide of an alkaline earth metal is used as the insulator.

Specifically, examples of the preferable alkali metal chalcogenide include Li₂O, Na₂S, and Na₂Se, and examples of the preferable alkaline earth metal chalcogenide include CaO, BaO, SrO, BeO, BaS, and CaSe. Examples of the preferable halide of an alkali metal include LiF, NaF, KF, LiCl, KCl, and NaCl. Examples of the preferable halide of an alkaline earth metal include fluorides such as CaF₂, BaF₂, SrF₂, MgF₂, and BeF₂, and halides other than fluorides.

For the electron injection layer, a complex of an organic substance and a metal is also suitably used from the viewpoint of easy film thickness adjustment. In the above-mentioned organic metal complex, preferred examples of the organic substance include quinolinol, benzoquinolinol, pyridylphenol, flavonol, hydroxyimidazopyridine, hydroxybenzazole, and hydroxytriazole. In the organometallic complex, a complex of an alkali metal and an organic substance is preferred, and a complex of lithium and an organic substance is more preferred.

(Charge Generation Layer)

In the light-emitting element according to the embodiment of the present invention, the charge generation layer is an intermediate layer existing between an anode and a cathode in the tandem structure-type element, and in the charge generation layer, holes and electrons are generated by charge separation. Generally, the charge generation layer is formed from a P-type layer on the cathode side and an N-type layer on the anode side. These layers are desired to perform efficient charge separation, and efficiently transport generated carriers.

For the P-type charge generation layer, materials to be used for the above-mentioned hole injection layer and hole transporting layer can be used. For example, benzidine derivatives such as HAT-CN6, NPD and TBDB; materials called starburst arylamine such as m-MTDATA and 1-TNATA; and materials having skeletons represented by general formulae (12) and (13) and the like can be suitably used.

For the N-type charge generation layer, materials to be used for the above-mentioned electron injection layer and electron transporting layer can be used, and a compound having a heteroaryl ring structure containing electron-accepting nitrogen may be used, or a layer containing the above-mentioned donor material may be used.

Examples of a method for forming each of the aforementioned layers constituting the light-emitting element include, but are not particularly limited to, resistance heating deposition, electron beam deposition, sputtering, a molecular lamination method, and a coating method, but usually, resistance heating deposition or electron beam deposition is preferred from the viewpoint of element property.

The light-emitting element according to the embodiment of the present invention has a function of being able to convert electrical energy into light. Herein, a direct current is mainly used as the electrical energy, but a pulse current or an alternate current can also be used. A current value and a voltage value are not particularly limited, but when the power consumed and life of the element are considered, they should be selected so that the maximum luminance is obtained by energy as low as possible.

The light-emitting element according to the embodiment of the present invention is suitably used for a display. Specifically, for example, the light-emitting element is suitably used as a display which displays in a matrix and/or segment system.

In the matrix system, pixels for display are arranged two-dimensionally such as lattice-like arrangement or mosaic-like arrangement, and the collection of pixels displays letters and images. The shape and size of the pixel are determined depending on applications. For example, for displaying images and letters on personal computers, monitors and televisions, a square pixel being 300 μm or less at each side is usually used and, in the case of a large display such as a display panel, a pixel being millimeter order at each side is used. In the case of a monochromatic display, pixels having the same color may be arranged, and in the case of a color display, pixels having red, green and blue colors are arranged to perform display. In this case, typically, there are a delta type and a stripe type. A method for driving this matrix may be either a passive matrix driving method or an active matrix. The passive matrix driving has a simple structure, but when operation property is considered, the active matrix is more excellent in some cases, and it is necessary to use them properly depending on applications.

The segment system is a system by which a pattern is formed so as to display predetermined information, and a region determined by arrangement of this pattern is made to emit light. Examples thereof include time and temperature displays in digital watches and thermometers, operating-state displays in audio equipment, IH cookers and the like, and panel displays of automobiles. The above-mentioned matrix display and segment display may exist together in the same panel.

The light-emitting element according to the embodiment of the present invention can also be preferably used as a backlight of various displays. Examples of the display include liquid crystal displays, display parts in watches or audio devices, automobile panels, display boards, and marks. In particular, the light-emitting element of the present invention is preferably used in a backlight for liquid crystal displays, particularly televisions, tablets, smartphones, and personal computers, and the like whose a thickness reduction is being studied. Thereby, the backlight which is thinner and lighter than the conventional one can be provided.

The light-emitting element according to the embodiment of the present invention is also preferably used as various illuminators. The light-emitting element according to the embodiment of the present invention can achieve both a high luminous efficiency and high color purity, and further can be reduced in thickness and weight, whereby an illuminator having low power consumption, a bright emitted color and high design properties can be achieved.

The light-emitting element according to the embodiment of the present invention is also preferably used for a sensor. In particular, the light-emitting element of the present invention is preferably used for wearable sensors which require low power consumption and a reduction in size and weight, and a small sensor can be provided, which can visualize changes due to stimulations such as heat, pressure, and light, and chemical reactions in bright colors.

### EXAMPLES

Hereinafter, the present invention will be described by way of Examples, but the present invention is not limited thereto. Compounds used were synthesized using known methods except for commercially available compounds.

In Examples below, compounds B-1 to B-5 and D-1 to D-5 are compounds shown below.

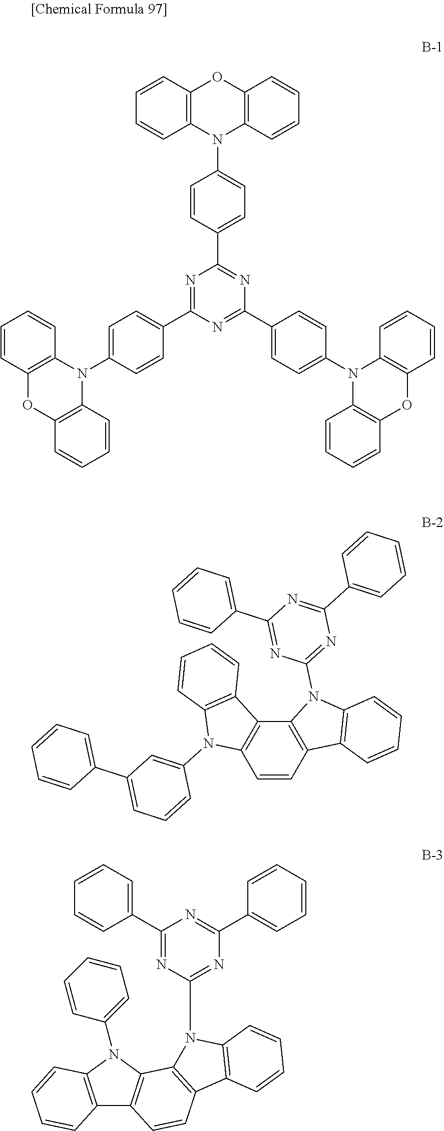 [SMILES: c1ccc2c(c1)Oc1ccccc1N2c1ccc(-c2nc(-c3ccc(N4c5ccccc5Oc5ccccc54)cc3)nc(-c3ccc(N4c5ccccc5Oc5ccccc54)cc3)n2)cc1 ; c1ccc(-c2cccc(-n3c4ccccc4c4c3ccc3c5ccccc5n(-c5nc(-c6ccccc6)nc(-c6ccccc6)n5)c34)c2)cc1 ; c1ccc(-c2nc(-c3ccccc3)nc(-n3c4ccccc4c4ccc5c6ccccc6n(-c6ccccc6)c5c43)n2)cc1]

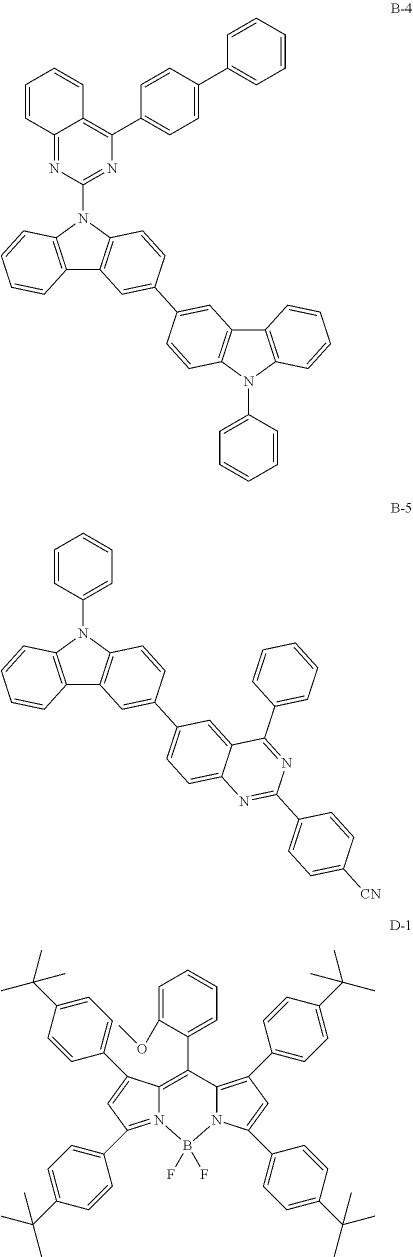

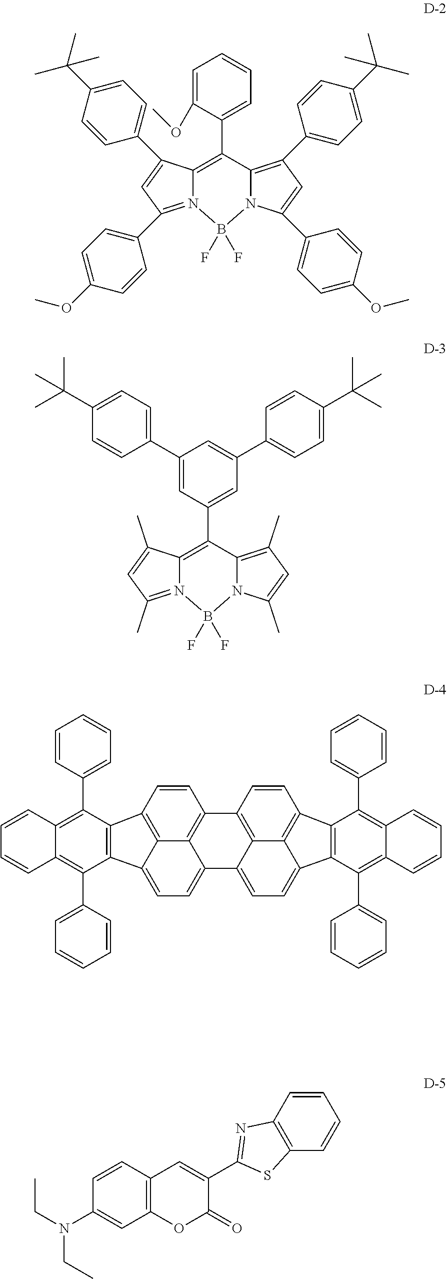

λ1 (abs) and λ2 (FL) were determined by measuring an absorption spectrum and a fluorescence spectrum according to the following method.

<Measurement of Absorption Spectrum>

Absorption spectra of the compounds were measured with the compounds dissolved in 2-methyltetrahydrofuran at a concentration of 1×10⁻⁶ mol/L using U-3200 type spectrophotometer (manufactured by Hitachi, Ltd.).

<Measurement of Fluorescence Spectrum>

For fluorescence spectra of the compounds, fluorescence spectra when the compounds were dissolved in 2-methyltetrahydrofuran at a concentration of 1×10⁻⁶ mol/L and were excited at a wavelength of 350 nm were measured using F-2500 type fluorescence spectrophotometer (manufactured by Hitachi, Ltd.).

### Example 1

A glass substrate with an Ag_{0.98}Pd_{0.01}Cu_{0.01} alloy deposited thereon in a thickness of 100 nm and an ITO transparent electroconductive film deposited thereon in a thickness of 10 nm (manufactured by GEOMATEC Co., Ltd., 11 Ω/□, sputtered product) was cut into 38×46 mm, and etched. The resulting substrate was ultrasonically washed with “SEMICOCLEAN 56” (trade name, manufactured by Furuuchi Chemical Corporation) for 15 minutes, and then washed with ultrapure water. This substrate was treated with UV-ozone for 1 hour immediately before preparation of an element, and placed in a vacuum deposition apparatus. The air was evacuated until the degree of vacuum in the apparatus was 5×10⁻⁴ Pa or lower. By a resistance heating method, first, HAT-CN6 was deposited as a hole injection layer in a thickness of 10 nm, and HT-1 was deposited as a hole transporting layer in a thickness of 180 nm. Next, as an emissive layer, a host material H-1, a compound D-1 represented by general formula (1), and a compound B-1 represented by general formula (2) were deposited in a thickness of 40 nm so that the weight ratio was 80:1:20. Further, as an electron transporting layer, a compound ET-1 used as an electron transporting material and 2E-1 used as a donor material were laminated in a thickness of 35 nm so that the deposition rate ratio of the compound ET-1 and 2E-1 was 1:1. Next, lithium fluoride was deposited in a thickness of 0.5 nm, and magnesium and silver were then co-deposited in a thickness of 15 nm to form a cathode, so that a 5×5 mm square top emission element was prepared. This light-emitting element exhibited high color purity light emission having a light emission peak wavelength of 625 nm and a half-value width of 46 nm. The external quantum efficiency of this light-emitting element when it was made to emit light at luminance of 1000 cd/m² was 5.0%. The results are shown in Table 2. HAT-CN6, HT-1, ET-1, and 2E-1 are compounds shown below.

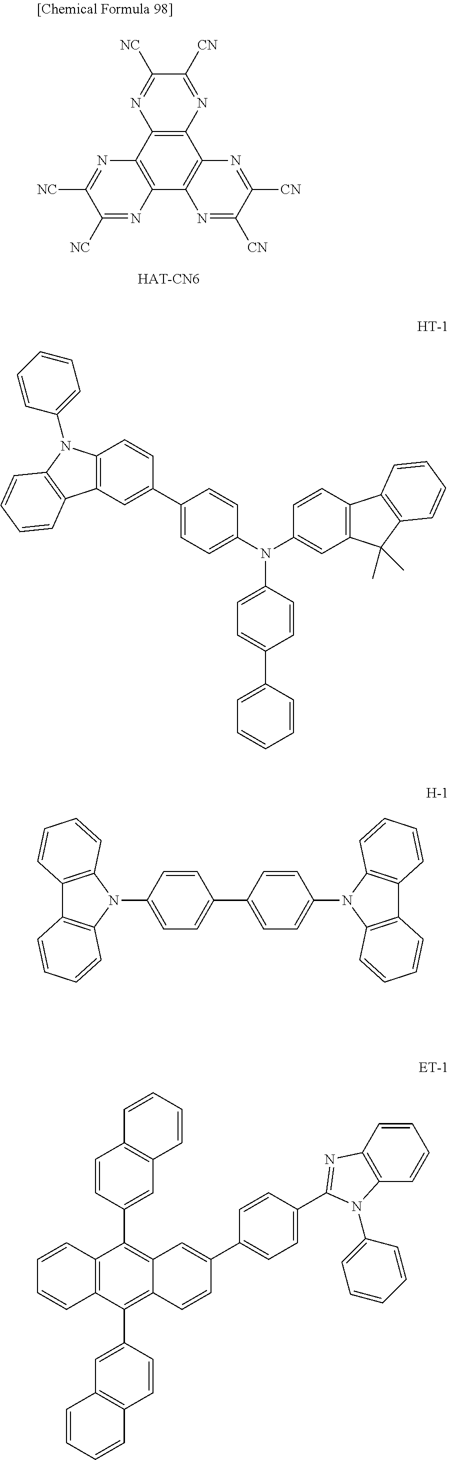 [SMILES: N#Cc1nc2c3nc(C#N)c(C#N)nc3c3nc(C#N)c(C#N)nc3c2nc1C#N ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc2c(c1)c1ccccc1n2-c1ccc(-c2ccc(-n3c4ccccc4c4ccccc43)cc2)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

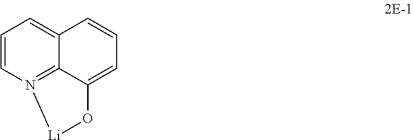

### Examples 2 to 20 and Comparative Examples 1 to 6

In the same manner as in Example 1 except that compounds described in Tables 2 and 3 were used as materials for emissive layers, light-emitting elements were prepared and evaluated. The results are shown in Tables 2 and 3. H-2 to H-10, D-6 and D-7 are compounds shown below.

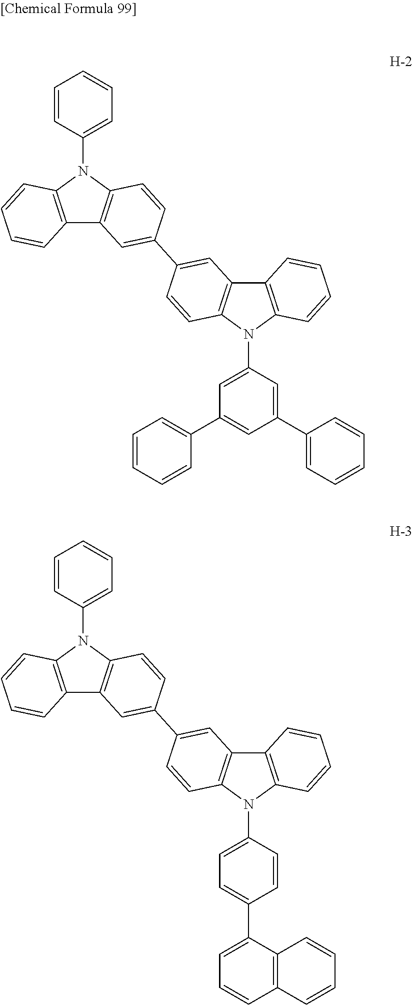 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4ccc(-c5cccc6ccccc56)cc4)ccc32)cc1]

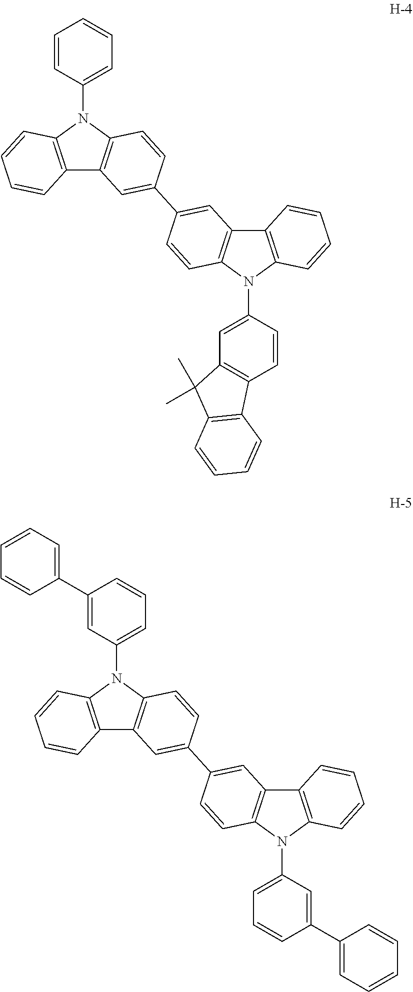 [SMILES: CC1(C)c2ccccc2-c2ccc(-n3c4ccccc4c4cc(-c5ccc6c(c5)c5ccccc5n6-c5ccccc5)ccc43)cc21 ; c1ccc(-c2cccc(-n3c4ccccc4c4cc(-c5ccc6c(c5)c5ccccc5n6-c5cccc(-c6ccccc6)c5)ccc43)c2)cc1]

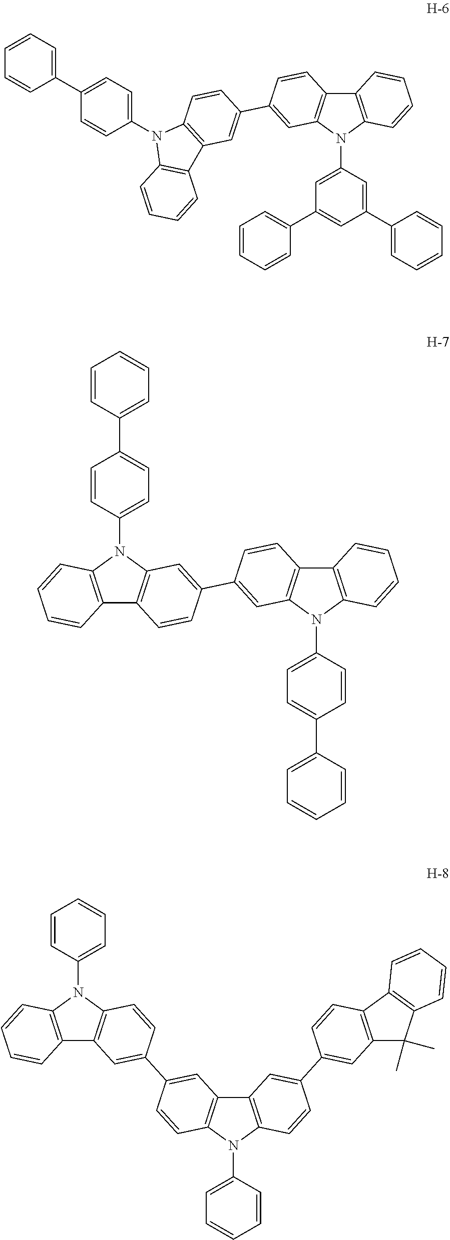 [SMILES: c1ccc(-c2ccc(-n3c4ccccc4c4cc(-c5ccc6c7ccccc7n(-c7cc(-c8ccccc8)cc(-c8ccccc8)c7)c6c5)ccc43)cc2)cc1 ; c1ccc(-c2ccc(-n3c4ccccc4c4ccc(-c5ccc6c7ccccc7n(-c7ccc(-c8ccccc8)cc7)c6c5)cc43)cc2)cc1 ; CC1(C)c2ccccc2-c2ccc(-c3ccc4c(c3)c3cc(-c5ccc6c(c5)c5ccccc5n6-c5ccccc5)ccc3n4-c3ccccc3)cc21]

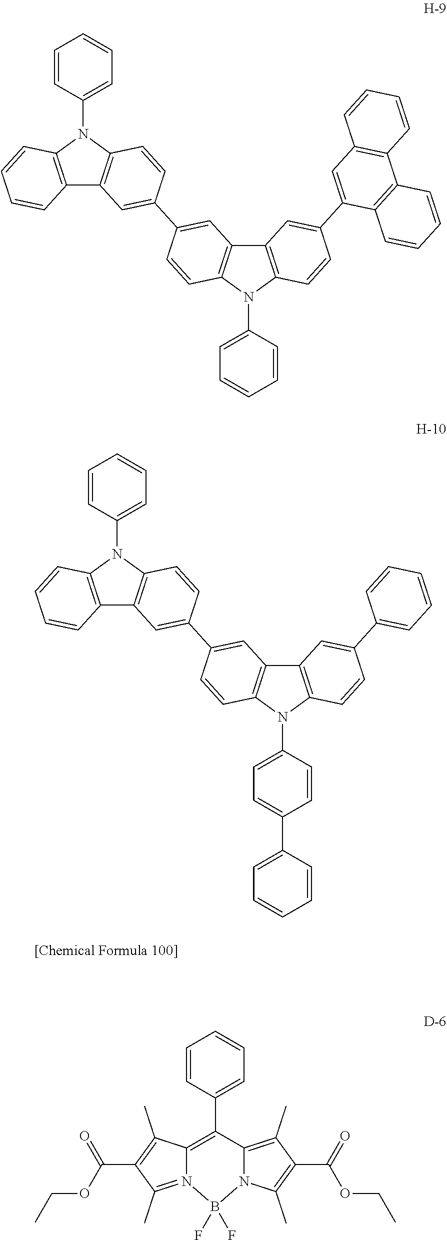

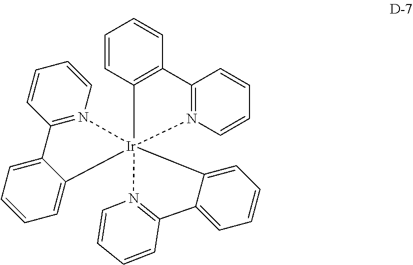

### Example 21

A glass substrate with an ITO transparent electroconductive film deposited thereon in a thickness of 165 nm (manufactured by GEOMATEC Co., Ltd., 11 Ω/□, sputtered product) was cut into 38×46 mm, and etched. The resulting substrate was ultrasonically washed with “SEMICOCLEAN 56” (trade name, manufactured by Furuuchi Chemical Corporation) for 15 minutes, and then washed with ultrapure water. This substrate was treated with UV-ozone for 1 hour immediately before preparation of an element, and placed in a vacuum deposition apparatus, and the air was evacuated until the degree of vacuum in the apparatus was 5×10⁻⁴ Pa or lower. By a resistance heating method, first, HAT-CN6 was deposited as a hole injection layer in a thickness of 10 nm, and HT-1 was deposited as a hole transporting layer in a thickness of 180 nm. Next, as an emissive layer, a host material H-1, a compound D-3 represented by general formula (1), and a compound B-1 represented by general formula (2) were deposited in a thickness of 40 nm so that the weight ratio was 80:1:20. Further, as an electron transporting layer, a compound ET-1 used as an electron transporting material and 2E-1 used as a donor material were laminated in a thickness of 35 nm so that the deposition rate ratio of the compound ET-1 and 2E-1 was 1:1. Next, lithium fluoride was deposited in a thickness of 0.5 nm, and aluminum was then deposited in a thickness of 1000 nm to form a cathode, so that a 5×5 mm square bottom emission element was prepared. This light-emitting element exhibited high color purity light emission having a light emission peak wavelength of 519 nm and a half-value width of 30 nm. The external quantum efficiency of this light-emitting element when it was made to emit light at luminance of 1000 cd/m² was 4.4%. The results are shown in Table 2.

### Comparative Example 7

In the same manner as in Example 21 except that a compound described in Table 2 was used as a material for an emissive layer, a light-emitting element was prepared and evaluated. The results are shown in Table 2.

TABLE 2

| | Emissive Layer | | | | | | | | | |
|---|---|---|---|---|---|---|---|---|---|---|
| | *Numbers in parentheses below | | | | | | | | | |
| | compound names: weight ratios | | | | Light | | | | | |
| | | General | | | emission | Half- | | | | External |
| | | formula (1) | General | | peak | value | | | \|λ1 | quantum |
| | | or other | formula | Emitted | wavelength | width | λ1 | λ2 | (abs) − | efficiency |
| | Host | dopants | (2) | color | (nm) | (nm) | (abs) | (FL) | λ2 (FL)\| | (%) |
| Example 1 | H-1 | D-1 | B-1 | Red | 625 | 46 | 577 | 563 | 14 | 5.0 |
| | (80) | (1) | (20) | | | | | | | |
| Example 2 | H-1 | D-1 | B-2 | Red | 625 | 46 | 577 | 519 | 58 | 3.5 |
| | (80) | (1) | (20) | | | | | | | |
| Example 3 | H-1 | D-1 | B-4 | Red | 625 | 46 | 577 | 522 | 55 | 3.0 |
| | (80) | (1) | (20) | | | | | | | |
| Example 4 | H-1 | D-2 | B-1 | Red | 635 | 48 | 590 | 563 | 27 | 4.8 |
| | (80) | (1) | (20) | | | | | | | |
| Example 5 | H-1 | D-2 | B-4 | Red | 635 | 48 | 590 | 522 | 68 | 2.7 |
| | (80) | (1) | (20) | | | | | | | |
| Example 6 | H-2 | D-1 | B-1 | Red | 625 | 46 | 577 | 563 | 14 | 5.2 |
| | (80) | (1) | (20) | | | | | | | |
| Example 7 | H-1 | D-3 | B-2 | Green | 519 | 27 | 504 | 519 | 15 | 4.4 |
| | (80) | (1) | (20) | | | | | | | |
| Example 8 | H-1 | D-3 | B-3 | Green | 519 | 27 | 504 | 518 | 14 | 4.2 |
| | (80) | (1) | (20) | | | | | | | |
| Example 9 | H-1 | D-3 | B-5 | Green | 519 | 27 | 504 | 454 | 50 | 3.6 |
| | (80) | (1) | (20) | | | | | | | |
| Example 10 | H-2 | D-3 | B-2 | Green | 519 | 27 | 504 | 519 | 15 | 4.7 |
| | (80) | (1) | (20) | | | | | | | |
| Comparative | H-1 | D-1 | — | Red | 625 | 46 | 577 | — | — | 2.1 |
| Example 1 | (100) | (1) | | | | | | | | |
| Comparative | H-1 | D-4 | B-1 | Red | 593, 644 | 19, 28 | 587 | 563 | 24 | 2.1 |
| Example 2 | (80) | (1) | (20) | | | | | | | |
| Comparative | H-1 | D-3 | — | Green | 519 | 27 | 504 | — | — | 2.0 |
| Example 3 | (80) | (1) | | | | | | | | |
| Comparative | H-1 | D-5 | B-1 | Green | 490 | 70 | 478 | 563 | 85 | 1.7 |
| Example 4 | (80) | (1) | (20) | | | | | | | |
| Comparative | H-1 | D-5 | B-2 | Green | 490 | 70 | 478 | 519 | 41 | 2.0 |
| Example 5 | (80) | (1) | (20) | | | | | | | |
| Comparative | H-1 | D-7 | — | Green | 525 | 42 | 376 | — | — | 5.4 |
| Example 6 | (80) | (5) | | | | | | | | |
| Comparative | H-1 | D-7 | — | Green | 520 | 75 | 376 | — | — | 8.4 |
| Example 7 | (80) | (5) | | | | | | | | |

TABLE 3

| | Emissive Layer | | | | | | | | | |
|---|---|---|---|---|---|---|---|---|---|---|
| | *Numbers in parentheses below | | | | | | | | | |
| | compound names: weight ratios | | | | Light | | | | | |
| | | General | | | emission | Half- | | | | External |
| | | formula (1) | General | | peak | value | | | \|λ1 | quantum |
| | | or other | formula | Emitted | wavelength | width | λ1 | λ2 | (abs) − | efficiency |
| | Host | dopants | (2) | color | (nm) | (nm) | (abs) | (FL) | λ2 (FL)\| | (%) |
| Example 11 | H-3 | D-2 | B-1 | Red | 635 | 48 | 590 | 563 | 27 | 5.1 |
| | (80) | (1) | (20) | | | | | | | |
| Example 12 | H-7 | D-2 | B-1 | Red | 635 | 48 | 590 | 563 | 27 | 5.1 |
| | (80) | (1) | (20) | | | | | | | |
| Example 13 | H-8 | D-2 | B-1 | Red | 635 | 48 | 590 | 563 | 27 | 5.2 |
| | (80) | (1) | (20) | | | | | | | |
| Example 14 | H-9 | D-2 | B-1 | Red | 635 | 48 | 590 | 563 | 27 | 5.1 |
| | (80) | (1) | (20) | | | | | | | |
| Example 15 | H-4 | D-3 | B-2 | Green | 519 | 27 | 504 | 519 | 15 | 4.9 |
| | (80) | (1) | (20) | | | | | | | |
| Example 16 | H-5 | D-3 | B-2 | Green | 519 | 27 | 504 | 519 | 15 | 4.6 |
| | (80) | (1) | (20) | | | | | | | |
| Example 17 | H-6 | D-3 | B-2 | Green | 519 | 27 | 504 | 519 | 15 | 4.6 |
| | (80) | (1) | (20) | | | | | | | |
| Example 18 | H-7 | D-3 | B-2 | Green | 519 | 27 | 504 | 519 | 15 | 4.5 |
| | (80) | (1) | (20) | | | | | | | |
| Example 19 | H-10 | D-3 | B-2 | Green | 519 | 27 | 504 | 519 | 15 | 4.7 |
| | (80) | (1) | (20) | | | | | | | |
| Example 20 | H-10 | D-6 | B-2 | Green | 520 | 27 | 505 | 519 | 16 | 4.9 |
| | (80) | (1) | (20) | | | | | | | |
| Example 21 | H-1 | D-3 | B-2 | Green | 519 | 30 | 504 | 519 | 15 | 4.4 |
| | (80) | (1) | (20) | | | | | | | |

Examples 1 to 3 achieved a higher external quantum efficiency than that of Comparative Example 1 not containing the compound represented by general formula (2). Among these, Example 1 satisfying numerical expression (i-1) achieved a higher external quantum efficiency than that of each of Examples 2 and 3 not satisfying numerical expression (i-1).

Examples 1 to 3 achieved a higher external quantum efficiency than that of Comparative Example 2 using the compound D-4 other than the compound represented by general formula (1) as a dopant. Further, Comparative Example 2 showed two light emission peaks, and resulted in poorer color purity than that of each of Examples 1 to 3 showing a single peak.

Examples 4 and 5 using D-2 as the compound represented by general formula (1) achieved a higher external quantum efficiency than that of Comparative Example 1. Among these, Example 4 satisfying numerical expression (i-1) achieved a higher external quantum efficiency than that of Example 5 not satisfying numerical expression (i-1).

Examples 6 and 11 to 14 in which H-2 as a compound represented by general formula (14) was used as the host material of the emissive layer achieved a higher external quantum efficiency than that of Example 1.

Examples 7 to 9 using D-3 as the compound represented by general formula (1) achieved a higher external quantum efficiency than that of each of Comparative Example 3 not containing the compound represented by general formula (2) and Comparative Examples 4 and 5 using the compound D-5 other than the compound represented by general formula (1) as a dopant. Among these, Examples 7 and 8 satisfying numerical expression (i-2) achieved a higher external quantum efficiency than that of Example 9 not satisfying numerical expression (i-2).

Examples 10 and 15 to 20 using H-2 as the compound represented by general formula (14) as the host material of the emissive layer achieved a higher external quantum efficiency than that of Example 7.

When Example 21 as the bottom emission element using D-3 as the compound represented by general formula (1) and Comparative Example 7 as the bottom emission element using the phosphorescent compound D-7 other than the compound represented by general formula (1) are compared with each other, it is found that the use of D-7 which is phosphorescent provides an excellent external quantum efficiency, but the use of the compound D-3 represented by general formula (1) provides significantly excellent color purity.

When the bottom emission element and the top emission element are compared with each other, it is found that the top emission element provides improved color purity from Comparative Example 6 and Comparative Example 7 using D-7, but the external quantum efficiency is largely reduced. Meanwhile, in Example 7 and Example 21 using D-3 as the compound represented by general formula (1), it is found that very high color purity of the top emission element can be achieved without the external quantum efficiency being largely decreased.

### Examples 22 to 35

In the same manner as in Example 1 except that compounds described in Table 4 were used as materials for electron transporting layers, light-emitting elements were prepared and evaluated. The results are shown in Table 4. ET-2 to ET-8 are compounds shown below.

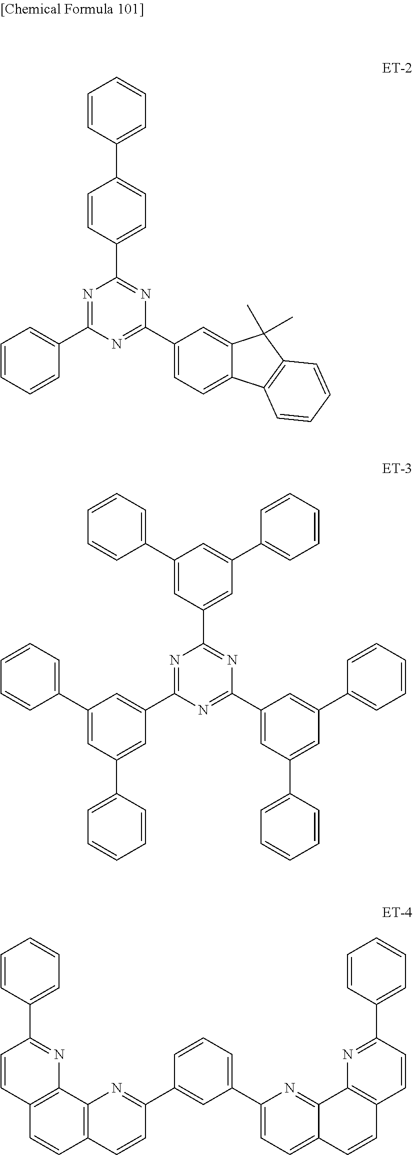 [SMILES: CC1(C)c2ccccc2-c2ccc(-c3nc(-c4ccccc4)nc(-c4ccc(-c5ccccc5)cc4)n3)cc21 ; c1ccc(-c2cc(-c3ccccc3)cc(-c3nc(-c4cc(-c5ccccc5)cc(-c5ccccc5)c4)nc(-c4cc(-c5ccccc5)cc(-c5ccccc5)c4)n3)c2)cc1 ; c1ccc(-c2ccc3ccc4ccc(-c5cccc(-c6ccc7ccc8ccc(-c9ccccc9)nc8c7n6)c5)nc4c3n2)cc1]

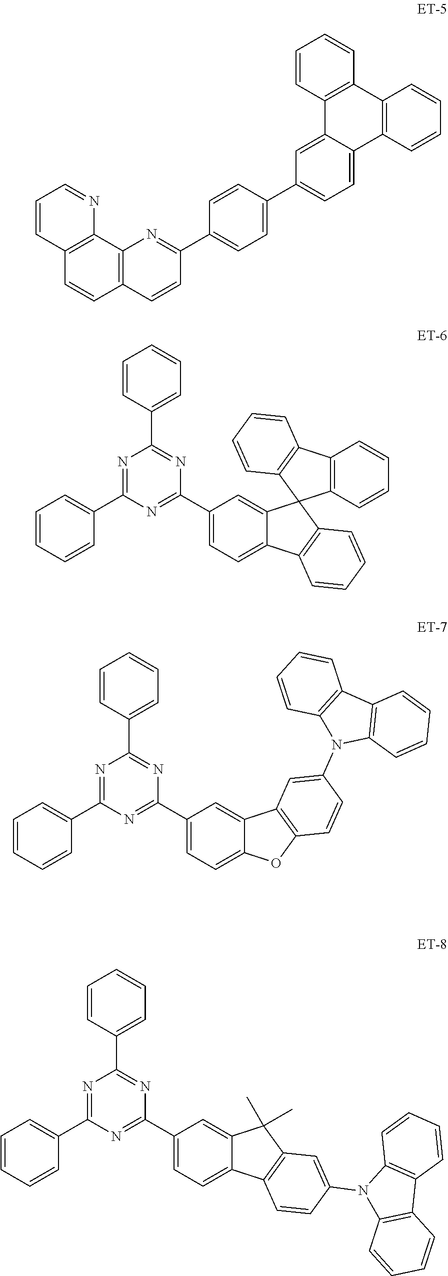

TABLE 4

| | Emissive Layer | | | | | | |
|---|---|---|---|---|---|---|---|
| | *Numbers in parentheses below | | | | | | |
| | compound names: weight ratios | | | | | | |
| | | General | | | Electron transporting layer | | External |
| | | formula (1) | General | | Electron | | quantum |
| | | or other | formula | Emitted | transporting | Donor | efficiency |
| | Host | dopants | (2) | color | material | material | (%) |
| Example 22 | H-1 | D-1 | B-1 | Red | ET-2 | 2E-1 | 5.2 |
| | (80) | (1) | (20) | | | | |
| Example 23 | H-1 | D-1 | B-1 | Red | ET-3 | 2E-1 | 5.1 |
| | (80) | (1) | (20) | | | | |
| Example 24 | H-1 | D-3 | B-2 | Green | ET-2 | 2E-1 | 4.8 |
| | (80) | (1) | (20) | | | | |
| Example 25 | H-1 | D-3 | B-2 | Green | ET-3 | 2E-1 | 4.6 |
| | (80) | (1) | (20) | | | | |
| Example 26 | H-1 | D-1 | B-1 | Red | ET-4 | 2E-1 | 5.2 |
| | (80) | (1) | (20) | | | | |
| Example 27 | H-1 | D-1 | B-1 | Red | ET-5 | 2E-1 | 5.1 |
| | (80) | (1) | (20) | | | | |
| Example 28 | H-1 | D-3 | B-2 | Green | ET-4 | 2E-1 | 4.7 |
| | (80) | (1) | (20) | | | | |
| Example 29 | H-2 | D-3 | B-2 | Green | ET-5 | 2E-1 | 4.5 |
| | (80) | (1) | (20) | | | | |
| Example 30 | H-1 | D-1 | B-1 | Red | ET-6 | 2E-1 | 5.2 |
| | (80) | (1) | (20) | | | | |
| Example 31 | H-1 | D-1 | B-1 | Red | ET-7 | 2E-1 | 5.1 |
| | (80) | (1) | (20) | | | | |
| Example 32 | H-1 | D-1 | B-1 | Red | ET-8 | 2E-1 | 5.1 |
| | (80) | (1) | (20) | | | | |
| Example 33 | H-1 | D-3 | B-2 | Green | ET-6 | 2E-1 | 4.8 |
| | (80) | (1) | (20) | | | | |
| Example 34 | H-2 | D-3 | B-2 | Green | ET-7 | 2E-1 | 4.6 |
| | (80) | (1) | (20) | | | | |
| Example 35 | H-2 | D-3 | B-2 | Green | ET-8 | 2E-1 | 4.5 |
| | (80) | (1) | (20) | | | | |

Examples 22 to 25 and 30 to 35 achieved a higher external quantum efficiency than that of each of Examples 1 and 7 not containing the compound represented by general formula (15).

Examples 26 to 29 achieved a higher external quantum efficiency than that of each of Examples 1 and 7 not containing the compound represented by general formula (16).

### Example 36

In the same manner as in Example 1 except that, after a hole injection layer was formed, HT-1 was deposited in a thickness of 170 nm as a first hole transporting layer, and a compound described in Table 5 was then deposited in a thickness of 10 nm as a second hole transporting layer, to form a hole transporting layer having a total thickness of 180 nm, a light-emitting element was prepared and evaluated. The results are shown in Table 5. HT-2 to HT-6 are compounds shown below.

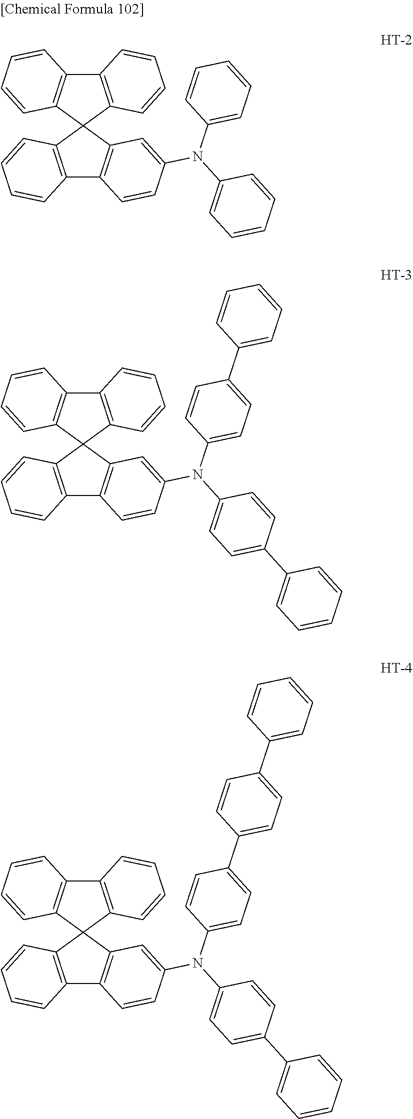 [SMILES: c1ccc(N(c2ccccc2)c2ccc3c(c2)C2(c4ccccc4-c4ccccc42)c2ccccc2-3)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; c1ccc(-c2ccc(-c3ccc(N(c4ccc(-c5ccccc5)cc4)c4ccc5c(c4)C4(c6ccccc6-c6ccccc64)c4ccccc4-5)cc3)cc2)cc1]

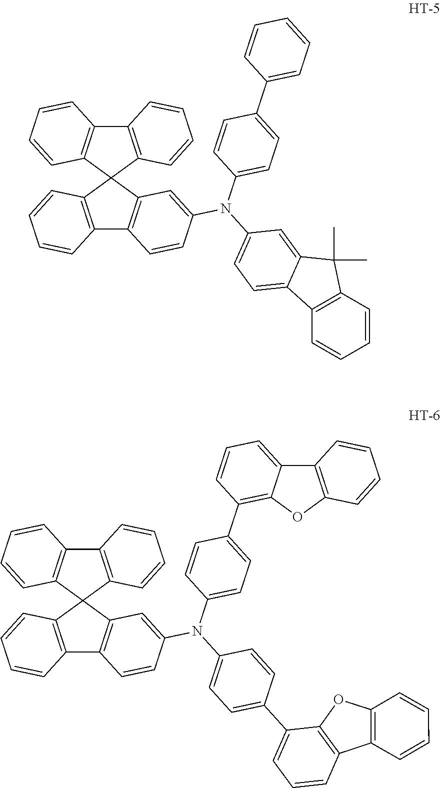 [SMILES: CC1(C)c2ccccc2-c2ccc(N(c3ccc(-c4ccccc4)cc3)c3ccc4c(c3)C3(c5ccccc5-c5ccccc53)c3ccccc3-4)cc21 ; c1ccc2c(c1)-c1ccccc1C21c2ccccc2-c2ccc(N(c3ccc(-c4cccc5c4oc4ccccc45)cc3)c3ccc(-c4cccc5c4oc4ccccc45)cc3)cc21]

TABLE 5

| | | | Emissive Layer | | | | |
|---|---|---|---|---|---|---|---|
| | | | *Numbers in parentheses below | | | | |
| | | | compound names: weight ratios | | | | |
| | Hole transporting layer | | | General | | | External |
| | First hole | Second hole | | formula (1) | General | | quantum |
| | transporting | transporting | | or other | formula | Emitted | efficiency |
| | layer | layer | Host | dopants | (2) | color | (%) |
| Example 36 | HT-1 | HT-2 | H-1 | D-1 | B-1 | Red | 5.2 |
| | | | (80) | (1) | (20) | | |
| Example 37 | HT-1 | HT-3 | H-1 | D-1 | B-1 | Red | 5.4 |
| | | | (80) | (1) | (20) | | |
| Example 38 | HT-1 | HT-4 | H-1 | D-1 | B-1 | Red | 5.4 |
| | | | (80) | (1) | (20) | | |
| Example 39 | HT-1 | HT-5 | H-1 | D-1 | B-1 | Red | 5.4 |
| | | | (80) | (1) | (20) | | |
| Example 40 | HT-1 | HT-6 | H-1 | D-1 | B-1 | Red | 5.4 |
| | | | (80) | (1) | (20) | | |
| Example 41 | HT-1 | HT-5 | H-2 | D-1 | B-1 | Red | 5.8 |
| | | | (80) | (1) | (20) | | |
| Example 42 | HT-1 | HT-2 | H-1 | D-3 | B-2 | Green | 4.6 |
| | | | (80) | (1) | (20) | | |
| Example 43 | HT-1 | HT-3 | H-1 | D-3 | B-2 | Green | 4.8 |
| | | | (80) | (1) | (20) | | |
| Example 44 | HT-1 | HT-4 | H-1 | D-3 | B-2 | Green | 4.8 |
| | | | (80) | (1) | (20) | | |
| Example 45 | HT-1 | HT-5 | H-1 | D-3 | B-2 | Green | 4.9 |
| | | | (80) | (1) | (20) | | |
| Example 46 | HT-1 | HT-6 | H-1 | D-3 | B-2 | Green | 4 8 |
| | | | (80) | (1) | (20) | | |
| Example 47 | HT-1 | HT-5 | H-2 | D-3 | B-2 | Green | 5.3 |
| | | | (80) | (1) | (20) | | |

Examples 36 to 40 and 42 to 46 achieved a higher external quantum efficiency than that of each of Examples 1 and 7 not containing a monoamine compound having a spirofluorene skeleton in the hole transporting layer on the anode side of the emissive layer.

Examples 41 and 47 achieved a higher external quantum efficiency higher than that of each of Example 39 and Example 45 using H-2 as the compound represented by general formula (14) as the host material of the emissive layer.

1. A light-emitting element comprising:
an anode;
a cathode; and
a plurality of organic layers including an emissive layer between the anode and the cathode, and emitting light by means of electrical energy,
wherein
the emissive layer contains a compound represented by general formula (1) and a delayed fluorescent compound: 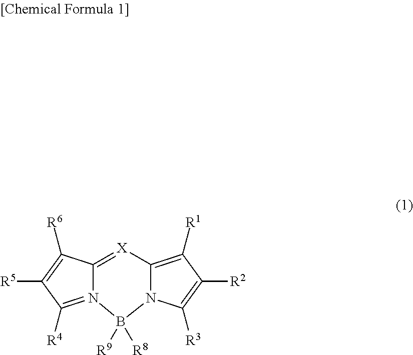
wherein X represents C—R⁷ or N; R¹ to R⁹ are the same or different from each other, and each are selected from a hydrogen atom, an alkyl group, a cycloalkyl group, a heterocyclic group, an alkenyl group, a cycloalkenyl group, an alkynyl group, a hydroxyl group, a thiol group, an alkoxy group, an alkylthio group, an aryl ether group, an aryl thioether group, an aryl group, a heteroaryl group, a halogen, a cyano group, an aldehyde group, a carbonyl group, a carboxyl group, an ester group, a carbamoyl group, an amino group, a nitro group, a silyl group, a siloxanyl group, a boryl group, —P(═O)R¹⁰R¹¹, and a fused ring and an aliphatic ring formed with an adjacent substituent; and R¹⁰ and R¹¹ each are an aryl group or a heteroaryl group.

2. The light-emitting element according to claim 1, wherein the delayed fluorescent compound is a compound represented by general formula (2): 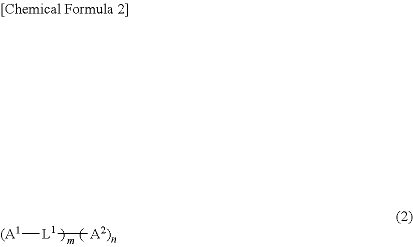
wherein A¹ is an electron-donating moiety, and A² is an electron-accepting moiety; L¹s each are a linking group, the same or different from each other, and each represent a single bond or a phenylene group; m and n each are a natural number of 1 or more and 10 or less; when m is 2 or more, a plurality of A¹s and L¹s are the same or different from each other; and when n is 2 or more, a plurality of A²s are the same or different from each other.

3. The light-emitting element according to claim 1, wherein the light-emitting element emits fluorescence exhibiting a single peak in a wavelength range of 400 nm or more and 900 nm or less.

4. The light-emitting element according to claim 3, wherein the single peak has a half-value width of 60 nm or less.

5. The light-emitting element according to claim 1, wherein the light-emitting element is of a top emission type.

6. The light-emitting element according to claim 2, wherein the light-emitting element satisfies numerical expression (i-1):

|λ1 (abs)−λ2 (FL)|≤50 (i-1)
wherein λ1 (abs) represents a peak wavelength (nm) of a longest wavelength side peak in an absorption spectrum of the compound represented by the general formula (1) at a wavelength of 400 nm or more and 900 nm or less; and λ2 (FL) represents a peak wavelength (nm) of a longest wavelength side peak in a fluorescence spectrum of the compound represented by the general formula (2) at a wavelength of 400 nm or more and 900 nm or less.

7. The light-emitting element according to claim 2, wherein a content of the compound represented by the general formula (1) in the emissive layer is 5 wt % or less, and a content of the compound represented by the general formula (2) is 70 wt % or less.

8. The light-emitting element according to claim 2, wherein A¹ is selected from general formula (3) or (4): 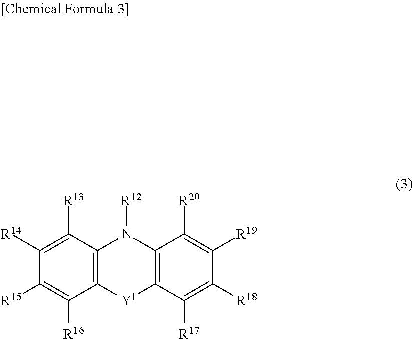
wherein Y¹ is selected from a single bond, CR²¹R²², NR²³, O, or S; R¹² to R²³ are the same or different from each other, and each are selected from a hydrogen atom, an alkyl group, a cycloalkyl group, a heterocyclic group, an alkenyl group, a cycloalkenyl group, an alkynyl group, a hydroxyl group, a thiol group, an alkoxy group, an alkylthio group, an aryl ether group, an aryl thioether group, an aryl group, a heteroaryl group, a halogen, a cyano group, an aldehyde group, a carbonyl group, a carboxyl group, an ester group, a carbamoyl group, an amino group, a nitro group, a silyl group, a siloxanyl group, a boryl group, —P(═O)R¹⁰R¹¹, and a fused ring and an aliphatic ring formed with an adjacent substituent; L¹ is bonded to at least one position of R¹² to R²³; and R¹⁰ and R¹¹ each are an aryl group or a heteroaryl group, 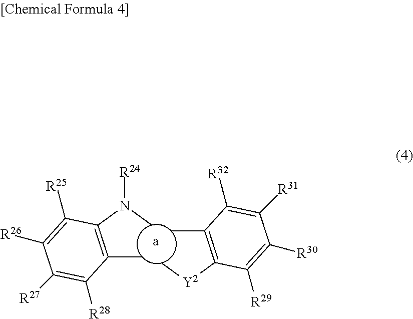
wherein ring a is a benzene ring or a naphthalene ring; Y² is selected from CR³³R³⁴, NR³⁵, O, or S; R²¹ to R³⁵ are the same or different from each other, and each are selected from a hydrogen atom, an alkyl group, a cycloalkyl group, a heterocyclic group, an alkenyl group, a cycloalkenyl group, an alkynyl group, a hydroxyl group, a thiol group, an alkoxy group, an alkylthio group, an aryl ether group, an aryl thioether group, an aryl group, a heteroaryl group, a halogen, a cyano group, an aldehyde group, a carbonyl group, a carboxyl group, an ester group, a carbamoyl group, an amino group, a nitro group, a silyl group, a siloxanyl group, a boryl group, —P(═O)R¹⁰R¹¹, and a fused ring and an aliphatic ring formed with an adjacent substituent; L¹ is bonded to at least one position of R²¹ to R³⁵; and R¹⁰ and R¹¹ each are an aryl group or a heteroaryl group.

9. The light-emitting element according to claim 2, wherein, in the general formula (2), A¹ is represented by the general formula (3).

10. The light-emitting element according to claim 2, wherein A² is a group represented by general formula (5): 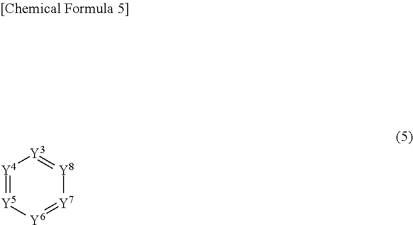
wherein Y³ to Y⁸ are the same or different from each other, and each are selected from CR³⁶ or N; At least one of Y³ to Y⁸ is N, and all of Y³ to Y⁸ are not N; R³⁶s are the same or different from each other, and each are selected from the group consisting of a hydrogen atom, an aryl group, a heteroaryl group, and a fused ring and an aliphatic ring formed with an adjacent substituent; and L¹ is bonded to at least one position of Y³ to Y⁸.

11. The light-emitting element according to claim 2, wherein, in the general formula (2), A² is represented by general formula (6) or (7): 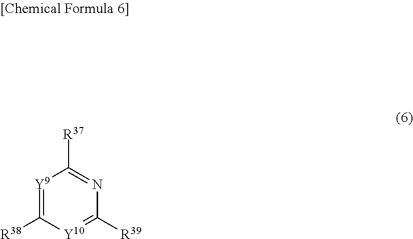
wherein Y⁹ and Y¹⁰ are the same or different from each other, and each are selected from CR⁴⁰ or N; at least one of Y⁹ and Y¹⁰ is N; R³⁷ to R⁴⁰ are the same or different from each other, and each are selected from a hydrogen atom, an aryl group, or a heteroaryl group; and L¹ is bonded to at least one position of R³⁷ to R⁴⁰; 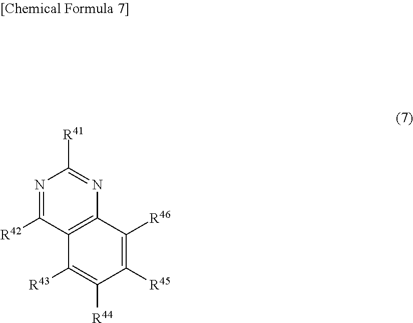
R⁴¹ to R⁴⁶ are the same or different from each other, and each are selected from a hydrogen atom, an aryl group, or a heteroaryl group; and L¹ is bonded to at least one position of R⁴¹ or R⁴².

12. The light-emitting element according to claim 2, wherein, in the general formula (2), A² is represented by the general formula (6).

13. The light-emitting element according to claim 1, wherein the emissive layer further contains a compound represented by general formula (14): 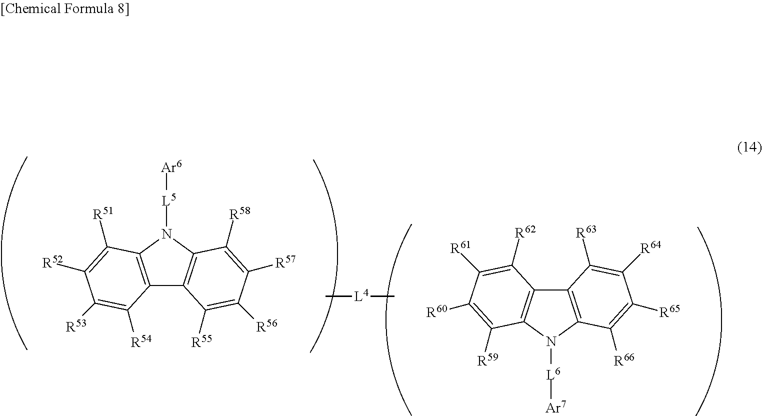
wherein R⁵¹ to R⁶⁶ are the same or different from each other, and each are selected from a hydrogen atom, an alkyl group, a cycloalkyl group, a heterocyclic group, an alkenyl group, a cycloalkenyl group, an alkynyl group, a hydroxyl group, a thiol group, an alkoxy group, an alkylthio group, an aryl ether group, an aryl thioether group, an aryl group, a heteroaryl group, a halogen, a cyano group, an aldehyde group, a carbonyl group, a carboxyl group, an ester group, a carbamoyl group, an amino group, a nitro group, a silyl group, a siloxanyl group, a boryl group, —P(═O)R¹⁰R¹¹, and a fused ring and an aliphatic ring formed with an adjacent substituent; L⁴ is connected to one position of R⁵¹ to R⁵⁸ and one position of R⁵⁹ to R⁶⁶; L⁴ to L⁶ each are a single bond or a phenylene group; L⁴ is connected to one position of R⁵¹ to R⁵⁸ and one position of R⁵⁹ to R⁶⁶; R¹⁰ and R¹¹ each are an aryl group or a heteroaryl group; Ar⁶ and Ar⁷ are the same or different from each other, and each represent a substituted or unsubstituted aryl group.

14. The light-emitting element according to claim 13, wherein, in the general formula (14), L⁴ is connected to one position of R⁵⁶ and R⁵⁷ and one position of R⁶⁰ and R⁶¹.

15. The light-emitting element according to claim 13, wherein, in the general formula (14), L⁴ is a single bond.

16. The light-emitting element according to claim 13, wherein, in the general formula (14), Ar⁶ and Ar⁷ are different from each other.

17. The light-emitting element according to claim 13, wherein, in the general formula (14), Ar⁶ and Ar⁷ are the same or different from each other, and each are selected from a substituted or unsubstituted phenyl group, biphenyl group, terphenyl group, naphthyl group, fluorenyl group, phenanthryl group, and triphenylenyl group.

18. The light-emitting element according to claim 13, wherein, in the general formula (14), Ar⁶ and Ar⁷ are the same or different from each other, and each are selected from

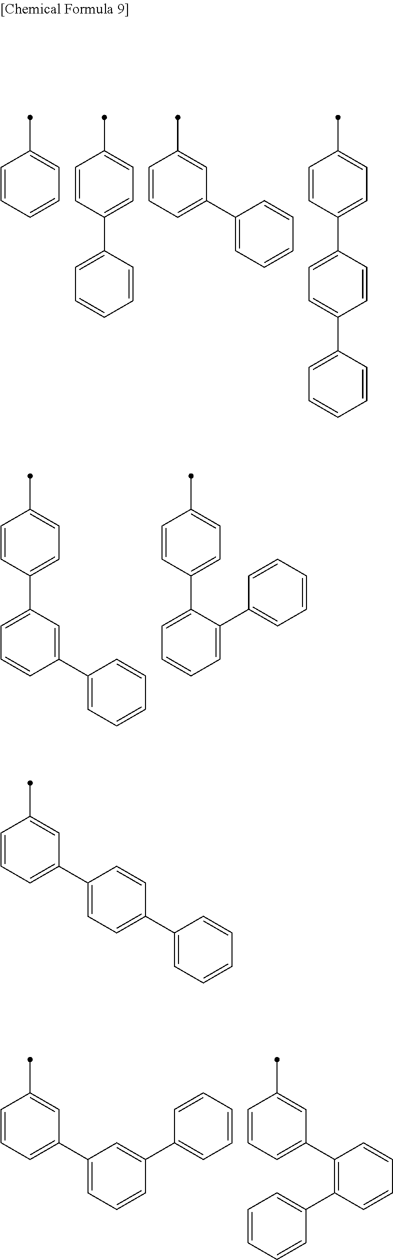

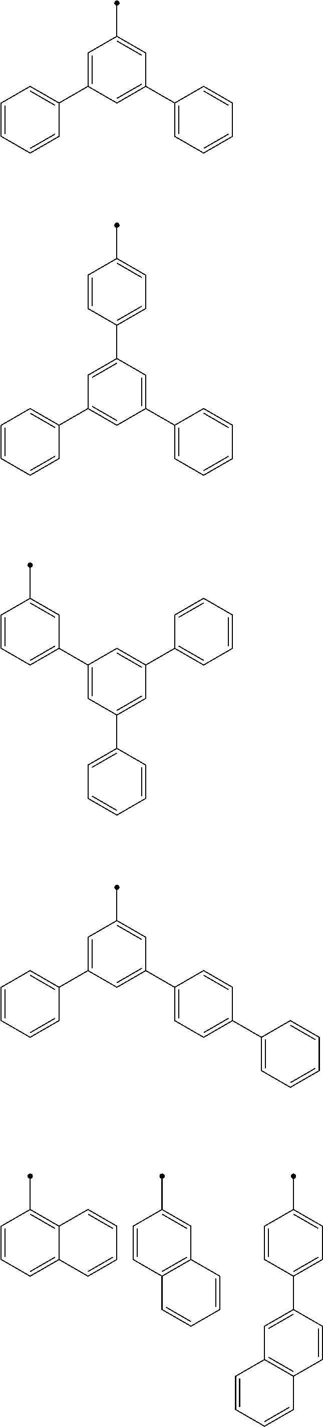

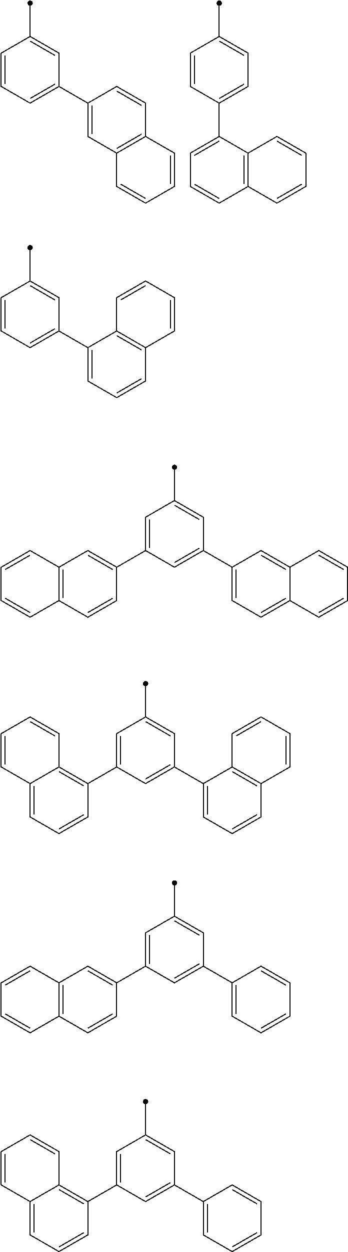

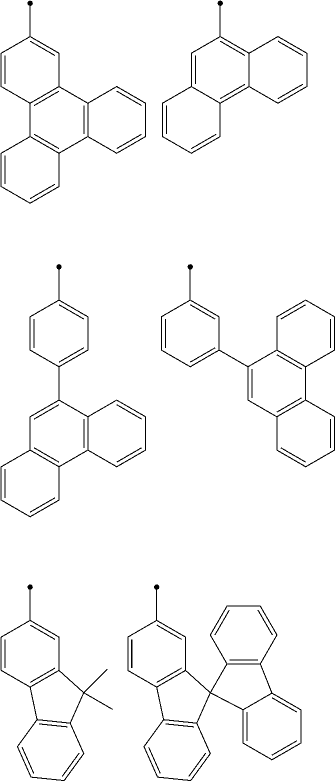

19. The light-emitting element according to claim 13, wherein, in the general formula (14), R⁶⁴ is an aryl group.

20. The light-emitting element according to claim 13, wherein, in the general formula (14), R⁶⁴ is a substituted or unsubstituted phenyl group, biphenyl group, terphenyl group, naphthyl group, fluorenyl group, phenanthryl group, or triphenylenyl group.

21. The light-emitting element according to claim 1, further comprising a hole transporting layer containing a monoamine compound having a spirofluorene skeleton on an anode side of the emissive layer.

22. The light-emitting element according to claim 21, wherein at least one of nitrogen atom substituents of the monoamine compound having a spirofluorene skeleton is a substituted or unsubstituted p-biphenyl group, a substituted or unsubstituted p-terphenyl group, a substituted or unsubstituted 2-fluorenyl group, or a group containing a substituted or unsubstituted dibenzofuranyl group.

23. The light-emitting element according to claim 1, further comprising an electron transporting layer containing a compound represented by general formula (15) on a cathode side of the emissive layer: 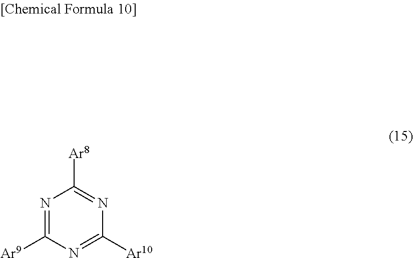
wherein Ar⁸ to Ar¹⁰ are the same or different from each other, and each are a substituted or unsubstituted aryl group or a substituted or unsubstituted heteroaryl group.

24. The light-emitting element according to claim 23, wherein, in the general formula (15), at least one of Ar⁸ to Ar¹⁰ is a substituted or unsubstituted phenyl group, biphenyl group, naphthyl group, or fluorenyl group.

25. The light-emitting element according to claim 1, further comprising an electron transporting layer containing a compound having a phenanthroline skeleton on a cathode side of the emissive layer.

26. The light-emitting element according to claim 25, wherein the compound having a phenanthroline skeleton is a compound represented by general formula (16): 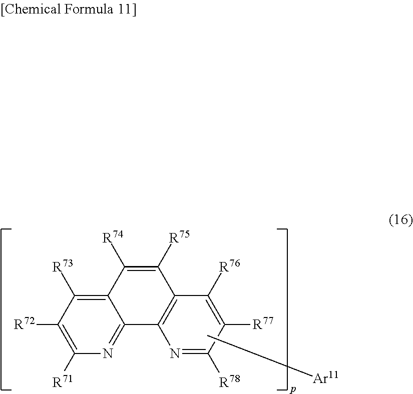
wherein R⁷¹ to R⁷⁸ are the same or different from each other, and each are a hydrogen atom, a substituted or unsubstituted aryl group, or a substituted or unsubstituted heteroaryl group; Ar¹¹ is a substituted or unsubstituted aryl group; and p is a natural number of 1 to 3.

27. The light-emitting element according to claim 26, wherein, in the general formula (16), p is 2.

28. The light-emitting element according to claim 1, wherein, in the general formula (1), X is C—R⁷, and R⁷ is a substituted or unsubstituted phenyl group.

29. The light-emitting element according to claim 1, wherein, in the general formula (1), all of R¹, R³, R⁴, and R⁶ are the same or different from each other, and R¹, R³, R⁴, and R⁶ each are a substituted or unsubstituted phenyl group.

30. The light-emitting element according to claim 1, wherein, in the general formula (1), all of R¹, R³, R⁴, and R⁶ are the same or different from each other, and R¹, R³, R⁴, and R⁶ each are a substituted or unsubstituted alkyl group.

31. The light-emitting element according to claim 1, wherein at least one of R¹ to R⁷ is an electron withdrawing group.

32. The light-emitting element according to claim 1, wherein the light-emitting element is a tandem structure-type element, further comprising a P-type charge generation layer, and a N-type charge generation layer containing a compound having a phenanthroline skeleton.

33. A display comprising the light-emitting element according to claim 32.

34. An illuminator comprising the light-emitting element according to claim 32.

35. A sensor comprising the light-emitting element according to claim 32.
